SEQUENCE LISTING (270 sequences)

SEQ ID NO: 1 - protein, 120 aa, Artificial Sequence
  DL3B279
QVQLVQSGAEVKKPGASVKVSCKASGNTFTNYYIHWVRQAPGQGLEWMGIINPSGGSTSYAQKLQGRMTMTRDTSTSTVYMELSSLRSEDTAVYFCARQGPFIGDAFDIWGQGTMVTVSS

SEQ ID NO: 2 - protein, 107 aa, Artificial Sequence
  DL3B279
DIQMTQSPSSLSASVGDRVTITCRASQGISNYLAWFQQKPGKAPKSLIYAASSLQSGVPSKFSGSGSGTDFTLTISSLQPEDFATYYCQQYNSYPYTFAQGTKLEIK

SEQ ID NO: 3 - protein, 120 aa, Artificial Sequence
  DL3B279
QVQLVQSGAEVKKPGASVKVSCKASGQTFTNYYIHWVRQAPGQGLEWMGIINPSGGSTSYAQKLQGRMTMTRDTSTSTVYMELSSLRSEDTAVYFCARQGPFIGDAFDIWGQGTTVTVSS

SEQ ID NO: 4 - protein, 107 aa, Artificial Sequence
  DL3B279
DIQMTQSPSSLSASVGDRVTITCRASQGISNYLAWFQQKPGKAPKSLIYAASSLQSGVPSKFSGSGSGTDFTLTISSLQPEDFATYYCQQYNSYPYTFGQGTKLEIK

SEQ ID NO: 5 - protein, 118 aa, Artificial Sequence
  DL3B332
QVQLQESGPGLVKPSETLSLSCTVSGDSIRSYYWSWIRQPPGKGLEWIGYIYYSGTTNYKSSLKSRVTISLDTSKKQFSLNLDSVTAADTAVYYCARIGPAGFYFDYWGQGTLVTVSS

SEQ ID NO: 6 - protein, 108 aa, Artificial Sequence
  DL3B332
EIVLTQSPGTLSLSPGERATLSCRASQSVSRSYLAWYQQKPGQAPRFLIYGASSRATGIPDRFSGSGSGTDFTLTISRLEPEDFAVYYCQQYGTSPITFGQGTRLEIK

SEQ ID NO: 7 - protein, 123 aa, Artificial Sequence
  DL3B358
QVQLVQSGAEVKKPGASVKVSCKASGHIFISYYIHWVRQAPGQGLEWMGIIDPSGGSKSYAQKFQGRVTMTRDTSTSTVYMELSSLRSEDTAVYYCARQGMIVGTTGDAFDIWGQGTMVTVSS

SEQ ID NO: 8 - protein, 107 aa, Artificial Sequence
  DL3B358
EIVLTQSPGTLSLSPGERATLSCRASQSASSYLAWYQQKPGQAPRLLIYGASSRATGIPDRFSGSGSGTDFTLTISRLEPEDFAVYYCQQYNSSPYTFGQGTKLEIK

SEQ ID NO: 9 - protein, 121 aa, Artificial Sequence
  DL3B409
EVQLVQSGAEVKKPGASVKVSCKASGYTFTTYYIHWVRQAPGQGLEWMGIIDPSGGRTSYAQKFLGRVTMTRDTSTSTVYMELRSLRSEDTAVYYCARGGDGTWYYGMDVWGQGTTVTVSS

SEQ ID NO: 10 - protein, 107 aa, Artificial Sequence
  DL3B409
DIVMTQSPSFLSASVGDRVTITCRASQGISNYLAWYQQKPGKAPKLLIYAASTLQSGVPSRFSGSGSGTEFTLTISSLQPEDFATYYCQQLNSYPLTFGGGTKVEIK

SEQ ID NO: 11 - protein, 126 aa, Artificial Sequence
  DL3B450
QVQLQQSGPGLVKPSETLSLTCTVSGGSISSYYWSWIRQPAGKGLEWIGRIYTSGSTNYNPSLKSRVTMSVDTSKNQFSLKLSSVTAADTAVYYCARDQAYSGYDWSYYYYGMDVWGQGTMVTVSS

SEQ ID NO: 12 - protein, 112 aa, Artificial Sequence
  DL3B450
ETTLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 13 - protein, 115 aa, Artificial Sequence
  DL3B461
QVQLVQSGAEVKKPGSSVKVSCKASGGTLSSYVISWVRQAPGQGLEWMGGIIPIFGTANYAQKFQDRVTITADKSTNTAYMELTSLTSEDTAVYYCARDPFSDLWGRGTMVTVSS

SEQ ID NO: 14 - protein, 112 aa, Artificial Sequence
  DL3B461
DIVMTQSPLSSPVTLGQPASISCRSSQSLVHSDGNTYLNWLQQRPGQPPRLLIYQISNPFSGVPDRFSGSGAGTDFTLKISRVEAEDVGVYYCMQATQFPHTFGPGTKVEIK

SEQ ID NO: 15 - protein, 5 aa, Artificial Sequence
  DL3B279
NYYIH

SEQ ID NO: 16 - protein, 17 aa, Artificial Sequence
  DL3B279
IINPSGGSTSYAQKLQG

SEQ ID NO: 17 - protein, 11 aa, Artificial Sequence
  DL3B279
QGPFIGDAFDI

SEQ ID NO: 18 - protein, 5 aa, Artificial Sequence
  DL3B332 DL3B450
SYYWS

SEQ ID NO: 19 - protein, 16 aa, Artificial Sequence
  DL3B332
YIYYSGTTNYKSSLKS

SEQ ID NO: 20 - protein, 10 aa, Artificial Sequence
  DL3B332
IGPAGFYFDY

SEQ ID NO: 21 - protein, 5 aa, Artificial Sequence
  DL3B358
SYYIH

SEQ ID NO: 22 - protein, 17 aa, Artificial Sequence
  DL3B358
IIDPSGGSKSYAQKFQG

SEQ ID NO: 23 - protein, 14 aa, Artificial Sequence
  DL3B358
QGMIVGTTGDAFDI

SEQ ID NO: 24 - protein, 5 aa, Artificial Sequence
  TYYIH
TYYIH

SEQ ID NO: 25 - protein, 17 aa, Artificial Sequence
  TYYIH
IIDPSGGRTSYAQKFLG

SEQ ID NO: 26 - protein, 12 aa, Artificial Sequence
  TYYIH
GGDGTWYYGMDV

SEQ ID NO: 27 - protein, 8 aa, Artificial Sequence
  Linker 2
GGGSGGGS

SEQ ID NO: 28 - protein, 16 aa, Artificial Sequence
  DL3B450
RIYTSGSTNYNPSLKS

SEQ ID NO: 29 - protein, 18 aa, Artificial Sequence
  DL3B450
DQAYSGYDWSYYYYGMDV

SEQ ID NO: 30 - protein, 5 aa, Artificial Sequence
  DL3B461
SYVIS

SEQ ID NO: 31 - protein, 17 aa, Artificial Sequence
  DL3B461
GIIPIFGTANYAQKFQD

SEQ ID NO: 32 - protein, 6 aa, Artificial Sequence
  DL3B461
DPFSDL

SEQ ID NO: 33 - protein, 11 aa, Artificial Sequence
  DL3B279
RASQGISNYLA

SEQ ID NO: 34 - protein, 7 aa, Artificial Sequence
  DL3B279
AASSLQS

SEQ ID NO: 35 - protein, 9 aa, Artificial Sequence
  DL3B279
QQYNSYPYT

SEQ ID NO: 36 - protein, 12 aa, Artificial Sequence
  DL3B332
RASQSVSRSYLA

SEQ ID NO: 37 - protein, 7 aa, Artificial Sequence
  DL3B332 DL3B358
GASSRAT

SEQ ID NO: 38 - protein, 9 aa, Artificial Sequence
  DL3B332
QQYGTSPIT

SEQ ID NO: 39 - protein, 11 aa, Artificial Sequence
  DL3B358
RASQSASSYLA

SEQ ID NO: 40 - protein, 9 aa, Artificial Sequence
  DL3B358
QQYNSSPYT

SEQ ID NO: 41 - protein, 11 aa, Artificial Sequence
  DL3B409
RASQGISNYLA

SEQ ID NO: 42 - protein, 7 aa, Artificial Sequence
  DL3B409
AASTLQS

SEQ ID NO: 43 - protein, 9 aa, Artificial Sequence
  DL3B409
QQLNSYPLT

SEQ ID NO: 44 - protein, 16 aa, Artificial Sequence
  DL3B450
RSSQSLLHSNGYNYLD

SEQ ID NO: 45 - protein, 7 aa, Artificial Sequence
  DL3B450
LGSNRAS

SEQ ID NO: 46 - protein, 9 aa, Artificial Sequence
  DL3B450
MQALQTPLT

SEQ ID NO: 47 - protein, 16 aa, Artificial Sequence
  DL3B461
RSSQSLVHSDGNTYLN

SEQ ID NO: 48 - protein, 7 aa, Artificial Sequence
  DL3B461
QISNPFS

SEQ ID NO: 49 - protein, 9 aa, Artificial Sequence
  DL3B461
MQATQFPHT

SEQ ID NO: 50 - protein, 10 aa, Artificial Sequence
  DL3B279
GNTFTNYYIH

SEQ ID NO: 51 - protein, 10 aa, Artificial Sequence
  DL3B279
IINPSGGSTS

SEQ ID NO: 52 - protein, 10 aa, Artificial Sequence
  DL3B279
GQTFTNYYIH

SEQ ID NO: 53 - protein, 10 aa, Artificial Sequence
  DL3B332
GDSIRSYYWS

SEQ ID NO: 54 - protein, 9 aa, Artificial Sequence
  DL3B332
YIYYSGTTN

SEQ ID NO: 55 - protein, 10 aa, Artificial Sequence
  DL3B358
GHIFISYYIH

SEQ ID NO: 56 - protein, 10 aa, Artificial Sequence
  DL3B358
IIDPSGGSKS

SEQ ID NO: 57 - protein, 10 aa, Artificial Sequence
  DL3B409
GYTFTTYYIH

SEQ ID NO: 58 - protein, 10 aa, Artificial Sequence
  DL3B409
IIDPSGGRTS

SEQ ID NO: 59 - protein, 10 aa, Artificial Sequence
  DL3B450
GGSISSYYWS

SEQ ID NO: 60 - protein, 9 aa, Artificial Sequence
  DL3B450
RIYTSGSTN

SEQ ID NO: 61 - protein, 10 aa, Artificial Sequence
  DL3B461
GGTLSSYVIS

SEQ ID NO: 62 - protein, 10 aa, Artificial Sequence
  DL3B461
GIIPIFGTAN

SEQ ID NO: 63 - protein, 247 aa, Artificial Sequence
  DL3B279 scFv_LH
DIQMTQSPSSLSASVGDRVTITCRASQGISNYLAWFQQKPGKAPKSLIYAASSLQSGVPSKFSGSGSGTDFTLTISSLQPEDFATYYCQQYNSYPYTFAQGTKLEIKGGSEGKSSGSGSESKSTGGSQVQLVQSGAEVKKPGASVKVSCKASGNTFTNYYIHWVRQAPGQGLEWMGIINPSGGSTSYAQKLQGRMTMTRDTSTSTVYMELSSLRSEDTAVYFCARQGPFIGDAFDIWGQGTMVTVSS

SEQ ID NO: 64 - protein, 247 aa, Artificial Sequence
  DL3B279 scFv_LH variant
DIQMTQSPSSLSASVGDRVTITCRASQGISNYLAWFQQKPGKAPKSLIYAASSLQSGVPSKFSGSGSGTDFTLTISSLQPEDFATYYCQQYNSYPYTFGQGTKLEIKGGSEGKSSGSGSESKSTGGSQVQLVQSGAEVKKPGASVKVSCKASGQTFTNYYIHWVRQAPGQGLEWMGIINPSGGSTSYAQKLQGRMTMTRDTSTSTVYMELSSLRSEDTAVYFCARQGPFIGDAFDIWGQGTTVTVSS

SEQ ID NO: 65 - protein, 246 aa, Artificial Sequence
  DL3B332 scFv _LH
EIVLTQSPGTLSLSPGERATLSCRASQSVSRSYLAWYQQKPGQAPRFLIYGASSRATGIPDRFSGSGSGTDFTLTISRLEPEDFAVYYCQQYGTSPITFGQGTRLEIKGGSEGKSSGSGSESKSTGGSQVQLQESGPGLVKPSETLSLSCTVSGDSIRSYYWSWIRQPPGKGLEWIGYIYYSGTTNYKSSLKSRVTISLDTSKKQFSLNLDSVTAADTAVYYCARIGPAGFYFDYWGQGTLVTVSS

SEQ ID NO: 66 - protein, 250 aa, Artificial Sequence
  DL3B358 scFv_LH
EIVLTQSPGTLSLSPGERATLSCRASQSASSYLAWYQQKPGQAPRLLIYGASSRATGIPDRFSGSGSGTDFTLTISRLEPEDFAVYYCQQYNSSPYTFGQGTKLEIKGGSEGKSSGSGSESKSTGGSQVQLVQSGAEVKKPGASVKVSCKASGHIFISYYIHWVRQAPGQGLEWMGIIDPSGGSKSYAQKFQGRVTMTRDTSTSTVYMELSSLRSEDTAVYYCARQGMIVGTTGDAFDIWGQGTMVTVSS

SEQ ID NO: 67 - protein, 248 aa, Artificial Sequence
  DL3B409 scFv_LH
DIVMTQSPSFLSASVGDRVTITCRASQGISNYLAWYQQKPGKAPKLLIYAASTLQSGVPSRFSGSGSGTEFTLTISSLQPEDFATYYCQQLNSYPLTFGGGTKVEIKGGSEGKSSGSGSESKSTGGSEVQLVQSGAEVKKPGASVKVSCKASGYTFTTYYIHWVRQAPGQGLEWMGIIDPSGGRTSYAQKFLGRVTMTRDTSTSTVYMELRSLRSEDTAVYYCARGGDGTWYYGMDVWGQGTTVTVSS

SEQ ID NO: 68 - protein, 258 aa, Artificial Sequence
  DL3B450 scFv_LH
ETTLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIKGGSEGKSSGSGSESKSTGGSQVQLQQSGPGLVKPSETLSLTCTVSGGSISSYYWSWIRQPAGKGLEWIGRIYTSGSTNYNPSLKSRVTMSVDTSKNQFSLKLSSVTAADTAVYYCARDQAYSGYDWSYYYYGMDVWGQGTMVTVSS

SEQ ID NO: 69 - protein, 247 aa, Artificial Sequence
  DL3B461 scFv_LH
DIVMTQSPLSSPVTLGQPASISCRSSQSLVHSDGNTYLNWLQQRPGQPPRLLIYQISNPFSGVPDRFSGSGAGTDFTLKISRVEAEDVGVYYCMQATQFPHTFGPGTKVEIKGGSEGKSSGSGSESKSTGGSQVQLVQSGAEVKKPGSSVKVSCKASGGTLSSYVISWVRQAPGQGLEWMGGIIPIFGTANYAQKFQDRVTITADKSTNTAYMELTSLTSEDTAVYYCARDPFSDLWGRGTMVTVSS

SEQ ID NO: 70 - protein, 231 aa, Artificial Sequence
  example 3
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 479 aa, Artificial Sequence
  DL3B279-VL-A99G-VH-N27Q_M105T-LH-scFv-Fc (ZW)
DIQMTQSPSSLSASVGDRVTITCRASQGISNYLAWFQQKPGKAPKSLIYAASSLQSGVPSKFSGSGSGTDFTLTISSLQPEDFATYYCQQYNSYPYTFGQGTKLEIKGGSEGKSSGSGSESKSTGGSQVQLVQSGAEVKKPGASVKVSCKASGQTFTNYYIHWVRQAPGQGLEWMGIINPSGGSTSYAQKLQGRMTMTRDTSTSTVYMELSSLRSEDTAVYFCARQGPFIGDAFDIWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 72 - protein, 12 aa, Artificial Sequence
  Linker 3
GGGSGGGSGGGS

SEQ ID NO: 73 - protein, 16 aa, Artificial Sequence
  Linker 4
GGGSGGGSGGGSGGGS

SEQ ID NO: 74 - protein, 20 aa, Artificial Sequence
  Linker 5
GGGSGGGSGGGSGGGSGGGS

SEQ ID NO: 75 - protein, 15 aa, Artificial Sequence
  Linker 6
GGGGSGGGGSGGGGS

SEQ ID NO: 76 - protein, 20 aa, Artificial Sequence
  Linker 7
GGGGSGGGGSGGGGSGGGGS

SEQ ID NO: 77 - protein, 116 aa, Artificial Sequence
  CD3B815_VH
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 78 - protein, 107 aa, Artificial Sequence
  CD3B815_VL
DILLTQSPGILSVSPGERVSFSCRARQSIGTAIHWYQQRTNGSPRLLIKYASESISGIPSRFSGSGSGTDFTLTINSVESEDIADYYCQQSNSWPYTFGGGTKLEIK

SEQ ID NO: 79 - protein, 25 aa, Artificial Sequence
  Linker 8
GGGGSGGGGSGGGGSGGGGSGGGGS

SEQ ID NO: 80 - protein, 107 aa, Artificial Sequence
  CD3W245_VL
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIK

SEQ ID NO: 81 - protein, 18 aa, Artificial Sequence
  Linker 9
GSTSGSGKPGSGEGSTKG

SEQ ID NO: 82 - protein, 14 aa, Artificial Sequence
  Linker 10
IRPRAIGGSKPRVA

SEQ ID NO: 83 - protein, 15 aa, Artificial Sequence
  Linker 11
GKGGSGKGGSGKGGS

SEQ ID NO: 84 - protein, 106 aa, Artificial Sequence
  VH amino acid sequence of CD3B376 Fab
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSS

SEQ ID NO: 85 - protein, 110 aa, Artificial Sequence
  VL amino acid sequence of CD3B376 Fab
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVL

SEQ ID NO: 86 - DNA, 348 bp, Artificial Sequence
  CD3B815
gaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctca

SEQ ID NO: 87 - DNA, 321 bp, Artificial Sequence
  CD3B815
gatatacttcttacccagagtcccggcatcctctccgttagccctggggagagagtctcattctcatgccgagccagacagtcaattggtaccgcaatacactggtatcaacagcggaccaatggttctccccgacttctgataaagtacgcatcagaatcaattagtggaataccatcaagatttagtggctcagggagtggaaccgattttactctgaccatcaactcagtggaatctgaggacattgccgactactactgtcaacaaagcaatagttggccatataccttcggaggcggaactaaattggagataaaa

SEQ ID NO: 88 - protein, 15 aa, Artificial Sequence
  Linker 12
GGKGSGGKGSGGKGS

SEQ ID NO: 89 - DNA, 321 bp, Artificial Sequence
  CD3W245
gacatacaaatgacacaatcaccctcttctctttctgcaagcgttggcgaccgtgtcactatcacttgtcgagcccgccagtccataggtactgccattcactggtatcaacagaagcctggcaaggctcccaaactcctgattaagtatgccagcgagagcatttccggcgtaccttcaagattttccggctccggtagtgggacagatttcactctcactatatctagcctccaaccagaagatttcgccacttactactgtcaacaatcaggttcatggccttacactttcggccaggggacaaaattggagatcaag

SEQ ID NO: 90 - protein, 15 aa, Artificial Sequence
  Linker 13
GGGKSGGGKSGGGKS

SEQ ID NO: 91 - protein, 15 aa, Artificial Sequence
  Linker 14
GKGKSGKGKSGKGKS

SEQ ID NO: 92 - protein, 15 aa, Artificial Sequence
  Linker 15
GGGKSGGKGSGKGGS

SEQ ID NO: 93 - DNA, 360 bp, Artificial Sequence
  VH nucleic acid sequence of CD3B376 Fab
caggtgcagctgcagcagtctggccctagactcgtgcggccttcccagaccctgtctctgacctgtgccatctccggcgactccgtgttcaacaacaacgccgcctggtcctggatccggcagagcccttctagaggcctggaatggctgggccggacctactaccggtccaagtggctgtacgactacgccgtgtccgtgaagtcccggatcaccgtgaaccctgacacctcccggaaccagttcaccctgcagctgaactccgtgacccctgaggacaccgccctgtactactgcgccagaggctactcctcctccttcgactattggggccagggcaccctcgtgaccgtgtcctct

SEQ ID NO: 94 - DNA, 329 bp, Artificial Sequence
  VL nucleic acid sequence of CD3B376 Fab
agtctgctctgacccagcctgcctccgtgtctggctctcccggccagtccatcaccatcagctgtaccggcacctcctccaacatcggcacctacaagttcgtgtcctggtatcagcagcaccccgacaaggcccccaaagtgctgctgtacgaggtgtccaagcggccctctggcgtgtcctccagattctccggctccaagtctggcaacaccgcctccctgaccatcagcggactgcaggctgaggaccaggccgactaccactgtgtgtcctacgctggctctggcaccctgctgtttggcggaggcaccaagctgaccgtgctg

SEQ ID NO: 95 - protein, 5 aa, Artificial Sequence
  CD3B815
RYNMN

SEQ ID NO: 96 - protein, 17 aa, Artificial Sequence
  CD3B815
SISTSSNYIYYADSVKG

SEQ ID NO: 97 - protein, 7 aa, Artificial Sequence
  CD3B815
GWGPFDY

SEQ ID NO: 98 - protein, 7 aa, Artificial Sequence
  CD3B376 HCDR1
NNNAAWS

SEQ ID NO: 99 - protein, 18 aa, Artificial Sequence
  CD3B376HCDR2
RTYYRSKWLYDYAYSYKS

SEQ ID NO: 100 - protein, 14 aa, Artificial Sequence
  CD3B376 HCDR3
TGTSSNIGTYKFVS

SEQ ID NO: 101 - protein, 11 aa, Artificial Sequence
  CD3B815
RARQSIGTAIH

SEQ ID NO: 102 - protein, 7 aa, Artificial Sequence
  CD3B815
YASESIS

SEQ ID NO: 103 - protein, 9 aa, Artificial Sequence
  CD3B815
QQSNSWPYT

SEQ ID NO: 104 - protein, 9 aa, Artificial Sequence
  LCDR3
QQSGSWPYT

SEQ ID NO: 105 - protein, 243 aa, Artificial Sequence
  CD3W245-scFv-LH
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 106 - protein, 14 aa, Artificial Sequence
  CD3B376 LCDR1
TGTSSNIGTYKFVS

SEQ ID NO: 107 - protein, 7 aa, Artificial Sequence
  CD3B376 LCDR2
EVSKRPS

SEQ ID NO: 108 - protein, 10 aa, Artificial Sequence
  CD3B376 LCDR3
VSYAGSGTLL

SEQ ID NO: 109 - protein, 449 aa, Artificial Sequence
  DL3B279-Fab-Fc HC1
QVQLVQSGAEVKKPGASVKVSCKASGNTFTNYYIHWVRQAPGQGLEWMGIINPSGGSTSYAQKLQGRMTMTRDTSTSTVYMELSSLRSEDTAVYFCARQGPFIGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 110 - protein, 214 aa, Artificial Sequence
  DL3B279-Fab-Fc LC1
DIQMTQSPSSLSASVGDRVTITCRASQGISNYLAWFQQKPGKAPKSLIYAASSLQSGVPSKFSGSGSGTDFTLTISSLQPEDFATYYCQQYNSYPYTFAQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 476 aa, Artificial Sequence
  DL3B279-LH-scFv
DIQMTQSPSSLSASVGDRVTITCRASQGISNYLAWFQQKPGKAPKSLIYAASSLQSGVPSKFSGSGSGTDFTLTISSLQPEDFATYYCQQYNSYPYTFAQGTKLEIKGGSEGKSSGSGSESKSTGGSQVQLVQSGAEVKKPGASVKVSCKASGNTFTNYYIHWVRQAPGQGLEWMGIINPSGGSTSYAQKLQGRMTMTRDTSTSTVYMELSSLRSEDTAVYFCARQGPFIGDAFDIWGQGTMVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLS

SEQ ID NO: 112 - protein, 474 aa, Artificial Sequence
  CD3W245-LH-scFv-Fc
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 113 - protein, 474 aa, Artificial Sequence
  CD3W245-HL-scFv-Fc
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 114 - protein, 445 aa, Artificial Sequence
  CD3W245-Fab-Fc HC2
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 115 - protein, 214 aa, Artificial Sequence
  CD3W245-Fab-Fc LC2
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 449 aa, Artificial Sequence
  CD3B376-Fab-Fc HC2
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 117 - protein, 216 aa, Artificial Sequence
  CD3B376-Fab-Fc LC2
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 118 - protein, 450 aa, Artificial Sequence
  CD3B376-Fab-Fc K477 HC2
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 119 - protein, 243 aa, Artificial Sequence
  CD3W245-scFv-HL
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIK

SEQ ID NO: 120 - protein, 20 aa, Artificial Sequence
  Linker 1
GGSEGKSSGSGSESKSTGGS

SEQ ID NO: 121 - protein, 15 aa, Artificial Sequence
  Linker 16
GKPGSGKPGSGKPGS

SEQ ID NO: 122 - protein, 20 aa, Artificial Sequence
  Linker 17
GKPGSGKPGSGKPGSGKPGS

SEQ ID NO: 123 - protein, 20 aa, Artificial Sequence
  Linker 18
GKGKSGKGKSGKGKSGKGKS

SEQ ID NO: 124 - protein, 14 aa, Artificial Sequence
  Linker 19
STAGDTHLGGEDFD

SEQ ID NO: 125 - protein, 15 aa, Artificial Sequence
  Linker 20
GEGGSGEGGSGEGGS

SEQ ID NO: 126 - protein, 15 aa, Artificial Sequence
  linker 21
GGEGSGGEGSGGEGS

SEQ ID NO: 127 - protein, 15 aa, Artificial Sequence
  Linker 22
GEGESGEGESGEGES

SEQ ID NO: 128 - protein, 15 aa, Artificial Sequence
  Linker 23
GGGESGGEGSGEGGS

SEQ ID NO: 129 - protein, 20 aa, Artificial Sequence
  linker 24
GEGESGEGESGEGESGEGES

SEQ ID NO: 130 - protein, 18 aa, Artificial Sequence
  Linker 25
GSTSGSGKPGSGEGSTKG

SEQ ID NO: 131 - protein, 19 aa, Artificial Sequence
  Linker 26
PRGASKSGSASQTGSAPGS

SEQ ID NO: 132 - protein, 19 aa, Artificial Sequence
  Linker 27
GTAAAGAGAAGGAAAGAAG

SEQ ID NO: 133 - protein, 19 aa, Artificial Sequence
  Linker 28
GTSGSSGSGSGGSGSGGGG

SEQ ID NO: 134 - protein, 20 aa, Artificial Sequence
  Linker 29
GKPGSGKPGSGKPGSGKPGS

SEQ ID NO: 135 - protein, 4 aa, Artificial Sequence
  Linker 30
GSGS

SEQ ID NO: 136 - protein, 10 aa, Artificial Sequence
  Linker 31
APAPAPAPAP

SEQ ID NO: 137 - protein, 20 aa, Artificial Sequence
  Linker 32
APAPAPAPAPAPAPAPAPAP

SEQ ID NO: 138 - protein, 32 aa, Artificial Sequence
  Linker 33
AEAAAKEAAAKEAAAAKEAAAAKEAAAAKAAA

SEQ ID NO: 139 - protein, 16 aa, Artificial Sequence
  Linker 34
GTEGKSSGSGSESKST

SEQ ID NO: 140 - protein, 7 aa, Artificial Sequence
  DL3B279
GNTFTNY

SEQ ID NO: 141 - protein, 6 aa, Artificial Sequence
  DL3B279
NPSGGS

SEQ ID NO: 142 - protein, 10 aa, Artificial Sequence
  DL3B279
QGPFIGDAFD

SEQ ID NO: 143 - protein, 7 aa, Artificial Sequence
  DL3B279
GQTFTNY

SEQ ID NO: 144 - protein, 5 aa, Artificial Sequence
  DL3B332
YYSGT

SEQ ID NO: 145 - protein, 9 aa, Artificial Sequence
  DL3B332
IGPAGFYFD

SEQ ID NO: 146 - protein, 7 aa, Artificial Sequence
  DL3B358
GHIFISY

SEQ ID NO: 147 - protein, 6 aa, Artificial Sequence
  DL3B358
DPSGGS

SEQ ID NO: 148 - protein, 13 aa, Artificial Sequence
  DL3B358
QGMIVGTTGDAFD

SEQ ID NO: 149 - protein, 7 aa, Artificial Sequence
  DL3B409
GYTFTTY

SEQ ID NO: 150 - protein, 6 aa, Artificial Sequence
  DL3B409
DPSGGR

SEQ ID NO: 151 - protein, 11 aa, Artificial Sequence
  DL3B409
GGDGTWYYGMD

SEQ ID NO: 152 - protein, 7 aa, Artificial Sequence
  DL3B450
GGSISSY

SEQ ID NO: 153 - protein, 5 aa, Artificial Sequence
  DL3B450
YTSGS

SEQ ID NO: 154 - protein, 17 aa, Artificial Sequence
  DL3B450
DQAYSGYDWSYYYYGMD

SEQ ID NO: 155 - protein, 7 aa, Artificial Sequence
  DL3B461
GGTLSSY

SEQ ID NO: 156 - protein, 6 aa, Artificial Sequence
  DL3B461
IPIFGT

SEQ ID NO: 157 - protein, 5 aa, Artificial Sequence
  DL3B461
DPFSD

SEQ ID NO: 158 - protein, 7 aa, Artificial Sequence
  DL3B279
SQGISNY

SEQ ID NO: 159 - protein, 4 aa, Artificial Sequence
  DL3B279
AASS

SEQ ID NO: 160 - protein, 6 aa, Artificial Sequence
  DL3B279
YNSYPY

SEQ ID NO: 161 - protein, 8 aa, Artificial Sequence
  DL3B332
SQSVSRSY

SEQ ID NO: 162 - protein, 4 aa, Artificial Sequence
  DL3B332
GASS

SEQ ID NO: 163 - DNA, 327 bp, Artificial Sequence
  DL3B279 VH cDNA
caggttcagttggtccagtctggcgccgaggtaaaaaaacctggagcatcagtcaaggtaagctgtaaagcctccggggatacctttacaagctattacatgcattgggtccgtcaagctccaggccagggacttgaatggatgggaatcatcaacccaagtggtggttctacctcctatgctcaaaaattccaaggccgcgttactatgacacgtgatacatctacaagcaccgtgtacatggagttgagcagcctgcgtagcgaggataccgccgtttattattgtgctcgctggggtcagggtacaacagttaccgtttccagc

SEQ ID NO: 164 - DNA, 309 bp, Artificial Sequence
  DL3B279 VL cDNA
gacattcagatgacacaatctccctcaagtcttagcgccagtgttggggatcgagtaaccatcacttgtagagcctcccagggaattagcaattacttggcatggttccagcaaaaacctggaaaggcccccaaaagcctcatttacgcagctagttctcttcagagcggtgtgccctctaaattttcaggatctggttcaggcaccgatttcacactcacaatctctagccttcaacctgaagatttcgctacttattattgtcaacaatacaacagcttcggtcaaggaactaaacttgaaatcaag

SEQ ID NO: 165 - DNA, 360 bp, Artificial Sequence
  DL3B279 variant -VH cDNA
caggttcagctggttcagtctggcgccgaagtgaagaaacctggcgcctctgtgaaggtgtcctgcaaggcttctggacagaccttcaccaactactacatccactgggtccgacaggcccctggacaaggattggagtggatgggcatcatcaacccttccggcggctctacctcttacgcccagaaactgcagggcagaatgaccatgaccagagacacctccaccagcaccgtgtacatggaactgtccagcctgagatccgaggataccgccgtgtacttctgtgccagacagggaccttttatcggcgacgccttcgacatctggggccagggaacaacagtgaccgtgtcctct

SEQ ID NO: 166 - DNA, 321 bp, Artificial Sequence
  DL3B279 variant -VL cDNA
gacatccagatgacccagtctccatcctctctgtccgcctctgtgggcgacagagtgaccatcacctgtagagcctctcagggcatctccaactacctggcctggttccagcagaagcctggcaaggctcccaagagcctgatctacgctgcttccagtctgcagtctggcgtgccctctaagttctccggctctggctctggcaccgactttaccctgacaatctccagcctgcagcctgaggacttcgccacctactactgccagcagtacaacagctacccctacacctttggccagggcaccaagctggaaatcaag

SEQ ID NO: 167 - DNA, 354 bp, Artificial Sequence
  DL3B332 VH cDNA
caggtgcagctgcaggagagcggcccaggcctggtgaagccaagcgagaccctgagcctgagctgcaccgtgagcggcgacagcatccgcagctactactggagctggatccgccagccaccaggtaagggcctggaatggatcggctacatctactacagcggcaccaccaactacaagagcagcctgaagagccgcgtgaccatcagcctcgacaccagcaagaagcagttcagcctgaacctggacagcgtgaccgccgccgacaccgctgtgtactactgcgcccgcatcggcccagccggcttctacttcgactactggggccagggcaccctggtgaccgtgagcagc

SEQ ID NO: 168 - DNA, 324 bp, Artificial Sequence
  DL3B332 VL cDNA
gagatcgtgctgacccagagcccaggcaccctgagcctgagcccaggcgagcgcgccaccctgagctgccgcgccagccagagcgtgagccgcagctacctcgcttggtaccagcagaagccaggccaagccccacgcttcctgatctacggtgctagcagccgcgctaccggcatcccagaccgcttcagcggcagcggcagcggcaccgacttcaccctgaccatcagccgcctggagccagaggacttcgccgtgtactactgccagcagtacggcaccagcccaatcaccttcggccagggcacccgcctggagatcaag

SEQ ID NO: 169 - DNA, 369 bp, Artificial Sequence
  DL3B358 VH cDNA
caggttcagctcgtccagagtggcgcagaagtgaaaaaaccaggtgcgtcagtaaaagtttcctgtaaagcaagcggacatattttcatcagttactacattcactgggtccgacaagctccagggcaagggctggagtggatggggatcatcgatccctctggaggcagcaaatcctacgcccaaaaatttcaagggcgggtgaccatgaccagggatacatcaacctctaccgtttacatggaactgtcatcacttcgatcagaagataccgctgtgtactattgcgcgcggcagggcatgatcgtgggcactactggtgatgctttcgacatttggggccagggcactatggtgaccgtttccagt

SEQ ID NO: 170 - DNA, 321 bp, Artificial Sequence
  DL3B358 VL cDNA
gagatcgtgttgacacagtctccgggaacgcttagtctgagcccgggtgagagagccactttgagctgtcgagcttcccagtcagctagcagctatctcgcttggtatcagcaaaaacctggacaggcgcccaggttgctcatttacggagctagtagccgcgctacgggaatcccggacaggtttagcggcagtggttctgggactgactttacgctgacaataagtaggctggaacccgaggacttcgcggtgtattactgccaacagtataactcaagcccctacacctttggtcaaggcaccaaactggaaataaag

SEQ ID NO: 171 - DNA, 363 bp, Artificial Sequence
  DL3B409 VH cDNA
gaagtgcagcttgttcaatctggtgctgaggttaaaaagccaggagcttccgtcaaagttagctgtaaggccagcggatatacatttactacctattatatccattgggttcgtcaagctccaggacaaggccttgagtggatgggaataatagaccctagcggcggtcggacaagctacgcacaaaagtttctgggcagggtgaccatgactagggatacctcaacctcaactgtgtatatggagctgcgttctctgcggtcagaggatactgccgtctactattgcgccagaggtggtgacggcacctggtattacggaatggatgtatggggccaggggaccacagtcaccgtctcatct

SEQ ID NO: 172 - DNA, 321 bp, Artificial Sequence
  DL3B409 VL cDNA
gatattgtaatgactcagtccccaagcttcctgtcagccagtgtaggagatcgggttacaataacatgcagggcatcacaagggatctctaactatctggcctggtaccaacaaaagcctggaaaagcccctaagcttctcatatacgcagcatcaactttgcaatccggcgtcccctcaaggtttagtggatcaggatcaggcacagaatttactcttactatctctagcttgcaaccagaagatttcgctacatactactgtcagcagcttaacagttaccccttgacctttgggggcggcaccaaggtagagataaaa

SEQ ID NO: 173 - DNA, 378 bp, Artificial Sequence
  DL3B450 VH cDNA
caagtccaacttcaacaatctggaccaggacttgtgaagccttctgagaccctgtcccttacatgcacagtgagcgggggtagcataagcagctactactggtcttggattaggcaacccgccggcaaagggcttgagtggatcggaaggatttacacatcaggaagcaccaactataaccctagcctcaagtctcgagtaaccatgtccgttgatacctcaaaaaaccaatttagccttaaactttcctccgtgaccgcagccgatacagcagtgtattattgtgctcgagaccaagcctattctggttacgattggtcttactattattatggaatggatgtttggggacagggaactatggttacagtttccagc

SEQ ID NO: 174 - DNA, 336 bp, Artificial Sequence
  DL3B450 VL cDNA
gaaaccacactgacacagtccccacttagcctgcctgtgactcccggcgaaccagctagcattagttgcaggtcctcacaaagccttcttcactcaaacggatacaattatctcgattggtatctccaaaaaccaggacagtctccccaacttcttatctatttgggttctaatcgagcaagtggtgtaccagacagattcagtggaagcggaagcggcactgatttcactttgaaaatatcacgagtggaggccgaagatgtcggggtttactattgtatgcaggctttgcaaacacctctgacctttgggggaggaactaaggtagagataaaa

SEQ ID NO: 175 - DNA, 345 bp, Artificial Sequence
  DL3B461 VH cDNA
caagtccagctcgtccaatctggtgctgaggtgaagaagcctggctcatctgttaaggtctcctgcaaggcttcaggcggcaccctgtccagctacgtgatctcttgggtacggcaggcccctggtcaaggactcgaatggatgggtggtattattcccatttttggcaccgcaaactacgcacagaaattccaagacagagtaaccataaccgccgacaagtcaaccaatactgcctacatggagctgacctcactgactagtgaagataccgcagtttactactgcgcccgcgatccctttagcgacctttggggacggggtacaatggttaccgtatccagt

SEQ ID NO: 176 - DNA, 336 bp, Artificial Sequence
  DL3B461 VL cDNA
gacatagtaatgactcaatcacctctctcaagcccagtcacacttgggcaaccagcctctatttcatgtaggtcttcacagagtctcgtacattcagatggtaacacctatcttaattggctccaacagagacccggccagccacctcgacttttgatttatcaaatctctaacccattcagtggagtacctgaccgcttcagcggatcaggcgcaggtaccgattttactctcaagataagcagagtggaagcagaagacgtaggtgtatattactgtatgcaggccacacaattccctcacactttcgggccaggtactaaggtggagattaaa

SEQ ID NO: 177 - DNA, 1335 bp, Artificial Sequence
  CD3W245 Fab-Fc HC2 cDNA
gaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggt

SEQ ID NO: 178 - protein, 527 aa, Artificial Sequence
  DL3W33
SGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHGYVCECARGYGGPNCQFLLPELPPGPAVVDLTEKLEGQGDIHHHHHH

SEQ ID NO: 179 - protein, 508 aa, Artificial Sequence
  DL3W34
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPHHHHHHHHHH

SEQ ID NO: 180 - protein, 473 aa, Artificial Sequence
  Recombinant human DLL3 27-479  C-Terminal-6xHis HPGGGSGGGSGGGS Linker
AGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAHPGGGSGGGSGGGSHHHHHH

SEQ ID NO: 181 - protein, 659 aa, Artificial Sequence
  DL3W36
RADPCAARPCAHGGRCYAHFSGLVCACAPGYMGARCEFPVHPDGASALPAAPPGLRPGDPQRYLGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHH

SEQ ID NO: 182 - protein, 632 aa, Artificial Sequence
  DL3W37
RADPCAARPCAHGGRCYAHFSGLVCACAPGYMGARCEGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHH

SEQ ID NO: 183 - protein, 631 aa, Artificial Sequence
  DL3W38
LDDCAGRACANGGTCVEGGGAHRCSCALGFGGRDCRGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHH

SEQ ID NO: 184 - protein, 632 aa, Artificial Sequence
  DL3W39
RVDRCSLQPCRNGGLCLDLGHALRCRCRAGFAGPRCEGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHH

SEQ ID NO: 185 - protein, 635 aa, Artificial Sequence
  DL3W40
SGVTCADGPCFNGGLCVGGADPDSAYICHCPPGFQGSNCEGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHH

SEQ ID NO: 186 - protein, 632 aa, Artificial Sequence
  DL3W41
GPGPCDGNPCANGGSCSETPRSFECTCPRGFYGLRCEGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHH

SEQ ID NO: 187 - protein, 690 aa, Artificial Sequence
  DL3W42
APLVCRAGCSPEHGFCEQPGECRCLEGWTGPLCTVPVSTSSCLSPRGPSSATTGCLVPGPGPCDGNPCANGGSCSETPRSFECTCPRGFYGLRCEGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHH

SEQ ID NO: 188 - protein, 629 aa, Artificial Sequence
  DL3W43
APLVCRAGCSPEHGFCEQPGECRCLEGWTGPLCTGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHH

SEQ ID NO: 189 - protein, 784 aa, Artificial Sequence
  DL3W44
AGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHH

SEQ ID NO: 190 - protein, 247 aa, Artificial Sequence
  DL3B279 scFv_HL
QVQLVQSGAEVKKPGASVKVSCKASGNTFTNYYIHWVRQAPGQGLEWMGIINPSGGSTSYAQKLQGRMTMTRDTSTSTVYMELSSLRSEDTAVYFCARQGPFIGDAFDIWGQGTMVTVSSGGSEGKSSGSGSESKSTGGSDIQMTQSPSSLSASVGDRVTITCRASQGISNYLAWFQQKPGKAPKSLIYAASSLQSGVPSKFSGSGSGTDFTLTISSLQPEDFATYYCQQYNSYPYTFAQGTKLEIK

SEQ ID NO: 191 - protein, 247 aa, Artificial Sequence
  DL3B279 scFv variant_HL
QVQLVQSGAEVKKPGASVKVSCKASGQTFTNYYIHWVRQAPGQGLEWMGIINPSGGSTSYAQKLQGRMTMTRDTSTSTVYMELSSLRSEDTAVYFCARQGPFIGDAFDIWGQGTTVTVSSGGSEGKSSGSGSESKSTGGSDIQMTQSPSSLSASVGDRVTITCRASQGISNYLAWFQQKPGKAPKSLIYAASSLQSGVPSKFSGSGSGTDFTLTISSLQPEDFATYYCQQYNSYPYTFGQGTKLEIK

SEQ ID NO: 192 - protein, 246 aa, Artificial Sequence
  DL3B332 scFv _HL
QVQLQESGPGLVKPSETLSLSCTVSGDSIRSYYWSWIRQPPGKGLEWIGYIYYSGTTNYKSSLKSRVTISLDTSKKQFSLNLDSVTAADTAVYYCARIGPAGFYFDYWGQGTLVTVSSGGSEGKSSGSGSESKSTGGSEIVLTQSPGTLSLSPGERATLSCRASQSVSRSYLAWYQQKPGQAPRFLIYGASSRATGIPDRFSGSGSGTDFTLTISRLEPEDFAVYYCQQYGTSPITFGQGTRLEIK

SEQ ID NO: 193 - protein, 250 aa, Artificial Sequence
  DL3B358 scFv_HL
QVQLVQSGAEVKKPGASVKVSCKASGHIFISYYIHWVRQAPGQGLEWMGIIDPSGGSKSYAQKFQGRVTMTRDTSTSTVYMELSSLRSEDTAVYYCARQGMIVGTTGDAFDIWGQGTMVTVSSGGSEGKSSGSGSESKSTGGSEIVLTQSPGTLSLSPGERATLSCRASQSASSYLAWYQQKPGQAPRLLIYGASSRATGIPDRFSGSGSGTDFTLTISRLEPEDFAVYYCQQYNSSPYTFGQGTKLEIK

SEQ ID NO: 194 - protein, 248 aa, Artificial Sequence
  DL3B409 scFv_HL
EVQLVQSGAEVKKPGASVKVSCKASGYTFTTYYIHWVRQAPGQGLEWMGIIDPSGGRTSYAQKFLGRVTMTRDTSTSTVYMELRSLRSEDTAVYYCARGGDGTWYYGMDVWGQGTTVTVSSGGSEGKSSGSGSESKSTGGSDIVMTQSPSFLSASVGDRVTITCRASQGISNYLAWYQQKPGKAPKLLIYAASTLQSGVPSRFSGSGSGTEFTLTISSLQPEDFATYYCQQLNSYPLTFGGGTKVEIK

SEQ ID NO: 195 - protein, 258 aa, Artificial Sequence
  DL3B450 scFv_HL
QVQLQQSGPGLVKPSETLSLTCTVSGGSISSYYWSWIRQPAGKGLEWIGRIYTSGSTNYNPSLKSRVTMSVDTSKNQFSLKLSSVTAADTAVYYCARDQAYSGYDWSYYYYGMDVWGQGTMVTVSSGGSEGKSSGSGSESKSTGGSETTLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 196 - protein, 247 aa, Artificial Sequence
  DL3B461 scFv_HL
QVQLVQSGAEVKKPGSSVKVSCKASGGTLSSYVISWVRQAPGQGLEWMGGIIPIFGTANYAQKFQDRVTITADKSTNTAYMELTSLTSEDTAVYYCARDPFSDLWGRGTMVTVSSGGSEGKSSGSGSESKSTGGSDIVMTQSPLSSPVTLGQPASISCRSSQSLVHSDGNTYLNWLQQRPGQPPRLLIYQISNPFSGVPDRFSGSGAGTDFTLKISRVEAEDVGVYYCMQATQFPHTFGPGTKVEIK

SEQ ID NO: 197 - protein, 432 aa, Artificial Sequence
  TRCW5
FKIPIEELEDRVFVNCNTSITWVEGTVGTLLSDITRLDLGKRILDPRGIYRCNGTDIYKDKESTVQVHYRMGSADDAKKDAAKKDDAKKDDAKKDGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGLNDIFEAQKIEWHE

SEQ ID NO: 198 - protein, 446 aa, Artificial Sequence
  CD3B815 Heavy Chain
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 214 aa, Artificial Sequence
  CD3B815  Light Chain
DILLTQSPGILSVSPGERVSFSCRARQSIGTAIHWYQQRTNGSPRLLIKYASESISGIPSRFSGSGSGTDFTLTINSVESEDIADYYCQQSNSWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 426 aa, Artificial Sequence
  CD3W147
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVGSADDAKKDAAKKDDAKKDDAKKDGSQSIKGNHLVKVYDYQEDGSVLLTCDAEAKNITWFKDGKMIGFLTEDKKKWNLGSNAKDPRGMYQCKGSQNKSKPLQVYYRMGSGSLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTFPPSQEEMTKNQVSLRCLVKGFYPSDIAVEWESNGQPENNYKTTKPVLDSDGSFRLESRLTVDKSRWQEGNVFSCSVMHEALHNHYTQKSLSLSGGHHHHHH

SEQ ID NO: 201 - protein, 6 aa, Artificial Sequence
  DL3B358
YGTSPI

SEQ ID NO: 202 - DNA, 642 bp, Artificial Sequence
  CD3W245 Fab-Fc LC2 cDNA
gacatacaaatgacacaatcaccctcttctctttctgcaagcgttggcgaccgtgtcactatcacttgtcgagcccgccagtccataggtactgccattcactggtatcaacagaagcctggcaaggctcccaaactcctgattaagtatgccagcgagagcatttccggcgtaccttcaagattttccggctccggtagtgggacagatttcactctcactatatctagcctccaaccagaagatttcgccacttactactgtcaacaatcaggttcatggccttacactttcggccaggggacaaaattggagatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 203 - protein, 6 aa, Artificial Sequence
  DL3B409
LNSYPL

SEQ ID NO: 204 - protein, 12 aa, Artificial Sequence
  DL3B450
SQSLLHSNGYNY

SEQ ID NO: 205 - protein, 4 aa, Artificial Sequence
  DL3B450
LGSN

SEQ ID NO: 206 - protein, 262 aa, Artificial Sequence
  CD3W245-HL-E.c.
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGTEGKSSGSGSESKSTDIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGPGGQHHHHHHGAYPYDVPDYAS

SEQ ID NO: 207 - protein, 6 aa, Artificial Sequence
  DL3B450
ALQTPL

SEQ ID NO: 208 - protein, 12 aa, Artificial Sequence
  DL3B461
SQSLVHSDGNTY

SEQ ID NO: 209 - protein, 4 aa, Artificial Sequence
  DL3B461
QISN

SEQ ID NO: 210 - protein, 6 aa, Artificial Sequence
  DL3B461
ATQFPH

SEQ ID NO: 211 - protein, 8 aa, Artificial Sequence
  DL3B279
GNTFTNYY

SEQ ID NO: 212 - protein, 8 aa, Artificial Sequence
  DL3B279
INPSGGST

SEQ ID NO: 213 - protein, 13 aa, Artificial Sequence
  DL3B279
ARQGPFIGDAFDI

SEQ ID NO: 214 - protein, 8 aa, Artificial Sequence
  DL3B279  varient
GQTFTNYY

SEQ ID NO: 215 - protein, 8 aa, Artificial Sequence
  DL3B332
GDSIRSYY

SEQ ID NO: 216 - protein, 7 aa, Artificial Sequence
  DL3B332
IYYSGTT

SEQ ID NO: 217 - protein, 12 aa, Artificial Sequence
  DL3B332
ARIGPAGFYFDY

SEQ ID NO: 218 - protein, 8 aa, Artificial Sequence
  DL3B358
GHIFISYY

SEQ ID NO: 219 - protein, 8 aa, Artificial Sequence
  DL3B358
IDPSGGSK

SEQ ID NO: 220 - protein, 16 aa, Artificial Sequence
  DL3B358
ARQGMIVGTTGDAFDI

SEQ ID NO: 221 - protein, 8 aa, Artificial Sequence
  DL3B409
GYTFTTYY

SEQ ID NO: 222 - protein, 8 aa, Artificial Sequence
  DL3B409
IDPSGGRT

SEQ ID NO: 223 - protein, 14 aa, Artificial Sequence
  DL3B409
ARGGDGTWYYGMDV

SEQ ID NO: 224 - protein, 8 aa, Artificial Sequence
  DL3B450
GGSISSYY

SEQ ID NO: 225 - protein, 7 aa, Artificial Sequence
  DL3B450
IYTSGST

SEQ ID NO: 226 - protein, 20 aa, Artificial Sequence
  DL3B450
ARDQAYSGYDWSYYYYGMDV

SEQ ID NO: 227 - protein, 8 aa, Artificial Sequence
  DL3B461
GGTLSSYV

SEQ ID NO: 228 - protein, 8 aa, Artificial Sequence
  DL3B461
IIPIFGTA

SEQ ID NO: 229 - protein, 479 aa, Artificial Sequence
  DL3B279-VL-A99G-VH-N27Q_M105T-LH-scFv-Fc (KIH)
DIQMTQSPSSLSASVGDRVTITCRASQGISNYLAWFQQKPGKAPKSLIYAASSLQSGVPSKFSGSGSGTDFTLTISSLQPEDFATYYCQQYNSYPYTFGQGTKLEIKGGSEGKSSGSGSESKSTGGSQVQLVQSGAEVKKPGASVKVSCKASGQTFTNYYIHWVRQAPGQGLEWMGIINPSGGSTSYAQKLQGRMTMTRDTSTSTVYMELSSLRSEDTAVYFCARQGPFIGDAFDIWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 450 aa, Artificial Sequence
  CD3B376-Fab-Fc HC2
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 231 - protein, 8 aa, Artificial Sequence
  DL3B461
ARDPFSDL

SEQ ID NO: 232 - protein, 6 aa, Artificial Sequence
  DL3B279
QGISNY

SEQ ID NO: 233 - DNA, 750 bp, Artificial Sequence
  DL3B358-scFv-LH DNA
gaaatcgttctcactcagtctcccggaactctcagtctttcaccaggggaacgagcaacactgtcctgccgcgcatctcaatccgcctcctcttaccttgcttggtatcaacaaaagccaggacaagctcctcgcttgcttatatacggggcatcaagcagggcaaccggtatcccagatagattttctggcagtggatcagggacagattttactcttactatctcccgattggaaccagaggattttgctgtttactactgccaacagtacaattcatccccatatacttttggtcaggggacaaaattggaaataaagggcggctccgagggcaagagcagcggcagcggcagcgagagcaagagcaccggcggcagccaggttcaacttgtgcaaagcggcgctgaggtgaagaaacctggagcctctgtcaaggtgtcttgtaaggcatctggccacatctttattagctattacatacactgggttcgccaagctccagggcagggtcttgagtggatgggtattatagacccctcaggaggaagcaaatcatacgctcagaaattccaaggacgagtaaccatgacacgagatacaagcacatcaaccgtgtatatggagctctctagtctgcgcagcgaggacacagccgtttattactgtgcccggcaggggatgattgtggggactaccggggatgctttcgacatttggggccaggggactatggttactgtttccagc

SEQ ID NO: 234 - DNA, 744 bp, Artificial Sequence
  DL3B409-scFv-LH DNA
gatattgtaatgactcagtccccaagcttcctgtcagccagtgtaggagatcgggttacaataacatgcagggcatcacaagggatctctaactatctggcctggtaccaacaaaagcctggaaaagcccctaagcttctcatatacgcagcatcaactttgcaatccggcgtcccctcaaggtttagtggatcaggatcaggcacagaatttactcttactatctctagcttgcaaccagaagatttcgctacatactactgtcagcagcttaacagttaccccttgacctttgggggcggcaccaaggtagagataaaaggcggctccgagggcaagagcagcggcagcggcagcgagagcaagagcaccggcggcagcgaagtgcagcttgttcaatctggtgctgaggttaaaaagccaggagcttccgtcaaagttagctgtaaggccagcggatatacatttactacctattatatccattgggttcgtcaagctccaggacaaggccttgagtggatgggaataatagaccctagcggcggtcggacaagctacgcacaaaagtttctgggcagggtgaccatgactagggatacctcaacctcaactgtgtatatggagctgcgttctctgcggtcagaggatactgccgtctactattgcgccagaggtggtgacggcacctggtattacggaatggatgtatggggccaggggaccacagtcaccgtctcatct

SEQ ID NO: 235 - DNA, 774 bp, Artificial Sequence
  DL3B450-scFv-LH DNA
gaaaccacactgacacagtccccacttagcctgcctgtgactcccggcgaaccagctagcattagttgcaggtcctcacaaagccttcttcactcaaacggatacaattatctcgattggtatctccaaaaaccaggacagtctccccaacttcttatctatttgggttctaatcgagcaagtggtgtaccagacagattcagtggaagcggaagcggcactgatttcactttgaaaatatcacgagtggaggccgaagatgtcggggtttactattgtatgcaggctttgcaaacacctctgacctttgggggaggaactaaggtagagataaaaggcggctccgagggcaagagcagcggcagcggcagcgagagcaagagcaccggcggcagccaagtccaacttcaacaatctggaccaggacttgtgaagccttctgagaccctgtcccttacatgcacagtgagcgggggtagcataagcagctactactggtcttggattaggcaacccgccggcaaagggcttgagtggatcggaaggatttacacatcaggaagcaccaactataaccctagcctcaagtctcgagtaaccatgtccgttgatacctcaaaaaaccaatttagccttaaactttcctccgtgaccgcagccgatacagcagtgtattattgtgctcgagaccaagcctattctggttacgattggtcttactattattatggaatggatgtttggggacagggaactatggttacagtttccagc

SEQ ID NO: 236 - DNA, 741 bp, Artificial Sequence
  DL3B461-scFv-LH DNA
gacatagtaatgactcaatcacctctctcaagcccagtcacacttgggcaaccagcctctatttcatgtaggtcttcacagagtctcgtacattcagatggtaacacctatcttaattggctccaacagagacccggccagccacctcgacttttgatttatcaaatctctaacccattcagtggagtacctgaccgcttcagcggatcaggcgcaggtaccgattttactctcaagataagcagagtggaagcagaagacgtaggtgtatattactgtatgcaggccacacaattccctcacactttcgggccaggtactaaggtggagattaaaggcggctccgagggcaagagcagcggcagcggcagcgagagcaagagcaccggcggcagccaagtccagctcgtccaatctggtgctgaggtgaagaagcctggctcatctgttaaggtctcctgcaaggcttcaggcggcaccctgtccagctacgtgatctcttgggtacggcaggcccctggtcaaggactcgaatggatgggtggtattattcccatttttggcaccgcaaactacgcacagaaattccaagacagagtaaccataaccgccgacaagtcaaccaatactgcctacatggagctgacctcactgactagtgaagataccgcagtttactactgcgcccgcgatccctttagcgacctttggggacggggtacaatggttaccgtatccagt

SEQ ID NO: 237 - DNA, 1350 bp, Artificial Sequence
  CD3B376 Fab-Fc HC2 KIH cDNA
caggtgcagctgcagcagtctggccctagactcgtgcggccttcccagaccctgtctctgacctgtgccatctccggcgactccgtgttcaacaacaacgccgcctggtcctggatccggcagtctccatctcgcggtctggagtggctcggtcgcacctactaccgctctaaatggctgtacgactacgccgtgtccgtgaagtcccggatcaccgtgaaccctgacacctcccggaaccagttcaccctgcagctgaactccgtgacccctgaggacaccgccctgtactactgcgccagaggctactcctcctccttcgactattggggccaaggcaccctcgtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 238 - DNA, 648 bp, Artificial Sequence
  CD3B376 Fab-Fc LC KIH cDNA
cagtctgctctgacccagcctgcctccgtgtctggctctcccggccagtccatcaccatcagctgtaccggcacctcctccaacatcggcacctacaagttcgtgtcctggtatcagcagcaccccgacaaggcccccaaagtgctgctgtacgaggtgtccaagcggccctctggcgtgtcctccagattctccggctccaagtctggcaacaccgcctccctgaccatcagcggactgcaggctgaggaccaggccgactaccactgtgtgtcctacgctggctctggcaccctgctgtttggcggaggcaccaagctgaccgtgctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 239 - DNA, 1437 bp, Artificial Sequence
  DL3B279 scFv-Fc variant KIH cDNA
gacatccagatgacccagtctccatcctctctgtccgcctctgtgggcgacagagtgaccatcacctgtagagcctctcagggcatctccaactacctggcctggttccagcagaagcctggcaaggctcccaagagcctgatctacgctgcttccagtctgcagtctggcgtgccctctaagttctccggctctggctctggcaccgactttaccctgacaatctccagcctgcagcctgaggacttcgccacctactactgccagcagtacaacagctacccctacacctttggccagggcaccaagctggaaatcaagggcggctccgagggcaagagcagcggcagcggcagcgagagcaagagcaccggcggcagccaggttcagctggttcagtctggcgccgaagtgaagaaacctggcgcctctgtgaaggtgtcctgcaaggcttctggacagaccttcaccaactactacatccactgggtccgacaggcccctggacaaggattggagtggatgggcatcatcaacccttccggcggctctacctcttacgcccagaaactgcagggcagaatgaccatgaccagagacacctccaccagcaccgtgtacatggaactgtccagcctgagatccgaggataccgccgtgtacttctgtgccagacagggaccttttatcggcgacgccttcgacatctggggccagggaacaacagtgaccgtgtcctctgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 240 - protein, 7 aa, Artificial Sequence
  DL3B332
QSVSRSY

SEQ ID NO: 241 - protein, 6 aa, Artificial Sequence
  DL3B358
QSASSY

SEQ ID NO: 242 - protein, 11 aa, Artificial Sequence
  DL3B450
QSLLHSNGYNY

SEQ ID NO: 243 - protein, 11 aa, Artificial Sequence
  DL3B450
QSLVHSDGNTY

SEQ ID NO: 244 - protein, 7 aa, Artificial Sequence
  DL3B332
GDSIRSY

SEQ ID NO: 245 - protein, 6 aa, Artificial Sequence
  DL3B358
YNSSPY

SEQ ID NO: 246 - protein, 38 aa, Artificial Sequence
  DLL3-DSL domain
ARCEPPAVGTACTRLCRPRSAPSRCGPGLRPCAPLEDE

SEQ ID NO: 247 - protein, 36 aa, Artificial Sequence
  DLL3-EGF-1 domain
CEAPLVCRAGCSPEHGFCEQPGECRCLEGWTGPLCT

SEQ ID NO: 248 - protein, 27 aa, Artificial Sequence
  DLL3 EGF-2 domain
GPGPCDGNPCANGGSCSETPRSFECTC

SEQ ID NO: 249 - protein, 40 aa, Artificial Sequence
  DLL3 EGF-3 domain
SGVTCADGPCFNGGLCVGGADPDSAYICHCPPGFQGSNCE

SEQ ID NO: 250 - protein, 37 aa, Artificial Sequence
  DLL3 EGF-4 domain
RVDRCSLQPCRNGGLCLDLGHALRCRCRAGFAGPRCE

SEQ ID NO: 251 - protein, 37 aa, Artificial Sequence
  DLL3 EGF-5 domain
DLDDCAGRACANGGTCVEGGGAHRCSCALGFGGRDCR

SEQ ID NO: 252 - protein, 36 aa, Artificial Sequence
  DLL3 EGF-6 domain
ADPCAARPCAHGGRCYAHFSGLVCACAPGYMGARCE

SEQ ID NO: 253 - protein, 207 aa, Artificial Sequence
  Human CD3 epsilon
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 254 - protein, 104 aa, Artificial Sequence
  Human CD3 epsilon extracellular domain
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 255 - protein, 618 aa, Artificial Sequence
  NP_058637.1
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 256 - DNA, 1347 bp, Artificial Sequence
  CD3B376 Fab-Fc HC2 cDNA
caggtgcagctccaacagagtggtcccagactcgtgagaccctctcaaacactcagtttgacttgtgccatctcaggcgattcagttttcaacaacaatgcagcttggagctggattaggcagtcacctagtcgcggtcttgaatggcttgggcgtacatactatcgctctaaatggttgtatgattacgctgtgtccgtgaagagccgaatcaccgtaaaccctgatacctccaggaatcagttcacattgcaactgaatagtgtgactcccgaggatactgcactctattattgtgcccgaggatatagcagtagcttcgactattggggacaagggacactcgttaccgttagttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggt

SEQ ID NO: 257 - protein, 330 aa, Artificial Sequence [sequence omitted]

SEQ ID NO: 258 - protein, 326 aa, Artificial Sequence
  wild-type IgG2
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 259 - protein, 327 aa, Artificial Sequence
  wild-type IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 260 - DNA, 741 bp, Artificial Sequence
  DL3B279-scFv-LH DNA
gatatacaaatgactcaaagtccctctagtctcagtgcttccgttggggatagggtaactatcacttgccgcgctagtcaaggcatctcaaattatttggcatggttccaacagaagcctggtaaggcaccaaaatcactcatctacgctgctagttctctgcaatcaggtgtgccatccaaattctcaggcagtgggtctgggacagactttaccctcaccataagttctctccaacctgaggattttgcaacatactattgtcagcagtataatagttatccatacacattcgcacaaggtactaaactggaaatcaagggcggctccgagggcaagagcagcggcagcggcagcgagagcaagagcaccggcggcagccaagttcaacttgtccaaagtggagctgaagtgaagaaaccaggggcaagcgtcaaagtatcatgtaaagccagcggaaacactttcaccaactactacattcattgggtaaggcaggcaccaggacaaggcttggaatggatgggtattataaacccttcaggtggcagtacatcctatgcccaaaaactgcaaggtcggatgaccatgactcgtgacacttcaacttctactgtttatatggagctttcatccctgcgatccgaagatacagccgtgtatttttgtgctcgccaggggcctttcatcggagatgccttcgatatatggggtcaaggcacaatggtaactgtgtcaagc

SEQ ID NO: 261 - DNA, 741 bp, Artificial Sequence
  DL3B279-scFv-LH variant DNA
gacatccagatgacccagtctccatcctctctgtccgcctctgtgggcgacagagtgaccatcacctgtagagcctctcagggcatctccaactacctggcctggttccagcagaagcctggcaaggctcccaagagcctgatctacgctgcttccagtctgcagtctggcgtgccctctaagttctccggctctggctctggcaccgactttaccctgacaatctccagcctgcagcctgaggacttcgccacctactactgccagcagtacaacagctacccctacacctttggccagggcaccaagctggaaatcaagggcggctccgagggcaagagcagcggcagcggcagcgagagcaagagcaccggcggcagccaggttcagctggttcagtctggcgccgaagtgaagaaacctggcgcctctgtgaaggtgtcctgcaaggcttctggacagaccttcaccaactactacatccactgggtccgacaggcccctggacaaggattggagtggatgggcatcatcaacccttccggcggctctacctcttacgcccagaaactgcagggcagaatgaccatgaccagagacacctccaccagcaccgtgtacatggaactgtccagcctgagatccgaggataccgccgtgtacttctgtgccagacagggaccttttatcggcgacgccttcgacatctggggccagggaacaacagtgaccgtgtcctct

SEQ ID NO: 262 - DNA, 738 bp, Artificial Sequence
  DL3B332-scFv-LH DNA
gaaatcgtactgacacagagtccaggaaccctttctctctctcccggagagcgtgctactctgagctgccgggcctctcaaagcgtgagtcgcagctacctcgcatggtatcagcagaaaccaggacaggcacctcggtttctcatatatggtgcttcctccagagccacagggatacccgatagattcagtggatccggctctggaactgattttactttgactatatcccgcttggagcctgaggatttcgcagtgtactattgccaacagtatggaacctctccaataactttcggccaggggaccagactcgaaataaaaggcggctccgagggcaagagcagcggcagcggcagcgagagcaagagcaccggcggcagccaggtccaactgcaagaatctggtcctggtctggtaaaaccttcagagaccctttccttgtcttgtactgtcagcggagactctatccggtcatattactggtcctggattcgccagccccccggaaagggactcgaatggattggctatatttattactcaggtacaacaaactataagtcatcattgaagtcaagggttaccatcagtcttgatacttctaagaaacagttttctttgaacttggacagtgttaccgcagccgacactgctgtctactactgcgctcgaatcggaccagccggtttctactttgattattgggggcagggcactctggtaactgtgagttca

SEQ ID NO: 263 - protein, 189 aa, Artificial Sequence
  DLL3 EGF6 + C-terminal domain
ADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 264 - DNA, 648 bp, Artificial Sequence
  CD3B376 Fab-Fc LC2 cDNA
cagtctgctctgacccagcctgcctccgtgtctggctctcccggccagtccatcaccatcagctgtaccggcacctcctccaacatcggcacctacaagttcgtgtcctggtatcagcaacaccccgacaaggcccccaaagtgctgctgtacgaggtgtccaagcggccctctggcgtgtcctccagattctccggctccaagtctggcaacaccgcctccctgaccatcagcggactgcaggctgaggaccaggccgactaccactgtgtgtcctacgctggctctggcaccctgctgtttggcggaggcaccaagctgactgtcctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtcgaaaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 265 - DNA, 1434 bp, Artificial Sequence
  DL3B279 LH scFv-Fc cDNA in DL3B585 and DL3B587
gatattcagatgacacagtctccatccagcttgtcagcaagcgtgggtgatagggttaccatcacttgtcgcgcaagtcaaggaattagtaactatttggcatggtttcagcagaaacccggtaaggctccaaaatcactcatatatgcagcatcctccctccagtctggtgttccaagtaagttttccgggagcggttccggcaccgatttcactctcacaatctctagccttcaacccgaggacttcgctacctattattgccaacagtataatagctacccatacacttttgctcaagggaccaaactcgagatcaaaggcggctccgagggcaagagcagcggcagcggcagcgagagcaagagcaccggcggcagccaggttcagttggtccagagtggtgccgaagtaaagaagcccggagcatccgtaaaggtgtcctgtaaagccagtggcaatactttcactaactattacatccattgggtccgacaagcccccggacaaggattggagtggatgggtattatcaacccctccggtgggtctacttcttacgctcaaaaactccagggccgaatgacaatgacacgcgacacctcaacttcaaccgtttacatggagcttagcagtcttcgatctgaggacactgctgtttacttttgcgctaggcaggggcctttcataggagacgcttttgacatctgggggcaaggaacaatggtcactgtcagttccgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 266 - DNA, 1437 bp, Artificial Sequence
  DL3B279 LH scFv variant-Fc cDNA
gacatccagatgacccagtctccatcctctctgtccgcctctgtgggcgacagagtgaccatcacctgtagagcctctcagggcatctccaactacctggcctggttccagcagaagcctggcaaggctcccaagagcctgatctacgctgcttccagtctgcagtctggcgtgccctctaagttctccggctctggctctggcaccgactttaccctgacaatctccagcctgcagcctgaggacttcgccacctactactgccagcagtacaacagctacccctacacctttggccagggcaccaagctggaaatcaagggcggctccgagggcaagagcagcggcagcggcagcgagagcaagagcaccggcggcagccaggttcagctggttcagtctggcgccgaagtgaagaaacctggcgcctctgtgaaggtgtcctgcaaggcttctggacagaccttcaccaactactacatccactgggtccgacaggcccctggacaaggattggagtggatgggcatcatcaacccttccggcggctctacctcttacgcccagaaactgcagggcagaatgaccatgaccagagacacctccaccagcaccgtgtacatggaactgtccagcctgagatccgaggataccgccgtgtacttctgtgccagacagggaccttttatcggcgacgccttcgacatctggggccagggaacaacagtgaccgtgtcctctgagcccaaatctagcgacaaaactcacacttgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtccagatggcagcaggggaacgtcttctcatgctccgtgatgcatgaggctctgcacaaccactacacgcagaagtctctctccctgtctccgggaaaa

SEQ ID NO: 267 - DNA, 1347 bp, Artificial Sequence
  DL3B279-Fab-Fc HC1 cDNA in DL3B582 and DL3B583
caggttcagttggtccagagtggtgccgaagtaaagaagcccggagcatccgtaaaggtgtcctgtaaagccagtggcaatactttcactaactattacatccattgggtccgacaagcccccggacaaggattggagtggatgggtattatcaacccctccggtgggtctacttcttacgctcaaaaactccagggccgaatgacaatgacacgcgacacctcaacttcaaccgtttacatggagcttagcagtcttcgatctgaggacactgctgtttacttttgcgctaggcaggggcctttcataggagacgcttttgacatctgggggcaaggaacaatggtcactgtcagttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 268 - DNA, 642 bp, Artificial Sequence
  DL3B279-Fab-Fc LC1 cDNA in DL3B582 and DL3B583
gacatccagatgacccagagccctagctctttaagcgctagcgtgggcgatcgtgtgaccatcacttgtcgtgccagccaaggtatcagcaactatttagcttggttccagcagaagcccggcaaggctcccaagtctttaatctatgccgctagctctttacagagcggagtgcccagcaagtttagcggcagcggtagcggaaccgacttcactttaaccatcagctctctgcagcccgaggacttcgccacctactactgccagcagtacaacagctacccctacaccttcgcccaaggtaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 269 - DNA, 1422 bp, Artificial Sequence
  CD3W245 LH scFv-Fc cDNA
gacatacaaatgacacaatcaccctcttctctttctgcaagcgttggcgaccgtgtcactatcacttgtcgagcccgccagtccataggtactgccattcactggtatcaacagaagcctggcaaggctcccaaactcctgattaagtatgccagcgagagcatttccggcgtaccttcaagattttccggctccggtagtgggacagatttcactctcactatatctagcctccaaccagaagatttcgccacttactactgtcaacaatcaggttcatggccttacactttcggccaggggacaaaattggagatcaagggcggctccgagggcaagagcagcggcagcggcagcgagagcaagagcaccggcggcagcgaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctcagagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggt

SEQ ID NO: 270 - DNA, 1422 bp, Artificial Sequence
  CD3W245 HL scFv-Fc cDNA
gaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctcaggcggctccgagggcaagagcagcggcagcggcagcgagagcaagagcaccggcggcagcgacatacaaatgacacaatcaccctcttctctttctgcaagcgttggcgaccgtgtcactatcacttgtcgagcccgccagtccataggtactgccattcactggtatcaacagaagcctggcaaggctcccaaactcctgattaagtatgccagcgagagcatttccggcgtaccttcaagattttccggctccggtagtgggacagatttcactctcactatatctagcctccaaccagaagatttcgccacttactactgtcaacaatcaggttcatggccttacactttcggccaggggacaaaattggagatcaaggagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggt

(19) United States
(12) Patent Application Publication
(10) Pub. No.: US 2024/0025992 A1
(43) Pub. Date: Jan. 25, 2024

(54) PROTEINS COMPRISING DELTA-LIKE LIGAND 3 (DLL3) ANTIGEN BINDING DOMAINS AND THEIR USES

(71) Applicant: Janssen Biotech, Inc., Horsham, PA (US)

(72) Inventors: Danlin YANG, Spring House, PA (US); Sanjaya SINGH, Spring House, PA (US); Scott R. BRODEUR, Spring House, PA (US); Jill M. CARTON, Spring House, PA (US); Rajkumar GANESAN, Spring House, PA (US); Jennifer HERTZOG, Spring House, PA (US); Theresa MCDEVITT, Spring House, PA (US); Kristen M. PICHA, Spring House, PA (US); Ryan M. SMITH, Spring House, PA (US); Adam ZWOLAK, Spring House, PA (US); Sathyadevi VENKATARAMANI, Spring House, PA (US); Gordon D. POWERS, Spring House, PA (US)

(21) Appl. No.: 18/249,807

(22) PCT Filed: Oct. 21, 2021
(86) PCT No.: PCT/IB2021/059724

§ 371 Date: Apr. 20, 2023

Related U.S. Application Data
(60) Provisional application No. 63/094,933, filed on Oct. 22, 2020.
(60) Provisional application No. 63/094,934, filed on Oct. 22, 2020.

(51) Int. Cl. C07K 16/28; A61K 47/68; A61P 35/00; A61K 49/00

(52) U.S. Cl. CPC C07K 16/28; A61K 47/6849; C07K 16/2809; C07K 2319/30; A61K 49/0002; C07K 2317/622; C07K 2317/31; A61P 35/00

(57) ABSTRACT

Antibodies and antigen binding regions that bind delta-like protein 3 (DLL3) are described. Multispecific antigen-binding constructs, such as bispecific antibodies, containing the antigen-binding regions that bind to DLL3 are also described. The application also describes methods of treatment or detection using the anti-DLL3 antibodies, antigen-binding fragments or multispecific antigen-binding constructs thereof, and related molecules, compositions and methods.

### CROSS REFERENCE TO RELATED APPLICATION

This application claims priority to U.S. Provisional Application No. 63/094,933 filed on Oct. 22, 2020, and U.S. Provisional Application No. 63/094,934 filed on Oct. 22, 2020, the disclosures of each are incorporated herein by reference in their entireties.

### REFERENCE TO SEQUENCE LISTING SUBMITTED ELECTRONICALLY

This application contains a sequence listing, which is submitted electronically via EFS-Web as an ASCII formatted sequence listing with a file name “sequence listing JBI6411” and a creation date of Oct. 7, 2021 and having a size of 275 KB. The sequence listing submitted via EFS-Web is part of the specification and is herein incorporated by reference in its entirety.

### TECHNICAL FIELD

The application relates to a protein comprising an antigen binding region that binds Delta-like canonical Notch Ligand 3 (DLL3), and related compositions and methods.

### BACKGROUND

Prostate cancer is the second most frequently diagnosed cancer and the sixth leading cause of cancer death in males, accounting for 14% (903,500) of the total new cancer cases and 6% (258,400) of the total cancer deaths in males worldwide. Metastatic prostate cancer is the second leading cause of cancer death in men in the United States. The course of prostate cancer from diagnosis to death is best categorized as a series of clinical stages based on the extent of disease, hormonal status, and absence or presence of detectable metastases: localized disease, rising levels of prostate-specific antigen (PSA) after radiation therapy or surgery with no detectable metastases, and clinical metastases in the non-castrate or castrate stage. Although surgery, radiation, or a combination of both can be curative for patients with localized disease, a significant proportion of these patients have recurrent disease as evidenced by a rising level of PSA, which can lead to the development of metastases, especially in the high-risk group—a transition to the lethal stage of the disease.

Androgen depletion therapy (ADT) is the standard treatment with a generally predictable outcome: decline in PSA, a period of stability in which the tumor does not proliferate, followed by rising PSA and regrowth as castration-resistant disease. Historically, ADT has been the standard of care for patients with metastatic prostate cancer.

However, recent clinical data suggests that androgen deprivation therapies can lead to the emergence of an androgen independent tumor phenotype known as neuroendocrine prostate cancer (NEPC) through the process of cellular trans-differentiation. Delta-like canonical Notch Ligand 3 (DLL3) has been shown to be enriched in NEPC tumors at both the RNA and protein level. Thus, strategies designed to target DLL3 can have clinical utility in NEPC/small cell carcinoma patient populations.

Small-cell lung cancer accounts for approximately 20% of all lung cancer incidence. The small-cell lung cancer rapidly progresses and is difficult to be surgically removed because lymph node metastasis or distant metastasis has already occurred at the time of diagnosis in many cases. This cancer exhibits high response rates to an anticancer agent in its early stage. Thus, chemotherapy is considered as the first choice for treating the cancer. The cancer, however, immediately becomes resistant to chemotherapy and recurs, resulting in a 3-year survival rate of 5% or lower.

Hence, there is a need for new therapy to treat cancers, such as NEPC, small cell carcinoma or small-cell lung cancer.

In normal cells, DLL3 regulates notch signaling intracellularly. In cancer cells, DLL3 is expressed extracellularly, e.g., with 618 amino acids and 8 extracellular domains including six EGF-like repeats in human. The human DLL3 is highly homologous to that of cynomolgus and mouse/rat sharing 96% and 83% amino acid sequence identity, respectively, while it only shares about <40% identity with DLL1 and DLL4. DLL3 has low to undetectable expression in normal tissues, but has been highly expressed on the cell surface of neuroendocrine tumors including small cell lung cancer, prostate, large cell carcinoma, and bladder, and has become a target in T-cell redirection for the treatment of neuroendocrine cancers.

### SUMMARY OF THE INVENTION

In one general aspect, the disclosure relates to an isolated protein comprising an antigen binding region that binds delta-like protein 3 (DLL3), wherein the antigen binding region binds to an epitope within residues 429-618 of human DLL3 as set forth in SEQ ID NO:263.

In some embodiments, the isolated protein comprises an antigen binding region that competes for binding to DLL3 with a reference antibody comprising: a) a heavy chain variable region (VH) having the heavy chain complementarity determining region (HCDR)1, the HCDR2 and the HCDR3 of a VH having the amino acid sequence of SEQ ID NO:1, and a light chain variable region (VL) having the light chain complementarity determining region (LCDR)1, the LCDR2 and the LCDR3 of a VL having the amino acid sequence of SEQ ID NO:2; b) a VH having the HCDR1, the HCDR2 and the HCDR3 of a VH having the amino acid sequence of SEQ ID NO:3, and a VL having the LCDR1, the LCDR2 and the LCDR3 of a VL having the amino acid sequence of SEQ ID NO:4; c) a VH having the HCDR1, the HCDR2 and the HCDR3 of the VH of SEQ ID NO:5 and the LCDR1, the LCDR2 and the LCDR3 of the VL of SEQ ID NO:6; d) the HCDR1, the HCDR2 and the HCDR3 of the VH of SEQ ID NO:7 and the LCDR1, the LCDR2 and the LCDR3 of the VL of SEQ ID NO:8; e) the HCDR1, the HCDR2 and the HCDR3 of the VH of SEQ ID NO:9 and the LCDR1, the LCDR2 and the LCDR3 of the VL of SEQ ID NO: 10; f) the HCDR1, the HCDR2 and the HCDR3 of the VH of SEQ ID NO: 11 and the LCDR1, the LCDR2 and the LCDR3 of the VL of SEQ ID NO: 12; or g) the HCDR1, the HCDR2 and the HCDR3 of the VH of SEQ ID NO: 13 and the LCDR1, the LCDR2 and the LCDR3 of the VL of SEQ ID NO:14. Optionally, the reference antibody comprises the HCDR1, the HCDR2 and the HCDR3 of the VH of SEQ ID NO:3 and the LCDR1, the LCDR2 and the LCDR3 of the VL of SEQ ID NO:4.

Optionally, the isolated protein comprises the HCDR1, the HCDR2, the HCDR3, the LCDR1, the LCDR2 and the LCDR3 of: a) SEQ ID NOs:15, 16, 17, 33, 34, 35, respectively; b) SEQ ID NOs:18, 19, 20, 36, 37, 38, respectively; c) SEQ ID NOs:21, 22, 23, 39, 37, 40, respectively; d) SEQ ID NOs:24, 25, 26, 41, 42, 43, respectively; e) SEQ ID NOs:18, 28, 29, 44, 45, 46, respectively; f) SEQ ID NOs:30, 31, 32, 47, 48, 49, respectively; g) SEQ ID NOs:50, 51, 17, 33, 34, 35, respectively; h) SEQ ID NOs:52, 51, 17, 33, 34, 35, respectively; i) SEQ ID NOs:53, 54, 20, 36, 37, 38, respectively; j) SEQ ID NOs:55, 56, 23, 39, 37, 40, respectively; k) SEQ ID NOs:57, 58, 26, 41, 42, 43, respectively; 1) SEQ ID NOs:59, 60, 29, 44, 45, 46, respectively; or m) SEQ ID NOs:61, 62, 32, 47, 48, 49, respectively. Optionally, the isolated protein comprises the HCDR1, the HCDR2, the HCDR3, the LCDR1, the LCDR2 and the LCDR3 of SEQ ID NOs:15, 16, 17, 33, 34, and 35, respectively. Optionally, the antigen binding region that binds DLL3 is a scFv, a (scFv)₂, a Fv, a Fab, a F(ab′)₂, a Fd, a dAb or a VHH. Optionally, the antigen binding region that binds DLL3 is the Fab. Optionally, the antigen binding region that binds DLL3 is the scFv. Optionally, the scFv comprises, from the N- to C-terminus, a VH, a first linker (L1) and a VL (VH-L1-VL) or the VL, the L1 and the VH (VL-L1-VH). Optionally, the L1 comprises a) about 5-50 amino acids; b) about 5-40 amino acids; c) about 10-30 amino acids; or d) about 10-20 amino acids. Optionally, the L1 comprises an amino acid sequence of SEQ ID NOs:27, 72, 73, 74, 75, 76, 79, 81, 82, 83, 88, 90, 91, 92, 120, 121, 122, 123, 124, 125, 126, 127, 128, 129, 130, 131, 132, 133, 134, 135, 136, 137, 138, or 139. Optionally, the L1 comprises the amino acid sequence of SEQ ID NO: 120.

The disclosure also provides an antigen binding region that binds DLL3 comprising the VH of SEQ ID NOs:1, 3, 5, 7, 9, 11, or 13 and the VL of SEQ ID NOs:2, 4, 6, 8, 10, 12, or 14. Optionally, the antigen binding region that binds DLL3 comprises: a) the VH of SEQ ID NO:1 and the VL of SEQ ID NO:2; b) the VH of SEQ ID NO:3 and the VL of SEQ ID NO:4; c) the VH of SEQ ID NO:5 and the VL of SEQ ID NO:6; d) the VH of SEQ ID NO:7 and the VL of SEQ ID NO:8; e) the VH of SEQ ID NO:9 and the VL of SEQ ID NO: 10; f) the VH of SEQ ID NO:11 and the VL of SEQ ID NO:12; and/or g) the VH of SEQ ID NO:13 and the VL of SEQ ID NO:14. Optionally, the antigen binding region that binds DLL3 comprises a VH which is at least 80% (e.g., at least 85%, at least 90%, at least 95%, at least 99% or 100%) identical to the VH of SEQ ID NO:3 and a VL which is at least 80% (e.g., at least 85%, at least 90%, at least 95%, at least 99% or 100%) identical to the VL of SEQ ID NO:4. Optionally, the antigen binding region that binds DLL3 comprises an amino acid sequence at least 80% (e.g., at least 85%, at least 90%, at least 95%, at least 99% or 100%) identical to the amino acid sequence of SEQ ID NOs:63 or 64.

The disclosure provides an isolated protein that is a monospecific protein or a multispecific antigen-binding construct. Optionally, the isolated protein is a multispecific antigen-binding construct. Optionally, the multispecific antigen-binding construct is a bispecific protein. Optionally, the multispecific antigen-binding construct is a trispecific protein. Optionally, the multispecific antigen-binding construct comprises an antigen binding region that binds an antigen on a lymphocyte. Optionally, the lymphocyte is a T cell. Optionally, the T cell is a CD8+ T cell. Optionally, the lymphocyte is a natural killer (NK) cell. Optionally, in the multispecific antigen-binding construct, the antigen on the lymphocyte is CD3, CD3 epsilon (CD3E), CD8, KI2L4, NKG2E, NKG2D, NKG2F, BTNL3, CD186, BTNL8, PD-1, CD195, or NKG2C. Optionally, the antigen on the lymphocyte is CD3E.

In some embodiments, the multispecific antigen-binding construct comprises an antigen binding region that binds CD3ε comprising a) a heavy chain complementarity determining region (HCDR)1 of SEQ ID NO:98, a HCDR2 of SEQ ID NO:99, a HCDR3 of SEQ ID NO: 100, a light chain complementarity determining region (LCDR)1 of SEQ ID NO: 106, a LCDR2 of SEQ ID NO:107 and a LCDR3 of SEQ ID NO: 108; or b) the VH of SEQ ID NO:84 and the VL of SEQ ID NO:85. In some embodiments, in the multispecific antigen-binding construct, the antigen binding region that binds CD3ε comprises a HCDR1 of SEQ ID NO:98, a HCDR2 of SEQ ID NO:99, a HCDR3 of SEQ ID NO:100, a LCDR1 of SEQ ID NO: 106, a LCDR2 of SEQ ID NO:107 and a LCDR3 of SEQ ID NO:108. In some embodiments, in the multispecific antigen-binding construct, the antigen binding region that binds CD3ε comprises a VH which is at least 80% (e.g., at least 85%, at least 90%, at least 95%, at least 99% or 100%) identical to the VH of SEQ ID NO:84 and a VL which is at least 80% (e.g., at least 85%, at least 90%, at least 95%, at least 99% or 100%) identical to the VL of SEQ ID NO:85.

In some embodiments, the multispecific antigen-binding construct comprises the antigen binding region that binds CD3ε comprising a) a heavy chain complementarity determining region (HCDR)1 of SEQ ID NO:95, a HCDR2 of SEQ ID NO:96, a HCDR3 of SEQ ID NO:97, a light chain complementarity determining region (LCDR)1 of SEQ ID NO: 101, a LCDR2 of SEQ ID NO:102 and a LCDR3 of SEQ ID NO: 104; or b) the VH of SEQ ID NO:77 and the VL of SEQ ID NO:80. In some embodiments, the multispecific antigen-binding construct comprises the antigen binding region that binds CD3ε comprising a HCDR1 of SEQ ID NO:95, a HCDR2 of SEQ ID NO:96, a HCDR3 of SEQ ID NO:97, a LCDR1 of SEQ ID NO:101, a LCDR2 of SEQ ID NO:102 and a LCDR3 of SEQ ID NO:104. In some embodiments, the multispecific antigen-binding construct comprises the antigen binding region that binds CD3E comprising a VH which is at least 80% (e.g., at least 85%, at least 90%, at least 95%, at least 99% or 100%) identical to the VH of SEQ ID NO:77 and a VL which is at least 80% (e.g., at least 85%, at least 90%, at least 95%, at least 99% or 100%) identical to the VL of SEQ ID NO:80.

The disclosure also provides an isolated multispecific antigen-binding construct comprising an antigen binding region that binds delta-like protein 3 (DLL3), wherein the antigen binding region that binds DLL3 comprising the HCDR1, the HCDR2, the HCDR3, the LCDR1, the LCDR2 and the LCDR3 of a) SEQ ID NOs:15, 16, 17, 33, 34, 35, respectively; b) SEQ ID NOs:18, 19, 20, 36, 37, 38, respectively; c) SEQ ID NOs:21, 22, 23, 39, 37, 40, respectively; d) SEQ ID NOs:24, 25, 26, 41, 42, 43, respectively; e) SEQ ID NOs:18, 28, 29, 44, 45, 46, respectively; f) SEQ ID NOs:30, 31, 32, 47, 48, 49, respectively; g) SEQ ID NOs:50, 51, 17, 33, 34, 35, respectively; h) SEQ ID NOs:52, 51, 17, 33, 34, 35, respectively; i) SEQ ID NOs:53, 54, 20, 36, 37, 38, respectively; j) SEQ ID NOs:55, 56, 23, 39, 37, 40, respectively; k) SEQ ID NOs:57, 58, 26, 41, 42, 43, respectively; 1) SEQ ID NOs:59, 60, 29, 44, 45, 46, respectively; m) SEQ ID NOs:61, 62, 32, 47, 48, 49, respectively; n) the VH of SEQ ID NO:1 and the VL of SEQ ID NO:2; o) the VH of SEQ ID NO:3 and the VL of SEQ ID NO:4; p) the VH of SEQ ID NO:5 and the VL of SEQ ID NO:6; q) the VH of SEQ ID NO:7 and the VL of SEQ ID NO:8; r) the VH of SEQ ID NO:9 and the VL of SEQ ID NO: 10; s) the VH of SEQ ID NO:11 and the VL of SEQ ID NO:12; or t) the VH of SEQ ID NO: 13 and the VL of SEQ ID NO:14. Optionally, the multispecific antigen-binding construct comprises the binding domain that binds DLL3 comprising the HCDR1, the HCDR2, the HCDR3, the LCDR1, the LCDR2 and the LCDR3 of SEQ ID NOs:15, 16, 17, 33, 34 and 35, respectively.

In a particular embodiment, the disclosure provides an isolated multispecific antigen-binding construct comprising an antigen binding region that binds delta-like protein 3 (DLL3), wherein the antigen binding region that binds DLL3 comprises a heavy chain complementarity determining region (HCDR)1, a HCDR2 and a HCDR3 of a heavy chain variable region (VH) of SEQ ID NO:3 and a light chain complementarity determining region (LCDR)1, a LCDR2 and a LCDR3 of a light chain variable region (VL) of SEQ ID NO:4.

The disclosure also provides an isolated multispecific antigen-binding construct comprising an antigen binding region that binds delta-like protein 3 (DLL3), wherein the antigen binding region that binds DLL3 comprises a heavy chain variable region (VH) of SEQ ID NO:3 and a light chain variable region (VL) of SEQ ID NO:4.

In some embodiments, the isolated protein is conjugated to a half-life extending moiety. Optionally, the half-life extending moiety is an immunoglobulin (Ig), a fragment of the Ig, an Ig constant region, a fragment of the Ig constant region, a Fc region, transferrin, albumin, an albumin binding domain or polyethylene glycol. Optionally, the fragment of the Ig constant region comprises a Fc region. Optionally, the antigen binding region that binds DLL3 is conjugated to the N-terminus of the Ig constant region or the fragment of the Ig constant region. Optionally, the antigen binding region that binds DLL3 is conjugated to the C-terminus of the Ig constant region or the fragment of the Ig constant region. Optionally, the antigen binding region that binds DLL3 is conjugated to the Ig constant region or the fragment of the Ig constant region via a second linker (L2). Optionally, the L2 comprises the amino acid sequence of SEQ ID NOs:27, 72, 73, 74, 75, 76, 79, 81, 82, 83, 88, 90, 91, 92, 120, 121, 122, 123, 124, 125, 126, 127, 128, 129, 130, 131, 132, 133, 134, 135, 136, 137, 138, or 139. Optionally, the Ig constant region or the fragment of the Ig constant region is an IgG1, an IgG2, an IgG3 or an IgG4 isotype. Optionally, the Ig constant region or the fragment of the Ig constant region is an IgG1 isotype. Optionally, the Ig constant region or the fragment of the Ig constant region comprises at least one mutation that results in reduced binding of the protein to a Fcγ receptor (FcγR). Optionally, the at least one mutation that results in reduced binding of the protein to the FcγR is selected from the group consisting of F234A/L235A, L234A/L235A, L234A/L235A/D265 S, V234A/G237A/P238 S/H268A/V309L/A330 S/P33IS, F234A/L235A, S228P/F234A/L235A, N297A, V234A/G237A, K214T/E233P/L234V/L235A/G236-deleted/A327G/P331A/D365E/L358M, H268Q/V309L/A330 S/P331 S, S267E/L328F, L234F/L235E/D265A, L234A/L235A/G237A/P238 S/H268A/A330 S/P331 S, S228P/F234A/L235A/G237A/P238 S and S228P/F234A/L235A/G236-deleted/G237A/P238 S, wherein residue numbering is according to the EU index. Optionally, the mutations that results in reduced binding of the protein to the FcγR are L234A_L235A_D265 S.

The disclosure provides an isolated protein comprising an antigen binding region that binds DLL3, wherein the antigen binding region comprises a) a HCDR1, a HCDR2, a HCDR3, a LCDR1, a LCDR2 and a LCDR3 of SEQ ID NOs:15, 16, 17, 33, 34, 35, respectively; b) a VH of SEQ ID NO: 1 and a VL of SEQ ID NO:2; c) a VH of SEQ ID NO:3 and a VL of SEQ ID NO:4; d) a scFv of SEQ ID NO:63; and/or e) a scFv of SEQ ID NO:64. Optionally, the isolated protein comprises an antigen binding region that binds DLL3, wherein the antigen binding region comprises a) a HCDR1, a HCDR2, a HCDR3, a LCDR1, a LCDR2 and a LCDR3 of SEQ ID NOs:15, 16, 17, 33, 34, 35, respectively; and/or b) a VH of SEQ ID NO:1 and a VL of SEQ ID NO:2. Optionally, the isolated protein comprises an antigen binding region that binds DLL3, wherein the antigen binding region comprises a) a HCDR1, a HCDR2, a HCDR3, a LCDR1, a LCDR2 and a LCDR3 of SEQ ID NOs:18, 19, 20, 36, 37, 38, respectively; b) a VH of SEQ ID NO:5 and a VL of SEQ ID NO:6; and/or c) a scFv of SEQ ID NO:65. Optionally, the isolated protein comprises an antigen binding region that binds DLL3, wherein the antigen binding region comprises a) a HCDR1, a HCDR2, a HCDR3, a LCDR1, a LCDR2 and a LCDR3 of SEQ ID NOs:21, 22, 23, 39, 37, 40, respectively; b) a VH of SEQ ID NO:7 and a VL of SEQ ID NO:8; and/or c) a scFv of SEQ ID NO:66. Optionally, the isolated protein comprising an antigen binding region that binds DLL3, wherein the antigen binding region comprises a) a HCDR1, a HCDR2, a HCDR3, a LCDR1, a LCDR2 and a LCDR3 of SEQ ID NOs:24, 25, 26, 41, 42, 43, respectively; b) a VH of SEQ ID NO:9 and a VL of SEQ ID NO:10; and/or c) a scFv of SEQ ID NO:67. Optionally, the isolated protein comprising an antigen binding region that binds DLL3, wherein the antigen binding region comprises a) a HCDR1, a HCDR2, a HCDR3, a LCDR1, a LCDR2 and a LCDR3 of SEQ ID NOs:27, 28, 29, 44, 45, 46, respectively; b) a VH of SEQ ID NO:11 and a VL of SEQ ID NO: 12; and/or c) a scFv of SEQ ID NO:68. Optionally, the isolated protein comprises an antigen binding region that binds DLL3, wherein the antigen binding region comprises a) a HCDR1, a HCDR2, a HCDR3, a LCDR1, a LCDR2 and a LCDR3 of SEQ ID NOs: 30, 31, 32, 47, 48, 49, respectively; b) a VH of SEQ ID NO:13 and a VL of SEQ ID NO:14; and/or c) a scFv of SEQ ID NO:69.

Optionally, the isolated protein is a multispecific antigen-binding construct comprising an antigen binding region that binds CD3E. Optionally, the multispecific antigen-binding construct comprises an antigen binding region that binds CD3ε comprising a) a heavy chain complementarity determining region (HCDR)1 of SEQ ID NO:98, a HCDR2 of SEQ ID NO:99, a HCDR3 of SEQ ID NO: 100, a light chain complementarity determining region (LCDR)1 of SEQ ID NO:106, a LCDR2 of SEQ ID NO:107 and a LCDR3 of SEQ ID NO: 108; and/or b) the VH of SEQ ID NO:84 and the VL of SEQ ID NO:85. Optionally, the multispecific antigen-binding construct comprises an antigen binding region that binds CD3ε comprising a) a heavy chain complementarity determining region (HCDR)1 of SEQ ID NO:95, a HCDR2 of SEQ ID NO:96, a HCDR3 of SEQ ID NO:97, a light chain complementarity determining region (LCDR)1 of SEQ ID NO:101, a LCDR2 of SEQ ID NO: 102 and a LCDR3 of SEQ ID NO:104; and/or b) the VH of SEQ ID NO:77 and the VL of SEQ ID NO:80.

The disclosure provides an isolated anti-DLL3/anti-CD3 protein comprising a first antigen binding region that binds DLL3 and a second antigen binding region that binds a lymphocyte antigen. Optionally, the lymphocyte antigen is a T cell antigen. Optionally, the T cell antigen is a CD8⁺ T cell antigen. Optionally, the lymphocyte antigen is a NK cell antigen. Optionally, the lymphocyte antigen is CD3, CD3 epsilon (CD3E), CD8, KI2L4, NKG2E, NKG2D, NKG2F, BTNL3, CD186, BTNL8, PD-1, CD195, or NKG2C. Optionally, the lymphocyte antigen is CD3E.

Optionally, in an isolated anti-DLL3/anti-CD3 protein, the first antigen binding region that binds DLL3 and/or the second antigen binding region that binds the lymphocyte antigen comprise a scFv, a (scFv)₂, a Fv, a Fab, a F(ab′)₂, a Fd, a dAb or a VHH. Optionally, the first antigen binding region that binds DLL3 and/or the second antigen binding region that binds the lymphocyte antigen comprise the Fab. Optionally, the first antigen binding region that binds DLL3 and/or the second antigen binding region that binds the lymphocyte antigen comprise the scFv. Optionally, the first antigen binding region that binds DLL3 comprises the scFv and the second antigen binding region that binds the lymphocyte antigen comprise the Fab. Optionally, the first antigen binding region that binds DLL3 comprises the Fab and the second antigen binding region that binds the lymphocyte antigen comprise the scFv. Optionally, the scFv comprises, from the N- to C-terminus, a VH, a first linker (L1) and a VL (VH-L1-VL) or the VL, the L1 and the VH (VL-L1-VH). Optionally, the L1 comprises a) about 5-50 amino acids; b) about 5-40 amino acids; c) about 10-30 amino acids; or d) about 10-20 amino acids. Optionally, the L1 comprises the amino acid sequence of SEQ ID NOs:27, 72, 73, 74, 75, 76, 79, 81, 82, 83, 88, 90, 91, 92, 120, 121, 122, 123, 124, 125, 126, 127, 128, 129, 130, 131, 132, 133, 134, 135, 136, 137, 138, or 139. Optionally, the L1 comprises the amino acid sequence of SEQ ID NO:120.

Optionally, in an isolated anti-DLL3/anti-CD3 protein, the first antigen binding region that binds DLL3 comprises a HCDR1 of SEQ ID NOs:15, 18, 21, 24, 27, 30, 50, 52, 53, 55, 57, 59, or 61, a HCDR2 of SEQ ID NOs:16, 19, 22, 25, 28, 31, 51, 54, 56, 58, 60, or 62, a HCDR3 of SEQ ID NOs:17, 20, 23, 26, 29, 32, 17, 20, 23, 26, 29, or 32, a LCDR1 of SEQ ID NOs:33, 36, 39, 41, 44, or 47, a LCDR2 of SEQ ID NOs:34, 37, 42, 45, or 48, and a LCDR3 of SEQ ID NOs:35, 38, 40, 43, 46, or 49. Optionally, the first antigen binding region that binds DLL3 comprises the HCDR1, the HCDR2, the HCDR3, the LCDR1, the LCDR2 and the LCDR3 of a. SEQ ID NOs:15, 16, 17, 33, 34, 35, respectively; b. SEQ ID NOs:18, 19, 20, 36, 37, 38, respectively; c. SEQ ID NOs:21, 22, 23, 39, 37, 40, respectively; d. SEQ ID NOs:24, 25, 26, 41, 42, 43, respectively; e. SEQ ID NOs:18, 28, 29, 44, 45, 46, respectively; f. SEQ ID NOs:30, 31, 32, 47, 48, 49, respectively; g. SEQ ID NOs:50, 51, 17, 33, 34, 35, respectively; h. SEQ ID NOs:52, 51, 17, 33, 34, 35, respectively; i. SEQ ID NOs:53, 54, 20, 36, 37, 38, respectively; j. SEQ ID NOs:55, 56, 23, 39, 37, 40, respectively; k. SEQ ID NOs:57, 58, 26, 41, 42, 43, respectively; 1. SEQ ID NOs:59, 60, 29, 44, 45, 46, respectively; or m. SEQ ID NOs:61, 62, 32, 47, 48, 49, respectively. Optionally, the first antigen binding region that binds DLL3 comprises the HCDR1, the HCDR2, the HCDR3, the LCDR1, the LCDR2 and the LCDR3 of SEQ ID NOs:15, 16, 17, 33, 34, 35, respectively.

In some embodiments, the first antigen binding region that binds DLL3 comprises a. the VH of SEQ ID NO:1 and the VL of SEQ ID NO:2; b. the VH of SEQ ID NO:3 and the VL of SEQ ID NO:4; c. the VH of SEQ ID NO:5 and the VL of SEQ ID NO:6; d. the VH of SEQ ID NO:7 and the VL of SEQ ID NO:8; e. the VH of SEQ ID NO:9 and the VL of SEQ ID NO:10; f. the VH of SEQ ID NO: 11 and the VL of SEQ ID NO: 12; or g. the VH of SEQ ID NO: 13 and the VL of SEQ ID NO:14. Optionally, the first antigen binding region that binds DLL3 comprises the amino acid sequence of SEQ ID NOs:63 or 64. Optionally, the first antigen binding region that binds DLL3 comprises an amino acid sequence at least 80% (e.g., at least 85%, at least 90%, at least 95%, at least 99% or 100%) identical to the amino acid sequence of SEQ ID NO:64. Optionally, the first antigen binding region that binds DLL3 comprises a VH which is at least 80% (e.g., at least 85%, at least 90%, at least 95%, at least 99% or 100%) identical to the VH of SEQ ID NO:3 and a VL which is at least 80% (e.g., at least 85%, at least 90%, at least 95%, at least 99% or 100%) identical to the VL of SEQ ID NO:4. Optionally, the second antigen binding region that binds CD3 comprises a HCDR1 of SEQ ID NOs:95 or 98, a HCDR2 of SEQ ID NOs:96 or 99, a HCDR3 of SEQ ID NOs:97 or 100, a LCDR1 of SEQ ID NOs:101 or 106, a LCDR2 of SEQ ID NOs:102 or 107, and a LCDR3 of SEQ ID NOs:104 or 108. Optionally, the second antigen binding region that binds CD3 comprises a HCDR1 of SEQ ID NO:95, a HCDR2 of SEQ ID NO:96, a HCDR3 of SEQ ID NO:97, a LCDR1 of SEQ ID NO: 101, a LCDR2 of SEQ ID NO: 102, and a LCDR3 of SEQ ID NO: 104. Optionally, the second antigen binding region that binds CD3 comprises a HCDR1 of SEQ ID NO:98, a HCDR2 of SEQ ID NO:99, a HCDR3 of SEQ ID NO: 100, a LCDR1 of SEQ ID NO: 106, a LCDR2 of SEQ ID NO: 107, and a LCDR3 of SEQ ID NO:108. Optionally, the second antigen binding region that binds CD3 comprises a VH which is at least 80% (e.g., at least 85%, at least 90%, at least 95%, at least 99% or 100%) identical to the VH of SEQ ID NO:77 and a VL which is at least 80% (e.g., at least 85%, at least 90%, at least 95%, at least 99% or 100%) identical to the VL of SEQ ID NO:80. Optionally, the second antigen binding region that binds CD3 comprises a VH of SEQ ID NO:77 and a VL of SEQ ID NO:80. Optionally, the second antigen binding region that binds CD3 comprises a VH which is at least 80% (e.g., at least 85%, at least 90%, at least 95%, at least 99% or 100%) identical to the VH of SEQ ID NO:84 and a VL which is at least 80% (e.g., at least 85%, at least 90%, at least 95%, at least 99% or 100%) identical to the VL of SEQ ID NO:85. Optionally, the second antigen binding region that binds the lymphocyte antigen comprises a VH of SEQ ID NO:84 and a VL of SEQ ID NO:85.

In some embodiments, the first antigen binding region that binds DLL3 is conjugated to a first immunoglobulin (Ig) constant region or a fragment of the first Ig constant region and/or the second antigen binding region that binds the lymphocyte antigen is conjugated to a second immunoglobulin (Ig) constant region or a fragment of the second Ig constant region. Optionally, the isolated anti-DLL3/anti-CD3 protein further comprises second linker (L2) between the first antigen binding region that binds DLL3 and the first Ig constant region or the fragment of the first Ig constant region and the second antigen binding region that binds the lymphocyte antigen and the second Ig constant region or the fragment of the second Ig constant region. Optionally, the L2 comprises the amino acid sequence of SEQ ID NOs:27, 72, 73, 74, 75, 76, 79, 81, 82, 83, 88, 90, 91, 92, 120, 121, 122, 123, 124, 125, 126, 127, 128, 129, 130, 131, 132, 133, 134, 135, 136, 137, or 138. Optionally, the fragment of the Ig constant region comprises a Fc region. Optionally, wherein the first Ig constant region or the fragment of the first Ig constant region and the second Ig constant region or the fragment of the second Ig constant region is an IgG1, an IgG2, and IgG3 or an IgG4 isotype. Optionally, the first Ig constant region or the fragment of the first Ig constant region and the second Ig constant region or the fragment of the second Ig constant region is an IgG1. Optionally, the first Ig constant region or the fragment of the first Ig constant region and the second Ig constant region or the fragment of the second Ig constant region comprises at least one mutation that results in reduced binding of the multispecific antigen-binding construct to a FcγR. Optionally, the at least one mutation that results in reduced binding of the multispecific antigen-binding construct to the FcγR is selected from the group consisting of F234A/L235A, L234A/L235A, L234A/L235A/D265 S, V234A/G237A/P238 S/H268A/V309L/A330 S/P331 S, F234A/L235A, S228P/F234A/L235A, N297A, V234A/G237A, K214T/E233P/L234V/L235A/G236-deleted/A327G/P331A/D365E/L358M, H268Q/V309L/A330 S/P331 S, S267E/L328F, L234F/L235E/D265A, L234A/L235A/G237A/P238 S/H268A/A330 S/P331 S, S228P/F234A/L235A/G237A/P238 S and S228P/F234A/L235A/G236-deleted/G237A/P238 S, wherein residue numbering is according to the EU index. Optionally, the mutations that results in reduced binding of the multispecific antigen-binding construct to the FcγR are L234A_L235A_D265 S. Optionally, the protein comprises at least one mutation in a CH3 domain of the Ig constant region. Optionally, the at least one mutation in the CH3 domain of the Ig constant region is selected from the group consisting of T350V, L351Y, F405A, Y407V, T366Y, T366W, F405W, T394W, T394 S, Y407T, Y407A, T366 S/L368A/Y407V, L351Y/F405A/Y407V, T3661/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V and T350V/T366L/K392L/T394W, wherein residue numbering is according to the EU index.

In one general aspect, the application relates to a bispecific antigen-binding construct comprising:
(1) a first antigen binding region that binds DLL3, wherein the first antigen binding region comprises a first VH having a HCDR1, a HCDR2 and a HCDR3, and a first VL having a LCDR1, a LCDR2 and a LCDR3, and the HCDR1, the HCDR2, the HCDR3, the LCDR1, the LCDR2 and the LCDR3 comprise the amino acid sequences of
   (a) SEQ ID NOs:15, 16, 17, 33, 34, 35, respectively;
   (b) SEQ ID NOs:18, 19, 20, 36, 37, 38, respectively;
   (c) SEQ ID NOs:21, 22, 23, 39, 37, 40, respectively;
   (d) SEQ ID NOs:24, 25, 26, 41, 42, 43, respectively;
   (e) SEQ ID NOs:18, 28, 29, 44, 45, 46, respectively;
   (f) SEQ ID NOs:30, 31, 32, 47, 48, 49, respectively;
   (g) SEQ ID NOs:50, 51, 17, 33, 34, 35, respectively;
   (h) SEQ ID NOs:52, 51, 17, 33, 34, 35, respectively;
   (i) SEQ ID NOs:53, 54, 20, 36, 37, 38, respectively;
   (j) SEQ ID NOs:55, 56, 23, 39, 37, 40, respectively;
   (k) SEQ ID NOs:57, 58, 26, 41, 42, 43, respectively;
   (l) SEQ ID NOs:59, 60, 29, 44, 45, 46, respectively; or
   (m)SEQ ID NOs:61, 62, 32, 47, 48, 49, respectively.
(2) a second antigen binding region that binds CD3E, wherein the second antigen binding region comprises:
   (a) a second VH having a HCDR1, a HCDR2 and a HCDR3 of the amino acid sequences of SEQ ID NOs: 95, 96 and 97, respectively, and a second VL having a LCDR1, a LCDR2 and a LCDR3 of the amino acid sequences of SEQ ID NOs: 101, 102 and 104, respectively; or
   (b) a second VH having a HCDR1, a HCDR2 and a HCDR3 of the amino acid sequences of SEQ ID NOs: 98, 99 and 100, respectively, and a second VL having a LCDR1, a LCDR2 and a LCDR3 of the amino acid sequences of SEQ ID NOs: 106, 107 and 108, respectively.

The bispecific antigen-binding construct is referred to herein as an “anti-DLL3/anti-CD3 construct” or “an anti-DLL3/anti-CD3”.

In some embodiments, an isolated anti-DLL3/anti-CD3 protein comprising a first antigen binding region that binds DLL3 and a second antigen binding region that binds CD3, wherein a) the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds the lymphocyte antigen comprises the HCDR1, the HCDR2, the HCDR3, the LCDR1, the LCDR2 and the LCDR3 of SEQ ID NOs:95, 96, 97, 101, 102, 104, respectively; and/or b) the first antigen binding region that binds DLL3 comprises a Fab comprising a VH of SEQ ID NO:1 and a VL of SEQ ID NO:2 and the second antigen binding region that binds CD3 comprises a scFv of SEQ ID NO: 105; and/or c) the isolated anti-DLL3/anti-CD3 protein comprises a HC1 of SEQ ID NO:109, a LC1 of SEQ ID NO:110, and a HC1 of SEQ ID NO:112.

In some embodiments, an isolated anti-DLL3/anti-CD3 protein comprising a first antigen binding region that binds DLL3 and a second antigen binding region that binds CD3, wherein a) the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs:95, 96, 97, 101, 102, 104, respectively; and/or b) the first antigen binding region that binds DLL3 comprises a Fab comprising a VH of SEQ ID NO:1 and a VL of SEQ ID NO:2, and the second antigen binding region that binds CD3 comprises a scFv of SEQ ID NO: 119; and/or c) the isolated anti-DLL3/anti-CD3 protein comprises a HC1 of SEQ ID NO:109, a LC1 of SEQ ID NO: 110, and a HC1 of SEQ ID NO: 113.

In some embodiments, an isolated anti-DLL3/anti-CD3 protein comprising a first antigen binding region that binds DLL3 and a second antigen binding region that binds CD3, wherein a. The first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs:98, 99, 100, 106, 107, 108, respectively; and/or b. the first antigen binding region that binds DLL3 comprises a scFv of SEQ ID NO:63, and the second antigen binding region that binds CD3 comprises a Fab comprising a VH of SEQ ID NO:84 and a VL of SEQ ID NO:85; and/or c. the isolated anti-DLL3/anti-CD3 protein comprises a HC1 of SEQ ID NO:111, a HC2 of SEQ ID NO: 116, and a LC2 of SEQ ID NO: 117.

In some embodiments, an isolated anti-DLL3/anti-CD3 protein comprising a first antigen binding region that binds DLL3 and a second antigen binding region that binds CD3, wherein a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs:95, 96, 97, 101, 102, 104, respectively; and/or b. the first antigen binding region that binds DLL3 comprises a scFv of SEQ ID NO:63 and the second antigen binding region that binds CD3 comprises a Fab comprising a VH of SEQ ID NO:77 and a VL of SEQ ID NO:80; and/or c. the isolated anti-DLL3/anti-CD3 protein comprises a HC1 of SEQ ID NO:111, a HC2 of SEQ ID NO: 114, and a LC2 of SEQ ID NO: 115.

In some embodiments, an isolated anti-DLL3/anti-CD3 protein comprising a first antigen binding region that binds DLL3 and a second antigen binding region that binds CD3, wherein a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs:98, 99, 100, 106, 107, 108, respectively; and/or b. the first antigen binding region that binds DLL3 comprises a scFv of SEQ ID NO:64 and the second antigen binding region that binds the lymphocyte antigen comprises a Fab comprising a VH of SEQ ID NO:84 and a VL of SEQ ID NO:85; and/or c. the isolated anti-DLL3/anti-CD3 protein comprises a HC1 of SEQ ID NO:71, a HC2 of SEQ ID NO:118, and a LC2 of SEQ ID NO:117.

In some embodiments, an isolated anti-DLL3/anti-CD3 protein comprising a first antigen binding region that binds DLL3 and a second antigen binding region that binds CD3, wherein a. The first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs: 98, 99, 100, 106, 107, 108, respectively; and/or b. the first antigen binding region that binds DLL3 comprises a scFv which is at least 80% (e.g., at least 85%, at least 90%, at least 95%, at least 99% or 100%) identical to the scFv of SEQ ID NO:64, and the second antigen binding region that binds CD3 comprises a Fab comprising a VH which is at least 80% (e.g., at least 85%, at least 90%, at least 95%, at least 99% or 100%) identical to the VH of SEQ ID NO:84 and a VL which is at least 80% (e.g., at least 85%, at least 90%, at least 95%, at least 99% or 100%) identical to the VL of SEQ ID NO:85; and/or c. the isolated anti-DLL3/anti-CD3 protein comprises a HC1 which is at least 80% (e.g., at least 85%, at least 90%, at least 95%, at least 99% or 100%) identical to the HC1 of SEQ ID NO:71, a HC2 which is at least 80% (e.g., at least 85%, at least 90%, at least 95%, at least 99% or 100%) identical to the HC2 of SEQ ID NO: 118, and a LC2 which is at least 80% (e.g., at least 85%, at least 90%, at least 95%, at least 99% or 100%) identical to the of SEQ ID NO:117.

In some embodiments, an isolated anti-DLL3/anti-CD3 protein comprising a first antigen binding region that binds DLL3 and a second antigen binding region that binds CD3, wherein a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs:98, 99, 100, 106, 107, 108, respectively; and/or b. the first antigen binding region that binds DLL3 comprises a scFv of SEQ ID NO:64 and the second antigen binding region that binds the lymphocyte antigen comprises a Fab comprising a VH of SEQ ID NO:84 and a VL of SEQ ID NO:85; and/or c. the isolated anti-DLL3/anti-CD3 protein comprises a HC1 of SEQ ID NO:229, a HC2 of SEQ ID NO:230, and a LC2 of SEQ ID NO:117.

In some embodiments, an isolated anti-DLL3/anti-CD3 protein comprising a first antigen binding region that binds DLL3 and a second antigen binding region that binds CD3, wherein a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs: 98, 99, 100, 106, 107, 108, respectively; and/or b. the first antigen binding region that binds DLL3 comprises a scFv which is at least 80% (e.g., at least 85%, at least 90%, at least 95%, at least 99% or 100%) identical to the scFv of SEQ ID NO:64, and the second antigen binding region that binds CD3 comprises a Fab comprising a VH which is at least 80% (e.g., at least 85%, at least 90%, at least 95%, at least 99% or 100%) identical to the VH of SEQ ID NO:84 and a VL which is at least 80% (e.g., at least 85%, at least 90%, at least 95%, at least 99% or 100%) identical to the VL of SEQ ID NO:85; and/or c. the isolated anti-DLL3/anti-CD3 protein comprises a HC1 which is at least 80% (e.g., at least 85%, at least 90%, at least 95%, at least 99% or 100%) identical to the HC1 of SEQ ID NO:229, a HC2 which is at least 80% (e.g., at least 85%, at least 90%, at least 95%, at least 99% or 100%) identical to the HC2 of SEQ ID NO:230, and a LC2 which is at least 80% (e.g., at least 85%, at least 90%, at least 95%, at least 99% or 100%) identical to the of SEQ ID NO:117.

The disclosure also provides an immunoconjugate comprising the isolated antigen binding region that binds DLL3 of the disclosure.

The disclosure also provides an immunoconjugate comprising the isolated protein comprising the antigen binding region that binds DLL3 of the disclosure.

The disclosure also provides an immunoconjugate comprising the isolated multispecific antigen-binding construct comprising the antigen binding region that binds DLL3 of the disclosure.

The disclosure also provides a pharmaceutical composition comprising the isolated antigen binding region that binds DLL3 of the disclosure.

The disclosure also provides a pharmaceutical composition comprising the isolated protein comprising the antigen binding region that binds DLL3 of the disclosure.

The disclosure also provides a pharmaceutical composition comprising the isolated multispecific antigen-binding construct comprising the antigen binding region that binds DLL3 of the disclosure.

The disclosure also provides an isolated polynucleotide encoding the isolated antigen binding region that binds DLL3 of the disclosure.

The disclosure also provides an isolated polynucleotide encoding the isolated protein comprising the antigen binding region that binds DLL3 of the disclosure.

The disclosure also provides an isolated polynucleotide encoding the isolated multispecific antigen-binding construct comprising the antigen binding region that binds DLL3 of the disclosure.

The disclosure also provides a vector comprising the polynucleotide of the disclosure.

The disclosure also provides a host cell comprising the polynucleotide or the vector of the disclosure.

The disclosure also provides a method of treating a DLL3 expressing cancer in a subject, comprising administering a therapeutically effective amount of the antigen binding region that binds DLL3, the protein comprising the antigen binding region that binds DLL3, the multispecific antigen-binding construct comprising the antigen binding region that binds DLL3, the immunoconjugate of the disclosure or the pharmaceutical composition of the disclosure to the subject in need thereof for a time sufficient to treat the DLL3 expressing cancer.

The disclosure also provides a method of reducing the amount of DLL3 expressing tumor cells in a subject, comprising administering to the subject the antigen binding region that binds DLL3, the protein comprising the antigen binding region that binds DLL3, the multispecific antigen-binding construct comprising the antigen binding region that binds DLL3, the immunoconjugate of the disclosure or the pharmaceutical composition of the disclosure for a time sufficient to reduce the amount of DLL3 expressing tumor cells.

The disclosure also provides a method of preventing establishment of a DLL3 expressing cancer in a subject, comprising administering the antigen binding region that binds DLL3, the protein comprising the antigen binding region that binds DLL3, the multispecific antigen-binding construct comprising the antigen binding region that binds DLL3, the immunoconjugate of the disclosure or the pharmaceutical composition of the disclosure to the subject in need thereof to prevent establishment of the DLL3 expressing cancer in the subject.

The disclosure also provides a method of treating a noncancerous condition in a subject at risk of developing a DLL3 expressing cancerous condition, comprising administering the antigen binding region that binds DLL3, the protein comprising the antigen binding region that binds DLL3, the multispecific antigen-binding construct comprising the antigen binding region that binds DLL3, the immunoconjugate of the disclosure or the pharmaceutical composition of the disclosure to the subject in need thereof to treat the noncancerous condition.

The disclosure also provides a method of treating prostate cancer in a subject, comprising administering a therapeutically effective amount of the antigen binding region that binds DLL3, the protein comprising the antigen binding region that binds DLL3, the multispecific antigen-binding construct comprising the antigen binding region that binds DLL3, the immunoconjugate of the disclosure or the pharmaceutical composition of the disclosure to the subject in need thereof for a time sufficient to treat the prostate cancer.

The disclosure also provides a method of treating small cell lung cancer in a subject, comprising administering a therapeutically effective amount of the antigen binding region that binds DLL3, the protein comprising the antigen binding region that binds DLL3, the multispecific antigen-binding construct comprising the antigen binding region that binds DLL3, the immunoconjugate of the disclosure or the pharmaceutical composition of the disclosure to the subject in need thereof for a time sufficient to treat the small cell lung cancer.

The disclosure also provides a method of detecting prostate cancer or small cell lung cancer in a subject, comprising administering to the subject the immunoconjugate of the disclosure, and detecting binding of the immunoconjugate to DLL3, thereby detecting prostate cancer or small cell lung cancer.

The disclosure also provides a kit comprising the antigen binding region that binds DLL3, the protein comprising the antigen binding region that binds DLL3, the multispecific antigen-binding construct comprising the antigen binding region that binds DLL3, the immunoconjugate of the disclosure or the pharmaceutical composition of the disclosure.

The disclosure also provides an anti-idiotypic antibody binding to the antigen binding region that binds DLL3 of the disclosure.

As shown in the Examples, the isolated multispecific antigen-binding constructs disclosed herein may be particularly effective at mediating T cell mediated cytotoxicity, promoting T cell activation and proliferation, increasing T cell cytokine release and/or displaying increased anti-tumor efficacy.

### BRIEF DESCRIPTION OF THE DRAWINGS

The foregoing will be apparent from the following more particular descriptions of example embodiments, as illustrated in the accompanying drawings.

FIG. 1 shows the schematic view of a DLL3 extracellular domain including a DSL domain and 6 EGF domains. The amino acid sequence shown represents residues 176-215 of DSL domain (SEQ ID NO:246), residues 216-249 of EGF-1 domain (SEQ ID NO:247), residues 274-310 of EGF-2 domain (SEQ ID NO:248), residues 312-351 of EGF-3 domain (SEQ ID NO:249), residues 353-389 of EGF-4 domain (SEQ ID NO:250), residues 391-427 of EGF-5 domain (SEQ ID NO:251), residues 429-465 of EGF-6 domain (SEQ ID NO:252) and residues 429-618 of EGF-6 domain+C-terminal domain (SEQ ID NO:263).

FIG. 2A and FIG. 2B show cells binding of bispecific anti-DLL3×CD3 antibodies to DLL3+ tumor cell lines. FIG. 2A shows cells binding of bispecific anti-DLL3×CD3 antibodies to DLL3+ tumor cell lines, SHP77 cells. FIG. 2B shows cells binding of bispecific anti-DLL3×CD3 antibodies to DLL3+ tumor cell lines, HCC1833 cells.

FIG. 3 shows binding of bispecific anti-DLL3×CD3 antibodies on human pan T cells using FACS.

FIG. 4 shows tumor lysis of anti-DLL3×CD3 bispecific antibodies with and without optimized anti-DLL3 sequence evaluated in an IncuCyte-based cytotoxicity assay.

FIG. 5A and FIG. 5B show in vitro target cytotoxicity of bispecific anti-DLL3×CD3 antibodies measured by incuCyte imaging system in real-time for quantifying target cell death.

FIG. 5A shows in vitro target cytotoxicity of anti-DLL3×CD3 bispecific molecules measured by incuCyte imaging system in real-time for quantifying target cell death. Isolated pan-T cells were co-incubated with DLL3+ SHP77 cells in the presence of bispecific anti-DLL3×CD3 antibodies for 120 hours. FIG. 5B shows in vitro target cytotoxicity of anti-DLL3×CD3 bispecific molecules measured by incuCyte imaging system in real-time for quantifying target cell death. Isolated pan-T cells were co-incubated with DLL3⁻ HEK293 cells in the presence of bispecific anti-DLL3×CD3 antibodies for 120 hours.

FIG. 6 shows isolated pan-T cells were co-incubated with DLL3+ SHP77 cells in the presence of bispecific anti-DLL3/CD3 antibodies for 120 hours.

FIG. 7 shows in vitro T cell IFN-γ release by bispecific anti-DLL3×CD3 antibodies. IFN-γ concentration was measured from supernatants collected at the indicated time points.

FIGS. 8A-8C show the cytotoxicity against DLL3+ target cell lines in PBMCs mediated by bispecific anti-DLL3×CD3 antibodies. FIG. 8A shows the cytotoxicity against DLL3+ target cell lines in PBMCs mediated by bispecific anti-DLL3×CD3 antibodies with an E:T ratio of 10:1. FIG. 8B shows the cytotoxicity against DLL3+ target cell lines in PBMCs mediated by bispecific anti-DLL3×CD3 antibodies with an E:T ratio of 5:1. FIG. 8C shows the cytotoxicity against DLL3+ target cell lines in PBMCs mediated by bispecific anti-DLL3×CD3 antibodies with an E:T ratio of 1:1.

FIG. 9 shows proliferation of CD3+ T cells in response to bispecific anti-DLL3×CD3 antibodies in whole PBMC cytotoxicity assay.

FIGS. 10A-10C show activation of T cells in response to bispecific anti-DLL3×CD3 antibodies. FIG. 10A shows activation of T cells in response to bispecific anti-DLL3×CD3 antibodies % CD25⁺ cells. FIG. 10B shows activation of T cells in response to bispecific anti-DLL3×CD3 antibodies % CD69⁺ cells. FIG. 10C shows activation of T cells in response to bispecific anti-DLL3×CD3 antibodies % CD71⁺ cells.

FIG. 11A shows dose response curves for IFNy concentrations at 48 hours.

FIG. 11B shows dose response curves for IFNy concentrations at 120 hours.

FIG. 12A shows dose response curves for CD8+CD25+ T-cells as a percentage of total CD8+ T-cells at 48 hours.

FIG. 12B shows dose response curves for CD8+CD25+ T-cells as a percentage of total CD8+ T-cells at 120 hours.

FIG. 13A shows dose response curves for CD8+ T-cells proliferation at 72 hours.

FIG. 13B shows dose response curves for CD8+ T-cells proliferation at 120 hours.

### DETAILED DESCRIPTION OF THE INVENTION

Various publications, articles, patents and patent applications are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Although any methods and materials similar or equivalent to those described herein can be used in the practice for testing of the present application, exemplary materials and methods are described herein.

Unless defined otherwise, all technical and scientific terms used herein have the same meaning as commonly understood to one of ordinary skill in the art to which this application pertains. Otherwise, certain terms used herein have the meanings as set in the specification. All patents, published patent applications, and publications cited herein are incorporated by reference as if set forth fully herein. It must be noted that as used herein and in the appended claims, the singular forms “a,” “an,” and “the” include plural reference unless the context clearly dictates otherwise. Thus, for example, reference to “a cell” includes a combination of two or more cells, and the like.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Unless otherwise stated, any numerical value, such as a concentration or a concentration range described herein, are to be understood as being modified in all instances by the term “about.” Thus, a numerical value typically includes ±10% of the recited value. For example, a dosage of 10 mg includes 9 mg to 11 mg. As used herein, the use of a numerical range expressly includes all possible subranges, all individual numerical values within that range, including integers within such ranges and fractions of the values unless the context clearly indicates otherwise.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Activation” or “stimulation” or “activated” or “stimulated” refers to induction of a change in the biologic state of a cell resulting in expression of activation markers, cytokine production, proliferation or mediating cytotoxicity of target cells. Cells may be activated by primary stimulatory signals.

“Alternative scaffold” refers to a single chain protein framework that contains a structured core associated with variable domains of high conformational tolerance. The variable domains tolerate variation to be introduced without compromising scaffold integrity, and hence the variable domains can be engineered and selected for binding to a specific antigen.

“Antibody-dependent cellular cytotoxicity”, “antibody-dependent cell-mediated cytotoxicity” or “ADCC” refers to the mechanism of inducing cell death that depends upon the interaction of antibody-coated target cells with effector cells possessing lytic activity, such as natural killer cells (NK), monocytes, macrophages and neutrophils via Fc gamma receptors (FcγR) expressed on effector cells.

“Antibody-dependent cellular phagocytosis” or “ADCP” refers to the mechanism of elimination of antibody-coated target cells by internalization by phagocytic cells, such as macrophages or dendritic cells.

“Antigen” refers to any molecule (e.g., protein, peptide, polysaccharide, glycoprotein, glycolipid, nucleic acid, portions thereof, or combinations thereof) capable of being bound by an antigen binding region or a T-cell receptor that is capable of mediating an immune response. Exemplary immune responses include antibody production and activation of immune cells, such as T cells, B cells or NK cells. Antigens may be expressed by genes, synthetized, or purified from biological samples such as a tissue sample, a tumor sample, a cell or a fluid with other biological components, organisms, subunits of proteins/antigens, killed or inactivated whole cells or lysates.

An “antigen binding region” or “antigen binding fragment” or “antigen binding domain” each refers to a portion of a full-length antibody that binds an antigen. An antigen binding region can be synthetic, enzymatically obtainable or genetically engineered polypeptides. An antigen binding region typically comprises one or more portions of at least the VH region. Antigen-binding fragments include multivalent molecules comprising one, two, three, or more antigen-binding portions of an antibody, and single-chain constructs wherein the VL and VH regions, or selected portions thereof, are joined by synthetic linkers or by recombinant methods to form a functional, antigen-binding molecule. Antigen-binding fragments can also be a single-domain antibody (sdAb), also known as a nanobody, which is an antibody fragment consisting of a single monomeric variable antibody domain (VHH). Examples of antigen-binding fragments include Fab, Fab′, F(ab)₂, F(ab′)₂, F(ab)₃, Fv (typically the VL and VH domains of a single arm of an antibody), single-chain Fv (scFv, see e.g., Bird et al., Science 1988; 242:423-426; and Huston et al. PNAS 1988; 85:5879-5883), dsFv, Fd (typically the VH and CH1 domain), and dAb (typically a VH domain) fragments; VH, VL, VHH, and V-NAR domains; monovalent molecules comprising a single VH and a single VL chain; minibodies, diabodies, triabodies, tetrabodies, and kappa bodies (see, e.g., Ill et al., Protein Eng 1997; 10:949-57); camel IgG; IgNAR; as well as one or more isolated CDRs or a functional paratope, where the isolated CDRs or antigen-binding residues or polypeptides can be associated or linked together so as to form a functional antibody fragment, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific antigen-binding constructs comprising the antigen binding regions. Various types of antibody fragments have been described or reviewed in, e.g., Holliger and Hudson, Nat Biotechnol 2005; 23:1126-1136; WO2005040219, and published U.S. Patent Applications 20050238646 and 20020161201. Antibody fragments can be obtained using conventional recombinant or protein engineering techniques, and the fragments can be screened for antigen-binding or other function in the same manner as are intact antibodies. Various techniques have been developed for the production of antibody fragments. Traditionally, these fragments were derived via proteolytic digestion of full-length antibodies (see, e.g., Morimoto et al., Journal of Biochemical and Biophysical Methods, 24:107-117 (1992); and Brennan et al., Science, 229:81 (1985)). However, these fragments can now be produced directly by recombinant host cells. Alternatively, Fab′-SH fragments can be directly recovered from E. coli and chemically coupled to form F(ab′)2 fragments (Carter et al., Bio/Technology, 10:163-167 (1992)). According to another approach, F(ab′)2 fragments can be isolated directly from recombinant host cell culture. In other embodiments, the antibody of choice is a single-chain Fv fragment (scFv). See WO 1993/16185; U.S. Pat. Nos. 5,571,894 and 5,587,458. The antibody fragment may also be a “linear antibody”, e.g., as described in U.S. Pat. No. 5,641,870, for example. Such linear antibody fragments can be monospecific or bispecific. Antigen binding regions (such as VH and VL) cane be linked together via a synthetic linker to form various types of single antibody designs where the VH/VL domains may pair intramolecularly, or intermolecularly in those cases when the VH and VL domains are expressed by separate single chains, to form a monovalent antigen binding region, such as single chain Fv (scFv) or diabody. Antigen binding regions can also be conjugated to other antibodies, proteins, antigen binding regions or alternative scaffolds which may be monospecific or multispecific to engineer bispecific and multispecific antigen-binding constructs.

“Antibody” or “Antibodies” is meant in a broad sense and includes immunoglobulin molecules including polyclonal antibodies, monoclonal antibodies including murine, human, humanized, chimeric monoclonal antibodies, antigen binding regions, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions can be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins can be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (k), based on the amino acid sequences of their constant domains. General principles of antibody molecule structure and various techniques relevant to the production of antibodies are provided in, e.g., Harlow and Lane, ANTIBODIES: A LABORATORY MANUAL, Cold Spring Harbor Laboratory Press, Cold Spring Harbor, N. Y., (1988).

An isolated protein or construct of the application can also comprise an antibody derivative. The term “antibody derivative” as used herein refers to a molecule comprising a full-length antibody or an antigen-binding fragment thereof, wherein one or more amino acids are chemically modified or substituted. Chemical modifications that can be used in antibody derivative includes, e.g., alkylation, PEGylation, acylation, ester formation or amide formation or the like, e.g., for linking the antibody to a second molecule. Exemplary modifications include PEGylation (e.g., cysteine-PEGylation), biotinylation, radiolabeling, and conjugation with a second agent (such as a cytotoxic agent).

Antibodies herein include “amino acid sequence variants” with altered antigen-binding or biological activity. Examples of such amino acid alterations include antibodies with enhanced affinity for antigen (e.g. “affinity matured” antibodies), and antibodies with altered Fc region, if present, e.g. with altered (increased or diminished) antibody dependent cellular cytotoxicity (ADCC) and/or complement dependent cytotoxicity (CDC) (see, for example, WO 00/42072, Presta, L. and WO 99/51642, Iduosogie et al); and/or increased or diminished serum half-life (see, for example, WO00/42072, Presta, L.).

A “bispecific antigen-binding construct” or “bispecific construct” refers to a construct that specifically binds two distinct antigens or two distinct epitopes within the same antigen. A bispecific antigen-binding construct can be a protein, a protein complex, or an antibody. The bispecific construct can have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

A “bispecific anti-DLL3/anti-CD3 antibody,” “anti-DLL3×CD3,” “DLL3/CD3 antibody,” “DLL3×CD3 antibody,” “anti-DLL3/anti-CD3 protein”, and the like refer to a construct or antibody that binds DLL3 and CD3 and that comprises at least one binding domain specifically binding DLL3 and at least one binding domain specifically binding CD3. The domains specifically binding DLL3 and CD3 are typically V_{H}/V_{L} pairs. The bispecific anti-DLL3×CD3 antibody can be monovalent in terms of its binding to either DLL3 or CD3.

The term “hypervariable region” when used herein refers to the amino acid residues of an antibody that are responsible for antigen binding. The hypervariable region generally comprises amino acid residues from a “complementarity-determining region” or “CDR” (residues 24-34 (L1), 50-56 (L2) and 89-97 (L3) in the light-chain variable domain and 31-35 (H1), 50-65 (H2) and 95-102 (H3) in the heavy-chain variable domain; (Kabat et al. (1991) Sequences of Proteins of Immunological Interest, Fifth Edition, U.S. Department of Health and Human Services, NIH Publication No. 91-3242) and/or those residues from a “hypervariable loop” (residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol. 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide can contain fewer or additional amino acids corresponding to a shortening of, or insertion into, a FR or CDR of the variable domain. For example, a heavy chain variable domain can include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

The term “administering” with respect to the methods of the invention, means a method for therapeutically or prophylactically preventing, treating or ameliorating a syndrome, disorder or disease as described herein by using a conjugate of the invention or a form, composition or medicament thereof. Such methods include administering an effective amount of said antibody, antigen-binding fragment thereof, or conjugate, or a form, composition or medicament thereof at different times during the course of a therapy or concurrently in a combination form. The methods of the invention are to be understood as embracing all known therapeutic treatment regimens.

The ability of a target antibody to “block” the binding of a target molecule to a natural target ligand, means that the antibody, in an assay using soluble or cell-surface associated target and ligand molecules, can detectably reduce the binding of a target molecule to the ligand in a dose-dependent fashion, where the target molecule detectably binds to the ligand in the absence of the antibody.

“Cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

“Complement-dependent cytotoxicity” or “CDC”, refers to the mechanism of inducing cell death in which the Fc effector domain of a target-bound protein binds and activates complement component C1q which in turn activates the complement cascade leading to target cell death. Activation of complement may also result in deposition of complement components on the target cell surface that facilitate CDC by binding complement receptors (e.g., CR3) on leukocytes.

“Complementarity determining regions” (CDR) are antibody regions that bind an antigen.

There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132: 211-50; Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton J Bmol Biol 263: 800-15, 1996). The correspondence between the various delineations and variable region numbering is described (see e.g., Lefranc et al. (2003) Dev Comp Immunol 27: 55-77; Honegger and Pluckthun (2001), J Mol Biol 309:657-70; International ImMunoGeneTics (IMGT) database; Web resources, http://www_imgt_org). Available programs such as abYsis by UCL Business PLC may be used to delineate CDRs. The terms “CDR”, “HCDR1”, “HCDR2”, “HCDR3”, “LCDR1”, “LCDR2” and “LCDR3” as used herein include CDRs defined by any of the methods described supra, Kabat, Chothia, IMGT or AbM, unless otherwise explicitly stated in the specification. Correspondence between the numbering system, including, for example, the Kabat numbering and the IMGT unique numbering system, is well known to one skilled in the art (see, e.g., Kabat, supra; Chothia, supra; Martin, supra; Lefranc et al., supra).

TABLE 1

| | IMGT | Kabat | AbM | Chothia |
|---|---|---|---|---|
| V_{H} CDR1 | 27-38 | 31-35 | 26-35 | 26-32 |
| V_{H} CDR2 | 56-65 | 50-65 | 50-58 | 53-55 |
| V_{H} CDR3 | 105-117 | 95-102 | 95-102 | 96-101 |
| V_{L} CDR1 | 27-38 | 24-34 | 24-34 | 26-32 |
| V_{L} CDR2 | 56-65 | 50-56 | 50-56 | 50-52 |
| V_{L} CDR3 | 105-117 | 89-97 | 89-97 | 91-96 |

“CD3” refers to an antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. Human CD3 epsilon comprises the amino acid sequence of SEQ ID NO:253. All references to proteins, polypeptides and protein fragments herein are intended to refer to the human version of the respective protein, polypeptide or protein fragment unless explicitly specified as being from a non-human species. Thus, “CD3” means human CD3 unless specified as being from a non-human species, e.g., “mouse CD3,” “monkey CD3,” etc.

Throughout the specification, “CD3-specific” or “specifically binds CD3” or “anti-CD3 antibody” refers to antibodies that bind specifically to the CD3-epsilon polypeptide (SEQ ID NO:253), including antibodies that bind specifically to the CD3-epsilon extracellular domain (ECD) (SEQ ID NO:254). CD3-epsilon, together with CD3-gamma, -delta and-zeta, and the T-cell receptor alpha/beta and gamma/delta heterodimers, forms the T-cell receptor-CD3 complex. This complex plays an important role in coupling antigen recognition to several intracellular signal-transduction pathways. The CD3 complex mediates signal transduction, resulting in T cell activation and proliferation. CD3 is required for the immune response.

A “conjugate” as used herein refer to a protein covalently linked to one or more heterologous molecule(s), including but not limited to a therapeutic peptide or protein, an antibody, a label, or a neurological disorder drug. When one protein is conjugated to another protein, it is also referred to the two proteins are fused together. By way of a non-limiting example, an antibody or antigen-binding fragment of the application can be conjugated to another polypeptide to form a fusion protein. In certain embodiments, an antibody or antigen-binding fragment of the application can be fused or conjugated to another polypeptide through a linker.

“Decrease,” “lower,” “lessen,” “reduce,” or “abate” refers generally to the ability of a test molecule to mediate a reduced response (i.e., downstream effect) when compared to the response mediated by a control or a vehicle. Exemplary responses are T cell expansion, T cell activation or T-cell mediated tumor cell killing or binding of a protein to its antigen or receptor, enhanced binding to a Fey or enhanced Fc effector functions such as enhanced ADCC, CDC and/or ADCP. Decrease may be a statistically significant difference in the measured response between the test molecule and the control (or the vehicle), or a decrease in the measured response, such as a decrease of about 1.1, 1.2, 1.5, 2, 3, 4, 5, 6, 7, 8, 9, 10, 15, 20 or 30 fold or more, such as 500, 600, 700, 800, 900 or 1000 fold or more (including all integers and decimal points in between and above 1, e.g., 1.5, 1.6, 1.7. 1.8, etc.).

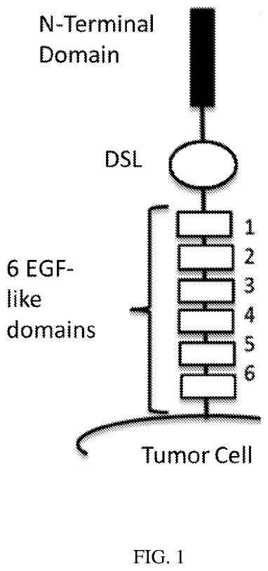

“Delta-like protein 3” or “DLL3” refers to a known protein which is also called delta-like 3, delta 3, or drosophila Delta homolog 3. Unless specified, as used herein, DLL3 refers to human DLL3. All DLL3 isoforms and variants are encompassed in “DLL3”. The amino acid sequences of the various isoforms are retrievable from databases, such as NCBI accession numbers NP_058637.1 (isoform 1 precursor, 618 amino acids) and NP 982353.1(isoform 2 precursor, 587 amino acids). The amino acid sequence of a full length human DLL3 is shown in SEQ ID NO:255. The sequence of DLL3 includes the DSL domain (residues 176-215), EGF-1 domain (residues 216-249), EGF-2 domain (residues 274-310), EGF-3 domain (residues 312-351), EGF-4 domain (residues 353-389), EGF-5 domain (residues 391-427), EGF-6 domain (residues 429-465) and C-terminal domain (residues 466-618) (FIG. 1). The amino acid sequence of the DLL3 DSL domain is shown in SEQ ID NO: 246. The amino acid sequence of the DLL3 EGF-1 domain is shown in SEQ ID NO:247. The amino acid sequence of the DLL3 EGF-2 domain is shown in SEQ ID NO:248. The amino acid sequence of the DLL3 EGF-3 domain is shown in SEQ ID NO:249. The amino acid sequence of the DLL3 EGF-4 domain is shown in SEQ ID NO:250. The amino acid sequence of the DLL3 EGF-5 domain is shown in SEQ ID NO:251. The amino acid sequence of the DLL3 EGF-6 domain is shown in SEQ ID NO:252. The amino acid sequence of the DLL3 EGF-6+C-terminal domain is shown in SEQ ID NO:263.

“Differentiation” refers to a method of decreasing the potency or proliferation of a cell or moving the cell to a more developmentally restricted state.

“Encode” or “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA.

“Enhance,” “promote,” “increase,” “expand” or “improve” refers generally to the ability of a test molecule to mediate a greater response (i.e., downstream effect) when compared to the response mediated by a control or a vehicle. Exemplary responses are T cell expansion, T cell activation or T-cell mediated tumor cell killing or binding of a protein to its antigen or receptor, enhanced binding to a Fcγ or enhanced Fc effector functions such as enhanced ADCC, CDC and/or ADCP. Enhance may be a statistically significant difference in the measured response between the test molecule and control (or vehicle), or an increase in the measured response, such as an increase of about 1.1, 1.2, 1.5, 2, 3, 4, 5, 6, 7, 8, 9, 10, 15, 20 or 30 fold or more, such as 500, 600, 700, 800, 900 or 1000 fold or more (including all integers and decimal points in between and above 1, e.g., 1.5, 1.6, 1.7. 1.8, etc.).

“Epitope” refers to a portion of an antigen to which an antibody, or the antigen binding portion thereof, specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontinuous amino acids that form a conformational spatial unit. For a discontinuous epitope, amino acids from differing portions of the linear sequence of the antigen come in close proximity in 3-dimensional space through the folding of the protein molecule. Antibody “epitope” depends on the methodology used to identify the epitope.

“Expansion” refers to the outcome of cell division and cell death.

“Express” and “expression” refers to the well-known transcription and translation occurring in cells or in vitro. The expression product, e.g., the protein, is thus expressed by the cell or in vitro and may be an intracellular, extracellular or a transmembrane protein.

“Expression vector” refers to a vector that can be utilized in a biological system or in a reconstituted biological system to direct the translation of a polypeptide encoded by a polynucleotide sequence present in the expression vector.

“dAb” or “dAb fragment” refers to an antibody fragment composed of a VH domain (Ward et al. (1989), Nature 341:544 546).

“Fab” or “Fab fragment” refers to an antibody fragment composed of VH, CH1, VL and CL domains.

“F(ab′)₂” or “F(ab′)₂ fragment” refers to an antibody fragment containing two Fab fragments connected by a disulfide bridge in the hinge region.

“Fd” or “Fd fragment” refers to an antibody fragment composed of VH and CH1 domains.

“Fv” or “Fv fragment” refers to an antibody fragment composed of the VH and the VL domains from a single arm of the antibody. Fv fragments lack the constant regions of Fab (CH1 and CL) regions. The VH and VL in Fv fragments are held together by non-covalent interactions.

“Framework region” or “FR” residues are those VH or VL residues other than the CDRs as herein defined.

“Full length antibody” is comprised of two heavy chains (HC) and two light chains (LC) inter-connected by disulfide bonds as well as multimers thereof (e.g., IgM). Each heavy chain is comprised of a heavy chain variable domain (VH) and a heavy chain constant domain, the heavy chain constant domain comprised of subdomains CH1, hinge, CH2 and CH3. Each light chain is comprised of a light chain variable domain (VL) and a light chain constant domain (CL). The VH and the VL may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Genetic modification” refers to the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

“Heterologous” refers to two or more polynucleotides or two or more polypeptides that are not found in the same relationship to each other in nature.

“Heterologous polynucleotide” refers to a non-naturally occurring polynucleotide that encodes two or more neoantigens as described herein.

“Heterologous polypeptide” refers to a non-naturally occurring polypeptide comprising two or more neoantigen polypeptides as described herein.

“Host cell” refers to any cell that contains a heterologous nucleic acid. An exemplary heterologous nucleic acid is a vector (e.g., an expression vector).

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“In combination with” means that two or more therapeutic agents are to be administered to a subject together in a mixture, concurrently as single agents or sequentially as single agents in any order.

“Isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step.

“Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity. An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al, J. Chromatogr. B 848:79-87 (2007).

A “linker” as used herein refers to a chemical linker or a single chain peptide linker that covalently connects two different entities. A linker can be used to connect any two of an antibody or a fragment thereof, a fusion protein and a conjugate of the present invention. The linker can connect, for example, the VH and VL in scFv, or the monoclonal antibody or antigen-binding fragment thereof with a therapeutic molecule, such as a second antibody. Single chain peptide linkers, comprised of from 1 to 25 amino acids, such as 1, 2, 3, 4, 5, 6, 7, 8, 9, 10, 11, 12, 13, 14, 15, 16, 17, 18, 19, 20, 21, 22, 23, 24, or 25 amino acids, joined by peptide bonds, can be used. In certain embodiments, the amino acids are selected from the twenty naturally occurring amino acids. In certain other embodiments, one or more of the amino acids are selected from glycine, alanine, proline, asparagine, glutamine and lysine. Chemical linkers, such as a hydrocarbon linker, a polyethylene glycol (PEG) linker, a polypropylene glycol (PPG) linker, a polysaccharide linker, a polyester linker, a hybrid linker consisting of PEG and an embedded heterocycle, and a hydrocarbon chain can also be used.

“Modulate” refers to either enhanced or decreased ability of a test molecule to mediate an enhanced or a reduced response (i.e., downstream effect) when compared to the response mediated by a control or a vehicle.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. A bispecific monoclonal antibody binds two distinct antigenic epitopes. Monoclonal antibodies may have heterogeneous glycosylation within the antibody population. Monoclonal antibodies may be monospecific or multispecific such as bispecific, monovalent, bivalent or multivalent.

A “multispecific antigen-binding construct” or “multispecific molecules” refers to a construct that specifically binds more than one distinct antigens or more than one distinct epitopes within the same antigen. A multispecific antigen-binding construct can be a protein, a protein complex, or an antibody. It comprises an antibody, or an antigen-binding fragment thereof, which is associated with or linked to at least one other functional molecule (e.g. another peptide or protein such as another antibody or ligand for a receptor) thereby forming a molecule that binds to at least two different binding sites or target molecules. Multispecific molecule may have cross-reactivity to other related antigens, for example to the same antigen from other species (homologs), such as human or monkey, for example Macaca fascicularis (cynomolgus, cyno) or Pan troglodytes, or may bind an epitope that is shared between two or more distinct antigens. Exemplary multispecific molecules include tr-specific or bi-specific antibodies and antibodies linked to soluble receptor fragments or ligands.

“Natural killer cell” and “NK cell” are used interchangeably and synonymously herein. NK cell refers to a differentiated lymphocyte with a CD16+ CD56+ and/or CD57⁺ TCR⁻ phenotype. NK cells are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

“Operatively linked” and similar phrases, when used in reference to nucleic acids or amino acids, refer to the operational linkage of nucleic acid sequences or amino acid sequences, respectively, placed in functional relationships with each other. For example, an operatively linked promoter, enhancer elements, open reading frame, 5′ and 3′ UTR, and terminator sequences result in the accurate production of a nucleic acid molecule (e.g., RNA) and in some instances to the production of a polypeptide (i.e., expression of the open reading frame). Operatively linked peptide refers to a peptide in which the functional domains of the peptide are placed with appropriate distance from each other to impart the intended function of each domain.

The term “paratope” refers to the area or region of an antibody molecule which is involved in binding of an antigen and comprise residues that interact with an antigen. A paratope may composed of continuous and/or discontinuous amino acids that form a conformational spatial unit. The paratope for a given antibody can be defined and characterized at different levels of details using a variety of experimental and computational methods. The experimental methods include hydrogen/deuterium exchange mass spectrometry (HX-MS). The paratope will be defined differently depending on the mapping method employed. A paratope can comprise amino acid residues directly involved in epitope binding, several of which are typically in CDRs, and other amino acid residues, which are not directly involved in the binding, such as amino acid residues which are effectively blocked by the specifically bound antigen (in other words, the amino acid residue is within the “solvent-excluded surface” and/or “footprint” of the specifically bound antigen).

“Pharmaceutical combination” refers to a combination of two or more active ingredients administered either together or separately.

“Pharmaceutical composition” refers to a composition that results from combining an active ingredient and a pharmaceutically acceptable carrier.

“Pharmaceutically acceptable carrier” or “excipient” refers to an ingredient in a pharmaceutical composition, other than the active ingredient, which is nontoxic to a subject. Exemplary pharmaceutically acceptable carriers are a buffer, stabilizer or preservative.

“Polynucleotide” or “nucleic acid” refers to a synthetic molecule comprising a chain of nucleotides covalently linked by a sugar-phosphate backbone or other equivalent covalent chemistry. cDNA is a typical example of a polynucleotide. Polynucleotide may be a DNA or a RNA molecule.

“Prevent,” “preventing,” “prevention,” or “prophylaxis” of a disease or disorder means preventing that a disorder occurs in a subject.

“Proliferation” refers to an increase in cell division, either symmetric or asymmetric division of cells.

“Promoter” refers to the minimal sequences required to initiate transcription. Promoter may also include enhancers or repressor elements which enhance or suppress transcription, respectively.

“Protein” or “polypeptide” are used interchangeably herein and refers to a molecule that comprises one or more polypeptides each comprised of at least two amino acid residues linked by a peptide bond. Protein may be a monomer, or may be protein complex of two or more subunits, the subunits being identical or distinct. Small polypeptides of less than 50 amino acids may be referred to as “peptides”. Protein may be a heterologous fusion protein, a glycoprotein, or a protein modified by post-translational modifications such as phosphorylation, acetylation, myristoylation, palmitoylation, glycosylation, oxidation, formylation, amidation, citrullination, polyglutamylation, ADP-ribosylation, pegylation or biotinylation. Protein may be recombinantly expressed.

“Recombinant” refers to polynucleotides, polypeptides, vectors, viruses and other macromolecules that are prepared, expressed, created or isolated by recombinant means.

“Regulatory element” refers to any cis- or trans acting genetic element that controls some aspect of the expression of nucleic acid sequences.

“Relapsed” refers to the return of a disease or the signs and symptoms of a disease after a period of improvement after prior treatment with a therapeutic.

“Refractory” refers to a disease that does not respond to a treatment. A refractory disease can be resistant to a treatment before or at the beginning of the treatment, or a refractory disease can become resistant during a treatment.

The phrases “sequence identity” or “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 91%, 92%, 93%, 94%, 95%, 97%, 98%, 99%, or 100% identity over the full-length of the amino acid sequences) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g. using the NCBI BLAST algorithm (Altschul S F, et al (1997), Nucleic Acids Res. 25:3389-3402).

“Single chain Fv” or “scFv” refers to a fusion protein comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. Unless specified, as used herein, a scFv may have the VL and VH variable regions in either order, e.g., with respect to the N-terminal and C-terminal ends of the polypeptide, the scFv may comprise VL-linker-VH or may comprise VH-linker-VL.

“(scFv)₂” or “tandem scFv” or “bis-scFv” fragments refers to a fusion protein comprising two light chain variable regions (VL) and two heavy chain variable regions (VH), wherein the two VL and the two VH regions are contiguously linked via polypeptide linkers, and capable of being expressed as a single chain polypeptide. The two VL and two VH regions fused by peptide linkers form a bivalent molecule VL_{A}-linker-VH_{A}-linker-VL_{B}-linker-VH_{B} to form two binding sites, capable of binding two different antigens or epitopes concurrently.

“Specifically binds,” “specific binding,” “specifically binding” or “binds” refer to a proteinaceous molecule binding to an antigen or an epitope within the antigen with greater affinity than for other antigens. Typically, the proteinaceous molecule binds to the antigen or the epitope within the antigen with an equilibrium dissociation constant (K_{D}) of about 1×10⁻⁷ M or less, for example about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a non-specific antigen (e.g., BSA, casein).

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“T cell” and “T lymphocyte” are interchangeable and used synonymously herein. T cell includes thymocytes, naïve T lymphocytes, memory T cells, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD⁴⁺ T cell) CD⁴⁺ T cell, a cytotoxic T cell (CTL; CD8⁺ T cell), a tumor infiltrating cytotoxic T cell (TIL; CD8⁺ T cell), CD⁴⁺ CD8⁺ T cell, or any other subset of T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1⁺ and NK1.1⁻, as well as CD⁴⁺, CD4⁻, CD8⁺ and CD8⁻ cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation and were found to be an important source of IL-17 and to induce robust CD8⁺ cytotoxic T cell response. Also included are “regulatory T cells” or “Tregs” which refer to T cells that suppress an abnormal or excessive immune response and play a role in immune tolerance. Tregs are typically transcription factor Foxp3-positive CD⁴⁺ T cells and can also include transcription factor Foxp3-negative regulatory T cells that are IL-10-producing CD⁴⁺ T cells.

“Therapeutically effective amount” or “effective amount” used interchangeably herein, refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics that include, for example, improved well-being of the patient, reduction of a tumor burden, arrested or slowed growth of a tumor, and/or absence of metastasis of cancer cells to other locations in the body.

“Transduction” refers to the introduction of a foreign nucleic acid into a cell using a viral vector.

“Treat,” “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene.

Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

“Variant,” “mutant” or “altered” refers to a polypeptide or a polynucleotide that differs from a reference polypeptide or a reference polynucleotide by one or more modifications, for example one or more substitutions, insertions or deletions.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated.

“VHH” refers to a single-domain antibody or nanobody, exclusively composed of the antigen binding region of a heavy chain. A VHH single domain antibody lacks the light chain and the CH1 domain of the heavy chain of conventional Fab region.

### Compositions of Matter

Antigen Binding Regions that Bind DLL3

The disclosure provides antigen binding regions that bind DLL3, monospecific and multispecific antigen-binding constructs comprising the antigen binding regions that bind DLL3, polynucleotides encoding the foregoing, vectors, host cells and methods of making and using the foregoing. The antigen binding regions that bind DLL3 identified herein demonstrated improved properties in terms of improved thermostability. The multispecific antigen-binding constructs disclosed herein can be particularly effective at mediating T cell mediated cytotoxicity, promoting T cell activation and proliferation, increasing T cell cytokine release and/or displaying increased anti-tumor efficacy.

The disclosure provides an isolated protein comprising an antigen binding region that binds delta-like protein 3 (DLL3), wherein the antigen binding region that binds DLL3 binds to an epitope within the EGF-6+C-terminal domain of DLL3 set forth in SEQ ID NO:263 (residues 429-618 of DLL3). As shown in the examples, multispecific antigen-binding constructs targeting an epitope within the EGF-6 domain or closer to the C-terminus of DLL3 achieved potent levels of anti-tumor cytotoxicity.

Any method known in the art can be used to identify the region within DLL3 an antibody of the application binds in view of the present disclosure. For example, an ELISA assay can be used to identify the domain(s) within DLL3 to which an antibody binds. In a domain mapping ELISA assay, anti-DLL3 antibodies were evaluated for binding to recombinant DLL3 domain antigens spanning the N-terminal DSL fusion domain (DL3W44, SEQ ID NO: 189), EGF-1+2 fusion (DL3W42, SEQ ID NO:187), EGF-2 (DL3W41, SEQ ID NO:186), EGF-3 (DL3W40, SEQ ID NO:185), EGF-4 (DL3W39, SEQ ID NO:184), EGF-5 (DL3W38, SEQ ID NO:183), EGF-6 (DL3W37, SEQ ID NO: 182) and EGF-6+C-terminal domain fusion (DL3W36, SEQ ID NO:181). MesoScale Discovery high bind plates were coated overnight at 4° C. with 20 nM antigen. The plates were washed with PBS with 0.1% Tween and then blocked with Starting block solution for 30 minutes. The antibodies were added and incubated for 60 minutes at ambient temperature and then excess antibodies were removed by washing 3 times with PBS (Gibco, #14190-136). Antigen bound antibody was detected with sulfo-tagged anti-human antibody (Meso Scale Discovery, R32AJ) for 60 minutes at ambient temperature followed by another PBS wash. Signal acquisition was done in the presence of 1× MSD read buffer T (MSD, Cat #R92TC-1) on the MSD Sector 600 imager with appropriate plate settings. Data was analyzed for the highest binding signal per domain indicating the preferential domain binding.

An H/D exchange assay can be used to determine the residues within DLL3 to which an antibody binds. In an H/D exchange assay, recombinantly expressed soluble DLL3 is incubated in the presence or absence of the antibody in deuterated water for predetermined times resulting in deuterium incorporation at exchangeable hydrogen atoms which are unprotected by the antibody, followed by protease digestion of the protein and analyses of the peptide fragments using LC-MS. H/D exchange assay can be performed using known protocols. In some embodiments, the H/D exchange mixture is quenched by the addition of a quenching buffer (e.g., 8 M urea, 1M TCEP, pH 3.0) before being passed over an equilibrated immobilized pepsin/FPXIII column at room temperature (e.g., 600 μL/min). The peptic fragments are then loaded onto a reverse phase trap column (e.g., at 600 μL/min) and desalted (e.g., for 1 min at 600 μL), separated (e.g., on a C18 column) and analyzed by mass spectrometry (e.g., using an LTQ™ Orbitrap Fusion Lumos mass spectrometer (Thermo Fisher Scientific) with the capillary temperature at 275° C., resolution 150,000, and mass range (m/z) 300-1,800).

In some embodiments, the application provides an isolated protein, such as an antibody, comprising an antigen binding region, wherein the antigen binding region that binds DLL3 competes for binding to DLL3 with a reference antibody disclosed herein. In some embodiments, the reference antibody comprises a VH having a HCDR1, a HCDR2 and a HCDR3, and a VL having a LCDR1, a LCDR2 and a LCDR3, and the HCDR1, HCDR2, HCDR3, LCDR1, LCDR2 and LCDR3 are:
a. the HCDR1, the HCDR2 and the HCDR3 of a VH of SEQ ID NO:1 and the LCDR1, the LCDR2 and the LCDR3 of a VL of SEQ ID NO:2;
b. the HCDR1, the HCDR2 and the HCDR3 of a VH of SEQ ID NO:3 and the LCDR1, the LCDR2 and the LCDR3 of a VL of SEQ ID NO:4;
c. the HCDR1, the HCDR2 and the HCDR3 of a VH of SEQ ID NO:5 and the LCDR1, the LCDR2 and the LCDR3 of a VL of SEQ ID NO:6;
d. the HCDR1, the HCDR2 and the HCDR3 of a VH of SEQ ID NO:7 and the LCDR1, the LCDR2 and the LCDR3 of a VL of SEQ ID NO:8;
e. the HCDR1, the HCDR2 and the HCDR3 of a VH of SEQ ID NO:9 and the LCDR1, the LCDR2 and the LCDR3 of a VL of SEQ ID NO: 10;
f. the HCDR1, the HCDR2 and the HCDR3 of a VH of SEQ ID NO:11 and the LCDR1, the LCDR2 and the LCDR3 of a VL of SEQ ID NO: 12; or
g. the HCDR1, the HCDR2 and the HCDR3 of a VH of SEQ ID NO:13 and the LCDR1, the LCDR2 and the LCDR3 of a VL of SEQ ID NO: 14.

In certain such embodiments, the reference antibody comprises a HCDR1, a HCDR2 and a HCDR3 of a VH of SEQ ID NO:3 and a LCDR1, a LCDR2 and a LCDR3 of a VL of SEQ ID NO:4.

Competition for binding of a test antibody that binds to SEQ ID NO:263 of soluble DLL3 with a reference antibody of the application can be assayed in vitro using well known methods in view of the present disclosure. For example, binding of labeled antibody to DLL3, e.g., the membrane proximal region of DLL3, in the presence of an unlabeled reference antibody can be assessed by ELISA. Bioacore analyses or flow cytometry can be used to demonstrate competition. The test antibody competes for binding to DLL3 with the reference antibody when the test antibody inhibits binding of the reference antibody to soluble DLL3 by 85% or more, for example 90% or more, or 95% or more.

In some embodiments, the application provides an isolated protein, such as an antibody, comprising an antigen binding region that binds DLL3, wherein the antigen binding region that binds DLL3 comprises a VH having a HCDR1, a HCDR2 and a HCDR3, and a VL having a LCDR1, a LCDR2 and a LCDR3, and the HCDR1, HCDR2, HCDR3, LCDR1, LCDR2 and LCDR3 are the HCDR1, the HCDR2 and the HCDR3 of a VH of SEQ ID NO:1 and the LCDR1, the LCDR2 and the LCDR3 of a VL of SEQ ID NO:2; or the HCDR1, the HCDR2 and the HCDR3 of a VH of SEQ ID NO:3 and the LCDR1, the LCDR2 and the LCDR3 of a VL of SEQ ID NO:4; or the HCDR1, the HCDR2 and the HCDR3 of a VH of SEQ ID NO:5 and the LCDR1, the LCDR2 and the LCDR3 of a VL of SEQ ID NO:6; or the HCDR1, the HCDR2 and the HCDR3 of a VH of SEQ ID NO:7 and the LCDR1, the LCDR2 and the LCDR3 of a VL of SEQ ID NO:8; or the HCDR1, the HCDR2 and the HCDR3 of a VH of SEQ ID NO:9 and the LCDR1, the LCDR2 and the LCDR3 of a VL of SEQ ID NO:10; or the HCDR1, the HCDR2 and the HCDR3 of a VH of SEQ ID NO:11 and the LCDR1, the LCDR2 and the LCDR3 of a VL of SEQ ID NO:12; or the HCDR1, the HCDR2 and the HCDR3 of a VH of SEQ ID NO:13 and the LCDR1, the LCDR2 and the LCDR3 of a VL of SEQ ID NO:14. In a particular embodiment, the isolated protein comprising an antigen binding region that binds DLL3, wherein the antigen binding region that binds DLL3 comprises the HCDR1, the HCDR2 and the HCDR3 of the VH of SEQ ID NO:3 and the LCDR1, the LCDR2 and the LCDR3 of the VL of SEQ ID NO:4.

In some embodiments, the application provides an isolated protein, such as an antibody, comprising an antigen binding region that binds DLL3, wherein the antigen binding region that binds DLL3 comprises the HCDR1, the HCDR2, the HCDR3, the LCDR1, the LCDR2 and the LCDR3 having the amino acid sequences of:
SEQ ID NOs:15, 16, 17, 33, 34, 35, respectively;
SEQ ID NOs:18, 19, 20, 36, 37, 38, respectively;
SEQ ID NOs:21, 22, 23, 39, 37, 40, respectively;
SEQ ID NOs:24, 25, 26, 41, 42, 43, respectively;
SEQ ID NOs:18, 28, 29, 44, 45, 46, respectively;
SEQ ID NOs:30, 31, 32, 47, 48, 49, respectively;
SEQ ID NOs:50, 51, 17, 33, 34, 35, respectively;
SEQ ID NOs:52, 51, 17, 33, 34, 35, respectively;
SEQ ID NOs:53, 54, 20, 36, 37, 38, respectively;
SEQ ID NOs:55, 56, 23, 39, 37, 40, respectively;
SEQ ID NOs:57, 58, 26, 41, 42, 43, respectively;
SEQ ID NOs:59, 60, 29, 44, 45, 46, respectively; or
SEQ ID NOs:61, 62, 32, 47, 48, 49, respectively.

In one embodiment, the disclosure provides an isolated protein comprising an antigen binding region that binds DLL3, wherein the antigen binding region that binds DLL3 comprises the HCDR1, the HCDR2, the HCDR3, the LCDR1, the LCDR2 and the LCDR3 having the amino acid sequences of SEQ ID NOs:15, 16, 17, 33, 34, 35, respectively.

In another embodiment, the disclosure provides an isolated protein comprising an antigen binding region that binds DLL3, wherein the antigen binding region that binds DLL3 comprises a VH having the amino acid sequence of SEQ ID NOs:1, 3, 5, 7, 9, 11, or 13 and a VL having the amino acid sequence of SEQ ID NOs:2, 4, 6, 8, 10, 12, or 14.

In one embodiment, the disclosure provides an isolated protein comprising an antigen binding region that binds DLL3, wherein the antigen binding region that binds DLL3 comprises: a VH of the amino acid sequence of SEQ ID NO:1 and a VL of the amino acid sequence of SEQ ID NO:2 (also referred to as a VH of SEQ ID NO:1 and a VL of SEQ ID NO:2);
a VH of SEQ ID NO:1 and a VL of SEQ ID NO:4;
a VH of SEQ ID NO:1 and a VL of SEQ ID NO:6;
a VH of SEQ ID NO:1 and a VL of SEQ ID NO:8;
a VH of SEQ ID NO:1 and a VL of SEQ ID NO:10;
a VH of SEQ ID NO:1 and a VL of SEQ ID NO: 12;
a VH of SEQ ID NO:1 and a VL of SEQ ID NO: 14;
a VH of SEQ ID NO:3 and a VL of SEQ ID NO:2;
a VH of SEQ ID NO:3 and a VL of SEQ ID NO:4;
a VH of SEQ ID NO:3 and a VL of SEQ ID NO:6;
a VH of SEQ ID NO:3 and a VL of SEQ ID NO:8;
a VH of SEQ ID NO:3 and a VL of SEQ ID NO: 10;
a VH of SEQ ID NO:3 and a VL of SEQ ID NO: 12;
a VH of SEQ ID NO:3 and a VL of SEQ ID NO: 14;
a VH of SEQ ID NO:3 and a VL of SEQ ID NO:2;
a VH of SEQ ID NO:3 and a VL of SEQ ID NO:4;
a VH of SEQ ID NO:3 and a VL of SEQ ID NO:6;
a VH of SEQ ID NO:3 and a VL of SEQ ID NO:8;
a VH of SEQ ID NO:3 and a VL of SEQ ID NO: 10;
a VH of SEQ ID NO:3 and a VL of SEQ ID NO:12;
a VH of SEQ ID NO:3 and a VL of SEQ ID NO: 14;
a VH of SEQ ID NO:5 and a VL of SEQ ID NO:2;
a VH of SEQ ID NO:5 and a VL of SEQ ID NO:4;
a VH of SEQ ID NO:5 and a VL of SEQ ID NO:6;
a VH of SEQ ID NO:5 and a VL of SEQ ID NO:8;
a VH of SEQ ID NO:5 and a VL of SEQ ID NO: 10;
a VH of SEQ ID NO:5 and a VL of SEQ ID NO: 12;
a VH of SEQ ID NO:5 and a VL of SEQ ID NO: 14;
a VH of SEQ ID NO:7 and a VL of SEQ ID NO:2;
a VH of SEQ ID NO:7 and a VL of SEQ ID NO:4;
a VH of SEQ ID NO:7 and a VL of SEQ ID NO:6;
a VH of SEQ ID NO:7 and a VL of SEQ ID NO:8;
a VH of SEQ ID NO:7 and a VL of SEQ ID NO: 10;
a VH of SEQ ID NO:7 and a VL of SEQ ID NO: 12;
a VH of SEQ ID NO:7 and a VL of SEQ ID NO: 14;
a VH of SEQ ID NO:9 and a VL of SEQ ID NO:2;
a VH of SEQ ID NO:9 and a VL of SEQ ID NO:4;
a VH of SEQ ID NO:9 and a VL of SEQ ID NO:6;
a VH of SEQ ID NO:9 and a VL of SEQ ID NO:8;
a VH of SEQ ID NO:9 and a VL of SEQ ID NO: 10;
a VH of SEQ ID NO:9 and a VL of SEQ ID NO:12;
a VH of SEQ ID NO:9 and a VL of SEQ ID NO: 14;
a VH of SEQ ID NO:11 and a VL of SEQ ID NO:2;
a VH of SEQ ID NO:11 and a VL of SEQ ID NO:4;
a VH of SEQ ID NO:11 and a VL of SEQ ID NO:6;
a VH of SEQ ID NO:11 and a VL of SEQ ID NO:8;
a VH of SEQ ID NO: 11 and a VL of SEQ ID NO: 10;
a VH of SEQ ID NO:11 and a VL of SEQ ID NO:12;
a VH of SEQ ID NO: 11 and a VL of SEQ ID NO: 14;
a VH of SEQ ID NO: 13 and a VL of SEQ ID NO:2;
a VH of SEQ ID NO:13 and a VL of SEQ ID NO:4;
a VH of SEQ ID NO: 13 and a VL of SEQ ID NO:6;
a VH of SEQ ID NO:13 and a VL of SEQ ID NO:8;
a VH of SEQ ID NO: 13 and a VL of SEQ ID NO: 10;
a VH of SEQ ID NO: 13 and a VL of SEQ ID NO: 12; or
a VH of SEQ ID NO:13 and a VL of SEQ ID NO:14.

In a particular embodiment, the disclosure provides an isolated protein comprising an antigen binding region that binds DLL3, wherein the antigen binding region that binds DLL3 comprises a VH of SEQ ID NO:3 and a VL of SEQ ID NO:4, or a derivative thereof.

The disclosure also provides an isolated protein comprising an antigen binding region that binds DLL3, wherein the antigen binding region that binds DLL3 comprises a VH which is at least 80% (e.g., at least 85%, at least 90%, at least 95% or at least 99%) identical to the VH of SEQ ID NO:3 and a VL which is at least 80% (e.g., at least 85%, at least 90%, at least 95% or at least 99%) identical to the VL of SEQ ID NO:4.

In some embodiments, the antigen binding region that binds DLL3 comprises a VH which is at least 80% (e.g., at least 85%, at least 90%, at least 95% or at least 99%) identical to the VH of SEQ ID NO:3 and a VL of SEQ ID NO:4.

In some embodiments, the antigen binding region that binds DLL3 comprises a VH of SEQ ID NO:3 and a VL which is at least 80% (e.g., at least 85%, at least 90%, at least 95% or at least 99%) identical to the VL of SEQ ID NO:4.

In some embodiments, the antigen binding region that binds DLL3 comprises a VH which is at least 95% identical to the VH of SEQ ID NO:3 and a VL which is at least 95% identical to the VL of SEQ ID NO:4.

In some embodiments, the antigen binding region that binds DLL3 comprises a VH which is at least 95% identical to the VH of SEQ ID NO:3 and a VL which is at least 99% identical to the VL of SEQ ID NO:4.

In some embodiments, the antigen binding region that binds DLL3 comprises a VH which is at least 99% identical to the VH of SEQ ID NO:3 and a VL which is at least 99% identical to the VL of SEQ ID NO:4.

In some embodiments, the antigen binding region that binds DLL3 comprises a VH which is at least 99% identical to the VH of SEQ ID NO:3 and a VL which is at least 95% identical to the VL of SEQ ID NO:4.

The disclosure provides an isolated protein comprising an antigen binding region that binds DLL3, wherein the antigen binding region that binds DLL3 comprises the amino acid sequence of SEQ ID NOs:63, 64, 65, 66, 67, 68, or 69.

In a particular embodiment, the disclosure provides an isolated protein comprising an antigen binding region that binds DLL3, wherein the antigen binding region that binds DLL3 comprises the amino acid sequence of SEQ ID NO:63.

In a particular embodiment, the disclosure provides an isolated protein comprising an antigen binding region that binds DLL3, wherein the antigen binding region that binds DLL3 comprises the amino acid sequence of SEQ ID NO:64.

The disclosure also provides an isolated protein comprising an antigen binding region that binds DLL3, wherein the antigen binding region that binds DLL3 comprises an amino acid sequence at least 80% (e.g., at least 85%, at least 90%, at least 95% or at least 99%) identical to the amino acid sequence of SEQ ID NO:63.

The disclosure also provides an isolated protein comprising an antigen binding region that binds DLL3, wherein the antigen binding region that binds DLL3 comprises an amino acid sequence at least 80% (e.g., at least 85%, at least 90%, at least 95% or at least 99%) identical to the amino acid sequence of SEQ ID NO:64.

In some embodiments, the antigen binding region that binds DLL3 is a scFv.

In some embodiments, the antigen binding region that binds DLL3 is a (scFv)₂.

In some embodiments, the antigen binding region that binds DLL3 is a Fv.

In some embodiments, the antigen binding region that binds DLL3 is a Fab.

In some embodiments, the antigen binding region that binds DLL3 is a F(ab′)₂.

In some embodiments, the antigen binding region that binds DLL3 is a Fd.

In some embodiments, the antigen binding region that binds DLL3 is a dAb.

In some embodiments, the antigen binding region that binds DLL3 is a VHH.

In a particular embodiment, the antigen binding region that binds DLL3 is a scFv.

DLL3 Binding scFvs

Any of the VH and the VL or components thereof identified herein that bind DLL3 can be engineered into scFv format in either VH-linker-VL or VL-linker-VH orientation. Any of the VH and the VL identified herein can also be used to generate sc(Fv)₂ structures, such as VH-linker-VL-linker-VL-linker-VH, VH-linker-VL-linker-VH-linker-VL, VH-linker-VH-linker-VL-linker-VL, VL-linker-VH-linker-VH-linker-VL, VL-linker-VH-linker-VL-linker-VH or VL-linker-VL-linker-VH-linker-VH.

The VH and the VL or components thereof identified herein can be incorporated into a scFv format and the binding and thermostability of the resulting scFv to DLL3 can be assessed using known methods in view of the present disclosure. Binding can be assessed using ProteOn XPR36, Biacore 3000 or KinExA instrumentation, ELISA or competitive binding assays known to those skilled in the art. Binding can be evaluated using purified scFvs or Ecoli supernatants or lysed cells containing the expressed scFv. The measured affinity of a test scFv to DLL3 can vary if measured under different conditions (e.g., osmolarity, pH). Thus, measurements of affinity and other binding parameters (e.g., K_{D}, Kₒₙ, K_{off}) are typically made with standardized conditions and standardized buffers. Thermostability may be evaluated by heating the test scFv at elevated temperatures, such as at 50° C., 55° C. or 60° C. for a period of time, such as 5 minutes (min), 10 min, 15 min, 20 min, 25 min or 30 min and measuring binding of the test scFv to DLL3. The scFvs retaining comparable binding to DLL3 when compared to a non-heated scFv sample are referred to as being thermostable.

In recombinant expression systems, the linker is a peptide linker and may include any naturally occurring amino acid. Exemplary amino acids that may be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to DLL3.

The linker can be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. In some embodiments, the linker is 6 amino acids long. In some embodiments, the linker is 7 amino acids long. In some embodiments, the linker is 8 amino acids long. In some embodiments, the linker is 9 amino acids long. In some embodiments, the linker is 10 amino acids long. In some embodiments, the linker is 11 amino acids long. In some embodiments, the linker is 12 amino acids long. In some embodiments, the linker is 13 amino acids long. In some embodiments, the linker is 14 amino acids long. In some embodiments, the linker is 15 amino acids long. In some embodiments, the linker is 16 amino acids long. In some embodiments, the linker is 17 amino acids long. In some embodiments, the linker is 18 amino acids long. In some embodiments, the linker is 19 amino acids long. In some embodiments, the linker is 20 amino acids long. In some embodiments, the linker is 21 amino acids long. In some embodiments, the linker is 22 amino acids long. In some embodiments, the linker is 23 amino acids long. In some embodiments, the linker is 24 amino acids long. In some embodiments, the linker is 25 amino acids long. In some embodiments, the linker is 26 amino acids long. In some embodiments, the linker is 27 amino acids long. In some embodiments, the linker is 28 amino acids long. In some embodiments, the linker is 29 amino acids long. In some embodiments, the linker is 30 amino acids long. In some embodiments, the linker is 31 amino acids long. In some embodiments, the linker is 32 amino acids long. In some embodiments, the linker is 33 amino acids long. In some embodiments, the linker is 34 amino acids long. In some embodiments, the linker is 35 amino acids long. In some embodiments, the linker is 36 amino acids long. In some embodiments, the linker is 37 amino acids long. In some embodiments, the linker is 38 amino acids long. In some embodiments, the linker is 39 amino acids long. In some embodiments, the linker is 40 amino acids long. Exemplary linkers that may be used are Gly rich linkers, Gly and Ser containing linkers, Gly and Ala containing linkers, Ala and Ser containing linkers, and other flexible linkers.

Other linker sequences can include portions of immunoglobulin hinge area, CL or CH1 derived from any immunoglobulin heavy or light chain isotype. Alternatively, a variety of non-proteinaceous polymers, including polyethylene glycol (PEG), polypropylene glycol, polyoxyalkylenes, or copolymers of polyethylene glycol and polypropylene glycol, may find use as linkers. Exemplary linkers that may be used are shown in Table 2. Additional linkers are described for example in Int. Pat. Publ. No. WO2019/060695.

In some embodiments, the scFv comprises, from the N- to C-terminus, a VH, a first linker (L1) and a VL (VH-L1-VL).

In some embodiments, the scFv comprises, from the N-to C-terminus, the VL, the L1 and the VH (VL-L1-VH). In some embodiments, the L1 comprises the amino acid sequence of SEQ ID NO:120, SEQ ID NO:27, SEQ ID NO:72, SEQ ID NO:73, SEQ ID NO:74, SEQ ID NO:75, SEQ ID NO:76, SEQ ID NO:79, SEQ ID NO:81, SEQ ID NO:82, SEQ ID NO:83, SEQ ID NO:88, SEQ ID NO:90, SEQ ID NO:91, SEQ ID NO:92, SEQ ID NO:121, SEQ ID NO:122, SEQ ID NO:123, SEQ ID NO: 124, SEQ ID NO: 125, SEQ ID NO: 126, SEQ ID NO: 127, SEQ ID NO:128, SEQ ID NO:129, SEQ ID NO:130, SEQ ID NO:131, SEQ ID NO:132, SEQ ID NO: 133, SEQ ID NO: 134, SEQ ID NO: 135, SEQ ID NO: 136, SEQ ID NO: 137, SEQ ID NO:138, or SEQ ID NO: 139.

TABLE 2
The amino acid sequences of linkers.

| | | SEQ ID |
|---|---|---|
| Linker name | Amino acid sequence | NO: |
| Linker 1 | GGSEGKSSGSGSESKSTGGS | 120 |
| Linker 2 | GGGSGGGS | 27 |
| Linker 3 | GGGSGGGSGGGS | 72 |
| Linker 4 | GGGSGGGSGGGSGGGS | 73 |
| Linker 5 | GGGSGGGSGGGSGGGSGGGS | 74 |
| Linker 6 | GGGGSGGGGSGGGGS | 75 |
| Linker 7 | GGGGSGGGGSGGGGSGGGGS | 76 |
| Linker 8 | GGGGSGGGGSGGGGSGGGGSGGGGS | 79 |
| Linker 9 | GSTSGSGKPGSGEGSTKG | 81 |
| Linker 10 | IRPRAIGGSKPRVA | 82 |
| Linker 11 | GKGGSGKGGSGKGGS | 83 |
| Linker 12 | GGKGSGGKGSGGKGS | 88 |
| Linker 13 | GGGKSGGGKSGGGKS | 90 |
| Linker 14 | GKGKSGKGKSGKGKS | 91 |
| Linker 15 | GGGKSGGKGSGKGGS | 92 |
| Linker 16 | GKPGSGKPGSGKPGS | 121 |
| Linker 17 | GKPGSGKPGSGKPGSGKPGS | 122 |
| Linker 18 | GKGKSGKGKSGKGKSGKGKS | 123 |
| Linker 19 | STAGDTHLGGEDFD | 124 |
| Linker 20 | GEGGSGEGGSGEGGS | 125 |
| Linker 21 | GGEGSGGEGSGGEGS | 126 |
| Linker 22 | GEGESGEGESGEGES | 127 |
| Linker 23 | GGGESGGEGSGEGGS | 128 |
| Linker 24 | GEGESGEGESGEGESGEGES | 129 |
| Linker 25 | GSTSGSGKPGSGEGSTKG | 130 |
| Linker 26 | PRGASKSGSASQTGSAPGS | 131 |
| Linker 27 | GTAAAGAGAAGGAAAGAAG | 132 |
| Linker 28 | GTSGSSGSGSGGSGSGGGG | 133 |
| Linker 29 | GKPGSGKPGSGKPGSGKPGS | 134 |
| Linker 30 | GSGS | 135 |
| Linker 31 | APAPAPAPAP | 136 |
| Linker 32 | APAPAPAPAPAPAPAPAPAP | 137 |
| Linker 33 | AEAAAKEAAAKEAAAAKEAAAAKEAA | 138 |
| | AAKAAA | |
| Linker 34 | GTEGKSSGSGSESKST | 139 |

In a particular embodiment, the L1 comprises or consists of the amino acid sequence of SEQ ID NO:120.

In some embodiments, the scFv comprises a heavy chain complementarity determining region (HCDR)1, a HCDR2 and a HCDR3 of a heavy chain variable region (VH) of SEQ ID NO:1 and a light chain complementarity determining region (LCDR)1, a LCDR2 and a LCDR3 of a light chain variable region (VL) of SEQ ID NO:2; or the HCDR1, the HCDR2 and the HCDR3 of the VH of SEQ ID NO:3 and the LCDR1, the LCDR2 and the LCDR3 of the VL of SEQ ID NO:4; or the HCDR1, the HCDR2 and the HCDR3 of the VH of SEQ ID NO:5 and the LCDR1, the LCDR2 and the LCDR3 of the VL of SEQ ID NO:6; or the HCDR1, the HCDR2 and the HCDR3 of the VH of SEQ ID NO:7 and the LCDR1, the LCDR2 and the LCDR3 of the VL of SEQ ID NO:8; or the HCDR1, the HCDR2 and the HCDR3 of the VH of SEQ ID NO:9 and the LCDR1, the LCDR2 and the LCDR3 of the VL of SEQ ID NO: 10; or the HCDR1, the HCDR2 and the HCDR3 of the VH of SEQ ID NO: 11 and the LCDR1, the LCDR2 and the LCDR3 of the VL of SEQ ID NO:12; or the HCDR1, the HCDR2 and the HCDR3 of the VH of SEQ ID NO:13 and the LCDR1, the LCDR2 and the LCDR3 of the VL of SEQ ID NO: 14. In a particular embodiment, the scFv comprises the HCDR1, the HCDR2 and the HCDR3 of the VH of SEQ ID NO:3 and the LCDR1, the LCDR2 and the LCDR3 of the VL of SEQ ID NO:4.

In some embodiments, the scFv comprises a VH having a HCDR1, a HCDR2 and a HCDR3, and a VL having a LCDR1, a LCDR2 and a LCDR3, and the HCDR1, the HCDR2, the HCDR3, the LCDR1, the LCDR2 and the LCDR3 comprises the amino acid sequences of:
SEQ ID NOs:15, 16, 17, 33, 34, 35, respectively;
SEQ ID NOs:18, 19, 20, 36, 37, 38, respectively;
SEQ ID NOs:21, 22, 23, 39, 37, 40, respectively;
SEQ ID NOs:24, 25, 26, 41, 42, 43, respectively;
SEQ ID NOs:18, 28, 29, 44, 45, 46, respectively;
SEQ ID NOs:30, 31, 32, 47, 48, 49, respectively;
SEQ ID NOs:50, 51, 17, 33, 34, 35, respectively;
SEQ ID NOs:52, 51, 17, 33, 34, 35, respectively;
SEQ ID NOs:53, 54, 20, 36, 37, 38, respectively;
SEQ ID NOs:55, 56, 23, 39, 37, 40, respectively;
SEQ ID NOs:57, 58, 26, 41, 42, 43, respectively;
SEQ ID NOs:59, 60, 29, 44, 45, 46, respectively; or
SEQ ID NOs:61, 62, 32, 47, 48, 49, respectively.

In a particular embodiment, the scFv comprises the HCDR1, the HCDR2, the HCDR3, the LCDR1, the LCDR2 and the LCDR3 of SEQ ID NOs:15, 16, 17, 33, 34, 35, respectively.

In some embodiments, the scFv comprises the VH of SEQ ID NO:1 and the VL of SEQ ID NO:2.

In some embodiments, the scFv comprises the VH of SEQ ID NO:1 and the VL of SEQ ID NO:4.

In some embodiments, the scFv comprises the VH of SEQ ID NO:1 and the VL of SEQ ID NO:6.

In some embodiments, the scFv comprises the VH of SEQ ID NO:1 and the VL of SEQ ID NO:8.

In some embodiments, the scFv comprises the VH of SEQ ID NO:1 and the VL of SEQ ID NO:10.

In some embodiments, the scFv comprises the VH of SEQ ID NO:1 and the VL of SEQ ID NO:12.

In some embodiments, the scFv comprises the VH of SEQ ID NO:1 and the VL of SEQ ID NO:14.

In some embodiments, the scFv comprises the VH of SEQ ID NO:3 and the VL of SEQ ID NO:2.

In some embodiments, the scFv comprises the VH of SEQ ID NO:3 and the VL of SEQ ID NO:4.

In some embodiments, the scFv comprises the VH of SEQ ID NO:3 and the VL of SEQ ID NO:6.

In some embodiments, the scFv comprises the VH of SEQ ID NO:3 and the VL of SEQ ID NO:8.

In some embodiments, the scFv comprises the VH of SEQ ID NO:3 and the VL of SEQ ID NO:10.

In some embodiments, the scFv comprises the VH of SEQ ID NO:3 and the VL of SEQ ID NO:12.

In some embodiments, the scFv comprises the VH of SEQ ID NO:3 and the VL of SEQ ID NO:14.

In some embodiments, the scFv comprises the VH of SEQ ID NO:5 and the VL of SEQ ID NO:2.

In some embodiments, the scFv comprises the VH of SEQ ID NO:5 and the VL of SEQ ID NO:4.

In some embodiments, the scFv comprises the VH of SEQ ID NO:5 and the VL of SEQ ID NO:6.

In some embodiments, the scFv comprises the VH of SEQ ID NO:5 and the VL of SEQ ID NO:8.

In some embodiments, the scFv comprises the VH of SEQ ID NO:5 and the VL of SEQ ID NO:10.

In some embodiments, the scFv comprises the VH of SEQ ID NO:5 and the VL of SEQ ID NO:12.

In some embodiments, the scFv comprises the VH of SEQ ID NO:5 and the VL of SEQ ID NO:14.

In some embodiments, the scFv comprises the VH of SEQ ID NO:7 and the VL of SEQ ID NO:2.

In some embodiments, the scFv comprises the VH of SEQ ID NO:7 and the VL of SEQ ID NO:4.

In some embodiments, the scFv comprises the VH of SEQ ID NO:7 and the VL of SEQ ID NO:6.

In some embodiments, the scFv comprises the VH of SEQ ID NO:7 and the VL of SEQ ID NO:8.

In some embodiments, the scFv comprises the VH of SEQ ID NO:7 and the VL of SEQ ID NO:10.

In some embodiments, the scFv comprises the VH of SEQ ID NO:7 and the VL of SEQ ID NO:12.

In some embodiments, the scFv comprises the VH of SEQ ID NO:7 and the VL of SEQ ID NO:14.

In some embodiments, the scFv comprises the VH of SEQ ID NO:9 and the VL of SEQ ID NO:2.

In some embodiments, the scFv comprises the VH of SEQ ID NO:9 and the VL of SEQ ID NO:4.

In some embodiments, the scFv comprises the VH of SEQ ID NO:9 and the VL of SEQ ID NO:6.

In some embodiments, the scFv comprises the VH of SEQ ID NO:9 and the VL of SEQ ID NO:8.

In some embodiments, the scFv comprises the VH of SEQ ID NO:9 and the VL of SEQ ID NO:10.

In some embodiments, the scFv comprises the VH of SEQ ID NO:9 and the VL of SEQ ID NO:12.

In some embodiments, the scFv comprises the VH of SEQ ID NO:9 and the VL of SEQ ID NO:14.

In some embodiments, the scFv comprises the VH of SEQ ID NO: 11 and the VL of SEQ ID NO:2.

In some embodiments, the scFv comprises the VH of SEQ ID NO: 11 and the VL of SEQ ID NO:4.

In some embodiments, the scFv comprises the VH of SEQ ID NO: 11 and the VL of SEQ ID NO:6.

In some embodiments, the scFv comprises the VH of SEQ ID NO: 11 and the VL of SEQ ID NO:8.

In some embodiments, the scFv comprises the VH of SEQ ID NO: 11 and the VL of SEQ ID NO:10.

In some embodiments, the scFv comprises the VH of SEQ ID NO: 11 and the VL of SEQ ID NO:12.

In some embodiments, the scFv comprises the VH of SEQ ID NO: 11 and the VL of SEQ ID NO:14.

In some embodiments, the scFv comprises the VH of SEQ ID NO: 13 and the VL of SEQ ID NO:2.

In some embodiments, the scFv comprises the VH of SEQ ID NO: 13 and the VL of SEQ ID NO:4.

In some embodiments, the scFv comprises the VH of SEQ ID NO: 13 and the VL of SEQ ID NO:6.

In some embodiments, the scFv comprises the VH of SEQ ID NO: 13 and the VL of SEQ ID NO:8.

In some embodiments, the scFv comprises the VH of SEQ ID NO: 13 and the VL of SEQ ID NO:10.

In some embodiments, the scFv comprises the VH of SEQ ID NO: 13 and the VL of SEQ ID NO:12.

In some embodiments, the scFv comprises the VH of SEQ ID NO: 13 and the VL of SEQ ID NO:14.

In a particular embodiment, the scFv comprises the VH of SEQ ID NO:3 and the VL of SEQ ID NO:4.

In some embodiments, the scFv comprises a VH which is at least 80% (e.g., at least 85%, at least 90%, at least 95% or at least 99%) identical to the VH of SEQ ID NO:1 and a VL which is at least 80% (e.g., at least 85%, at least 90%, at least 95% or at least 99%) identical to the VL of SEQ ID NO:2.

In some embodiments, the scFv comprises a VH which is at least 80% (e.g., at least 85%, at least 90%, at least 95% or at least 99%) identical to the VH of SEQ ID NO:3 and a VL which is at least 80% (e.g., at least 85%, at least 90%, at least 95% or at least 99%) identical to the VL of SEQ ID NO:4.

In some embodiments, the scFv comprises a VH which is at least 80% (e.g., at least 85%, at least 90%, at least 95% or at least 99%) identical to the VH of SEQ ID NO:5 and a VL which is at least 80% (e.g., at least 85%, at least 90%, at least 95% or at least 99%) identical to the VL of SEQ ID NO:6.

In some embodiments, the scFv comprises a VH which is at least 80% (e.g., at least 85%, at least 90%, at least 95% or at least 99%) identical to the VH of SEQ ID NO:7 and a VL which is at least 80% (e.g., at least 85%, at least 90%, at least 95% or at least 99%) identical to the VL of SEQ ID NO:8.

In some embodiments, the scFv comprises a VH which is at least 80% (e.g., at least 85%, at least 90%, at least 95% or at least 99%) identical to the VH of SEQ ID NO:9 and a VL which is at least 80% (e.g., at least 85%, at least 90%, at least 95% or at least 99%) identical to the VL of SEQ ID NO:10.

In some embodiments, the scFv comprises a VH which is at least 80% (e.g., at least 85%, at least 90%, at least 95% or at least 99%) identical to the VH of SEQ ID NO: 11 and a VL which is at least 80% (e.g., at least 85%, at least 90%, at least 95% or at least 99%) identical to the VL of SEQ ID NO:12.

In some embodiments, the scFv comprises a VH which is at least 80% (e.g., at least 85%, at least 90%, at least 95% or at least 99%) identical to the VH of SEQ ID NO: 13 and a VL which is at least 80% (e.g., at least 85%, at least 90%, at least 95% or at least 99%) identical to the VL of SEQ ID NO:14.

In some embodiments, the scFv comprises a VH which is at least 80% (e.g., at least 85%, at least 90%, at least 95% or at least 99%) identical to the VH of SEQ ID NO:3 and a VL of SEQ ID NO: 4.

In some embodiments, the scFv comprises a VH of SEQ ID NO:3 and a VL which is at least 80% (e.g., at least 85%, at least 90%, at least 95% or at least 99%) identical to the VL of SEQ ID NO:4.

In some embodiments, the scFv comprises a VH which is at least 95% identical to the VH of SEQ ID NO:3 and a VL which is at least 95% identical to the VL of SEQ ID NO:4.

In some embodiments, the scFv comprises a VH which is at least 99% identical to the VH of SEQ ID NO:3 and a VL which is at least 95% identical to the VL of SEQ ID NO:4.

In some embodiments, the scFv comprises a VH which is at least 99% identical to the VH of SEQ ID NO:3 and a VL which is at least 99% identical to the VL of SEQ ID NO:4.

In some embodiments, the scFv comprises a VH which is at least 95% identical to the VH of SEQ ID NO:3 and a VL which is at least 99% identical to the VL of SEQ ID NO:4.

In some embodiments, the scFv comprises the amino acid sequence of SEQ ID NOs:63, 64, 65, 66, 67, 68, or 69.

In some embodiments, the scFv comprises an amino acid sequence which is at least 80% (e.g., at least 85%, at least 90%, at least 95% or at least 99%) identical to the amino acid sequence of SEQ ID NOs:63, 64, 65, 66, 67, 68, or 69.

In some embodiments, the scFv comprises an amino acid sequence which is at least 80% (e.g., at least 85%, at least 90%, at least 95% or at least 99%) identical to the amino acid sequence of SEQ ID NO:63.

In some embodiments, the scFv comprises an amino acid sequence which is at least 80% (e.g., at least 85%, at least 90%, at least 95% or at least 99%) identical to the amino acid sequence of SEQ ID NO:64.

In a particular embodiment, the scFv comprises the amino acid sequence of SEQ ID NO:63.

In a particular embodiment, the scFv comprises the amino acid sequence of SEQ ID NO:64.

Other Antigen Binding Regions that Bind DLL3

Any of the VH and the VL or components thereof identified herein that bind DLL3 can also be engineered into Fab, F(ab′)₂, Fd or Fv format and their binding to DLL3 and thermostability can be assessed using the assays described herein.

In some embodiments, a Fab, F(ab′)₂, Fd or Fv comprises a VH having a HCDR1, a HCDR2 and a HCDR3, and a VL having a LCDR1, a LCDR2 and a LCDR3, and the HCDR1, the HCDR2, the HCDR3, the LCDR1, the LCDR2 and the LCDR3 comprises: the HCDR1, the HCDR2 and the HCDR3 of a VH of SEQ ID NO:1 and the LCDR1, the LCDR2 and the LCDR3 of a VL of SEQ ID NO:2; or the HCDR1, the HCDR2 and the HCDR3 of the VH of SEQ ID NO:3 and the LCDR1, the LCDR2 and the LCDR3 of the VL of SEQ ID NO:4; or the HCDR1, the HCDR2 and the HCDR3 of the VH of SEQ ID NO:5 and the LCDR1, the LCDR2 and the LCDR3 of the VL of SEQ ID NO:6; or the HCDR1, the HCDR2 and the HCDR3 of the VH of SEQ ID NO:7 and the LCDR1, the LCDR2 and the LCDR3 of the VL of SEQ ID NO:8; or the HCDR1, the HCDR2 and the HCDR3 of the VH of SEQ ID NO:9 and the LCDR1, the LCDR2 and the LCDR3 of the VL of SEQ ID NO: 10; or the HCDR1, the HCDR2 and the HCDR3 of the VH of SEQ ID NO: 11 and the LCDR1, the LCDR2 and the LCDR3 of the VL of SEQ ID NO:12; or the HCDR1, the HCDR2 and the HCDR3 of the VH of SEQ ID NO: 13 and the LCDR1, the LCDR2 and the LCDR3 of the VL of SEQ ID NO:14.

In a particular embodiment, a Fab, F(ab′)₂, Fd or Fv comprises the HCDR1, the HCDR2 and the HCDR3 of the VH of SEQ ID NO:3 and the LCDR1, the LCDR2 and the LCDR3 of the VL of SEQ ID NO:4.

In some embodiments, a Fab, F(ab′)₂, Fd or Fv comprises the HCDR1, the HCDR2, the HCDR3, the LCDR1, the LCDR2 and the LCDR3 of
SEQ ID NOs:15, 16, 17, 33, 34, and 35, respectively;
SEQ ID NOs:18, 19, 20, 36, 37, and 38, respectively;
SEQ ID NOs:21, 22, 23, 39, 37, and 40, respectively;
SEQ ID NOs:24, 25, 26, 41, 42, and 43, respectively;
SEQ ID NOs:18, 28, 29, 44, 45, and 46, respectively;
SEQ ID NOs:30, 31, 32, 47, 48, and 49, respectively;
SEQ ID NOs:50, 51, 17, 33, 34, and 35, respectively;
SEQ ID NOs:52, 51, 17, 33, 34, and 35, respectively;
SEQ ID NOs:53, 54, 20, 36, 37, and 38, respectively;
SEQ ID NOs:55, 56, 23, 39, 37, and 40, respectively;
SEQ ID NOs:57, 58, 26, 41, 42, and 43, respectively;
SEQ ID NOs:59, 60, 29, 44, 45, and 46, respectively; or
SEQ ID NOs:61, 62, 32, 47, 48, and 49, respectively.

In some embodiments, a Fab, F(ab′)₂, Fd or Fv comprises the VH of SEQ ID NO:1 and the VL of SEQ ID NO:2.

In some embodiments, a Fab, F(ab′)₂, Fd or Fv comprises the VH of SEQ ID NO:1 and the VL of SEQ ID NO:4.

In some embodiments, a Fab, F(ab′)₂, Fd or Fv comprises the VH of SEQ ID NO:1 and the VL of SEQ ID NO:6.

In some embodiments, a Fab, F(ab′)₂, Fd or Fv comprises the VH of SEQ ID NO:1 and the VL of SEQ ID NO:8.

In some embodiments, a Fab, F(ab′)₂, Fd or Fv comprises the VH of SEQ ID NO:1 and the VL of SEQ ID NO:10.

In some embodiments, a Fab, F(ab′)₂, Fd or Fv comprises the VH of SEQ ID NO:1 and the VL of SEQ ID NO:12.

In some embodiments, a Fab, F(ab′)₂, Fd or Fv comprises the VH of SEQ ID NO:1 and the VL of SEQ ID NO:14.

In some embodiments, a Fab, F(ab′)₂, Fd or Fv comprises the VH of SEQ ID NO:3 and the VL of SEQ ID NO:2.

In some embodiments, a Fab, F(ab′)₂, Fd or Fv comprises the VH of SEQ ID NO:3 and the VL of SEQ ID NO:4.

In some embodiments, a Fab, F(ab′)₂, Fd or Fv comprises the VH of SEQ ID NO:3 and the VL of SEQ ID NO:6.

In some embodiments, a Fab, F(ab′)₂, Fd or Fv comprises the VH of SEQ ID NO:3 and the VL of SEQ ID NO:8.

In some embodiments, a Fab, F(ab′)₂, Fd or Fv comprises the VH of SEQ ID NO:3 and the VL of SEQ ID NO:10.

In some embodiments, a Fab, F(ab′)₂, Fd or Fv comprises the VH of SEQ ID NO:3 and the VL of SEQ ID NO:12.

In some embodiments, a Fab, F(ab′)₂, Fd or Fv comprises the VH of SEQ ID NO:3 and the VL of SEQ ID NO:14.

In some embodiments, a Fab, F(ab′)₂, Fd or Fv comprises the VH of SEQ ID NO:5 and the VL of SEQ ID NO:2.

In some embodiments, a Fab, F(ab′)₂, Fd or Fv comprises the VH of SEQ ID NO:5 and the VL of SEQ ID NO:4.

In some embodiments, a Fab, F(ab′)₂, Fd or Fv comprises the VH of SEQ ID NO:5 and the VL of SEQ ID NO:6.

In some embodiments, a Fab, F(ab′)₂, Fd or Fv comprises the VH of SEQ ID NO:5 and the VL of SEQ ID NO:8.

In some embodiments, a Fab, F(ab′)₂, Fd or Fv comprises the VH of SEQ ID NO:5 and the VL of SEQ ID NO:10.

In some embodiments, a Fab, F(ab′)₂, Fd or Fv comprises the VH of SEQ ID NO:5 and the VL of SEQ ID NO:12.

In some embodiments, a Fab, F(ab′)₂, Fd or Fv comprises the VH of SEQ ID NO:5 and the VL of SEQ ID NO:14.

In some embodiments, a Fab, F(ab′)₂, Fd or Fv comprises the VH of SEQ ID NO:7 and the VL of SEQ ID NO:2.

In some embodiments, a Fab, F(ab′)₂, Fd or Fv comprises the VH of SEQ ID NO:7 and the VL of SEQ ID NO:4.

In some embodiments, a Fab, F(ab′)₂, Fd or Fv comprises the VH of SEQ ID NO:7 and the VL of SEQ ID NO:6.

In some embodiments, a Fab, F(ab′)₂, Fd or Fv comprises the VH of SEQ ID NO:7 and the VL of SEQ ID NO:8.

In some embodiments, a Fab, F(ab′)₂, Fd or Fv comprises the VH of SEQ ID NO:7 and the VL of SEQ ID NO:10.

In some embodiments, a Fab, F(ab′)₂, Fd or Fv comprises the VH of SEQ ID NO:7 and the VL of SEQ ID NO:12.

In some embodiments, a Fab, F(ab′)₂, Fd or Fv comprises the VH of SEQ ID NO:7 and the VL of SEQ ID NO:14.

In some embodiments, a Fab, F(ab′)₂, Fd or Fv comprises the VH of SEQ ID NO:9 and the VL of SEQ ID NO:2.

In some embodiments, a Fab, F(ab′)₂, Fd or Fv comprises the VH of SEQ ID NO:9 and the VL of SEQ ID NO:4.

In some embodiments, a Fab, F(ab′)₂, Fd or Fv comprises the VH of SEQ ID NO:9 and the VL of SEQ ID NO:6.

In some embodiments, a Fab, F(ab′)₂, Fd or Fv comprises the VH of SEQ ID NO:9 and the VL of SEQ ID NO:8.

In some embodiments, a Fab, F(ab′)₂, Fd or Fv comprises the VH of SEQ ID NO:9 and the VL of SEQ ID NO:10.

In some embodiments, a Fab, F(ab′)₂, Fd or Fv comprises the VH of SEQ ID NO:9 and the VL of SEQ ID NO:12.

In some embodiments, a Fab, F(ab′)₂, Fd or Fv comprises the VH of SEQ ID NO:9 and the VL of SEQ ID NO:14.

In some embodiments, a Fab, F(ab′)₂, Fd or Fv comprises the VH of SEQ ID NO:11 and the VL of SEQ ID NO:2.

In some embodiments, a Fab, F(ab′)₂, Fd or Fv comprises the VH of SEQ ID NO:11 and the VL of SEQ ID NO:4.

In some embodiments, a Fab, F(ab′)₂, Fd or Fv comprises the VH of SEQ ID NO:11 and the VL of SEQ ID NO:6.

In some embodiments, a Fab, F(ab′)₂, Fd or Fv comprises the VH of SEQ ID NO:11 and the VL of SEQ ID NO:8.

In some embodiments, a Fab, F(ab′)₂, Fd or Fv comprises the VH of SEQ ID NO:11 and the VL of SEQ ID NO:10.

In some embodiments, a Fab, F(ab′)₂, Fd or Fv comprises the VH of SEQ ID NO:11 and the VL of SEQ ID NO:12.

In some embodiments, a Fab, F(ab′)₂, Fd or Fv comprises the VH of SEQ ID NO:11 and the VL of SEQ ID NO:14.

In some embodiments, a Fab, F(ab′)₂, Fd or Fv comprises the VH of SEQ ID NO:13 and the VL of SEQ ID NO:2.

In some embodiments, a Fab, F(ab′)₂, Fd or Fv comprises the VH of SEQ ID NO:13 and the VL of SEQ ID NO:4.

In some embodiments, a Fab, F(ab′)₂, Fd or Fv comprises the VH of SEQ ID NO:13 and the VL of SEQ ID NO:6.

In some embodiments, a Fab, F(ab′)₂, Fd or Fv comprises the VH of SEQ ID NO:13 and the VL of SEQ ID NO:8.

In some embodiments, a Fab, F(ab′)₂, Fd or Fv comprises the VH of SEQ ID NO:13 and the VL of SEQ ID NO:10.

In some embodiments, a Fab, F(ab′)₂, Fd or Fv comprises the VH of SEQ ID NO:13 and the VL of SEQ ID NO:12.

In some embodiments, a Fab, F(ab′)₂, Fd or Fv comprises the VH of SEQ ID NO:13 and the VL of SEQ ID NO:14.

In a particular embodiment, a Fab, F(ab′)₂, Fd or Fv comprises a VH of SEQ ID NO:1 and a VL of SEQ ID NO:2.

In some embodiments, the Fab, F(ab′)₂, Fd or Fv comprises a VH which is at least 80% (e.g., at least 85%, at least 90%, at least 95% or at least 99%) identical to the VH of SEQ ID NO:1 and a VL which is at least 80% (e.g., at least 85%, at least 90%, at least 95% or at least 99%) identical to the VL of SEQ ID NO:2.

In some embodiments, the Fab, F(ab′)₂, Fd or Fv comprises a VH which is at least 80% (e.g., at least 85%, at least 90%, at least 95% or at least 99%) identical to the VH of SEQ ID NO:1 and a VL of SEQ ID NO:2.

In some embodiments, the Fab, F(ab′)₂, Fd or Fv comprises a VH of SEQ ID NO:1 and a VL which is at least 80% (e.g., at least 85%, at least 90%, at least 95% or at least 99%) identical to the VL of SEQ ID NO:2.

In some embodiments, the Fab, F(ab′)₂, Fd or Fv comprises a VH which is at least 95% identical to the VH of SEQ ID NO:1 and a VL which is at least 95% identical to the VL of SEQ ID NO:2.

In some embodiments, the Fab, F(ab′)₂, Fd or Fv comprises a VH which is at least 99% identical to the VH of SEQ ID NO:1 and a VL which is at least 95% identical to the VL of SEQ ID NO:2.

In some embodiments, the Fab, F(ab′)₂, Fd or Fv comprises a VH which is at least 99% identical to the VH of SEQ ID NO:1 and a VL which is at least 99% identical to the VL of SEQ ID NO:2.

In some embodiments, the Fab, F(ab′)₂, Fd or Fv comprises a VH which is at least 99% identical to the VH of SEQ ID NO:1 and a VL which is at least 95% identical to the VL of SEQ ID NO:2.

In a particular embodiment, the Fab, F(ab′)₂, Fd or Fv comprises a VH of SEQ ID NO:3 and a VL of SEQ ID NO:4.

In some embodiments, the Fab, F(ab′)₂, Fd or Fv comprises a VH which is at least 80% (e.g., at least 85%, at least 90%, at least 95% or at least 99%) identical to the VH of SEQ ID NO:3 and a VL which is at least 80% (e.g., at least 85%, at least 90%, at least 95% or at least 99%) identical to the VL of SEQ ID NO:4.

In some embodiments, the Fab, F(ab′)₂, Fd or Fv comprises a VH which is at least 80% (e.g., at least 85%, at least 90%, at least 95% or at least 99%) identical to the VH of SEQ ID NO:3 and a VL of SEQ ID NO:4.

In some embodiments, the Fab, F(ab′)₂, Fd or Fv comprises a VH of SEQ ID NO:3 and a VL which is at least 80% (e.g., at least 85%, at least 90%, at least 95% or at least 99%) identical to the VL of SEQ ID NO:4.

In some embodiments, the Fab, F(ab′)₂, Fd or Fv comprises a VH which is at least 95% identical to the VH of SEQ ID NO:3 and a VL which is at least 95% identical to the VL of SEQ ID NO:4.

In some embodiments, the Fab, F(ab′)₂, Fd or Fv comprises a VH which is at least 99% identical to the VH of SEQ ID NO:3 and a VL which is at least 95% identical to the VL of SEQ ID NO:4.

In some embodiments, the Fab, F(ab′)₂, Fd or Fv comprises a VH which is at least 99% identical to the VH of SEQ ID NO:3 and a VL which is at least 99% identical to the VL of SEQ ID NO:4.

In some embodiments, the Fab, F(ab′)₂, Fd or Fv comprises a VH which is at least 99% identical to the VH of SEQ ID NO:3 and a VL which is at least 95% identical to the VL of SEQ ID NO:4.

The VH and VL of the Fab comprising the antigen binding region that binds DLL3 can be engineered into Fab-Fc HC (VH-CH1-hinge-CH2-CH3) and Fab-Fc LC (VL-CL) formats respectively. In certain such embodiments, the Fab-Fc HC comprises an amino acid sequence which is at least 80% (e.g., at least 85%, at least 90%, at least 95% or at least 99%) identical to SEQ ID NO: 109. In a particular embodiment, the Fab-Fc HC comprises an amino acid sequence which is identical to SEQ ID NO:109.

In some embodiments, the Fab-Fc LC comprises an amino acid sequence which is at least 80% (e.g. at least 85%, at least 90%, at least 95% or at least 99%) identical to SEQ ID NO: 110.

In a particular embodiment, the Fab-Fc LC comprises an amino acid sequence which is identical to SEQ ID NO:110.

As shown in the examples, a particularly suitable antigen binding region that binds DLL3 for incorporating into a multispecific construct comprises a Fab-Fc HC having the amino acid sequence of SEQ ID NO: 109 and a Fab-Fc LC having the amino acid sequence of SEQ ID NO:110.

In some embodiments, the F(ab′)₂ comprises the amino acid sequence of SEQ ID NO:63.

In some embodiments, the F(ab′)₂ comprises the amino acid sequence of SEQ ID NO:64.

In some embodiments, the F(ab′)₂ comprises the amino acid sequence of SEQ ID NO:65.

In some embodiments, the F(ab′)₂ comprises the amino acid sequence of SEQ ID NO:66.

In some embodiments, the F(ab′)₂ comprises the amino acid sequence of SEQ ID NO:67.

In some embodiments, the F(ab′)₂ comprises the amino acid sequence of SEQ ID NO:68.

In some embodiments, the F(ab′)₂ comprises the amino acid sequence of SEQ ID NO:69.

In some embodiments, the Fv comprises the amino acid sequence of SEQ ID NO:63.

In some embodiments, the Fv comprises the amino acid sequence of SEQ ID NO:64.

In some embodiments, the Fv comprises the amino acid sequence of SEQ ID NO:65.

In some embodiments, the Fv comprises the amino acid sequence of SEQ ID NO:66.

In some embodiments, the Fv comprises the amino acid sequence of SEQ ID NO:67.

In some embodiments, the Fv comprises the amino acid sequence of SEQ ID NO:68.

In some embodiments, the Fv comprises the amino acid sequence of SEQ ID NO:69.

Homologous Antigen Binding Regions and Antigen Binding Regions with Conservative Substitutions

Variants of the antigen binding regions that bind DLL3 are within the scope of the disclosure. For example, variants may comprise 1, 2, 3, 4, 5, 6, 7, 8, 9, 10, 11, 12, 13, 14, 15, 16, 17, 18, 19, 20, 21, 22, 23, 24, 25, 26, 27, 28 or 29 amino acid substitutions in the antigen binding region that bind DLL3 as long as they retain or have improved functional properties when compared to the parent antigen binding regions. In some embodiments, the sequence identity may be about 80%, 81%, 82%, 83%, 84%, 85%, 86%, 87%, 88%, 89%, 90%, 91%, 92%, 93%, 94%, 95%, 96%, 97%, 98% or 99% to the antigen binding regions that bind DLL3 of the disclosure. In some embodiments, the variation is in the framework regions. In some embodiments, variants are generated by conservative substitutions.

In some embodiments, an isolated protein comprising an antigen binding region that binds DLL3 comprises a VH and a VL which are at least 80% (e.g., at least 85%, at least 90%, at least 95% or at least 99%) identical to the VH and VL, respectively, of an antigen binding region that binds DLL3 disclosed herein.

Also provided are antigen binding regions that bind DLL3 comprising the VH and the VL which are at least 80% identical to the VH of SEQ ID NO:1 and the VL of SEQ ID NO:2;
the VH of SEQ ID NO:3 and the VL of SEQ ID NO:4;
the VH of SEQ ID NO:5 and the VL of SEQ ID NO:6;
the VH of SEQ ID NO:7 and the VL of SEQ ID NO:8;
the VH of SEQ ID NO:9 and the VL of SEQ ID NO:10;
the VH of SEQ ID NO:11 and the VL of SEQ ID NO: 12; or
the VH of SEQ ID NO: 13 and the VL of SEQ ID NO: 14.

In some embodiments, the identity is 85%. In some embodiments, the identity is 90%. In some embodiments, the identity is 91%. In some embodiments, the identity is 91%. In some embodiments, the identity is 92%. In some embodiments, the identity is 93%. In some embodiments, the identity is 94%. In some embodiments, the identity is 94%. In some embodiments, the identity is 95%. In some embodiments, the identity is 96%. In some embodiments, the identity is 97%. In some embodiments, the identity is 98%. In some embodiments, the identity is 99%.

In some embodiments, the antigen binding region that binds DLL3 comprises a VH which is at least 80% (e.g., at least 85%, at least 90%, at least 95% or at least 99%) identical to the VH of SEQ ID NO:1 and a VL which is at least 80% (e.g., at least 85%, at least 90%, at least 95% or at least 99%) identical to the VL of SEQ ID NO:2.

In some embodiments, the antigen binding region that binds DLL3 comprises a VH which is at least 80% (e.g., at least 85%, at least 90%, at least 95% or at least 99%) identical to the VH of SEQ ID NO:3 and a VL which is at least 80% (e.g., at least 85%, at least 90%, at least 95% or at least 99%) identical to the VL of SEQ ID NO:4.

In some embodiments, the antigen binding region that binds DLL3 comprises a VH which is at least 80% (e.g., at least 85%, at least 90%, at least 95% or at least 99%) identical to the VH of SEQ ID NO:5 and a VL which is at least 80% (e.g., at least 85%, at least 90%, at least 95% or at least 99%) identical to the VL of SEQ ID NO:6.

In some embodiments, the antigen binding region that binds DLL3 comprises a VH which is at least 80% (e.g., at least 85%, at least 90%, at least 95% or at least 99%) identical to the VH of SEQ ID NO:7 and a VL which is at least 80% (e.g., at least 85%, at least 90%, at least 95% or at least 99%) identical to the VL of SEQ ID NO:8.

In some embodiments, the antigen binding region that binds DLL3 comprises a VH which is at least 80% (e.g., at least 85%, at least 90%, at least 95% or at least 99%) identical to the VH of SEQ ID NO:9 and a VL which is at least 80% (e.g., at least 85%, at least 90%, at least 95% or at least 99%) identical to the VL of SEQ ID NO: 10.

In some embodiments, the antigen binding region that binds DLL3 comprises a VH which is at least 80% (e.g., at least 85%, at least 90%, at least 95% or at least 99%) identical to the VH of SEQ ID NO: 11 and a VL which is at least 80% (e.g., at least 85%, at least 90%, at least 95% or at least 99%) identical to the VL of SEQ ID NO:12.

In some embodiments, the antigen binding regions that binds DLL3 comprises a VH which is at least 80% (e.g., at least 85%, at least 90%, at least 95% or at least 99%) identical to the VH of SEQ ID NO: 13 and a VL which is at least 80% (e.g., at least 85%, at least 90%, at least 95% or at least 99%) identical to the VL of SEQ ID NO:14.

In some embodiments, the antigen binding region that binds DLL3 comprises a VH which is at least 85% identical to the VH of SEQ ID NO:3 and the VL of SEQ ID NO:4.

In some embodiments, the antigen binding region that binds DLL3 comprises a VH which is at least 90% identical to the VH of SEQ ID NO:3 and the VL of SEQ ID NO:4.

In some embodiments, the antigen binding region that binds DLL3 comprises a VH which is at least 91% identical to the VH of SEQ ID NO:3 and the VL of SEQ ID NO:4.

In some embodiments, the antigen binding region that binds DLL3 comprises a VH which is at least 92% identical to the VH of SEQ ID NO:3 and the VL of SEQ ID NO:4.

In some embodiments, the antigen binding region that binds DLL3 comprises a VH which is at least 93% identical to the VH of SEQ ID NO:3 and the VL of SEQ ID NO:4.

In some embodiments, the antigen binding region that binds DLL3 comprises a VH which is at least 94% identical to the VH of SEQ ID NO:3 and the VL of SEQ ID NO:4.

In some embodiments, the antigen binding region that binds DLL3 comprises a VH which is at least 95% identical to the VH of SEQ ID NO:3 and the VL of SEQ ID NO:4.

In some embodiments, the antigen binding region that binds DLL3 comprises a VH which is at least 96% identical to the VH of SEQ ID NO:3 and the VL of SEQ ID NO:4.

In some embodiments, the antigen binding region that binds DLL3 comprises a VH which is at least 97% identical to the VH of SEQ ID NO:3 and the VL of SEQ ID NO:4.

In some embodiments, the antigen binding region that binds DLL3 comprises a VH which is at least 98% identical to the VH of SEQ ID NO:3 and the VL of SEQ ID NO:4.

In some embodiments, the antigen binding region that binds DLL3 comprises a VH which is at least 99% identical to the VH of SEQ ID NO:3 and the VL of SEQ ID NO:4.

In some embodiments, the antigen binding region that binds DLL3 comprises a VH of SEQ ID NO:3 and a VL which is at least 85% identical to the VL of SEQ ID NO:4.

In some embodiments, the antigen binding region that binds DLL3 comprises a VH of SEQ ID NO:3 and a VL which is at least 90% identical to the VL of SEQ ID NO:4.

In some embodiments, the antigen binding region that binds DLL3 comprises a VH of SEQ ID NO:3 and a VL which is at least 91% identical to the VL of SEQ ID NO:4.

In some embodiments, the antigen binding region that binds DLL3 comprises a VH of SEQ ID NO:3 and a VL which is at least 92% identical to the VL of SEQ ID NO:4.

In some embodiments, the antigen binding region that binds DLL3 comprises a VH of SEQ ID NO:3 and a VL which is at least 93% identical to the VL of SEQ ID NO:4.

In some embodiments, the antigen binding region that binds DLL3 comprises a VH of SEQ ID NO:3 and a VL which is at least 94% identical to the VL of SEQ ID NO:4.

In some embodiments, the antigen binding region that binds DLL3 comprises a VH of SEQ ID NO:3 and a VL which is at least 95% identical to the VL of SEQ ID NO:4.

In some embodiments, the antigen binding region that binds DLL3 comprises a VH of SEQ ID NO:3 and a VL which is at least 96% identical to the VL of SEQ ID NO:4.

In some embodiments, the antigen binding region that binds DLL3 comprises a VH of SEQ ID NO:3 and a VL which is at least 97% identical to the VL of SEQ ID NO:4.

In some embodiments, the antigen binding region that binds DLL3 comprises a VH of SEQ ID NO:3 and a VL which is at least 98% identical to the VL of SEQ ID NO:4.

In some embodiments, the antigen binding region that binds DLL3 comprises a VH of SEQ ID NO:3 and a VL which is at least 99% identical to the VL of SEQ ID NO:4.

The percent identity between the two sequences is a function of the number of identical positions shared by the sequences (i.e., % identity=number of identical positions/total number of positions ×100), taking into account the number of gaps, and the length of each gap, which need to be introduced for optimal alignment of the two sequences.

The percent identity between two amino acid sequences may be determined using the algorithm of E. Meyers and W. Miller (Comput Appl Biosci 4:11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch (J Mol Biol 48:444-453 (1970)) algorithm which has been incorporated into the GAP program in the GCG software package (available at http//www gcg com), using either a Blossum 62 matrix or a PAM250 matrix, and a gap weight of 16, 14, 12, 10, 8, 6, or 4 and a length weight of 1, 2, 3, 4, 5, or 6.

In some embodiments, variant antigen binding regions that bind DLL3 comprise one or two conservative substitutions in any of the CDR regions, while retaining desired functional properties of the parent antigen binding regions that bind DLL3.

“Conservative modifications” refer to amino acid modifications that do not significantly affect or alter the binding characteristics of the antibody containing the amino acid modifications. Conservative modifications include amino acid substitutions, additions and deletions. Conservative amino acid substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643:55-67; Sasaki et al., (1988) Adv Biophys 35:1-24). Amino acid substitutions to the antibodies of the application may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting variants may be tested for their characteristics using assays described herein.

Methods of Generating Antigen Binding Region that Bind DLL3

Antigen binding regions that bind DLL3 provided in the disclosure may be generated using various technologies. For example, the hybridoma method of Kohler and Milstein may be used to identify VH/VL pairs that bind DLL3. In the hybridoma method, a mouse or other host animal, such as a hamster, rat or chicken is immunized with human and/or cyno DLL3, followed by fusion of spleen cells from immunized animals with myeloma cells using standard methods to form hybridoma cells. Colonies arising from single immortalized hybridoma cells may be screened for production of the antibodies containing the antigen binding regions that bind DLL3 with desired properties, such as specificity of binding, cross-reactivity or lack thereof, affinity for the antigen, and any desired functionality.

Antigen binding regions that bind DLL3 generated by immunizing non-human animals may be humanized. Exemplary humanization techniques including selection of human acceptor frameworks include CDR grafting (U.S. Pat. No. 5,225,539), SDR grafting (U.S. Pat. No. 6,818,749), Resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) or superhumanization (U.S. Pat. No. 7,709,226). In these methods, CDRs or a subset of CDR residues of parental antibodies are transferred onto human frameworks that may be selected based on their overall homology to the parental frameworks, based on similarity in CDR length, or canonical structure identity, or a combination thereof.

Humanized antigen binding regions may be further optimized to improve their selectivity or affinity to a desired antigen by incorporating altered framework support residues to preserve binding affinity (backmutations) by techniques such as those described in Int. Patent Publ. Nos. WO1090/007861 and WO1992/22653, or by introducing variation at any of the CDRs for example to improve affinity of the antigen binding region.

Transgenic animals, such as mice, rat or chicken carrying human immunoglobulin (Ig) loci in their genome may be used to generate antigen binding regions that bind DLL3, and are described in for example U.S. Pat. No. 6,150,584, Int. Patent Publ. No. WO1999/45962, Int. Patent Publ. Nos. WO2002/066630, WO2002/43478, WO2002/043478 and WO1990/04036. The endogenous immunoglobulin loci in such animal may be disrupted or deleted, and at least one complete or partial human immunoglobulin locus may be inserted into the genome of the animal using homologous or non-homologous recombination, using transchromosomes, or using minigenes. Companies such as Regeneron (http://_www_regeneron_com), Harbour Antibodies (http://_www_harbourantibodies_com), Open Monoclonal Technology, Inc. (OMT) (http://_www_omtinc_net), KyMab (http://_www_kymab_com), Trianni (http://_www.trianni_com) and Ablexis (http://_www_ablexis_com) may be engaged to provide human antibodies directed against a selected antigen using technologies as described above. In some embodiments, Ablexis mice were immunized with soluble full length DLL3 protein.

Antigen binding regions that bind DLL3 may be selected from a phage display library, where the phage is engineered to express human immunoglobulins or portions thereof such as Fabs, single chain antibodies (scFv), or unpaired or paired antibody variable regions. The antigen binding regions that bind DLL3 may be isolated for example from phage display library expressing antibody heavy and light chain variable regions as fusion proteins with bacteriophage pIX coat protein as described in Shi et al., (2010) JMol Biol 397:385-96, and Int. Patent Publ. No. WO09/085462). The libraries may be screened for phage binding to human and/or cyno DLL3 and the obtained positive clones may be further characterized, the Fabs isolated from the clone lysates, and converted to scFvs or other configurations of antigen binding regions.

Preparation of immunogenic antigens and expression and production of antigen binding regions of the disclosure may be performed using any suitable technique, such as recombinant protein production. The immunogenic antigens may be administered to an animal in the form of purified protein, or protein mixtures including whole cells or cell or tissue extracts, or the antigen may be formed de novo in the animal's body from nucleic acids encoding said antigen or a portion thereof.

### Fusions or Conjugations to Half-Life Extending Moieties

The antigen binding regions that bind DLL3 of the disclosure can be fused or conjugated to a half-life extending moiety. Exemplary half-life extending moieties are albumin, albumin variants, albumin-binding proteins and/or domains, transferrin and fragments and analogues thereof, immunoglobulins (Ig) or fragments thereof, such as Fc regions. Amino acid sequences of the aforementioned half-life extending moieties are known. Ig or fragments thereof include all isotypes, i.e., IgG1, IgG2, IgG3, IgG4, IgM, IgA and IgE.

Additional half-life extending moieties that can be conjugated to the antigen binding regions that bind DLL3 of the disclosure include polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the antigen binding regions that bind DLL3 of the disclosure and may be generated by standard cloning and expression techniques. Alternatively, well known chemical coupling methods may be used to attach the moieties to recombinantly produced antigen binding regions that bind DLL3 of the disclosure.

A pegyl moiety can for example be conjugated to the antigen binding region that bind DLL3 of the disclosure by incorporating a cysteine residue to the C-terminus of the antigen binding region that bind DLL3 of the disclosure, or engineering cysteines into residue positions that face away from the DLL3 binding site and attaching a pegyl group to the cysteine using well known methods.

In some embodiments, the antigen binding region that binds DLL3 is fused or conjugated to a half-life extending moiety.

In some embodiments, the half-life extending moiety is an immunoglobulin (Ig), a fragment of the Ig, an Ig constant region, a fragment of the Ig constant region, a Fc region, transferrin, albumin, an albumin binding domain or polyethylene glycol. In some embodiments, the half-life extending moiety is an Ig constant region.

In some embodiments, the half-life extending moiety is the Ig.

In some embodiments, the half-life extending moiety is the fragment of the Ig.

In some embodiments, the half-life extending moiety is the Ig constant region.

In some embodiments, the half-life extending moiety is the fragment of the Ig constant region.

In some embodiments, the half-life extending moiety is the Fc region.

In some embodiments, the half-life extending moiety is albumin.

In some embodiments, the half-life extending moiety is the albumin binding domain.

In some embodiments, the half-life extending moiety is transferrin.

In some embodiments, the half-life extending moiety is polyethylene glycol.

The antigen binding regions that bind DLL3 fused or conjugated to a half-life extending moiety can be evaluated for their pharmacokinetic properties utilizing known in vivo models in view of the present disclosure.

### Fusion to Immunoglobulin (Ig) Constant Regions or Fragments of the Ig Constant Regions

The antigen binding regions that bind DLL3 of the disclosure can be fused to an Ig constant region or a fragment of the Ig constant region to impart antibody-like properties, including Fc effector functions C1q binding, complement dependent cytotoxicity (CDC), Fc receptor binding, antibody-dependent cell-mediated cytotoxicity (ADCC), phagocytosis or down regulation of cell surface receptors (e.g., B cell receptor; BCR). The Ig constant region or the fragment of the Ig constant region functions also as a half-life extending moiety as discussed herein. The antigen binding regions that bind DLL3 of the disclosure may be engineered into conventional full-length antibodies using standard methods. The full-length antibodies comprising the antigen binding region that binds DLL3 may further be engineered as described herein.

Immunoglobulin heavy chain constant region comprised of subdomains CH1, hinge, CH2 and CH3. The CH1 domain spans residues A118-V215, the CH2 domain residues A231-K340 and the CH3 domain residues G341-K447 on the heavy chain, residue numbering according to the EU Index. In some instances, G341 is referred as a CH2 domain residue. Hinge is generally defined as including E216 and terminating at P230 of human IgG1. Ig Fc region comprises at least the CH2 and the CH3 domains of the Ig constant region, and therefore comprises at least a region from about A231 to K447 of Ig heavy chain constant region.

The application also provides an antigen binding region that binds DLL3 conjugated to an immunoglobulin (Ig) constant region or a fragment of the Ig constant region.

In some embodiments, the Ig constant region is a heavy chain constant region In some embodiments, the Ig constant region is a light chain constant region.

In some embodiments, the fragment of the Ig constant region comprises a Fc region.

In some embodiments, the fragment of the Ig constant region comprises a CH2 domain.

In some embodiments, the fragment of the Ig constant region comprises a CH3 domain.

In some embodiments, the fragment of the Ig constant region comprises the CH2 domain and the CH3 domain.

In some embodiments, the fragment of the Ig constant region comprises at least portion of a hinge, the CH2 domain and the CH3 domain. Portion of the hinge refers to one or more amino acid residues of the Ig hinge.

In some embodiments, the fragment of the Ig constant region comprises the hinge, the CH2 domain and the CH3 domain.

In a particular embodiment, the fragment of the Ig constant region comprises the hinge, the CH2 domain and the CH3 domain.

In some embodiments, the antigen binding region that binds DLL3 is conjugated to the N-terminus of the Ig constant region or the fragment of the Ig constant region.

In some embodiments, the antigen binding region that binds DLL3 is conjugated to the C-terminus of the Ig constant region or the fragment of the Ig constant region.

In some embodiments, the antigen binding region that binds DLL3 is conjugated to the Ig constant region or the fragment of the Ig constant region via a second linker (L2).

In some embodiments, the L2 comprises the amino acid sequence of SEQ ID NOs:27, 72, 73, 74, 75, 76, 79, 81, 82, 83, 88, 90, 91, 92, 120, 121, 122, 123, 124, 125, 126, 127, 128, 129, 130, 131, 132, 133, 134, 135, 136, 137, 138, or 139.

In a particular embodiment, the L2 comprises the amino acid sequence of SEQ ID NO:120.

The antigen binding regions that bind DLL3 of the disclosure conjugated to Ig constant region or the fragment of the Ig constant region may be assessed for their functionality using several known assays. Binding to DLL3 may be assessed using methods described herein. Altered properties imparted by the Ig constant domain or the fragment of the Ig constant region such as Fc region may be assayed in Fc receptor binding assays using soluble forms of the receptors, such as the FcγRI, FcγRII, FcγRIII or FcRn receptors, or using cell-based assays measuring for example ADCC, CDC or ADCP.

ADCC can be assessed using an in vitro assay using DLL3 expressing cells as target cells and NK cells as effector cells. Cytolysis may be detected by the release of label (e.g., radioactive substrates, fluorescent dyes or natural intracellular proteins) from the lysed cells. In an exemplary assay, target cells are used with a ratio of 1 target cell to 4 effector cells. Target cells are pre-labeled with BATDA and combined with effector cells and the test antibody. The samples are incubated for 2 hours and cell lysis measured by measuring released BATDA into the supernatant. Data is normalized to maximal cytotoxicity with 0.67% Triton X-100 (Sigma Aldrich) and minimal control determined by spontaneous release of BATDA from target cells in the absence of any antibody.

ADCP can be evaluated by using monocyte-derived macrophages as effector cells and any DLL3 expressing cells as target cells which are engineered to express GFP or other labeled molecule. In an exemplary assay, effector:target cell ratio may be for example 4:1. Effector cells may be incubated with target cells for 4 hours with or without the antibody of the application. After incubation, cells may be detached using accutase. Macrophages can be identified with anti-CD11b and anti-CD14 antibodies coupled to a fluorescent label, and percent phagocytosis may be determined based on % GFP fluorescence in the CD11⁺ CD14⁺ macrophages using standard methods.

CDC of cells can be measured for example by plating Daudi cells at 1×10⁵ cells/well (50 μL/well) in RPMI-B (RPMI supplemented with 1% BSA), adding 50 μL of test protein to the wells at final concentration between 0-100 μg/mL, incubating the reaction for 15 min at room temperature, adding 11 μL of pooled human serum to the wells, and incubation the reaction for 45 min at 37° C. Percentage (%) lysed cells may be detected as % propidium iodide stained cells in FACS assay using standard methods.

In some embodiments, the first antigen binding region that binds DLL3 is fused to a first immunoglobulin (Ig) constant region or a fragment of the first Ig constant region and/or the second antigen binding region that binds the lymphocyte antigen is fused to a second immunoglobulin (Ig) constant region or a fragment of the second Ig constant region.

In some embodiments, the fragment of the first Ig constant region and/or the fragment of the second Ig constant region comprises a Fc region.

In some embodiments, the fragment of the first Ig constant region and/or the fragment of the second Ig constant region comprises a CH2 domain.

In some embodiments, the fragment of the first Ig constant region and/or the fragment of the second Ig constant region comprises a CH3 domain.

In some embodiments, the fragment of the first Ig constant region and/or the fragment of the second Ig constant region comprises the CH2 domain and the CH3 domain.

In some embodiments, the fragment of the first Ig constant region and/or the fragment of the second Ig constant region comprises at least portion of a hinge, the CH2 domain and the CH3 domain.

In some embodiments, the fragment of the Ig constant region comprises the hinge, the CH2 domain and the CH3 domain.

In some embodiments, the multispecific antigen-binding construct further comprises a second linker (L2) between the first antigen binding region that binds DLL3 and the first Ig constant region or the fragment of the first Ig constant region and the second antigen binding region that binds the lymphocyte antigen and the second Ig constant region or the fragment of the second Ig constant region.

In some embodiments, the L2 comprises the amino acid sequence of SEQ ID NOs:27, 72, 73, 74, 75, 76, 79, 81, 82, 83, 88, 90, 91, 92, 120, 121, 122, 123, 124, 125, 126, 127, 128, 129, 130, 131, 132, 133, 134, 135, 136, 137, 138, or 139.

In a particular embodiment, the L2 comprises the amino acid sequence of SEQ ID NO:120.

In some embodiments, the first Ig constant region or the fragment of the first Ig constant region and the second Ig constant region or the fragment of the second Ig constant region is an IgG1, an IgG2, and IgG3 or an IgG4 isotype.

In some embodiments, the first Ig constant region or the fragment of the first Ig constant region and the second Ig constant region or the fragment of the second Ig constant region is an IgG1 isotype.

In some embodiments, the first Ig constant region or the fragment of the first Ig constant region and the second Ig constant region or the fragment of the second Ig constant region is an IgG2 isotype.

In some embodiments, the first Ig constant region or the fragment of the first Ig constant region and the second Ig constant region or the fragment of the second Ig constant region is an IgG3 isotype.

In some embodiments, the first Ig constant region or the fragment of the first Ig constant region and the second Ig constant region or the fragment of the second Ig constant region is an IgG4 isotype.

In a particular embodiment, the first Ig constant region or the fragment of the first Ig constant region and the second Ig constant region or the fragment of the second Ig constant region is an IgG1 isotype.

The first Ig constant region or the fragment of the first Ig constant region and the second Ig constant region or the fragment of the second Ig constant region can further be engineered as described herein.

In some embodiments, the first Ig constant region or the fragment of the first Ig constant region and the second Ig constant region or the fragment of the second Ig constant region comprises at least one mutation that results in reduced binding of the multispecific antigen-binding construct to a FcγR.

In some embodiments, the at least one mutation that results in reduced binding of the multispecific antigen-binding construct to the FcγR is selected from the group consisting of F234A/L235A, L234A/L235A, L234A/L235A/D265 S, V234A/G237A/P238 S/H268A/V309L/A330 S/P331 S, F234A/L235A, S228P/F234A/L235A, N297A, V234A/G237A, K214T/E233P/L234V/L235A/G236-deleted/A327G/P331A/D365E/L358M, H268Q/V309L/A330 S/P331 S, S267E/L328F, L234F/L235E/D265A, L234A/L235A/G237A/P238 S/H268A/A330 S/P331 S, S228P/F234A/L235A/G237A/P238 S and S228P/F234A/L235A/G236-deleted/G237A/P238 S, wherein residue numbering is according to the EU index. In a particular embodiment, the first Ig constant region or the fragment of the first Ig constant region and/or the second Ig constant region or the fragment of the second Ig constant region comprise the following mutations: L234A_L235A_D265 S.

In some embodiments, the FcγR is FcγRI, FcγRIIA, FcγRIIB or FcγRIII, or any combination thereof.

In some embodiments, the first Ig constant region or the fragment of the first Ig constant region and the second Ig constant region or the fragment of the second Ig constant region comprises at least one mutation that modulates a half-life of the multispecific antigen-binding construct.

In some embodiments, the multispecific antigen-binding construct comprises at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region.

In some embodiments, the at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region is selected from the group consisting of
L351Y_F405A_Y407V/T394W, T3661_K392M_T394W/F405A_Y407V,
T366L_K392M_T394W/F405A_Y407V, L351Y_Y407A/T366A_K409F,
L351Y_Y407A/T366V_K409F, Y407A/T366A_K409F, or
T350V_L351Y_F405A_Y407V/T350V_T366L_K392L_T394W as described in US2012/0149876 or US2013/0195849 (Zymeworks).

In some embodiments, the at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region is selected from the group consisting of T366Y/F405A, T366W/F405W, F405W/Y407A, T394W/Y407T, T394 S/Y407A, T366W/T394 S, F405W/T394 S and T366W/T366 S_L368A_Y407V as described in WO1996/027011.

In some embodiment, a protein or multispecific antigen-binding construct of the application can comprise one or more amino acid modifications that reduces or eliminates the effector function, such as the ADCC or CDC, such as mutations that reduce or abolish the binding to Fc gamma receptor. Such mutations can be at positions L234, L235, D270, N297, E318, K320, K322, P331, and P329, such as one, two or three mutations of L234A, L235A and P331 S, wherein the numbering of amino acid residues is according to the EU index as set forth in Kabat.

Proteins Comprising the Antigen Binding Regions that Bind DLL3 of the Disclosure

The antigen binding regions that bind DLL3 of the disclosure can be engineered into monospecific or multispecific antigen-binding constructs of various designs using standard methods.

The disclosure also provides a monospecific protein comprising the antigen binding region that binds DLL3 of the disclosure.

In some embodiments, the monospecific protein is an antibody.

The disclosure also provides a multispecific antigen-binding construct comprising the antigen binding region that binds DLL3 of the disclosure.

In some embodiments, the multispecific antigen-binding construct is bispecific.

In some embodiments, the multispecific antigen-binding construct is trispecific.

In some embodiments, the multispecific antigen-binding construct is tetraspecific.

In some embodiments, the multispecific antigen-binding construct is monovalent for binding to DLL3.

In some embodiments, the multispecific antigen-binding construct is bivalent for binding to DLL3.

The disclosure also provides an isolated multispecific antigen-binding construct comprising a first antigen binding region that binds DLL3 and a second antigen binding region that binds a lymphocyte antigen (such as CD3).

In some embodiments, the lymphocyte antigen is a T cell antigen.

In some embodiments, the T cell antigen is a CD8⁺ T cell antigen.

In some embodiments, the lymphocyte antigen is a NK cell antigen.

In some embodiments, the lymphocyte antigen is CD3, CD3 epsilon (CD3E), CD8, KI2L4, NKG2E, NKG2D, NKG2F, BTNL3, CD186, BTNL8, PD-1, CD195, or NKG2C.

In some embodiments, the lymphocyte antigen is CD3ε.

In some embodiments, the first antigen binding region that binds DLL3 and/or the second antigen binding region that binds the lymphocyte antigen comprise a scFv, a (scFv)₂, a Fv, a Fab, a F(ab′)₂, a Fd, a dAb or a VHH.

In some embodiments, the first antigen binding region that binds DLL3 and/or the second antigen binding region that binds the lymphocyte antigen comprise the Fab.

In some embodiments, the first antigen binding region that binds DLL3 and/or the second antigen binding region that binds the lymphocyte antigen comprise the F(ab′)₂.

In some embodiments, the first antigen binding region that binds DLL3 and/or the second antigen binding region that binds the lymphocyte antigen comprise the VHH.

In some embodiments, the first antigen binding region that binds DLL3 and/or the second antigen binding region that binds the lymphocyte antigen comprise the Fv.

In some embodiments, the first antigen binding region that binds DLL3 and/or the second antigen binding region that binds the lymphocyte antigen comprise the Fd.

In some embodiments, the first antigen binding region that binds DLL3 and/or the second antigen binding region that binds the lymphocyte antigen comprise the scFv.

In a particular embodiment, the multispecific antigen-binding construct is bispecific, wherein the first antigen binding region that binds DLL3 comprises a scFv and the second antigen binding region that binds the lymphocyte antigen (e.g., CD3) comprises a Fab.

In a particular embodiment, the multispecific antigen-binding construct is bispecific, wherein the first antigen binding region that binds DLL3 comprises a Fab and the second antigen binding region that binds the lymphocyte antigen (e.g., CD3) comprises a scFv.

In some embodiments, the scFv comprises, from the N- to C-terminus, a VH, a first linker (L1) and a VL (VH-L1-VL) or the VL, the L1 and the VH (VL-L1-VH).

In some embodiments, the L1 comprises about 5-50 amino acids.

In some embodiments, the L1 comprises about 5-40 amino acids.

In some embodiments, the L1 comprises about 10-30 amino acids.

In some embodiments, the L1 comprises about 10-20 amino acids.

In some embodiments, the L1 comprises the amino acid sequence of SEQ ID NOs: 27, 72, 73, 74, 75, 76, 79, 81, 82, 83, 88, 90, 91, 92, 120, 121, 122, 123, 124, 125, 126, 127, 128, 129, 130, 131, 132, 133, 134, 135, 136, 137, 138, or 139.

In a particular embodiment, the L1 comprises the amino acid sequence of SEQ ID NO:120.

In some embodiments, the first antigen binding region that binds DLL3 comprises the HCDR1 of SEQ ID NOs:15, 18, 21, 24, 18, 30, 50, 52, 53, 55, 57, 59, or 61, a HCDR2 of SEQ ID NOs:16, 19, 22, 25, 28, 31, 51, 54, 56, 58, 60, or 62, a HCDR3 of SEQ ID NOs:17, 20, 23, 26, 29, 32, 17, 20, 23, 26, 29, or 32, a LCDR1 of SEQ ID NOs:33, 36, 39, 41, 44, or 47, a LCDR2 of SEQ ID NOs:34, 37, 42, 45, or 48, and a LCDR3 of SEQ ID NOs:35, 38, 40, 43, 46, or 49.

In a particular embodiment, the first antigen binding region that binds DLL3 comprises the HCDR1, the HCDR2, the HCDR3, the LCDR1, the LCDR2 and the LCDR3 of SEQ ID NOs:15, 16, 17, 33, 34 and 35, respectively. In some embodiments, the multispecific antigen-binding construct mediates T cell mediated cytotoxicity, promoting T cell activation and proliferation, increasing T cell cytokine release and/or displaying increased anti-tumor efficacy.

In some embodiments, the multispecific antigen-binding construct potently mediates the expansion of cytotoxic CD8 T cells. In some embodiments, the multispecific antigen-binding construct upregulates CD25, CD69 and CD71 expression on the surface of CD8 T cells. In some embodiments, the multispecific antigen-binding construct displays increased tumor killing.

In a particular embodiment, the first antigen binding region that binds DLL3 comprises the HCDR1, the HCDR2, the HCDR3, the LCDR1, the LCDR2 and the LCDR3 of SEQ ID NOs:15, 16, 17, 33, 34 and 35, respectively, and the second antigen binding region that binds a lymphocyte antigen, optionally which is CD3, CD3 epsilon (CD3ε), CD8, KI2L4, NKG2E, NKG2D, NKG2F, BTNL3, CD186, BTNL8, PD-1, CD195, or NKG2C, such as CD3.

In some embodiments, the first antigen binding region that binds DLL3 comprises a VH of SEQ ID NO:3 and a VL which is at least 80% (e.g., at least 85%, at least 90%, at least 95% or at least 99%) identical to the VL of SEQ ID NO:4.

In some embodiments, the first antigen binding region that binds DLL3 comprises a VH which is at least 80% (e.g., at least 85%, at least 90%, at least 95% or at least 99%) identical to the VH of SEQ ID NO:3 and a VL which is at least 80% (e.g., at least 85%, at least 90%, at least 95% or at least 99%) identical to the VL of SEQ ID NO:4, and the second antigen binding region that binds a lymphocyte antigen, optionally which is CD3, CD3 epsilon (CD3ε), CD8, KI2L4, NKG2E, NKG2D, NKG2F, BTNL3, CD186, BTNL8, PD-1, CD195, or NKG2C, such as CD3.

In some embodiments, the isolated multispecific antigen-binding construct mediates T cell mediated cytotoxicity, promoting T cell activation and proliferation, increasing T cell cytokine release and/or displaying increased anti-tumor efficacy. In some embodiments, the multispecific antigen-binding construct potently mediates the expansion of cytotoxic CD8 T cells. In some embodiments, the multispecific antigen-binding construct upregulates CD25, CD69 and CD71 expression on the surface of CD8 T cells. In some embodiments, the multispecific antigen-binding construct displays increased tumor killing.

In some embodiments, the bispecific anti-DLL3×CD3 antibody achieves >90% (e.g., 95%) tumor lysis by 5 days in a T cell cytotoxicity assay.

In some embodiments, the first antigen binding region that binds DLL3 comprises a VH which is at least 80% (e.g., at least 85%, at least 90%, at least 95% or at least 99%) identical to the VH of SEQ ID NO:3 and a VL of SEQ ID NO:4, and the second antigen binding region that binds a lymphocyte antigen, optionally which is CD3, CD3 epsilon (CD3ε), CD8, KI2L4, NKG2E, NKG2D, NKG2F, BTNL3, CD186, BTNL8, PD-1, CD195, or NKG2C, such as CD3.

In some embodiments, the first antigen binding region that binds DLL3 comprises a VH of SEQ ID NO:3 and a VL which is at least 80% (e.g., at least 85%, at least 90%, at least 95% or at least 99%) identical to the VL of SEQ ID NO:4, and the second antigen binding region that binds a lymphocyte antigen, optionally which is CD3, CD3 epsilon (CD3ε), CD8, KI2L4, NKG2E, NKG2D, NKG2F, BTNL3, CD186, BTNL8, PD-1, CD195, or NKG2C, such as CD3.

In some embodiments, the isolated multispecific antigen-binding constructs disclosed herein may be particularly effective at mediating T cell mediated cytotoxicity, promoting T cell activation and proliferation, increasing T cell cytokine release and/or displaying increased anti-tumor efficacy.

In a particular embodiment, the first antigen binding region that binds DLL3 comprises the VH of SEQ ID NO:3 and the VL of SEQ ID NO:4.

In a particular embodiment, the first antigen binding region that binds DLL3 comprises the VH of SEQ ID NO:3 and the VL of SEQ ID NO:4, and the second antigen binding region that binds a lymphocyte antigen, optionally which is CD3, CD3 epsilon (CD3ε), CD8, KI2L4, NKG2E, NKG2D, NKG2F, BTNL3, CD186, BTNL8, PD-1, CD195, or NKG2C, such as CD3.

In some embodiments, the multispecific antigen-binding construct mediates T cell mediated cytotoxicity. In some embodiments, the multispecific antigen-binding construct potently mediates the expansion of cytotoxic CD8 T cells. In some embodiments, the multispecific antigen-binding construct upregulates CD25, CD69 and CD71 expression on the surface of CD8 T cells. In some embodiments, the multispecific antigen-binding construct displays increased tumor killing. In some embodiments, the bispecific anti-DLL3×CD3 antibody achieves >90% (e.g., 95%) tumor lysis by 5 days in a T cell cytotoxicity assay.

In some embodiments, the first antigen binding region that binds DLL3 comprises the amino acid sequence of SEQ ID NOs:63, 64, 65, 66, 67, 68, or 69.

In some embodiments, the first antigen binding region that binds DLL3 comprises the amino acid sequence of SEQ ID NO:63.

In some embodiments, the first antigen binding region that binds DLL3 comprises the amino acid sequence of SEQ ID NO:64.

In some embodiments, the first antigen binding region that binds DLL3 comprises the amino acid sequence of SEQ ID NO:65.

In some embodiments, the first antigen binding region that binds DLL3 comprises the amino acid sequence of SEQ ID NO:66.

In some embodiments, the first antigen binding region that binds DLL3 comprises the amino acid sequence of SEQ ID NO:67.

In some embodiments, the first antigen binding region that binds DLL3 comprises the amino acid sequence of SEQ ID NO:68.

In some embodiments, the first antigen binding region that binds DLL3 comprises the amino acid sequence of SEQ ID NO:69.

In some embodiments, the first antigen binding region that binds DLL3 comprises an amino acid sequence at least 80% (e.g., at least 85%, at least 90%, at least 95% or at least 99%) identical to the amino acid sequence of SEQ ID NO:63.

In some embodiments, the first antigen binding region that binds DLL3 comprises an amino acid sequence at least 80% (e.g., at least 85%, at least 90%, at least 95% or at least 99%) identical to the amino acid sequence of SEQ ID NO:64.

In a particular embodiment, the first antigen binding region that binds DLL3 comprises an amino acid sequence of SEQ ID NOs:63 or 64.

The disclosure also provides a second antigen binding region that binds lymphocyte antigen (such as CD3), wherein the antigen binding region that binds lymphocyte comprises the heavy chain variable region (VH) of SEQ ID NO:77 and the light chain variable region (VL) of SEQ ID NO:80 or the VH of SEQ ID NO:84 and the VL of SEQ ID NO:85.

In some embodiments, the second antigen binding region that binds a lymphocyte antigen (such as CD3) comprises a VH which is at least 80% (e.g., at least 85%, at least 90%, at least 95% or at least 99%) identical to the VH of SEQ ID NO:77 and a VL which is at least 80% (e.g., at least 85%, at least 90%, at least 95% or at least 99%) identical to the VL of SEQ ID NO:80.

In some embodiments, the second antigen binding region that binds a lymphocyte antigen comprises a VH which is at least 80% (e.g., at least 85%, at least 90%, at least 95% or at least 99%) identical to the VH of SEQ ID NO:77 and a VL of SEQ ID NO:80.

In some embodiments, the second antigen binding region that binds a lymphocyte antigen comprises a VH of SEQ ID NO:77 and a VL which is at least 80% (e.g., at least 85%, at least 90%, at least 95% or at least 99%) identical to the VL of SEQ ID NO:80.

In a particular embodiment, the second antigen binding region that binds a lymphocyte antigen comprises a VH of SEQ ID NO:77 and a VL of SEQ ID NO:80.

In some embodiments, the second antigen binding region that binds a lymphocyte antigen (such as CD3) comprises a VH which is at least 80% (e.g., at least 85%, at least 90%, at least 95% or at least 99%) identical to the VH of SEQ ID NO:84 and a VL which is at least 80% (e.g., at least 85%, at least 90%, at least 95% or at least 99%) identical to the VL of SEQ ID NO:85.

In some embodiments, the second antigen binding region that binds a lymphocyte antigen comprises a VH which is at least 80% (e.g., at least 85%, at least 90%, at least 95% or at least 99%) identical to the VH of SEQ ID NO:84 and a VL of SEQ ID NO:85.

In some embodiments, the second antigen binding region that binds a lymphocyte antigen comprises a VH of SEQ ID NO:84 and a VL which is at least 80% (e.g., at least 85%, at least 90%, at least 95% or at least 99%) identical to the VL of SEQ ID NO:85.

In a particular embodiment, the second antigen binding region that binds a lymphocyte antigen comprises a VH of SEQ ID NO:84 and a VL of SEQ ID NO:85.

In some embodiments, the second antigen binding region that binds a lymphocyte antigen comprises:
a HCDR1 of SEQ ID NO:95, a HCDR2 of SEQ ID NO:96, a HCDR3 of SEQ ID NO:97, a LCDR1 of SEQ ID NO:101, a LCDR2 of SEQ ID NO: 102 and a LCDR3 of SEQ ID NO:104; or the VH of SEQ ID NO:77 and the VL of SEQ ID NO:80.

In some embodiments, the second antigen binding region that binds a lymphocyte antigen comprises:
a HCDR1 of SEQ ID NO:98, a HCDR2 of SEQ ID NO:99, a HCDR3 of SEQ ID NO:100, a LCDR1 of SEQ ID NO: 106, a LCDR2 of SEQ ID NO:107 and a LCDR3 of SEQ ID NO:108; or the VH of SEQ ID NO:84 and the VL of SEQ ID NO:85.

In a particular embodiment, the second antigen binding region that binds a lymphocyte antigen (such as CD3) comprises the HCDR1 of SEQ ID NO:95, the HCDR2 of SEQ ID NO:96, the HCDR3 of SEQ ID NO:97, the LCDR1 of SEQ ID NO:101, the LCDR2 of SEQ ID NO:102 and the LCDR3 of SEQ ID NO:104.

In a particular embodiment, the first antigen binding region that binds DLL3 comprises the HCDR1, the HCDR2, the HCDR3, the LCDR1, the LCDR2 and the LCDR3 of SEQ ID NOs:15, 16, 17, 33, 34 and 35, respectively and the second antigen binding region that binds a lymphocyte antigen (such as CD3) comprises the HCDR1 of SEQ ID NO:95, the HCDR2 of SEQ ID NO:96, the HCDR3 of SEQ ID NO:97, the LCDR1 of SEQ ID NO:101, the LCDR2 of SEQ ID NO:102 and the LCDR3 of SEQ ID NO:104.

In a particular embodiment, the first antigen binding region that binds DLL3 comprises the HCDR1, the HCDR2, the HCDR3, the LCDR1, the LCDR2 and the LCDR3 of SEQ ID NOs:15, 16, 17, 33, 34, and 35, respectively and the second antigen binding region that binds a lymphocyte antigen (such as CD3) comprises the HCDR1 of SEQ ID NO:98, the HCDR2 of SEQ ID NO:99, the HCDR3 of SEQ ID NO:100, the LCDR1 of SEQ ID NO:106, the LCDR2 of SEQ ID NO:107 and the LCDR3 of SEQ ID NO:108.

In a particular embodiment, the first antigen binding region that binds DLL3 comprises the HCDR1, the HCDR2, the HCDR3, the LCDR1, the LCDR2 and the LCDR3 of SEQ ID NOs:15, 16, 17, 33, 34, and 35, respectively and the second antigen binding region that binds a lymphocyte antigen comprises a VH of SEQ ID NO:77 and a VL of SEQ ID NO:80. In some embodiments, the multispecific antigen-binding construct mediates T cell mediated cytotoxicity.

In some embodiments, the multispecific antigen-binding construct potently mediates the expansion of cytotoxic CD8 T cells. In some embodiments, the multispecific antigen-binding construct upregulates CD25, CD69 and CD71 expression on the surface of CD8 T cells. In some embodiments, the multispecific antigen-binding construct displays increased tumor killing. In some embodiments, the bispecific anti-DLL3×CD3 antibody achieves >90% (e.g., 95%) tumor lysis by 5 days in a T cell cytotoxicity assay.

In a particular embodiment, the first antigen binding region that binds DLL3 comprises the HCDR1, the HCDR2, the HCDR3, the LCDR1, the LCDR2 and the LCDR3 of SEQ ID NOs:15, 16, 17, 33, 34, and 35, respectively and the second antigen binding region that binds a lymphocyte antigen (such as CD3) comprises a VH of SEQ ID NO:84 and a VL of SEQ ID NO:85. In some embodiments, the multispecific antigen-binding construct mediates T cell mediated cytotoxicity, promoting T cell activation, proliferation, and expansion, increasing T cell cytokine release and/or displaying increased anti-tumor efficacy. In some embodiments, the multispecific antigen-binding construct potently mediates the expansion of cytotoxic CD8 T cells. In some embodiments, the multispecific antigen-binding construct upregulates CD25, CD69 and CD71 expression on the surface of CD8 T cells. In some embodiments, the multispecific antigen-binding construct displays increased tumor killing. In some embodiments, the bispecific anti-DLL3×CD3 antibody achieves >90% (e.g., 95%) tumor lysis by 5 days in a T cell cytotoxicity assay.

In a particular embodiment, the first antigen binding region that binds DLL3 comprises a VH of SEQ ID NO:3 and a VL of SEQ ID NO:4 and the second antigen binding region that binds a lymphocyte antigen (such as CD3) comprises the HCDR1 of SEQ ID NO:95, the HCDR2 of SEQ ID NO:96, the HCDR3 of SEQ ID NO:97, the LCDR1 of SEQ ID NO:101, the LCDR2 of SEQ ID NO:102 and the LCDR3 of SEQ ID NO: 104.

In a particular embodiment, the first antigen binding region that binds DLL3 comprises a VH of SEQ ID NO:3 and a VL of SEQ ID NO:4 and the second antigen binding region that binds a lymphocyte antigen comprises a VH of SEQ ID NO:84 and a VL of SEQ ID NO:85.

In a particular embodiment, the first antigen binding region that binds DLL3 comprises a VH of SEQ ID NO:3 and a VL of SEQ ID NO:4 and the second antigen binding region that binds a lymphocyte antigen comprises a VH of SEQ ID NO:77 and a VL of SEQ ID NO:80.

Generation of Multispecific Antigen-Binding Constructs that Comprise Antigen Binding Regions that Bind DLL3

The antigen binding regions that bind DLL3 of the disclosure may be engineered into multispecific antibodies which are also encompassed within the scope of the application.

The antigen binding regions that bind DLL3 may be engineered into full length multispecific antibodies which are generated using Fab arm exchange, in which substitutions are introduced into two monospecific bivalent antibodies within the Ig constant region CH3 domain which promote Fab arm exchange in vitro. In the methods, two monospecific bivalent antibodies are engineered to have certain substitutions at the CH3 domain that promote heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

CH3 mutations that may be used include technologies such as Knob-in-Hole mutations (Genentech), electrostatically-matched mutations (Chugai, Amgen, NovoNordisk, Oncomed), the Strand Exchange Engineered Domain body (SEEDbody) (EMD Serono), Duobody® mutations (Genmab), and other asymmetric mutations (e.g., Zymeworks).

Knob-in-hole mutations are disclosed for example in WO1996/027011 and include mutations on the interface of CH3 region in which an amino acid with a small side chain (hole) is introduced into the first CH3 region and an amino acid with a large side chain (knob) is introduced into the second CH3 region, resulting in preferential interaction between the first CH3 region and the second CH3 region. Exemplary CH3 region mutations forming a knob and a hole are T366Y/F405A, T366W/F405W, F405W/Y407A, T394W/Y407T, T394 S/Y407A, T366W/T394 S, F405W/T394 S and T366W/T366 S_L368A_Y407V.

Heavy chain heterodimer formation may be promoted by using electrostatic interactions by substituting positively charged residues on the first CH3 region and negatively charged residues on the second CH3 region as described in US2010/0015133, US2009/0182127, US2010/028637 or US2011/0123532.

Other asymmetric mutations that can be used to promote heavy chain heterodimerization are L351Y_F405A_Y407V/T394W, T3661_K392M_T394W/F405A_Y407V,
T366L_K392M_T394W/F405A_Y407V, L351Y_Y407A/T366A_K409F,
L351Y_Y407A/T366V_K409F, Y407A/T366A_K409F, or
T350V_L351Y_F405A_Y407V/T350V_T366L_K392L_T394W as described in US2012/0149876 or US2013/0195849 (Zymeworks).

SEEDbody mutations involve substituting select IgG residues with IgA residues to promote heavy chai heterodimerization as described in US20070287170.

Other exemplary mutations that may be used are R409D_K370E/D399K_E357K, S354C_T366W/Y349C_T366 S_L368A_Y407V, Y349C_T366W/S354C_T366 S_L368A_Y407V, T366K/L351D, L351K/Y349E, L351K/Y349D, L351K/L368E, L351Y_Y407A/T366A_K409F, L351Y_Y407A/T366V_K409F, K392D/D399K, K392D/E356K, K253E_D282K_K322D/D239K_E240K_K292D, K392D_K409D/D356K_D399K as described in WO2007/147901, WO 2011/143545, WO2013157954, WO2013096291 and US2018/0118849.

Duobody® mutations (Genmab) are disclosed for example in U.S. Pat. No. 9,150,663 and US2014/0303356 and include mutations F405L/K409R, wild-type/F405L_R409K, T350I_K370T_F405L/K409R, K370W/K409R, D399AFGHILMNRSTVWY/K409R, T366ADEFGHILMQVY/K409R, L368ADEGHNRSTVQ/K409AGRH, D399FHKRQ/K409AGRH, F405IKLSTVW/K409AGRH and Y407LWQ/K409AGRH.

Additional bispecific or multispecific structures into which the antigen binding regions that bind DLL3 can be incorporated include Dual Variable Domain Immunoglobulins (DVD) (Int. Pat. Publ. No. WO2009/134776; DVDs are full length antibodies comprising the heavy chain having a structure VH1-linker-VH2-CH and the light chain having the structure VL1-linker-VL2-CL; linker being optional), structures that include various dimerization domains to connect the two antibody arms with different specificity, such as leucine zipper or collagen dimerization domains (Int. Pat. Publ. No. WO2012/022811; U.S. Pat. Nos. 5,932,448; 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)2-Fab (National Research Center for Antibody Medicine—China), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

The antigen binding regions that bind DLL3 of the disclosure may also be engineered into multispecific antigen-binding constructs which comprise three polypeptide chains. In such designs, at least one antigen binding region is in the form of a scFv. Exemplary designs include (in which “1” indicates the first antigen binding region, “2” indicates the second antigen binding region and “3” indicates the third antigen binding region):
Design 1: Chain A) scFv1-CH2-CH3; Chain B) VL2-CL; Chain C) VH2-CH1-hinge-CH2-CH3
Design 2: Chain A) scFv1-hinge-CH2-CH3; Chain B) VL2-CL; Chain C) VH2-CH1-hinge-CH2-CH3
Design 3: Chain A) scFv1-CH1-hinge-CH2-CH3; Chain B) VL2-CL; Chain C) VH2-CH1-hinge-CH2-CH3
Design 4: Chain A) CH2-CH3-scFv1; Chain B) VL2-CL; Chain C) VH2-CH1-hinge-CH2-CH3
CH3 engineering may be incorporated to the Designs 1-4, such as mutations
L351Y_F405A_Y407V/T394W, T3661_K392M_T394W/F405A_Y407V,
T366L_K392M_T394W/F405A_Y407V, L351Y_Y407A/T366A_K409F,
L351Y_Y407A/T366V_K409F, Y407A/T366A_K409F, or
T350V_L351Y_F405A_Y407V/T350V_T366L_K392L_T394W as described in US2012/0149876 or US2013/0195849 (Zymeworks).

In a particular embodiment, the design is Chain A) scFv1-hinge-CH2-CH3; Chain B) VL2-CL; Chain C) VH2-CH1-hinge-CH2-CH3.

In some embodiment, the isolated multispecific antigen-binding construct comprises a first antigen binding region that binds DLL3 and a second antigen binding region that binds a lymphocyte antigen (such as CD3), wherein the first antigen binding region that binds DLL3 comprises a HCDR1 of SEQ ID NOs:15, 18, 21, 24, 18, 30, 50, 52, 53, 55, 57, 59, or 61, a HCDR2 of SEQ ID NOs:16, 19, 22, 25, 28, 31, 51, 54, 56, 58, 60, or 62, a HCDR3 of SEQ ID NOs:17, 20, 23, 26, 29, 32, 17, 20, 23, 26, 29, or 32, a LCDR1 of SEQ ID NOs:33, 36, 39, 41, 44, or 47, a LCDR2 of SEQ ID NOs:34, 37, 42, 45, or 48, and a LCDR3 of SEQ ID NOs:35, 38, 40, 43, 46, or 49.

In some embodiment, the isolated multispecific antigen-binding construct comprises a first antigen binding region that binds DLL3 and a second antigen binding region that binds a lymphocyte antigen (such as CD3), wherein the first antigen binding region that binds DLL3 comprises the HCDR1, the HCDR2, the HCDR3, the LCDR1, the LCDR2 and the LCDR3 of
SEQ ID NOs:15, 16, 17, 33, 34, 35, respectively;
SEQ ID NOs:18, 19, 20, 36, 37, 38, respectively;
SEQ ID NOs:21, 22, 23, 39, 37, 40, respectively;
SEQ ID NOs:24, 25, 26, 41, 42, 43, respectively;
SEQ ID NOs:18, 28, 29, 44, 45, 46, respectively;
SEQ ID NOs:30, 31, 32, 47, 48, 49, respectively;
SEQ ID NOs:50, 51, 17, 33, 34, 35, respectively;
SEQ ID NOs:52, 51, 17, 33, 34, 35, respectively;
SEQ ID NOs:53, 54, 20, 36, 37, 38, respectively;
SEQ ID NOs:55, 56, 23, 39, 37, 40, respectively;
SEQ ID NOs:57, 58, 26, 41, 42, 43, respectively;
SEQ ID NOs:59, 60, 29, 44, 45, 46, respectively; or
SEQ ID NOs:61, 62, 32, 47, 48, 49, respectively.

In a particular embodiment, the isolated multispecific antigen-binding construct comprises a first antigen binding region that binds DLL3 and a second antigen binding region that binds a lymphocyte antigen (e.g., CD3), wherein the first antigen binding region that binds DLL3 comprises the HCDR1, the HCDR2, the HCDR3, the LCDR1, the LCDR2, and the LCDR3 of SEQ ID NOs:15, 16, 17, 33, 34, 35, respectively. In some embodiments, the isolated multispecific antigen-binding construct mediates T cell mediated cytotoxicity, promoting T cell activation and proliferation, increasing T cell cytokine release and/or displaying increased anti-tumor efficacy. In some embodiments, the isolated multispecific antigen-binding construct potently mediates the expansion of cytotoxic CD8 T cells. In some embodiments, the isolated multispecific antigen-binding construct upregulates CD25, CD69 and CD71 expression on the surface of CD8 T cells. In some embodiments, the isolated multispecific antigen-binding construct displays increased tumor killing. In some embodiments, the bispecific anti-DLL3×CD3 antibody achieves >90% (e.g., 95%) tumor lysis by 5 days in a T cell cytotoxicity assay.

In some embodiment, the isolated multispecific antigen-binding construct comprises a first antigen binding region that binds DLL3 and a second antigen binding region that binds a lymphocyte antigen (such as CD3), wherein the first antigen binding region that binds DLL3 comprises:
the VH of SEQ ID NO:1 and the VL of SEQ ID NO:2;
the VH of SEQ ID NO:1 and the VL of SEQ ID NO:4;
the VH of SEQ ID NO:1 and the VL of SEQ ID NO:6;
the VH of SEQ ID NO:1 and the VL of SEQ ID NO:8;
the VH of SEQ ID NO:1 and the VL of SEQ ID NO:10;
the VH of SEQ ID NO:1 and the VL of SEQ ID NO:12;
the VH of SEQ ID NO:1 and the VL of SEQ ID NO:14;
the VH of SEQ ID NO:3 and the VL of SEQ ID NO:2;
the VH of SEQ ID NO:3 and the VL of SEQ ID NO:4;
the VH of SEQ ID NO:3 and the VL of SEQ ID NO:6;
the VH of SEQ ID NO:3 and the VL of SEQ ID NO:8;
the VH of SEQ ID NO:3 and the VL of SEQ ID NO:10;
the VH of SEQ ID NO:3 and the VL of SEQ ID NO:12;
the VH of SEQ ID NO:3 and the VL of SEQ ID NO:14;
the VH of SEQ ID NO:3 and the VL of SEQ ID NO:2;
the VH of SEQ ID NO:3 and the VL of SEQ ID NO:4;
the VH of SEQ ID NO:3 and the VL of SEQ ID NO:6;
the VH of SEQ ID NO:3 and the VL of SEQ ID NO:8;
the VH of SEQ ID NO:3 and the VL of SEQ ID NO:10;
the VH of SEQ ID NO:3 and the VL of SEQ ID NO:12;
the VH of SEQ ID NO:3 and the VL of SEQ ID NO:14;
the VH of SEQ ID NO:5 and the VL of SEQ ID NO:2;
the VH of SEQ ID NO:5 and the VL of SEQ ID NO:4;
the VH of SEQ ID NO:5 and the VL of SEQ ID NO:6;
the VH of SEQ ID NO:5 and the VL of SEQ ID NO:8;
the VH of SEQ ID NO:5 and the VL of SEQ ID NO:10;
the VH of SEQ ID NO:5 and the VL of SEQ ID NO:12;
the VH of SEQ ID NO:5 and the VL of SEQ ID NO:14;
the VH of SEQ ID NO:7 and the VL of SEQ ID NO:2;
the VH of SEQ ID NO:7 and the VL of SEQ ID NO:4;
the VH of SEQ ID NO:7 and the VL of SEQ ID NO:6;
the VH of SEQ ID NO:7 and the VL of SEQ ID NO:8;
the VH of SEQ ID NO:7 and the VL of SEQ ID NO:10;
the VH of SEQ ID NO:7 and the VL of SEQ ID NO:12;
the VH of SEQ ID NO:7 and the VL of SEQ ID NO:14;
the VH of SEQ ID NO:9 and the VL of SEQ ID NO:2;
the VH of SEQ ID NO:9 and the VL of SEQ ID NO:4;
the VH of SEQ ID NO:9 and the VL of SEQ ID NO:6;
the VH of SEQ ID NO:9 and the VL of SEQ ID NO:8;
the VH of SEQ ID NO:9 and the VL of SEQ ID NO:10;
the VH of SEQ ID NO:9 and the VL of SEQ ID NO:12;
the VH of SEQ ID NO:9 and the VL of SEQ ID NO:14;
the VH of SEQ ID NO: 11 and the VL of SEQ ID NO:2;
the VH of SEQ ID NO: 11 and the VL of SEQ ID NO:4;
the VH of SEQ ID NO: 11 and the VL of SEQ ID NO:6;
the VH of SEQ ID NO: 11 and the VL of SEQ ID NO:8;
the VH of SEQ ID NO: 11 and the VL of SEQ ID NO: 10;
the VH of SEQ ID NO: 11 and the VL of SEQ ID NO: 12;
the VH of SEQ ID NO: 11 and the VL of SEQ ID NO: 14;
the VH of SEQ ID NO: 13 and the VL of SEQ ID NO:2;
the VH of SEQ ID NO: 13 and the VL of SEQ ID NO:4;
the VH of SEQ ID NO: 13 and the VL of SEQ ID NO:6;
the VH of SEQ ID NO: 13 and the VL of SEQ ID NO:8;
the VH of SEQ ID NO: 13 and the VL of SEQ ID NO: 10;
the VH of SEQ ID NO: 13 and the VL of SEQ ID NO: 12; or
the VH of SEQ ID NO: 13 and the VL of SEQ ID NO: 14.

In some embodiments, the isolated multispecific antigen-binding construct comprises a first antigen binding region that binds DLL3 and a second antigen binding region that binds a lymphocyte antigen (e.g., CD3), wherein the first antigen binding region that binds DLL3 comprises a VH which is at least 80% (e.g., at least 85%, at least 90%, at least 95% or at least 99%) identical to the VH of SEQ ID NO:3 and a VL which is at least 80% (e.g., at least 85%, at least 90%, at least 95% or at least 99%) identical to the VL of SEQ ID NO:4.

In a particular embodiment, the isolated multispecific antigen-binding construct comprises a first antigen binding region that binds DLL3 and a second antigen binding region that binds a lymphocyte antigen (e.g., CD3), wherein the first antigen binding region that binds DLL3 comprises a VH of SEQ ID NO:3 and a VL which is at least 80% (e.g., at least 85%, at least 90%, at least 95% or at least 99%) identical to the VL of SEQ ID NO:4. In some embodiments, the isolated multispecific antigen-binding constructs disclosed herein may be particularly effective at mediating T cell mediated cytotoxicity, promoting T cell activation and proliferation, increasing T cell cytokine release and/or displaying increased anti-tumor efficacy. In a particular embodiment, the isolated multispecific antigen-binding construct comprises a first antigen binding region that binds DLL3 and a second antigen binding region that binds a lymphocyte antigen (e.g., CD3), wherein the first antigen binding region that binds DLL3 comprises a VH which is at least 80% (e.g., at least 85%, at least 90%, at least 95% or at least 99%) identical to the VH of SEQ ID NO:3 and a VL of SEQ ID NO:4.

In a particular embodiment, the isolated multispecific antigen-binding construct comprises a first antigen binding region that binds DLL3 and a second antigen binding region that binds a lymphocyte antigen (e.g., CD3), wherein the first antigen binding region that binds DLL3 comprises a VH which is at least 80% (e.g., at least 85%, at least 90%, at least 95% or at least 99%) identical to the VH of SEQ ID NO:3 and a VL of SEQ ID NO:4.

In a particular embodiment, the isolated multispecific antigen-binding construct comprises a first antigen binding region that binds DLL3 and a second antigen binding region that binds a lymphocyte antigen (e.g., CD3), wherein the first antigen binding region that binds DLL3 comprises a VH of SEQ ID NO:3 and a VL which is at least 80% (e.g., at least 85%, at least 90%, at least 95% or at least 99%) identical to the VL of SEQ ID NO:4.

In a particular embodiment, the isolated multispecific antigen-binding construct comprises a first antigen binding region that binds DLL3 and a second antigen binding region that binds a lymphocyte antigen (e.g., CD3), wherein the first antigen binding region that binds DLL3 comprises a VH which is at least 80% (e.g., at least 85%, at least 90%, at least 95% or at least 99%) identical to the VH of SEQ ID NO:3 and a VL which is at least 80% (e.g., at least 85%, at least 90%, at least 95% or at least 99%) identical to the VL of SEQ ID NO:4. In some embodiments, the isolated multispecific antigen-binding construct mediates T cell mediated cytotoxicity. In some embodiments, the isolated multispecific antigen-binding construct potently mediates the expansion of cytotoxic CD8 T cells. In some embodiments, the isolated multispecific antigen-binding construct upregulates CD25, CD69 and CD71 expression on the surface of CD8 T cells. In some embodiments, the isolated multispecific antigen-binding construct displays increased tumor killing. In some embodiments, the bispecific anti-DLL3×CD3 antibody achieves >90% (e.g., 95%) tumor lysis by 5 days in a T cell cytotoxicity assay.

In a particular embodiment, the isolated multispecific antigen-binding construct comprises a first antigen binding region that binds DLL3 and a second antigen binding region that binds a lymphocyte antigen (e.g., CD3), wherein the first antigen binding region that binds DLL3 comprises a VH of SEQ ID NO:3 and a VL of SEQ ID NO:4.

In some embodiment, the isolated multispecific antigen-binding construct comprises a first antigen binding region that binds DLL3 and a second antigen binding region that binds a lymphocyte antigen, wherein the first antigen binding region that binds DLL3 comprises the amino acid sequence of SEQ ID NOs:63, 64, 65, 66, 67, 68, or 69.

In some embodiments, the isolated multispecific antigen-binding construct comprises a first antigen binding region that binds DLL3 and a second antigen binding region that binds a lymphocyte antigen (e.g., CD3), wherein the first antigen binding region that binds DLL3 comprises an amino acid sequence at least 80% (e.g., at least 85%, at least 90%, at least 95% or at least 99%) identical to the amino acid sequence of SEQ ID NOs:63 or 64.

In a particular embodiment, the isolated multispecific antigen-binding construct comprises a first antigen binding region that binds DLL3 and a second antigen binding region that binds a lymphocyte antigen (e.g., CD3), wherein the first antigen binding region that binds DLL3 comprises an amino acid sequence of SEQ ID NOs:63 or 64.

In some embodiments, the isolated multispecific antigen-binding construct comprises a first antigen binding region that binds DLL3 and a second antigen binding region that binds a lymphocyte antigen (e.g., CD3), wherein the second antigen binding region that binds the lymphocyte antigen comprises a HCDR1 of SEQ ID NOs:95 or 98, a HCDR2 of SEQ ID NOs:96 or 99, a HCDR3 of SEQ ID NOs:97 or 100, a LCDR1 of SEQ ID NO:101 or 106, a LCDR2 of SEQ ID NOs:102 or 107, and a LCDR3 of SEQ ID NOs:103, 104, or 108.

In some embodiments, the isolated multispecific antigen-binding construct comprises a first antigen binding region that binds DLL3 and a second antigen binding region that binds a lymphocyte antigen (e.g., CD3), wherein the second antigen binding region that binds the lymphocyte antigen comprises:
a HCDR1 of SEQ ID NO:95, a HCDR2 of SEQ ID NO:96, a HCDR3 of SEQ ID NO:97, a LCDR1 of SEQ ID NO:101, a LCDR2 of SEQ ID NO: 102 and a LCDR3 of SEQ ID NO:104; or
a HCDR1 of SEQ ID NO:98, a HCDR2 of SEQ ID NO:99, a HCDR3 of SEQ ID NO:100, a LCDR1 of SEQ ID NO: 106, a LCDR2 of SEQ ID NO:107 and a LCDR3 of SEQ ID NO:108.

In a particular embodiment, the isolated multispecific antigen-binding construct comprises a first antigen binding region that binds DLL3 and a second antigen binding region that binds a lymphocyte antigen (e.g., CD3), wherein the second antigen binding region that binds the lymphocyte antigen comprises a HCDR1 of SEQ ID NO:95, a HCDR2 of SEQ ID NO:96, a HCDR3 of SEQ ID NO:97, a LCDR1 of SEQ ID NO: 101, a LCDR2 of SEQ ID NO:102 and a LCDR3 of SEQ ID NO: 104.

In some embodiments, the isolated multispecific antigen-binding construct comprises a first antigen binding region that binds DLL3 and a second antigen binding region that binds a lymphocyte antigen (e.g., CD3), wherein the second antigen binding region that binds the lymphocyte antigen comprises: the VH of SEQ ID NO:77 and the VL of SEQ ID NO:80.

In some embodiment, the isolated multispecific antigen-binding construct comprises a first antigen binding region that binds DLL3 and a second antigen binding region that binds a lymphocyte antigen, wherein the second antigen binding region that binds the lymphocyte antigen comprises:
a HCDR1 of SEQ ID NO:98, a HCDR2 of SEQ ID NO:99, a HCDR3 of SEQ ID NO:100, a LCDR1 of SEQ ID NO: 106, a LCDR2 of SEQ ID NO:107 and a LCDR3 of SEQ ID NO:108; or the VH of SEQ ID NO:84 and the VL of SEQ ID NO:85.

The disclosure also provides an isolated anti-DLL3/anti-CD3 protein comprising a first antigen binding region that binds DLL3 and a second antigen binding region that binds CD3, wherein
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs:95, 96, 97, 101, 102, 104, respectively; and/or
b. the first antigen binding region that binds DLL3 comprises a Fab comprising a VH of SEQ ID NO:1 and a VL of SEQ ID NO:2, and the second antigen binding region that binds CD3 comprises a scFv of SEQ ID NO: 105; and/or
c. the isolated anti-DLL/anti-CD3 protein comprises a HC1 of SEQ ID NO:109, a LC1 of SEQ ID NO:110, and a HC1 of SEQ ID NO:112.

The disclosure also provides an isolated anti-DLL3/anti-CD3 protein comprising a first antigen binding region that binds DLL3 and a second antigen binding region that binds CD3, wherein
a. The first antigen binding region that binds DLL3 comprises a HCDR1, a HCDR2, a HCDR3, a LCDR1, a LCDR2 and a LCDR3 of SEQ ID NOs: 15, 16, 17, 33, 34, 35, respectively, and the second domain that binds CD3 comprises the HCDR1, the HCDR2, the HCDR3, the LCDR1, the LCDR2 and the LCDR3 of SEQ ID NOs:95, 96, 97, 101, 102, 104, respectively; and/or
b. the first antigen binding region that binds DLL3 comprises a Fab comprising a VH of SEQ ID NO:1 and a VL of SEQ ID NO:2, and the second antigen binding region that binds CD3 comprises a scFv of SEQ ID NO: 119; and/or
c. the isolated anti-DLL3/anti-CD3 protein comprises a HC1 of SEQ ID NO: 109, a LC1 of SEQ ID NO:110, and a HC1 of SEQ ID NO:113.

The disclosure also provides an isolated anti-DLL3/anti-CD3 protein comprising a first antigen binding region that binds DLL3 and a second antigen binding region that binds CD3, wherein
a. The first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs:98, 99, 100, 106, 107, 108, respectively; and/or
b. the first antigen binding region that binds DLL3 comprises a scFv of SEQ ID NO:63, and the second antigen binding region that binds CD3 comprises a VH of SEQ ID NO:84 and a VL of SEQ ID NO:85; and/or
c. the isolated anti-DLL3/anti-CD3 protein comprises a HC1 of SEQ ID NO:111, a HC2 of SEQ ID NO:116, and a LC2 of SEQ ID NO:117.

The disclosure also provides an isolated anti-DLL3/anti-CD3 protein comprising a first antigen binding region that binds DLL3 and a second antigen binding region that binds CD3, wherein
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs:95, 96, 97, 101, 102, 104, respectively; and/or
b. the first antigen binding region that binds DLL3 comprises a scFv of SEQ ID NO:63, and the second antigen binding region that binds CD3 comprises a VH of SEQ ID NO:77 and a VL of SEQ ID NO:80; optionally, and/or
c. the isolated anti-DLL3/anti-CD3 protein comprises a HC1 of SEQ ID NO:111, a HC2 of SEQ ID NO: 114, and a LC2 of SEQ ID NO:115.

The disclosure also provides an isolated anti-DLL3/anti-CD3 protein comprising a first antigen binding region that binds DLL3 and a second antigen binding region that binds CD3, wherein
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs: 98, 99, 100, 106, 107, 108, respectively; and/or
b. the first antigen binding region that binds DLL3 comprises a scFv of SEQ ID NO:64, and the second antigen binding region that binds CD3 comprises a Fab comprising a VH of SEQ ID NO:84 and a VL of SEQ ID NO:85; and/or
c. the isolated anti-DLL3/anti-CD3 protein comprises a HC1 of SEQ ID NO:71, a HC2 of SEQ ID NO:118, and a LC2 of SEQ ID NO:117.

The disclosure also provides an isolated anti-DLL3/anti-CD3 protein comprising a first antigen binding region that binds DLL3 and a second antigen binding region that binds CD3, wherein
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs:98, 99, 100, 106, 107, 108, respectively;
b. the first antigen binding region that binds DLL3 comprises a scFv of SEQ ID NO:64 and the second antigen binding region that binds the lymphocyte antigen comprises a Fab comprising a VH of SEQ ID NO:84 and a VL of SEQ ID NO:85; and/or
c. the isolated anti-DLL3/anti-CD3 protein comprises a HC1 of SEQ ID NO:229, a HC2 of SEQ ID NO:230, and a LC2 of SEQ ID NO:117.

The disclosure also provides an isolated anti-DLL3/anti-CD3 protein comprising a first antigen binding region that binds DLL3 and a second antigen binding region that binds CD3, wherein
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs: 98, 99, 100, 106, 107, 108, respectively;
b. the first antigen binding region that binds DLL3 comprises a scFv which is at least 80% (e.g., at least 85%, at least 90%, at least 95%, at least 99% or 100%) identical to the scFv of SEQ ID NO:64, and the second antigen binding region that binds CD3 comprises a Fab comprising a VH which is at least 80% (e.g., at least 85%, at least 90%, at least 95%, at least 99% or 100%) identical to the VH of SEQ ID NO:84 and a VL which is at least 80% (e.g., at least 85%, at least 90%, at least 95%, at least 99% or 100%) identical to the VL of SEQ ID NO:85; and/or
c. the isolated anti-DLL3/anti-CD3 protein comprises a HC1 which is at least 80% (e.g., at least 85%, at least 90%, at least 95%, at least 99% or 100%) identical to the HC1 of SEQ ID NO:71, a HC2 which is at least 80% (e.g., at least 85%, at least 90%, at least 95%, at least 99% or 100%) identical to the HC2 of SEQ ID NO:118, and a LC2 which is at least 80% (e.g., at least 85%, at least 90%, at least 95%, at least 99% or 100%) identical to the of SEQ ID NO:117.

The disclosure also provides an isolated anti-DLL3/anti-CD3 protein comprising a first antigen binding region that binds DLL3 and a second antigen binding region that binds CD3, wherein
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs: 98, 99, 100, 106, 107, 108, respectively;
b. the first antigen binding region that binds DLL3 comprises a scFv which is at least 80% (e.g., at least 85%, at least 90%, at least 95%, at least 99% or 100%) identical to the scFv of SEQ ID NO:64, and the second antigen binding region that binds CD3 comprises a Fab comprising a VH which is at least 80% (e.g., at least 85%, at least 90%, at least 95%, at least 99% or 100%) identical to the VH of SEQ ID NO:84 and a VL which is at least 80% (e.g., at least 85%, at least 90%, at least 95%, at least 99% or 100%) identical to the VL of SEQ ID NO:85; and/or
c. the isolated anti-DLL3/anti-CD3 protein comprises a HC1 which is at least 80% (e.g., at least 85%, at least 90%, at least 95%, at least 99% or 100%) identical to the HC1 of SEQ ID NO:229, a HC2 which is at least 80% (e.g., at least 85%, at least 90%, at least 95%, at least 99% or 100%) identical to the HC2 of SEQ ID NO:230, and a LC2 which is at least 80% (e.g., at least 85%, at least 90%, at least 95%, at least 99% or 100%) identical to the of SEQ ID NO: 117.

In a particular embodiment, the disclosure provides an isolated multispecific antigen-binding construct, comprising a first antigen binding region that binds DLL3 and a second antigen binding region that binds CD3, wherein
a) the first antigen binding region that binds DLL3 comprises a HCDR1, a HCDR2, a HCDR3, a LCDR1, a LCDR2 and a LCDR3 of SEQ ID NOs:15, 16, 17, 33, 34, and 35, respectively, and the second domain that binds CD3 comprises the HCDR1, the HCDR2, the HCDR3, the LCDR1, the LCDR2 and the LCDR3 of SEQ ID NOs:98, 99, 100, 106, 107, and 108, respectively; and/or
b) the first antigen binding region that binds DLL3 comprises a scFv of SEQ ID NO:64 and the second antigen binding region that binds CD3 comprises a VH of SEQ ID NO:77 and a VL of SEQ ID NO:80.

In some embodiments, the isolated anti-DLL3/anti-CD3 protein mediates T cell mediated cytotoxicity. In some embodiments, the isolated anti-DLL3/anti-CD3 protein potently mediates the expansion of cytotoxic CD8 T cells. In some embodiments, the isolated anti-DLL3/anti-CD3 protein upregulates CD25, CD69 and CD71 expression on the surface of CD8 T cells. In some embodiments, the isolated anti-DLL3/anti-CD3 protein displays increased tumor killing. In some embodiments, the isolated anti-DLL3/anti-CD3 protein achieves >90% (e.g., 95%) tumor lysis by 5 days in a T cell cytotoxicity assay.

The disclosure also provides an isolated anti-DLL3/anti-CD3 protein comprising a first antigen binding region that binds DLL3 and a second antigen binding region that binds CD3, wherein
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs: 98, 99, 100, 106, 107, 108, respectively;
b. the first antigen binding region that binds DLL3 comprises a scFv which is at least 80% (e.g., at least 85%, at least 90%, at least 95%, at least 99% or 100%) identical to the scFv of SEQ ID NO:64, and the second antigen binding region that binds CD3 comprises a Fab comprising a VH which is at least 80% (e.g., at least 85%, at least 90%, at least 95%, at least 99% or 100%) identical to the VH of SEQ ID NO:84 and a VL which is at least 80% (e.g., at least 85%, at least 90%, at least 95%, at least 99% or 100%) identical to the VL of SEQ ID NO:85; and/or
c. the isolated anti-DLL3/anti-CD3 protein comprises a HC1 which is at least 80% (e.g., at least 85%, at least 90%, at least 95%, at least 99% or 100%) identical to the HC1 of SEQ ID NO:229, a HC2 which is at least 80% (e.g., at least 85%, at least 90%, at least 95%, at least 99% or 100%) identical to the HC2 of SEQ ID NO:230, and a LC2 which is at least 80% (e.g., at least 85%, at least 90%, at least 95%, at least 99% or 100%) identical to the of SEQ ID NO:117.

In some embodiments, the isolated anti-DLL3/anti-CD3 protein mediates T cell mediated cytotoxicity. In some embodiments, the isolated anti-DLL3/anti-CD3 protein potently mediates the expansion of cytotoxic CD8 T cells. In some embodiments, the isolated anti-DLL3/anti-CD3 protein upregulates CD25, CD69 and CD71 expression on the surface of CD8 T cells. In some embodiments, the isolated anti-DLL3/anti-CD3 protein displays increased tumor killing. In some embodiments, the isolated anti-DLL3/anti-CD3 protein achieves >90% (e.g., 95%) tumor lysis by 5 days in a T cell cytotoxicity assay.

In a particular embodiment, the disclosure provides an isolated multispecific antigen-binding construct, comprising a first antigen binding region that binds DLL3 and a second antigen binding region that binds CD3, wherein
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, and 35, respectively, and the second domain that binds CD3 comprises the HCDR1, the HCDR2, the HCDR3, the LCDR1, the LCDR2 and the LCDR3 of SEQ ID NOs:98, 99, 100, 106, 107, and 108, respectively; and/or
b. the first antigen binding region that binds DLL3 comprises a scFv of SEQ ID NO:64 and the second antigen binding region that binds CD3 comprises a VH of SEQ ID NO:77 and a VL of SEQ ID NO:80.

In some embodiments, the isolated multispecific antigen-binding construct comprises a lysine (e.g., K477) at the C-terminus of both of the Fc domains (i.e. the HC1 and HC2 domains). An additional lysine may enhance expression of the construct.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 80% identical to the scFv of SEQ ID NO:64 and the second antigen binding region that binds CD3 comprises a VH which is at least 80% identical to the VH of SEQ ID NO:77 and a VL which is at least 80% identical to the VL of SEQ ID NO:80.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 85% identical to the scFv of SEQ ID NO:64 and the second antigen binding region that binds CD3 comprises a VH which is at least 85% identical to the VH of SEQ ID NO:77 and a VL which is at least 85% identical to the VL of SEQ ID NO:80.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 90% identical to the scFv of SEQ ID NO:64 and the second antigen binding region that binds CD3 comprises a VH which is at least 90% identical to the VH of SEQ ID NO:77 and a VL which is at least 90% identical to the VL of SEQ ID NO:80.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 95% identical to the scFv of SEQ ID NO:64 and the second antigen binding region that binds CD3 comprises a VH which is at least 95% identical to the VH of SEQ ID NO:77 and a VL which is at least 95% identical to the VL of SEQ ID NO:80.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 99% identical to the scFv of SEQ ID NO:64 and the second antigen binding region that binds CD3 comprises a VH which is at least 99% identical to the VH of SEQ ID NO:77 and a VL which is at least 99% identical to the VL of SEQ ID NO:80.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 95% identical to the scFv of SEQ ID NO:64 and the second antigen binding region that binds CD3 comprises a VH of SEQ ID NO:77 and a VL which is at least 95% identical to the VL of SEQ ID NO:80.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 99% identical to the scFv of SEQ ID NO:64 and the second antigen binding region that binds CD3 comprises a VH of SEQ ID NO:77 and a VL which is at least 99% identical to the VL of SEQ ID NO:80.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 95% identical to the scFv of SEQ ID NO:64 and the second antigen binding region that binds CD3 comprises a VH which is at least 95% identical to the VH of SEQ ID NO:77 and a VL of SEQ ID NO:80.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 99% identical to the scFv of SEQ ID NO:64 and the second antigen binding region that binds CD3 comprises a VH which is at least 99% identical to the VH of SEQ ID NO:77 and a VL of SEQ ID NO:80.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv of SEQ ID NO:64 and the second antigen binding region that binds CD3 comprises a VH which is at least 95% identical to the VH of SEQ ID NO:77 and a VL which is at least 95% identical to the VL of SEQ ID NO:80.

In a particular embodiment, the disclosure provides an isolated multispecific antigen-binding construct, comprising a first antigen binding region that binds DLL3 and a second antigen binding region that binds CD3, wherein the first antigen binding region that binds DLL3 comprises a scFv of SEQ ID NO:64 and the second antigen binding region that binds CD3 comprises a VH of SEQ ID NO:77 and a VL of SEQ ID NO:80.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 80% identical to the scFv of SEQ ID NO:64 and the second antigen binding region that binds CD3 comprises a VH which is at least 80% identical to the VH of SEQ ID NO:84 and a VL which is at least 80% identical to the VL of SEQ ID NO:85.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 85% identical to the scFv of SEQ ID NO:64 and the second antigen binding region that binds CD3 comprises a VH which is at least 85% identical to the VH of SEQ ID NO:84 and a VL which is at least 85% identical to the VL of SEQ ID NO:85.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 90% identical to the scFv of SEQ ID NO:64 and the second antigen binding region that binds CD3 comprises a VH which is at least 90% identical to the VH of SEQ ID NO:84 and a VL which is at least 90% identical to the VL of SEQ ID NO:85.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 95% identical to the scFv of SEQ ID NO:64 and the second antigen binding region that binds CD3 comprises a VH which is at least 95% identical to the VH of SEQ ID NO:84 and a VL which is at least 95% identical to the VL of SEQ ID NO:85.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 99% identical to the scFv of SEQ ID NO:64 and the second antigen binding region that binds CD3 comprises a VH which is at least 99% identical to the VH of SEQ ID NO:84 and a VL which is at least 99% identical to the VL of SEQ ID NO:85.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 95% identical to the scFv of SEQ ID NO:64 and the second antigen binding region that binds CD3 comprises a VH of SEQ ID NO:84 and a VL which is at least 95% identical to the VL of SEQ ID NO:85.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 99% identical to the scFv of SEQ ID NO:64 and the second antigen binding region that binds CD3 comprises a VH of SEQ ID NO:84 and a VL which is at least 99% identical to the VL of SEQ ID NO:85.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 95% identical to the scFv of SEQ ID NO:64 and the second antigen binding region that binds CD3 comprises a VH which is at least 95% identical to the VH of SEQ ID NO:84 and a VL of SEQ ID NO:85.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv which is at least 99% identical to the scFv of SEQ ID NO:64 and the second antigen binding region that binds CD3 comprises a VH which is at least 99% identical to the VH of SEQ ID NO:84 and a VL of SEQ ID NO:85.

In some embodiments, the disclosure provides an isolated multispecific antigen-binding construct, comprising a first antigen binding region that binds DLL3 and a second antigen binding region that binds CD3, wherein the first antigen binding region that binds DLL3 comprises a scFv of SEQ ID NO:64 and the second antigen binding region that binds CD3 comprises a VH which is at least 95% identical to the VH of SEQ ID NO:84 and a VL which is at least 95% identical to the VL of SEQ ID NO:85.

In a particular embodiment, the disclosure provides an isolated multispecific antigen-binding construct, comprising a first antigen binding region that binds DLL3 and a second antigen binding region that binds CD3, wherein the first antigen binding region that binds DLL3 comprises a scFv of SEQ ID NO:64 and the second antigen binding region that binds CD3 comprises a VH of SEQ ID NO:84 and a VL of SEQ ID NO:85.

As shown in the Examples, the isolated multispecific antigen-binding constructs disclosed herein may be particularly effective at mediating T cell mediated cytotoxicity, promoting T cell activation and proliferation, increasing T cell cytokine release and/or displaying increased anti-tumor efficacy. Accordingly, in some embodiments, the isolated multispecific antigen-binding constructs disclosed herein mediate T cell mediated cytotoxicity. In some embodiments, the isolated multispecific antigen-binding constructs disclosed herein potently mediate the expansion of cytotoxic CD8 T cells. In some embodiments, the isolated multispecific antigen-binding constructs disclosed herein upregulate CD25, CD69 and CD71 expression on the surface of CD8 T cells. In some embodiments, the isolated multispecific antigen-binding constructs disclosed herein display increased tumor killing. In some embodiments, the bispecific anti-DLL3×CD3 antibodies disclosed herein achieve >90% (e.g., 95%) tumor lysis by 5 days in a T cell cytotoxicity assay. Particularly surprising is the fact that the multispecific antigen-binding constructs demonstrating maximum tumor killing bind to an epitope on DLL3 most proximal to the cell membrane, a position thought to compromise the ability of the multispecific antibody to optimally arrange the tumor cell and cytotoxic T cell to achieve an immune synapse.

Isotypes, Allotypes and Fc Engineering

The Ig constant region or the fragment of the Ig constant region, such as the Fc region present in the proteins of the disclosure may be of any allotype or isotype.

In some embodiments, the Ig constant region or the fragment of the Ig constant region is an IgG1 isotype.

In some embodiments, the Ig constant region or the fragment of the Ig constant region is an IgG2 isotype.

In some embodiments, the Ig constant region or the fragment of the Ig constant region is an IgG3 isotype.

In some embodiments, the Ig constant region or the fragment of the Ig constant region is an IgG4 isotype.

The Ig constant region or the fragment of the Ig constant region may be of any allotype.

It is expected that allotype has no influence on properties of the Ig constant region, such as binding or Fc-mediated effector functions. Immunogenicity of therapeutic proteins comprising Ig constant regions of fragments thereof is associated with increased risk of infusion reactions and decreased duration of therapeutic response (Baert et al., (2003) N Engl J Med 348:602-08). The extent to which therapeutic proteins comprising Ig constant regions of fragments thereof induce an immune response in the host may be determined in part by the allotype of the Ig constant region (Stickler et al., (2011) Genes and Immunity 12:213-21). Ig constant region allotype is related to amino acid sequence variations at specific locations in the constant region sequences of the antibody. Table 3 shows selected IgG1, IgG2 and IgG4 allotypes.

TABLE 3
Selected IgG1, IgG2 and IgG4 allotypes

| | Amino acid residue at position of diversity | | | | | | | |
|---|---|---|---|---|---|---|---|---|
| | (residue numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n−) | P | V | | | | | | |
| G2m(n)/(n− | T | V | | | | | | |
| nG4m(a) | | | L | R | | | | |
| G1m(17) | | | | | K | E | M | A |
| G1m(17, 1) | | | | | K | D | L | A |

In a particular embodiment, the Ig constant region allotype is huIgG1_G1m(17).

C-terminal lysine (CTL) may be removed from the Ig constant region by endogenous circulating carboxypeptidases in the blood stream (Cai et al., (2011) Biotechnol Bioeng 108:404-412). During manufacturing, CTL removal may be controlled to less than the maximum level by control of concentration of extracellular Zn²⁺, EDTA or EDTA-Fe³⁺ as described in U.S. Pat. Publ. No. US20140273092. CTL content of proteins may be measured using known methods.

In some embodiments, the antigen binding region that binds DLL3 conjugated to the Ig constant region has a C-terminal lysine content from about 10% to about 90%. In some embodiments, the C-terminal lysine content is from about 20% to about 80%. In some embodiments, the C-terminal lysine content is from about 40% to about 70%. In some embodiments, the C-terminal lysine content is from about 55% to about 70%. In some embodiments, the C-terminal lysine content is about 60%.

Fc region mutations may be made to the antigen binding regions that bind DLL3 conjugated to the Ig constant region or to the fragment of the Ig constant region to modulate their effector functions such as ADCC, ADCP and/or ADCP and/or pharmacokinetic properties. This may be achieved by introducing mutation(s) into the Fc that modulate binding of the mutated Fc to activating FcγRs (FcγRI, FcγRIIa, FcγRIII), inhibitory FcγRIIb and/or to FcRn.

In some embodiments, the antigen binding region that binds DLL3 conjugated to the Ig constant region or the fragment of the Ig constant region comprises at least one mutation in the Ig constant region or in the fragment of the Ig constant region.

In some embodiments, the at least one mutation is in the Fc region.

In some embodiments, the antigen binding region that binds DLL3 conjugated to the Ig constant region or to the fragment of the Ig constant region comprises at least one, two, three, four, five, six, seven, eight, nine, ten, eleven, twelve, thirteen, fourteen or fifteen mutations in the Fc region.

In some embodiments, the antigen binding region that binds DLL3 conjugated to the Ig constant region or to the fragment of the Ig constant region comprises at least one mutation in the Fc region that modulates binding of the antibody to FcRn.

Fc positions that can be mutated to modulate half-life (e.g., binding to FcRn) include positions 250, 252, 253, 254, 256, 257, 307, 376, 380, 428, 434 and 435. Exemplary mutations that can be made singularly or in combination are mutations T250Q, M252Y, I253A, S254T, T256E, P2571, T307A, D376V, E380A, M428L, H433K, N434 S, N434A, N434H, N434F, H435A and H435R. Exemplary singular or combination mutations that can be made to increase the half-life are mutations M428L/N434 S, M252Y/S254T/T256E, T250Q/M428L, N434A and T307A/E380A/N434A. Exemplary singular or combination mutations that may be made to reduce the half-life are mutations H435A, P257I/N434H, D376V/N434H, M252Y/S254T/T256E/H433K/N434F, T308P/N434A and H435R.

In some embodiments, the antigen binding region that binds DLL3 conjugated to the Ig constant region or to the fragment of the Ig constant region comprises M252Y/S254T/T256E mutation.

In some embodiments, the antigen binding region that binds DLL3 conjugated to the Ig constant region or to the fragment of the Ig constant region comprises at least one mutation in the Fc region that reduces binding of the protein to an activating Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP).

Fc positions that may be mutated to reduce binding of the protein to the activating FcγR and subsequently to reduce effector function include positions 214, 233, 234, 235, 236, 237, 238, 265, 267, 268, 270, 295, 297, 309, 327, 328, 329, 330, 331 and 365. Exemplary mutations that may be made singularly or in combination are mutations K214T, E233P, L234V, L234A, deletion of G236, V234A, F234A, L235A, G237A, P238A, P238 S, D265A, S267E, H268A, H268Q, Q268A, N297A, A327Q, P329A, D270A, Q295A, V309L, A327 S, L328F, A330 S and P331 S in IgG1, IgG2, IgG3 or IgG4. Exemplary combination mutations that result in proteins with reduced ADCC are mutations L234A/L235A on IgG1, L234A/L235A/D265 S on IgG1, V234A/G237A/P238 S/H268A/V309L/A330 S/P331 S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330 S/P331 S on IgG2, S267E/L328F on IgG1, L234F/L235E/D265A on IgG1, L234A/L235A/G237A/P238 S/H268A/A330 S/P331 S on IgG1, S228P/F234A/L235A/G237A/P238 S on IgG4, and S228P/F234A/L235A/G236-deleted/G237A/P238 S on IgG4. Hybrid IgG2/4 Fc domains may also be used, such as Fc with residues 117-260 from IgG2 and residues 261-447 from IgG4.

Exemplary mutation that result in proteins with reduced CDC is a K322A mutation.

Well-known S228P mutation may be made in IgG4 to enhance IgG4 stability.

In some embodiments, the antigen binding region that binds DLL3 conjugated to the Ig constant region or to the fragment of the Ig constant region comprises at least one mutation selected from the group consisting of K214T, E233P, L234V, L234A, deletion of G236, V234A, F234A, L235A, G237A, P238A, P238 S, D265A, S267E, H268A, H268Q, Q268A, N297A, A327Q, P329A, D270A, Q295A, V309L, A327 S, L328F, K322, A330 S and P331 S.

In some embodiments, the antigen binding region that binds DLL3 conjugated to the Ig constant region or to the fragment of the Ig constant region comprises L234A/L235A/D265 S mutation. In a particular embodiment, the antigen binding region that binds DLL3 is conjugated to an IgG1 constant region or to the fragment of an IgG1 constant region comprising L234A_L235A_D265 S mutations.

In some embodiments, the antigen binding region that binds DLL3 conjugated to the Ig constant region or to the fragment of the Ig constant region comprises L234A/L235A mutation.

In some embodiments, the antigen binding region that binds DLL3 conjugated to the Ig constant region or to the fragment of the Ig constant region comprises at least one mutation in the Fc region that enhances binding of the protein to an Fcγ receptor (FcγR) and/or enhances Fc effector functions such as C1q binding, complement dependent cytotoxicity (CDC), antibody-dependent cell-mediated cytotoxicity (ADCC) and/or phagocytosis (ADCP).

Fc positions that can be mutated to increase binding of the protein to the activating FcγR and/or enhance Fc effector functions include positions 236, 239, 243, 256, 290, 292, 298, 300, 305, 312, 326, 330, 332, 333, 334, 345, 360, 339, 378, 396 or 430 (residue numbering according to the EU index). Exemplary mutations that may be made singularly or in combination are G236A, S239D, F243L, T256A, K290A, R292P, S298A, Y300L, V305L, K326A, A330K, 1332E, E333A, K334A, A339T and P396L. Exemplary combination mutations that result in proteins with increased ADCC or ADCP are a S239D/I332E, S298A/E333A/K334A, F243L/R292P/Y300L, F243L/R292P/Y300L/P396L, F243L/R292P/Y300L/V305I/P396L and G236A/S239D/I332E.

Fc positions that can be mutated to enhance CDC include positions 267, 268, 324, 326, 333, 345 and 430. Exemplary mutations that may be made singularly or in combination are S267E, F1268F, S324T, K326A, K326W, E333A, E345K, E345Q, E345R, E345Y, E430 S, E430F and E430T. Exemplary combination mutations that result in proteins with increased CDC are K326A/E333A, K326W/E333A, H268F/S324T, S267E/H268F, S267E/S324T and S267E/H268F/S324T.

The specific mutations described herein are mutations when compared to the IgG1, IgG2 and IgG4 wild-type amino acid sequences of SEQ ID NOs:257, 258, and 259, respectively.

Binding of the antibody to FcγR or FcRn can be assessed on cells engineered to express each receptor using flow cytometry. In an exemplary binding assay, 2×10⁵ cells per well are seeded in 96-well plate and blocked in BSA Stain Buffer (BD Biosciences, San Jose, USA) for 30 min at 4° C. Cells are incubated with a test antibody on ice for 1.5 hour at 4° C. After being washed twice with BSA stain buffer, the cells are incubated with R-PE labeled anti-human IgG secondary antibody (Jackson Immunoresearch Laboratories) for 45 min at 4° C. The cells are washed twice in stain buffer and then resuspended in 150 μL of Stain Buffer containing 1:200 diluted DRAQ7 live/dead stain (Cell Signaling Technology, Danvers, USA). PE and DRAQ7 signals of the stained cells are detected by Miltenyi MACSQuant flow cytometer (Miltenyi Biotec, Auburn, USA) using B2 and B4 channel respectively. Live cells are gated on DRAQ7 exclusion and the geometric mean fluorescence signals are determined for at least 10,000 live events collected. FlowJo software (Tree Star) is used for analysis. Data is plotted as the logarithm of antibody concentration versus mean fluorescence signals. Nonlinear regression analysis is performed.

Glycoengineering

The ability of the antigen binding region that binds DLL3 conjugated to the Ig constant region or to the fragment of the Ig constant region to mediate ADCC can be enhanced by engineering the Ig constant region or the fragment of the Ig constant region oligosaccharide component. Human IgG1 or IgG3 are N-glycosylated at Asn297 with the majority of the glycans in the well-known biantennary G0, G0F, G1, G1F, G2 or G2F forms. Ig constant region containing proteins may be produced by non-engineered CHO cells typically have a glycan fucose content of about at least 85%. The removal of the core fucose from the biantennary complex-type oligosaccharides attached to the antigen binding region that binds DLL3 conjugated to the Ig constant region or to the fragment of the Ig constant region enhances the ADCC of the protein via improved FcγRIIIa binding without altering antigen binding or CDC activity. Such proteins can be achieved using different methods reported to lead to the successful expression of relatively high defucosylated immunoglobulins bearing the biantennary complex-type of Fc oligosaccharides such as control of culture osmolality (Konno et al., Cytotechnology 64(: 249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4): 405-415, 2010; PMID: 20562582), application of a rat hybridoma cell line YB2/0 as the host cell line (Shinkawa et al., J Biol Chem 278:3466-3473, 2003), introduction of small interfering RNA specifically against the a 1,6-fucosyltrasferase (FUT8) gene (Mori et al., Biotechnol Bioeng 88:901-908, 2004), or coexpression of β-1,4-N-acetylglucosaminyltransferase III and Golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments, the antigen binding region that binds DLL3 conjugated to the Ig constant region or to the fragment of the Ig constant region of the disclosure has a biantennary glycan structure with fucose content of about between 1% to about 15%, for example about 15%, 14%, 13%, 12%, 11% 10%, 9%, 8%, 7%, 6%, 5%, 4%, 3%, 2% or 1%. In some embodiments, the antigen binding region that binds DLL3 conjugated to the Ig constant region or to the fragment of the Ig constant region has a glycan structure with fucose content of about 50%, 40%, 45%, 40%, 35%, 30%, 25%, or 20%.

“Fucose content” means the amount of the fucose monosaccharide within the sugar chain at Asn297. The relative amount of fucose is the percentage of fucose-containing structures related to all glycostructures. These may be characterized and quantified by multiple methods, for example: 1) using MALDI-TOF of N-glycosidase F treated sample (e.g., complex, hybrid and oligo- and high-mannose structures) as described in Int Pat. Publ. No. WO2008/077546; 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the mAb to constituent peptides by enzymatic digestion (e.g., trypsin or endopeptidase Lys-C), and subsequent separation, detection and quantitation by HPLC-MS (UPLC-MS); 5) Separation of the mAb oligosaccharides from the mAb protein by specific enzymatic deglycosylation with PNGase F at Asn 297. The oligosaccharides thus released can be labeled with a fluorophore, separated and identified by various complementary techniques which allow: fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosaccharide forms according to hydrophilicity criteria by normal-phase HPLC (GlycoSep N), and separation and quantification of the oligosaccharides by high performance capillary electrophoresis-laser induced fluorescence (HPCE-LIF).

“Low fucose” or “low fucose content” as used herein refers to the antigen binding region that bind DLL3 conjugated to the Ig constant region or to the fragment of the Ig constant region with fucose content of about between 1%-15%.

“Normal fucose” or “normal fucose content” as used herein refers to the antigen binding region that bind DLL3 conjugated to the Ig constant region or to the fragment of the Ig constant region with fucose content of about over 50%, typically about over 80% or over 85%.

Anti-Idiotypic Antibodies

Anti-idiotypic antibodies are antibodies that specifically bind to the antigen binding region that binds DLL3 of the disclosure.

The application also provides an anti-idiotypic antibody that specifically binds to the antigen binding region that binds DLL3 of the disclosure.

An anti-idiotypic (Id) antibody is an antibody which recognizes the antigenic determinants (e.g., the paratope or CDRs) of the antibody. The Id antibody may be antigen-blocking or non-blocking. The antigen-blocking Id may be used to detect the free antigen binding region in a sample (e.g., the antigen binding region that binds DLL3 of the disclosure). The non-blocking Id may be used to detect the total antibody (free, partially bond to antigen, or fully bound to antigen) in a sample. An Id antibody may be prepared by immunizing an animal with the antibody to which an anti-Id is being prepared.

An anti-Id antibody may also be used as an immunogen to induce an immune response in yet another animal, producing a so-called anti-anti-Id antibody. An anti-anti-Id may be epitopically identical to the original antigen binding region which induced the anti-Id. Thus, by using antibodies to the idiotypic determinants of the antigen binding region, it is possible to identify other clones expressing antigen binding regions of identical specificity. Anti-Id antibodies may be varied (thereby producing anti-Id antibody variants) and/or derivatized by any suitable technique, such as those described elsewhere herein.

Immunoconjugates

The antigen binding regions that bind DLL3 of the disclosure, the proteins comprising the antigen binding regions that bind DLL3 or the multispecific antigen-binding constructs that comprise the antigen binding regions that bind DLL3 (collectively referred herein as to DLL3 binding proteins) may be conjugated to a heterologous molecule.

In some embodiments, the heterologous molecule is a detectable label or a cytotoxic agent.

The application also provides an antigen binding region that binds DLL3 conjugated to a detectable label.

The application also provides a protein comprising an antigen binding region that binds DLL3 conjugated to a detectable label.

The application also provides a multispecific antigen-binding construct comprising an antigen binding region that binds DLL3 conjugated to a detectable label.

The application also provides an antigen binding region that binds DLL3 conjugated to a cytotoxic agent.

The application also provides a protein comprising an antigen binding region that binds DLL3 conjugated to a cytotoxic agent.

The application also provides a multispecific antigen-binding construct comprising an antigen binding region that binds DLL3 conjugated to a cytotoxic agent.

DLL3 binding proteins of the disclosure may be used to direct therapeutics to DLL3 expressing cells, such as DLL3-expressing prostate cancer cells or small-cell lung cancer cells. Alternatively, DLL3 expressing cells may be targeted with a DLL3 binding protein of the disclosure coupled to a therapeutic intended to modify cell function once internalized.

In some embodiments, the detectable label is also a cytotoxic agent.

The DLL3 binding proteins of the disclosure conjugated to a detectable label may be used to evaluate expression of DLL3 on a variety of samples.

Detectable label includes compositions that when conjugated to the DLL3 binding proteins of the disclosure renders the latter detectable, via spectroscopic, photochemical, biochemical, immunochemical, or chemical means.

Exemplary detectable labels include radioactive isotopes, magnetic beads, metallic beads, colloidal particles, fluorescent dyes, electron-dense reagents, enzymes (for example, as commonly used in an ELISA), biotin, digoxigenin, haptens, luminescent molecules, chemiluminescent molecules, fluorochromes, fluorophores, fluorescent quenching agents, colored molecules, radioactive isotopes, scintillates, avidin, streptavidin, protein A, protein G, antibodies or fragments thereof, polyhistidine, Ni²⁺, Flag tags, myc tags, heavy metals, enzymes, alkaline phosphatase, peroxidase, luciferase, electron donors/acceptors, acridinium esters, and colorimetric substrates.

A detectable label may emit a signal spontaneously, such as when the detectable label is a radioactive isotope. In other cases, the detectable label emits a signal as a result of being stimulated by an external field.

Exemplary radioactive isotopes may be γ-emitting, Auger-emitting, β-emitting, an alpha-emitting or positron-emitting radioactive isotope. Exemplary radioactive isotopes include ³H, 11C, 13c ¹⁵N, ¹⁸F ¹⁹F, ⁵⁵Co, ⁵⁷Co, ⁶⁰Co, ⁶¹Cu, ⁶²Cu, ⁶⁴Cu, ⁶⁷Cu, ⁶⁸Ga, ⁷²As, 75Br, ⁸⁶y, ⁸⁹Z, ⁹⁰Sr, ^{94m}Tc, ^{99m}Tc, ¹¹⁵In, ¹²³j, ¹²⁴l, ¹²⁵j, ¹³¹I, ²¹¹At, ²¹²Bi, ²¹³Bi, ²²³Ra, ²²⁶Ra, ²²⁵Ac and ²²⁷Ac.

Exemplary metal atoms are metals with an atomic number greater than 20, such as calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, lead atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkelium atoms, californium atoms, einsteinium atoms, fermium atoms, mendelevium atoms, nobelium atoms, or lawrencium atoms.

In some embodiments, the metal atoms may be alkaline earth metals with an atomic number greater than twenty.

In some embodiments, the metal atoms may be lanthanides.

In some embodiments, the metal atoms may be actinides.

In some embodiments, the metal atoms may be transition metals.

In some embodiments, the metal atoms may be poor metals.

In some embodiments, the metal atoms may be gold atoms, bismuth atoms, tantalum atoms, and gadolinium atoms.

In some embodiments, the metal atoms may be metals with an atomic number of 53 (i.e. iodine) to 83 (i.e. bismuth).

In some embodiments, the metal atoms may be atoms suitable for magnetic resonance imaging.

The metal atoms may be metal ions in the form of +1, +2, or +3 oxidation states, such as Ba²⁺, Bi³⁺, Cs⁺, Ca²⁺, Cr²⁺, Cr³⁺, Cr⁶⁺, Co²⁺, Co³⁺, Cu⁺, Cu²⁺, Cu³⁺, Ga³⁺, Gd³⁺, Au⁺, Au³⁺, Fe²⁺, Fe³⁺, F³⁺, Pb²⁺, Mn²⁺, Mn³⁺, Mn⁴⁺, Mn⁷⁺, Hg²⁺, Ni²⁺, Ni³⁺, Ag⁺, Sr²⁺, Sn²⁺, Sn⁴⁺, and Zn²⁺. The metal atoms may comprise a metal oxide, such as iron oxide, manganese oxide, or gadolinium oxide.

Suitable dyes include any commercially available dyes such as, for example, 5(6)-carboxyfluorescein, IRDye 680RD maleimide or IRDye 800CW, ruthenium polypyridyl dyes, and the like.

Suitable fluorophores are fluorescein isothiocyanate (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes.

The antigen binding region that binds DLL3 conjugated to a detectable label may be used as an imaging agent.

The protein comprising an antigen binding region that binds DLL3 conjugated to a detectable label may be used as an imaging agent.

The multispecific antigen-binding construct comprising an antigen binding region that binds DLL3 conjugated to a detectable label may be used as an imaging agent.

In some embodiments, the cytotoxic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

In some embodiments, the cytotoxic agent is daunomycin, doxorubicin, methotrexate, vindesine, bacterial toxins such as diphtheria toxin, ricin, geldanamycin, maytansinoids or calicheamicin. The cytotoxic agent may elicit their cytotoxic and cytostatic effects by mechanisms including tubulin binding, DNA binding, or topoisomerase inhibition.

In some embodiments, the cytotoxic agent is an enzymatically active toxin such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, the cytotoxic agent is a radionuclide, such as ²¹²Bi, ¹³¹I, ¹³¹In, ⁹⁰Y and ¹⁸⁶Re.

In some embodiments, the cytotoxic agent is dolastatins or dolostatin peptidic analogs and derivatives, auristatin or monomethyl auristatin phenylalanine. Exemplary molecules are disclosed in U.S. Pat. Nos. 5,635,483 and 5,780,588. Dolastatins and auristatins have been shown to interfere with microtubule dynamics, GTP hydrolysis, and nuclear and cellular division (Woyke et al (2001) Antimicrob Agents and Chemother. 45(12):3580-3584) and have anticancer and antifungal activity. The dolastatin or auristatin drug moiety may be attached to the antibody of the application through the N (amino) terminus or the C (carboxyl) terminus of the peptidic drug moiety (WO02/088172), or via any cysteine engineered into the antibody.

The DLL3 binding proteins of the disclosure can be conjugated to a detectable label using known methods.

In some embodiments, the detectable label is complexed with a chelating agent.

In some embodiments, the detectable label is conjugated to the DLL3 binding proteins of the disclosure via a linker.

The detectable label or the cytotoxic moiety may be linked directly, or indirectly, to the DLL3 binding proteins of the disclosure using known methods. Suitable linkers are known in the art and include, for example, prosthetic groups, non-phenolic linkers (derivatives of N-succimidyl-benzoates; dodecaborate), chelating moieties of both macrocyclics and acyclic chelators, such as derivatives of 1,4,7,10-tetraazacyclododecane-1,4,7,10, tetraacetic acid (DOTA), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA), N-succinimidyl-3-(2-pyridyldithiol) propionate (SPDP), iminothiolane (IT), bifunctional derivatives of imidoesters (such as dimethyl adipimidate HC1), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene) and other chelating moieties. Suitable peptide linkers are well known.

In some embodiments, the DLL3 binding proteins of the disclosure is removed from the blood via renal clearance.

Kits

The application also provides a kit comprising the antigen binding region that binds DLL3.

The application also provides a kit comprising the protein comprising an antigen binding region that binds DLL3.

The application also provides a kit comprising the multispecific antigen-binding construct comprising an antigen binding region that binds DLL3.

The kit may be used for therapeutic uses and as diagnostic kits.

The kit may be used to detect the presence of DLL3 in a sample.

In some embodiments, the kit comprises the DLL3 binding protein of the disclosure and reagents for detecting the DLL3 binding protein. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some embodiments, the kit comprises the antigen binding region that binds DLL3 in a container and instructions for use of the kit.

In some embodiments, the kit comprises the protein comprising an antigen binding region that binds DLL3 in a container and instructions for use of the kit.

In some embodiments, the kit comprises the multispecific antigen-binding construct comprising an antigen binding region that binds DLL3 in a container and instructions for use of the kit.

In some embodiments, the antigen binding region that binds DLL3 in the kit is labeled.

In some embodiments, the protein comprising an antigen binding region that binds DLL3 in the kit is labeled.

In some embodiments, the multispecific antigen-binding construct comprising an antigen binding region that binds DLL3 in the kit is labeled.

Methods of Detecting DLL3

The application also provides a method of detecting DLL3 in a sample, comprising obtaining the sample, contacting the sample with the antigen binding region that binds DLL3 of the disclosure and detecting the bound DLL3 in the sample.

In some embodiments, the sample may be derived from urine, blood, serum, plasma, saliva, ascites, circulating cells, synovial fluid, circulating cells, cells that are not tissue associated (i.e., free cells), tissues (e.g., surgically resected tissue, biopsies, including fine needle aspiration), histological preparations, and the like.

The antigen binding region that binds DLL3 of the disclosure may be detected using known methods. Exemplary methods include direct labeling of the antibodies using fluorescent or chemiluminescent labels, or radiolabels, or attaching to the antibodies of the application a moiety which is readily detectable, such as biotin, enzymes or epitope tags. Exemplary labels and moieties are ruthenium, ¹¹¹In-DOTA, ¹¹¹In-diethylenetriaminepentaacetic acid (DTPA), horseradish peroxidase, alkaline phosphatase and beta-galactosidase, poly-histidine (HIS tag), acridine dyes, cyanine dyes, fluorone dyes, oxazin dyes, phenanthridine dyes, rhodamine dyes and Alexafluor® dyes.

The antigen binding region that binds DLL3 of the disclosure may be used in a variety of assays to detect DLL3 in the sample. Exemplary assays are western blot analysis, radioimmunoassay, surface plasmon resonance, immunoprecipitation, equilibrium dialysis, immunodiffusion, electrochemiluminescence (ECL) immunoassay, immunohistochemistry, fluorescence-activated cell sorting (FACS) or ELISA assay.

Polynucleotides, Host Cells and Vectors

The disclosure also provides an isolated polynucleotide encoding any of the DLL3 binding proteins of the disclosure, including the antigen binding regions that bind DLL3, the proteins comprising the antigen binding regions that bind DLL3, the multispecific antigen-binding constructs that comprise the antigen binding regions that bind DLL3.

In some embodiments, the application provides an isolated polynucleotide encoding any of DLL3 biding proteins or fragments thereof disclosed herein.

In certain embodiments, the application provides an isolated polynucleotide encoding a VH of SEQ ID NO: 1, 3, 5, 7, 9, 11 or 13. In certain embodiments, the applications provides an isolated polynucleotide encoding a VL of SEQ ID NO:2, 4, 6, 8, 10, 12 or 14.

In certain embodiments, the application provides an isolated polynucleotide encoding the VH of SEQ ID NO:1 and the VL of SEQ ID NO:2;
the VH of SEQ ID NO:3 and the VL of SEQ ID NO:4;
the VH of SEQ ID NO:5 and the VL of SEQ ID NO:6;
the VH of SEQ ID NO:7 and the VL of SEQ ID NO:8;
the VH of SEQ ID NO:9 and the VL of SEQ ID NO:10;
the VH of SEQ ID NO: 11 and the VL of SEQ ID NO: 12; or
the VH of SEQ ID NO: 13 and the VL of SEQ ID NO: 14.

The application also provides an isolated polynucleotide encoding the polypeptide of SEQ ID NO:1, 2, 3, 4, 5, 6, 7, 8, 9, 10, 11, 12, 13, 14, 63, 64, 65, 66, 67, 68, 69, 71, 77, 78, 80, 84, 85, 105, 109, 110, 111, 112, 113, 114, 115, 116, 117, 118, 119, 229, 190, 191, 192, 193, 194, 195, 196, 230, or 196.

The application also provides an isolated polynucleotide of SEQ ID NO:86, 87, 89, 93, 94, 163, 164, 165, 166, 167, 168, 169, 170, 171, 172, 173, 174, 175, 176, 177, 202, 233, 234, 235, 236, 237, 238, 239, 256, 260, 261, 262, 264, 265, 266, 267, 268, 269, or 270.

In a particular embodiment, the disclosure provides isolated polynucleotide sequences encoding polypeptide sequences of SEQ ID NOs:71, 118 and 117.

In a particular embodiment, the disclosure provides isolated polynucleotide sequences encoding polypeptide sequences of SEQ ID NOs:229, 230 and 117.

In a particular embodiment, the disclosure provides isolated polynucleotide sequences which are at least 80% (e.g., at least 85%, at least 90%, at least 95%, at least 99% or 100%) identical to the polynucleotide of SEQ ID NO:266, at least 80% (e.g., at least 85%, at least 90%, at least 95%, at least 99% or 100%) identical to the polynucleotide of SEQ ID NO:235, and at least 80% (e.g., at least 85%, at least 90%, at least 95%, at least 99% or 100%) identical to the polynucleotide of SEQ ID NO:236.

In a particular embodiment, the disclosure provides an isolated polynucleotide sequence which is at least 80% (e.g., at least 85%, at least 90%, at least 95%, at least 99% or 100%) identical to the polynucleotide of SEQ ID NO:266.

In a particular embodiment, the disclosure provides an isolated polynucleotide sequence which is at least 80% (e.g., at least 85%, at least 90%, at least 95%, at least 99% or 100%) identical to the polynucleotide of SEQ ID NO:235.

In a particular embodiment, the disclosure provides an isolated polynucleotide sequence which is at least 80% (e.g., at least 85%, at least 90%, at least 95%, at least 99% or 100%) identical to the polynucleotide of SEQ ID NO:236.

In a particular embodiment, the disclosure provides isolated polynucleotide sequences which are at least 80% (e.g., at least 85%, at least 90%, at least 95%, at least 99% or 100%) identical to the polynucleotide of SEQ ID NO:239, at least 80% (e.g., at least 85%, at least 90%, at least 95%, at least 99% or 100%) identical to the polynucleotide of SEQ ID NO:237 and at least 80% (e.g., at least 85%, at least 90%, at least 95%, at least 99% or 100%) identical to the polynucleotide of SEQ ID NO:238.

In a particular embodiment, the disclosure provides an isolated polynucleotide sequence which is at least 80% (e.g., at least 85%, at least 90%, at least 95%, at least 99% or 100%) identical to the polynucleotide of SEQ ID NO:237.

In a particular embodiment, the disclosure provides an isolated polynucleotide sequence which is at least 80% (e.g., at least 85%, at least 90%, at least 95%, at least 99% or 100%) identical to the polynucleotide of SEQ ID NO:238.

In a particular embodiment, the disclosure provides an isolated polynucleotide sequence which is at least 80% (e.g., at least 85%, at least 90%, at least 95%, at least 99% or 100%) identical to the polynucleotide of SEQ ID NO:239.

In a particular embodiment, the disclosure provides isolated polynucleotide sequences encoding polypeptide sequences of SEQ ID NOs:64, 84, and 85. Some embodiments of the disclosure also provide an isolated or purified nucleic acid comprising a polynucleotide which is complementary to the polynucleotides encoding the DLL3 binding proteins of the disclosure or polynucleotides which hybridize under stringent conditions to the polynucleotides encoding the DLL3 binding proteins of the disclosure.

The polynucleotides which hybridize under stringent conditions may hybridize under high stringency conditions. By “high stringency conditions” is meant that the polynucleotide specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-12 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand. It is generally appreciated that conditions can be rendered more stringent by the addition of increasing amounts of formamide.

The polynucleotide sequences of the disclosure may be operably linked to one or more regulatory elements, such as a promoter or enhancer, that allow expression of the nucleotide sequence in the intended host cell. The polynucleotide may be a cDNA. The promoter may be a strong, weak, tissue-specific, inducible or developmental-specific promoter. Exemplary promoters that may be used are hypoxanthine phosphoribosyl transferase (HPRT), adenosine deaminase, pyruvate kinase, beta-actin, human myosin, human hemoglobin, human muscle creatine, and others. In addition, many viral promoters function constitutively in eukaryotic cells and are suitable for use with the described embodiments. Such viral promoters include Cytomegalovirus (CMV) immediate early promoter, the early and late promoters of SV40, the Mouse Mammary Tumor Virus (MMTV) promoter, the long terminal repeats (LTRs) of Maloney leukemia virus, Human Immunodeficiency Virus (HIV), Epstein Barr Virus (EBV), Rous Sarcoma Virus (RSV), and other retroviruses, and the thymidine kinase promoter of Herpes Simplex Virus. Inducible promoters such as the metallothionein promoter, tetracycline-inducible promoter, doxycycline-inducible promoter, promoters that contain one or more interferon-stimulated response elements (ISRE) such as protein kinase R 2′,5′-oligoadenylate synthetases, Mx genes, ADAR1, and the like may also be used.

The application also provides a vector comprising the polynucleotide of the application. The disclosure also provides an expression vector comprising the polynucleotide of the application. Such vectors may be plasmid vectors, viral vectors, vectors for baculovirus expression, transposon-based vectors or any other vector suitable for introduction of the synthetic polynucleotide of the application into a given organism or genetic background by any means. Polynucleotides encoding the DLL3 binding proteins of the disclosure may be operably linked to control sequences in the expression vector(s) that ensure the expression of the DLL3 binding proteins. Such regulatory elements may include a transcriptional promoter, sequences encoding suitable mRNA ribosomal binding sites, and sequences that control the termination of transcription and translation. Expression vectors may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

The expression vectors can comprise naturally-occurring or non-naturally-occurring internucleotide linkages, or both types of linkages. The non-naturally occurring or altered nucleotides or internucleotide linkages do not hinder the transcription or replication of the vector.

Once the vector has been incorporated into the appropriate host, the host is maintained under conditions suitable for high level expression of the DLL3 binding proteins of the disclosure encoded by the incorporated polynucleotides. The transcriptional and translational control sequences in expression vectors to be used in transforming vertebrate cells may be provided by viral sources. Exemplary vectors may be constructed as described by Okayama and Berg, 3 Mol. Cell. Biol. 280 (1983).

Vectors of the disclosure may also contain one or more Internal Ribosome Entry Site(s) (IRES). Inclusion of an IRES sequence into fusion vectors may be beneficial for enhancing expression of some proteins. In some embodiments, the vector system will include one or more polyadenylation sites (e.g., SV40), which may be upstream or downstream of any of the aforementioned nucleic acid sequences. Vector components may be contiguously linked or arranged in a manner that provides optimal spacing for expressing the gene products (i.e., by the introduction of “spacer” nucleotides between the ORFs) or positioned in another way. Regulatory elements, such as the IRES motif, may also be arranged to provide optimal spacing for expression.

Vectors of the disclosure may be circular or linear. They may be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

The vectors may also comprise selection markers, which are well known in the art. Selection markers include positive and negative selection marker. Marker genes include biocide resistance, e.g., resistance to antibiotics, heavy metals, etc., complementation in an auxotrophic host to provide prototrophy, and the like. Exemplary marker genes include antibiotic resistance genes (e.g., neomycin resistance gene, a hygromycin resistance gene, a kanamycin resistance gene, a tetracycline resistance gene, a penicillin resistance gene, histidinol resistance gene, histidinol x resistance gene), glutamine synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

Exemplary vectors that may be used are Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia), pEE6.4 (Lonza) and pEE12.4 (Lonza). Additional vectors include the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Exemplary plant expression vectors include pBI01, pBI01.2, pBIl21, pBI101.3, and pBIN19 (Clontech). Exemplary animal expression vectors include pEUK-Cl, pMAM, and pMAMneo (Clontech). The expression vector may be a viral vector, e.g., a retroviral vector, e.g., a gamma retroviral vector.

In some embodiments, a vector comprises a polynucleotide encoding a VH of SEQ ID NO:1, 3, 5, 7, 9, 11 or 13. In certain embodiments, the vector comprises a polynucleotide encoding a VL of SEQ ID NO:2, 4, 6, 8, 10, 12 or 14.

In some embodiments, a vector comprises a polynucleotide encoding polypeptide of SEQ ID NOs:63, 64, 65, 66, 67, 68, or 69.

The application also provides for a host cell comprising one or more vectors of the application. “Host cell” refers to a cell into which a vector has been introduced. It is understood that the term host cell is intended to refer not only to the particular subject cell but to the progeny of such a cell, and also to a stable cell line generated from the particular subject cell. Because certain modifications may occur in succeeding generations due to either mutation or environmental influences, such progeny may not be identical to the parent cell, but are still included within the scope of the term “host cell” as used herein. Such host cells may be eukaryotic cells, prokaryotic cells, plant cells or archeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells may be of mammalian, insect, avian or other animal origins. Mammalian eukaryotic cells include immortalized cell lines such as hybridomas or myeloma cell lines such as SP2/0 (American Type Culture Collection (ATCC), Manassas, VA, CRL-1581), NSO (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-K1SV (Lonza Biologics, Walkersville, MD), CHO-K1 (ATCC CRL-61) or DG44.

In another aspect, the application relates to a host cell transformed with the vector disclosed herein. In an embodiment, the host cell is a prokaryotic cell, for example, E. coli. In another embodiment, the host cell is a eukaryotic cell, for example, a protist cell, an animal cell, a plant cell, or a fungal cell. In an embodiment, the host cell is a mammalian cell including, but not limited to, CHO, COS, NSO, SP2, PER.C6, or a fungal cell, such as Saccharomyces cerevisiae, or an insect cell, such as Sf9.

A protein, an antibody or an antigen-binding fragment thereof, a conjugate, a multi-specific antibody/construct or fusion construct of the application can be produced by any of a number of techniques known in the art in view of the present disclosure. For example, it can be expressed from a recombinant host cells, wherein expression vector(s) encoding the heavy and light chains of the fusion construct or multi-specific antibody/construct is (are) transfected into a host cell by standard techniques. The host cells can be prokaryotic or eukaryotic host cells.

In an exemplary system, one or more recombinant expression vectors encoding the heterodimeric two heavy chains and the light chains of a fusion construct of the application is/are introduced into host cells by transfection or electroporation. The selected transformant host cells are cultured to allow for expression of the heavy and light chains under conditions sufficient to produce the fusion construct, and the fusion construct is recovered from the culture medium. Standard molecular biology techniques are used to prepare the recombinant expression vector, transfect the host cells, select for transformants, culture the host cells and recover the protein construct from the culture medium.

The disclosure also provides a method of producing the DLL3 binding protein of the disclosure comprising culturing the host cell of the disclosure in conditions that the DLL3 binding protein is expressed, and recovering the DLL3 binding protein produced by the host cell. Methods of making proteins and purifying them are known. Once synthesized (either chemically or recombinantly), the DLL3 binding proteins may be purified according to standard procedures, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). A subject protein may be substantially pure, e.g., at least about 80% to 85% pure, at least about 85% to 90% pure, at least about 90% to 95% pure, or at least about 98% to 99%, or more, pure, e.g., free from contaminants such as cell debris, macromolecules, etc. other than the subject protein.

The polynucleotides encoding the DLL3 binding proteins of the disclosure can be incorporated into vectors using standard molecular biology methods. Host cell transformation, culture, antibody expression and purification are done using well known methods.

Modified nucleotides may be used to generate the polynucleotides of the disclosure. Exemplary modified nucleotides are 5-fluorouracil, 5-bromouracil, 5-chlorouracil, 5-iodouracil, hypoxanthine, xanthine, 4-acetylcytosine, 5-(carboxyhydroxymethyl) uracil, carboxymethylaminomethyl-2-thiouridine, 5-carboxymethylaminomethyluracil, dihydrouracil, N⁶-substituted adenine, 7-methylguanine, 5-methylaminomethyluracil, 5-methoxyaminomethyl-2-thiouracil, beta-D-mannosylqueosine, 5″-methoxycarboxymethyluracil, 5-methoxyuracil, 2-methylthio-N⁶-isopentenyladenine, uracil-5-oxyacetic acid (v), wybutoxosine, pseudouracil, queuosine, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

Pharmaceutical Compositions/Administration

The disclosure also provides a pharmaceutical composition comprising the DLL3 binding protein of the disclosure and a pharmaceutically acceptable carrier.

The disclosure also provides a pharmaceutical composition comprising the antigen binding region that binds DLL3 of the disclosure and a pharmaceutically acceptable carrier.

The disclosure also provides a pharmaceutical composition comprising the protein comprising the antigen binding region that binds DLL3 of the disclosure and a pharmaceutically acceptable carrier.

The disclosure also provides a pharmaceutical composition comprising the multispecific antigen-binding construct comprising the antigen binding region that binds DLL3 of the disclosure and a pharmaceutically acceptable carrier.

For therapeutic use, the DLL3 binding protein of the disclosure may be prepared as pharmaceutical compositions containing an effective amount of the antibody as an active ingredient in a pharmaceutically acceptable carrier. “Carrier” refers to a diluent, adjuvant, excipient, or vehicle with which the antibody of the application is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the antibodies of the application in such pharmaceutical formulation may vary, from less than about 0.5%, usually to at least about 1% to as much as 15 or 20% by weight and may be selected primarily based on required dose, fluid volumes, viscosities, etc., according to the mode of administration selected. Suitable vehicles and formulations, inclusive of other human proteins, e.g., human serum albumin, are described, for example, in e.g., Remington: The Science and Practice of Pharmacy, 21st Edition, Troy, D. B. ed., Lipincott Williams and Wilkins, Philadelphia, PA 2006, Part 5, Pharmaceutical Manufacturing pp 691-1092, See especially pp. 958-989.

A pharmaceutically acceptable carrier can include a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation.

Pharmaceutical compositions may comprise buffers such as acetic acid, citric acid, formic acid, succinic acid, phosphoric acid, carbonic acid, malic acid, aspartic acid, histidine, boric acid, Tris buffers, HEPPSO, HEPES, neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

Pharmaceutical compositions of the present disclosure can be formulated for a variety of means of parenteral or non-parenteral administration. In one embodiment, the compositions can be formulated for infusion or intravenous administration. Pharmaceutical compositions disclosed herein can be provided, for example, as sterile liquid preparations, e.g., isotonic aqueous solutions, emulsions, suspensions, dispersions, or viscous compositions, which may be buffered to a desirable pH. Formulations suitable for oral administration can include liquid solutions, capsules, sachets, tablets, lozenges, and troches, powders liquid suspensions in an appropriate liquid and emulsions.

The term “pharmaceutically acceptable,” as used herein with regard to pharmaceutical compositions, means approved by a regulatory agency of the Federal or a state government or listed in the U.S. Pharmacopeia or other generally recognized pharmacopeia for use in animals and/or in humans.

Method of Treatment and Uses

The disclosure also provides a method of treating a DLL3 expressing cancer in a subject, comprising administering a therapeutically effective amount of the antigen binding region that binds DLL3 of the disclosure to the subject in need thereof for a time sufficient to treat the DLL3 expressing cancer.

The disclosure also provides a method of treating a DLL3 expressing cancer in a subject, comprising administering a therapeutically effective amount of the protein comprising the antigen biding domain that binds DLL3 of the disclosure to the subject for a time sufficient to treat the DLL3 expressing cancer The disclosure also provides a method of treating a DLL3 expressing cancer in a subject, comprising administering a therapeutically effective amount of the multispecific antigen-binding construct comprising the antigen biding domain that binds DLL3 of the disclosure to the subject for a time sufficient to treat the DLL3 expressing cancer.

The disclosure also provides a method of treating a DLL3 expressing cancer in a subject, comprising administering a therapeutically effective amount of the immunoconjugate comprising the antigen biding domain that binds DLL3 of the disclosure to the subject for a time sufficient to treat the DLL3 expressing cancer.

The disclosure also provides a method of treating a DLL3 expressing cancer in a subject, comprising administering a therapeutically effective amount of the pharmaceutical composition comprising the antigen biding domain that binds DLL3 of the disclosure to the subject for a time sufficient to treat the DLL3 expressing cancer.

In one aspect, the disclosure relates generally to the treatment of a subject at risk of developing cancer. The application also includes treating a malignancy or an autoimmune disease in which chemotherapy and/or immunotherapy results in significant immunosuppression in a subject, thereby increasing the risk of the subject developing cancer.

The disclosure also provides a method of treating a noncancerous condition in a subject at risk of developing a cancerous condition, comprising administering the antigen binding region that bind DLL3 of the disclosure to the subject to treat the noncancerous condition.

The disclosure also provides a method of treating a noncancerous condition in a subject at risk of developing a cancerous condition, comprising administering the protein comprising the antigen binding region that bind DLL3 of the disclosure to the subject to treat the noncancerous condition.

The disclosure also provides a method of treating a noncancerous condition in a subject at risk of developing a cancerous condition, comprising administering the multispecific antigen-binding construct comprising the antigen binding region that bind DLL3 of the disclosure to the subject to treat the noncancerous condition.

The disclosure also provides a method of treating a noncancerous condition in a subject at risk of developing a cancerous condition, comprising administering the immunoconjugate of the disclosure to the subject to treat the noncancerous condition.

The disclosure also provides a method of treating a noncancerous condition in a subject at risk of developing a cancerous condition, comprising administering the pharmaceutical composition of the disclosure to the subject to treat the noncancerous condition.

In some embodiments, the subject at risk of developing the cancerous condition has an enlarged prostate.

In some embodiments, the subject at risk of developing the cancerous condition has a benign prostate hyperplasia (BPH).

In some embodiments, the subject at risk of developing the cancerous condition has a and high PSA levels in absence of diagnosed prostate cancer.

The disclosure also provides a method of preventing DLL3 expressing cancer in a subject, comprising administering a therapeutically effective amount of the antigen biding domain that binds DLL3 of the disclosure to the subject for a time sufficient to prevent the DLL3 expressing cancer.

The disclosure also provides a method of preventing a DLL3 expressing cancer in a subject, comprising administering a therapeutically effective amount of the protein comprising the antigen biding domain that binds DLL3 of the disclosure to the subject for a time sufficient to prevent the DLL3 expressing cancer.

The disclosure also provides a method of preventing a DLL3 expressing cancer in a subject, comprising administering a therapeutically effective amount of the multispecific antigen-binding construct comprising the antigen biding domain that binds DLL3 of the disclosure to the subject for a time sufficient to prevent the DLL3 expressing cancer.

The disclosure also provides a method of preventing a DLL3 expressing cancer in a subject, comprising administering a therapeutically effective amount of the immunoconjugate comprising the antigen biding domain that binds DLL3 of the disclosure to the subject for a time sufficient to prevent the DLL3 expressing cancer.

The disclosure also provides a method of preventing a DLL3 expressing cancer in a subject, comprising administering a therapeutically effective amount of the pharmaceutical composition comprising the antigen biding domain that binds DLL3 of the disclosure to the subject for a time sufficient to prevent the DLL3 expressing cancer.

The disclosure also provides a method of reducing the amount of DLL3 expressing tumor cells in a subject, comprising administering the antigen biding domain that binds DLL3 of the disclosure to the subject for a time sufficient to reduce the amount of DLL3 expressing tumor cells.

The disclosure also provides a method of reducing the amount of DLL3 expressing tumor cells in a subject, comprising administering the protein comprising the antigen biding domain that binds DLL3 of the disclosure to the subject for a time sufficient to reduce the amount of DLL3 expressing tumor cells.

The disclosure also provides a method of reducing the amount of DLL3 expressing tumor cells in a subject, comprising administering the multispecific antigen-binding construct comprising the antigen biding domain that binds DLL3 of the disclosure to the subject for a time sufficient to reduce the amount of DLL3 expressing tumor cells.

The disclosure also provides a method of reducing the amount of DLL3 expressing tumor cells in a subject, comprising administering the immunoconjugate of the disclosure to the subject for a time sufficient to reduce the amount of DLL3 expressing tumor cells.

The disclosure also provides a method of reducing the amount of DLL3 expressing tumor cells in a subject, comprising administering the pharmaceutical composition of the disclosure to the subject for a time sufficient to reduce the amount of DLL3 expressing tumor cells.

In some embodiments, the DLL3 expressing cancer is prostate cancer.

In some embodiments, the DLL3 expressing cancer is neuroendocrine prostate cancer.

In some embodiments, the DLL3 expressing cancer is prostate derived cancer.

In some embodiments, the DLL3 expressing cancer has metastasized to bone.

In some embodiments, the DLL3 expressing cancer is lung cancer.

In some embodiments, the DLL3 expressing cancer is small cell lung cancer.

In some embodiments, the prostate cancer is relapsed, refractory, malignant or castration resistant prostate cancer, or any combination thereof.

In some embodiments, the lung cancer is relapsed, refractory or malignant lung cancer, or any combination thereof.

In some embodiments, the neuroendocrine prostate cancer is relapsed, refractory, malignant or castration resistant prostate cancer, or any combination thereof.

In some embodiments, the small cell lung cancer is relapsed, refractory or malignant lung cancer, or any combination thereof.

The disclosure also provides a method of treating prostate cancer in a subject, comprising administering a therapeutically effective amount of a multispecific antigen-binding construct according to an embodiment of the application comprising an antigen binding region that binds DLL3 to the subject for a time sufficient to treat the prostate cancer.

The disclosure also provides the use of a multispecific antibody according to an embodiment of the application in the manufacture of a medicament for the treatment of prostate cancer in a subject.

The disclosure also provides a method of treating prostate cancer in a subject, comprising administering a therapeutically effective amount of a multispecific antigen-binding construct according to an embodiment of the application to the subject for a time sufficient to treat the prostate cancer. In some embodiment, the method of treating prostate cancer in a subject comprises administering a therapeutically effective amount of a multispecific antigen-binding construct comprises an antigen binding region that binds DLL3 comprising the amino acid sequence of SEQ ID NOs:63, 64, 65, 66, 67, 68, or 69.

The disclosure also provides the use of a multispecific antibody according to an embodiment of the application in the manufacture of a medicament for the treatment of prostate cancer in a subject. In some embodiment, the use of a multispecific antibody comprising an antigen binding region that binds DLL3 comprising the amino acid sequence of SEQ ID NOs:63, 64, 65, 66, 67, 68, or 69.

The disclosure also provides a method of treating small cell lung cancer in a subject, comprising administering a therapeutically effective amount of the multispecific antigen-binding construct according to an embodiment of the application to the subject for a time sufficient to treat the small cell lung cancer.

The disclosure also provides the use of a multispecific antibody according to an embodiment of the application in the manufacture of a medicament for the treatment of small cell lung cancer in a subject.

The disclosure also provides a method of treating small cell lung cancer in a subject, comprising administering a therapeutically effective amount of a multispecific antigen-binding construct comprising an antigen binding region that binds DLL3 to the subject for a time sufficient to treat the small cell lung cancer, wherein the antigen binding region that binds DLL3 comprises the amino acid sequence of SEQ ID NOs:63, 64, 65, 66, 67, 68, or 69.

The disclosure also provides the use of a multispecific antibody according to an embodiment of the application in the manufacture of a medicament for the treatment of small cell lung cancer in a subject. In some embodiments, the multispecific antigen-binding construct comprises an antigen binding region that binds DLL3 having the amino acid sequence of SEQ ID NOs:63, 64, 65, 66, 67, 68, or 69.

Combination Therapies

The DLL3 binding proteins of the disclosure may be administered in combination with at least one additional therapeutics.

In some embodiments the at least one additional therapeutic is surgery, chemotherapy, androgen deprivation therapy or radiation, or any combination thereof.

In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The following examples are provided to further describe some of the embodiments disclosed herein. The examples are intended to illustrate, not to limit, the disclosed embodiments.

### Numbered Embodiments

The present disclosure also provides the following numbered embodiments:

1. An isolated protein comprising an antigen binding region that binds delta-like protein 3 (DLL3), wherein the antigen binding region binds to an epitope within residues 429-618 of human DLL3 as set forth in SEQ ID NO:263.

2. The isolated protein of embodiment 1, wherein the antigen binding region competes for binding to DLL3 with a reference antibody comprising a heavy chain variable region (VH) comprising heavy chain complementarity determining regions (HCDRs) HCDR1, HCDR2 and HCDR3, and a light chain variable region (VL) comprising light chain complementarity determining regions (LCDRs) LCDR1, LCDR2 and LCDR3, wherein the HCDR1, HCDR2, HCDR3, LCDR1, LCDR2 and LCDR3 have the amino acid sequences of:
a. the HCDR1, the HCDR2 and the HCDR3 of a VH of SEQ ID NO:1 and the LCDR1, the LCDR2 and the LCDR3 of a VL of SEQ ID NO:2;
b. the HCDR1, the HCDR2 and the HCDR3 of a VH of SEQ ID NO:3 and the LCDR1, the LCDR2 and the LCDR3 of a VL of SEQ ID NO:4;
c. the HCDR1, the HCDR2 and the HCDR3 of a VH of SEQ ID NO:5 and the LCDR1, the LCDR2 and the LCDR3 of a VL of SEQ ID NO:6;
d. the HCDR1, the HCDR2 and the HCDR3 of a VH of SEQ ID NO:7 and the LCDR1, the LCDR2 and the LCDR3 of a VL of SEQ ID NO:8;
e. the HCDR1, the HCDR2 and the HCDR3 of a VH of SEQ ID NO:9 and the LCDR1, the LCDR2 and the LCDR3 of a VL of SEQ ID NO: 10;
f. the HCDR1, the HCDR2 and the HCDR3 of a VH of SEQ ID NO:11 and the LCDR1, the LCDR2 and the LCDR3 of a VL of SEQ ID NO: 12; or
g. the HCDR1, the HCDR2 and the HCDR3 of a VH of SEQ ID NO:13 and the LCDR1, the LCDR2 and the LCDR3 of a VL of SEQ ID NO: 14.

3. The isolated protein of embodiment 2, wherein the reference antibody comprises the HCDR1, the HCDR2 and the HCDR3 of the VH of SEQ ID NO:3, and the LCDR1, the LCDR2 and the LCDR3 of the VL of SEQ ID NO:4.

4. The isolated protein of any one of embodiments 1-3, comprising the HCDR1, the HCDR2, the HCDR3, the LCDR1, the LCDR2 and the LCDR3 of
a. SEQ ID NOs:15, 16, 17, 33, 34, 35, respectively;
b. SEQ ID NOs:18, 19, 20, 36, 37, 38, respectively;
c. SEQ ID NOs:21, 22, 23, 39, 37, 40, respectively;
d. SEQ ID NOs:24, 25, 26, 41, 42, 43, respectively;
e. SEQ ID NOs:18, 28, 29, 44, 45, 46, respectively;
f. SEQ ID NOs:30, 31, 32, 47, 48, 49, respectively;
g. SEQ ID NOs:50, 51, 17, 33, 34, 35, respectively;
h. SEQ ID NOs:52, 51, 17, 33, 34, 35, respectively;
i. SEQ ID NOs:53, 54, 20, 36, 37, 38, respectively;
j. SEQ ID NOs:55, 56, 23, 39, 37, 40, respectively;
k. SEQ ID NOs:57, 58, 26, 41, 42, 43, respectively;
l. SEQ ID NOs:59, 60, 29, 44, 45, 46, respectively; or
m. SEQ ID NOs:61, 62, 32, 47, 48, 49, respectively.

5. The isolated protein of embodiment 4, comprising the HCDR1, the HCDR2, the HCDR3, the LCDR1, the LCDR2 and the LCDR3 of SEQ ID NOs:15, 16, 17, 33, 34, and 35, respectively.

6. The isolated protein of any one of embodiments 1-5, wherein the antigen binding region is a scFv, a (scFv)₂, a Fv, a Fab, a F(ab′)₂, a Fd, a dAb or a VHH.

7. The isolated protein of embodiment 6, wherein the antigen binding region is the Fab.

8. The isolated protein of embodiment 6, wherein the antigen binding region is the VHH.

9. The isolated protein of embodiment 6, wherein the antigen binding region is the scFv.

10. The isolated protein of embodiment 9, wherein the scFv comprises, from the N- to C-terminus, a VH, a first linker (L1) and a VL (VH-L1-VL) or the VL, the L1 and the VH (VL-L1-VH).

11. The isolated protein of embodiment 10, wherein the L1 comprises:
a. about 5-50 amino acids;
b. about 5-40 amino acids;
c. about 10-30 amino acids; or
d. about 10-20 amino acids.

12. The isolated protein of embodiment 11, wherein the L1 comprises an amino acid sequence of SEQ ID NO:27, 72, 73, 74, 75, 76, 79, 81, 82, 83, 88, 90, 91, 92, 120, 121, 122, 123, 124, 125, 126, 127, 128, 129, 130, 131, 132, 133, 134, 135, 136, 137, 138, or 139.

13. The isolated protein of embodiment 12, wherein the L1 comprises the amino acid sequence of SEQ ID NO:120.

14. The isolated protein of any one of embodiments 1-13, wherein the antigen binding region comprises the VH of SEQ ID NO: 1, 3, 5, 7, 9, 11, or 13 and the VL of SEQ ID NO: 2, 4, 6, 8, 10, 12, or 14.

15. The isolated protein of embodiment 14, wherein the antigen binding region comprises:
a. the VH of SEQ ID NO:1 and the VL of SEQ ID NO:2;
b. the VH of SEQ ID NO:3 and the VL of SEQ ID NO:4;
c. the VH of SEQ ID NO:5 and the VL of SEQ ID NO:6;
d. the VH of SEQ ID NO:7 and the VL of SEQ ID NO:8;
e. the VH of SEQ ID NO:9 and the VL of SEQ ID NO:10;
f. the VH of SEQ ID NO: 11 and the VL of SEQ ID NO: 12; or
g. the VH of SEQ ID NO: 13 and the VL of SEQ ID NO: 14.

16. The isolated protein of embodiment 14, wherein the antigen binding region comprises a VH which is at least 80% (e.g., at least 85%, at least 90%, at least 95%, at least 99% or 100%) identical to the VH of SEQ ID NO:3 and a VL which is at least 80% (e.g., at least 85%, at least 90%, at least 95%, at least 99% or 100%) identical to the VL of SEQ ID NO:4.

17. The isolated protein of any one of embodiments 1-16, wherein the antigen binding region comprises an amino acid sequence at least 80% (e.g., at least 85%, at least 90%, at least 95%, at least 99% or 100%) identical to the amino acid sequence of SEQ ID NO:63 or 64.

18. The isolated protein of any one of embodiments 1-17, wherein the isolated protein is a monospecific protein.

19. A multispecific antigen-binding construct comprising the protein of any one of embodiments 1-17.

20. The multispecific antigen-binding construct of embodiment 19, being a bispecific antigen-binding construct.

21. The multispecific antigen-binding construct of embodiment 19, being a trispecific antigen-binding construct.

22. The multispecific antigen-binding construct of embodiment 20 or 21, further comprising a second antigen binding region that binds an antigen on a lymphocyte.

23. The multispecific antigen-binding construct of embodiment 22, wherein the lymphocyte is a T cell.

24. The multispecific antigen-binding construct of embodiment 23, wherein the T cell is a CD8⁺ T cell.

25. The multispecific antigen-binding construct of embodiment 22, wherein the lymphocyte is a natural killer (NK) cell.

26. The multispecific antigen-binding construct of embodiment 22, wherein the antigen on the lymphocyte is CD3, CD3 epsilon (CD3ε), CD8, KI2L4, NKG2E, NKG2D, NKG2F, BTNL3, CD186, BTNL8, PD-1, CD195, or NKG2C.

27. The multispecific antigen-binding construct of embodiment 26, wherein the second antigen binding region binds CD3ε.

28. The multispecific antigen-binding construct of embodiment 27, wherein the second antigen binding region that binds CD3ε comprises:
a) a heavy chain complementarity determining region HCDR1 of SEQ ID NO:98, a HCDR2 of SEQ ID NO:99, a HCDR3 of SEQ ID NO: 100, a light chain complementarity determining region LCDR1 of SEQ ID NO: 106, a LCDR2 of SEQ ID NO:107 and a LCDR3 of SEQ ID NO:108; or
b) the VH of SEQ ID NO:84 and the VL of SEQ ID NO:85.

29. The multispecific antigen-binding construct of embodiment 28, wherein the second antigen binding region comprises a HCDR1 of SEQ ID NO:98, a HCDR2 of SEQ ID NO:99, a HCDR3 of SEQ ID NO: 100, a LCDR1 of SEQ ID NO: 106, a LCDR2 of SEQ ID NO:107 and a LCDR3 of SEQ ID NO: 108.

30. The multispecific antigen-binding construct of embodiment 28 or 29, wherein the second antigen binding region comprises a VH which is at least 80% (e.g., at least 85%, at least 90%, at least 95%, at least 99% or 100%) identical to the VH of SEQ ID NO:84 and a VL which is at least 80% (e.g., at least 85%, at least 90%, at least 95%, at least 99% or 100%) identical to the VL of SEQ ID NO:85.

31. The multispecific antigen-binding construct of embodiment 27, wherein the second antigen binding region comprises:
a) a heavy chain complementarity determining region (HCDR)1 of SEQ ID NO:95, a HCDR2 of SEQ ID NO:96, a HCDR3 of SEQ ID NO:97, a light chain complementarity determining region (LCDR)1 of SEQ ID NO:101, a LCDR2 of SEQ ID NO:102 and a LCDR3 of SEQ ID NO:104; or
b) the VH of SEQ ID NO:77 and the VL of SEQ ID NO:80.

32. The multispecific antigen-binding construct of embodiment 31, wherein the second antigen binding region comprises a HCDR1 of SEQ ID NO:95, a HCDR2 of SEQ ID NO:96, a HCDR3 of SEQ ID NO:97, a LCDR1 of SEQ ID NO: 101, a LCDR2 of SEQ ID NO: 102 and a LCDR3 of SEQ ID NO: 104.

33. The multispecific antigen-binding construct of embodiment 31 or 32, wherein the second antigen binding region comprises a VH which is at least 80% (e.g., at least 85%, at least 90%, at least 95%, at least 99% or 100%) identical to the VH of SEQ ID NO:77 and a VL which is at least 80% (e.g., at least 85%, at least 90%, at least 95%, at least 99% or 100%) identical to the VL of SEQ ID NO:80.

34. The multispecific antigen-binding construct of embodiment 33, comprising the antigen binding domain that binds DLL3 having the HCDR1, the HCDR2, the HCDR3, the LCDR1, the LCDR2 and the LCDR3 of SEQ ID NOs:15, 16, 17, 33, 34, and 35, respectively, and the second antigen binding region that binds CD3ε having the HCDR1, the HCDR2, the HCDR3, the LCDR1, the LCDR2 and the LCDR3 of SEQ ID NO:98, SEQ ID NO:99, SEQ ID NO: 100, SEQ ID NO: 106, SEQ ID NO:107 and SEQ ID NO:108, respectively.

35. The multispecific antigen-binding construct of embodiment 34, wherein the antigen binding domain that binds DLL3 comprises the VH of SEQ ID NO:3 and the VL of SEQ ID NO:4, and the second antigen binding region that binds CD3ε comprises the VH of SEQ ID NO:84 and the VL of SEQ ID NO:85.

36. A fusion or conjugate comprising a half-life extending moiety fused or covalently linked to the isolated protein of any one of embodiments 1-8 or the multispecific antigen-binding construct of any one of embodiments 19-35.

37. The fusion or conjugate of embodiment 36, wherein the half-life extending moiety is an immunoglobulin (Ig), a fragment of the Ig, an Ig constant region, a fragment of the Ig constant region, a Fc region, transferrin, albumin, an albumin binding domain or polyethylene glycol.

38. The fusion or conjugate of embodiment 37, wherein the fragment of the Ig constant region comprises a Fc region.

39. The fusion or conjugate of any one of embodiments 36-38, wherein the antigen binding region that binds DLL3 is fused to the N-terminus of the Ig constant region or the fragment of the Ig constant region.

40. The fusion or conjugate of any one of embodiments 36-38, wherein the antigen binding region that binds DLL3 is fused to the C-terminus of the Ig constant region or the fragment of the Ig constant region.

41. The fusion or conjugate of any one of embodiments 36-38, wherein the antigen binding region that binds DLL3 is fused to the Ig constant region or the fragment of the Ig constant region via a second linker (L2).

42. The fusion or conjugate of embodiment 41, wherein the L2 comprises the amino acid sequence of SEQ ID NO:27, 72, 73, 74, 75, 76, 79, 81, 82, 83, 88, 90, 91, 92, 120, 121, 122, 123, 124, 125, 126, 127, 128, 129, 130, 131, 132, 133, 134, 135, 136, 137, 138, or 139.

43. The fusion or conjugate of any one of embodiments 37-42, wherein the Ig constant region or the fragment of the Ig constant region is an IgG1, an IgG2, an IgG3 or an IgG4 isotype.

44. The fusion or conjugate of embodiment 43, wherein the Ig constant region or the fragment of the Ig constant region is an IgG1 isotype.

45. The fusion or conjugate of any one of embodiments 37-44, wherein the Ig constant region or the fragment of the Ig constant region comprises at least one mutation that results in reduced binding of the protein to a Fcγ receptor (FcγR).

46. The fusion or conjugate of embodiment 45, wherein the at least one mutation that results in reduced binding of the protein to the FcγR is selected from the group consisting of F234A/L235A, L234A/L235A, L234A/L235A/D265 S, V234A/G237A/P238 S/H268A/V309L/A330 S/P33IS, F234A/L235A, S228P/F234A/L235A, N297A, V234A/G237A, K214T/E233P/L234V/L235A/G236-deleted/A327G/P331A/D365E/L358M, H268Q/V309L/A330 S/P331 S, S267E/L328F, L234F/L235E/D265A, L234A/L235A/G237A/P238 S/H268A/A330 S/P331 S, S228P/F234A/L235A/G237A/P238 S and S228P/F234A/L235A/G236-deleted/G237A/P238 S, wherein residue numbering is according to the EU index.

47. The fusion or conjugate of embodiment 46, wherein the mutations that results in reduced binding of the protein to the FcγR are L234A_L235A_D265 S.

48. The fusion or conjugate of any one of embodiments 37-44, comprising at least one mutation in a CH3 domain of the Ig constant region.

49. The fusion or conjugate of embodiment 48, wherein the at least one mutation in the CH3 domain of the Ig constant region is selected from the group consisting of T350V, L351Y, F405A, Y407V, T366Y, T366W, F405W, T394W, T394 S, Y407T, Y407A, T366 S/L368A/Y407V, L351Y/F405A/Y407V, T3661/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V and T350V/T366L/K392L/T394W, wherein residue numbering is according to the EU index.

50. An isolated protein comprising an antigen binding region that binds DLL3, wherein the antigen binding region comprises:
a. a HCDR1, a HCDR2, a HCDR3, a LCDR1, a LCDR2, and a LCDR3 of SEQ ID NOs:15, 16, 17, 33, 34, 35, respectively;
b. a VH of SEQ ID NO:1 and a VL of SEQ ID NO:2;
c. a VH of SEQ ID NO:3 and a VL of SEQ ID NO:4;
d. a scFv of SEQ ID NO:63; and/or
e. a scFv of SEQ ID NO:64.

51. An isolated protein comprising an antigen binding region that binds DLL3, wherein the antigen binding region comprises:
a. a HCDR1, a HCDR2, a HCDR3, a LCDR1, a LCDR2 and a LCDR3 of SEQ ID NOs:15, 16, 17, 33, 34, 35, respectively; and/or
b. a VH of SEQ ID NO:1 and a VL of SEQ ID NO:2.

52. An isolated protein comprising an antigen binding region that binds DLL3, wherein the antigen binding region comprises:
a. a HCDR1, a HCDR2, a HCDR3, a LCDR1, a LCDR2, and a LCDR3 of SEQ ID NOs:18, 19, 20, 36, 37, 38, respectively;
b. a VH of SEQ ID NO:5 and a VL of SEQ ID NO:6; and/or
c. an scFv of SEQ ID NO:65.

53. An isolated protein comprising an antigen binding region that binds DLL3, wherein the antigen binding region comprises:
a. a HCDR1, a HCDR2, a HCDR3, a LCDR1, a LCDR2 and a LCDR3 of SEQ ID NOs:21, 22, 23, 39, 37, 40, respectively;
b. a VH of SEQ ID NO:7 and a VL of SEQ ID NO:8; and/or
c. an scFv of SEQ ID NO:66.

54. An isolated protein comprising an antigen binding region that binds DLL3, wherein the antigen binding region comprises:
a. a HCDR1, a HCDR2, a HCDR3, a LCDR1, a LCDR2, and a LCDR3 of SEQ ID NOs:24, 25, 26, 41, 42, 43, respectively;
b. a VH of SEQ ID NO:9 and a VL of SEQ ID NO:10; and/or
c. an scFv of SEQ ID NO:67.

55. An isolated protein comprising an antigen binding region that binds DLL3, wherein the antigen binding region comprises:
a. a HCDR1, a HCDR2, a HCDR3, a LCDR1, a LCDR2 and a LCDR3 of SEQ ID NOs:27, 28, 29, 44, 45, 46, respectively;
b. a VH of SEQ ID NO:11 and a VL of SEQ ID NO:12; and/or
c. an scFv of SEQ ID NO:68.

56. An isolated protein comprising an antigen binding region that binds DLL3, wherein the antigen binding region comprises:
a. a HCDR1, a HCDR2, a HCDR3, a LCDR1, a LCDR2, and a LCDR3 of SEQ ID NOs: 30, 31, 32, 47, 48, 49, respectively;
b. a VH of SEQ ID NO: 13 and a VL of SEQ ID NO: 14; and/or
c. an scFv of SEQ ID NO:69.

57. A multispecific antigen-binding construct comprising the isolated protein of any one of embodiments 50-56 and a second antigen binding region that binds CD3ε.

58. The multispecific antigen-binding construct of embodiment 57, wherein the second antigen binding region that binds CD3ε comprises:
a. a heavy chain complementarity determining region (HCDR)1 of SEQ ID NO:98, a HCDR2 of SEQ ID NO:99, a HCDR3 of SEQ ID NO: 100, a light chain complementarity determining region (LCDR)1 of SEQ ID NO:106, a LCDR2 of SEQ ID NO: 107 and a LCDR3 of SEQ ID NO:108; and/or
b. the VH of SEQ ID NO:84 and the VL of SEQ ID NO:85.

59. The multispecific antigen-binding construct of embodiment 57, wherein the second antigen binding region that binds CD3ε comprises:
a. a heavy chain complementarity determining region (HCDR)1 of SEQ ID NO:95, a HCDR2 of SEQ ID NO:96, a HCDR3 of SEQ ID NO:97, a light chain complementarity determining region (LCDR)1 of SEQ ID NO:101, a LCDR2 of SEQ ID NO: 102 and a LCDR3 of SEQ ID NO: 104; and/or
b. the VH of SEQ ID NO:77 and the VL of SEQ ID NO:80.

60. An isolated bispecific construct, comprising a first antigen binding region that binds DLL3 and a second antigen binding region that binds a lymphocyte antigen.

61. The isolated bispecific construct of embodiment 60, wherein the lymphocyte antigen is a T cell antigen.

62. The isolated bispecific construct of embodiment 61, wherein the T cell antigen is a CD8⁺ T cell antigen.

63. The isolated bispecific construct of embodiment 62, wherein the lymphocyte antigen is a NK cell antigen.

64. The isolated bispecific construct of any one of embodiments 60-63, wherein the lymphocyte antigen is CD3, CD3 epsilon (CD3ε), CD8, K12L4, NKG2E, NKG2D, NKG2F, BTNL3, CD186, BTNL8, PD-1, CD195, or NKG2C.

65. The isolated bispecific construct of embodiment 64, wherein the lymphocyte antigen is CD3ε.

66. The isolated bispecific construct of any one of embodiments 60-65, wherein the first antigen binding region that binds DLL3 and/or the second antigen binding region that binds the lymphocyte antigen comprise a scFv, a (scFv)₂, a Fv, a Fab, a F(ab′)₂, a Fd, a dAb or a VHH.

67. The isolated bispecific construct of embodiment 66, wherein the first antigen binding region that binds DLL3 and/or the second antigen binding region that binds the lymphocyte antigen comprise the Fab.

68. The isolated bispecific construct of embodiment 66, wherein the first antigen binding region that binds DLL3 and/or the second antigen binding region that binds the lymphocyte antigen comprise the scFv.

69. The isolated bispecific construct of embodiment 66, wherein the first antigen binding region that binds DLL3 and/or the second antigen binding region that binds the lymphocyte antigen comprise the VHH.

70. The isolated bispecific construct of embodiment 64, wherein the first antigen binding region that binds DLL3 and/or the second antigen binding region that binds the lymphocyte antigen comprise the scFv.

71. The isolated bispecific construct of embodiment 66, wherein the first antigen binding region that binds DLL3 comprises the scFv and the second antigen binding region that binds the lymphocyte antigen comprise the Fab.

72. The isolated bispecific construct of embodiment 66, wherein the first antigen binding region that binds DLL3 comprises the Fab and the second antigen binding region that binds the lymphocyte antigen comprise the scFv.

73. The isolated bispecific construct of any one of embodiments 66-72, wherein the scFv comprises, from the N- to C-terminus, a VH, a first linker (L1) and a VL (VH-L1-VL) or the VL, the L1 and the VH (VL-L1-VH).

74. The isolated bispecific construct of embodiment 73, wherein the L1 comprises
a. about 5-50 amino acids;
b. about 5-40 amino acids;
c. about 10-30 amino acids; or
d. about 10-20 amino acids.

75. The isolated bispecific construct of embodiment 74, wherein the L1 comprises the amino acid sequence of SEQ ID NOs:27, 72, 73, 74, 75, 76, 79, 81, 82, 83, 88, 90, 91, 92, 120, 121, 122, 123, 124, 125, 126, 127, 128, 129, 130, 131, 132, 133, 134, 135, 136, 137, 138, or 139.

76. The isolated bispecific construct of embodiment 75, wherein the L1 comprises the amino acid sequence of SEQ ID NO: 120.

77. The isolated bispecific construct of any one of embodiments 60-76 being an isolated anti-DLL3/anti-CD3 construct, wherein the first antigen binding region that binds DLL3 comprises a HCDR1 of SEQ ID NOs:15, 18, 21, 24, 27, 30, 50, 52, 53, 55, 57, 59, or 61, a HCDR2 of SEQ ID NOs:16, 19, 22, 25, 28, 31, 51, 54, 56, 58, 60, or 62, a HCDR3 of SEQ ID NOs:17, 20, 23, 26, 29, 32, 17, 20, 23, 26, 29, or 32, a LCDR1 of SEQ ID NOs:33, 36, 39, 41, 44, or 47, a LCDR2 of SEQ ID NOs: 34, 37, 42, 45, or 48, and a LCDR3 of SEQ ID NOs:35, 38, 40, 43, 46, or 49.

78. The isolated anti-DLL3/anti-CD3 construct of 77, wherein the first antigen binding region that binds DLL3 comprises the HCDR1, the HCDR2, the HCDR3, the LCDR1, the LCDR2 and the LCDR3 of
a. SEQ ID NOs:15, 16, 17, 33, 34, 35, respectively;
b. SEQ ID NOs:18, 19, 20, 36, 37, 38, respectively;
c. SEQ ID NOs:21, 22, 23, 39, 37, 40, respectively;
d. SEQ ID NOs:24, 25, 26, 41, 42, 43, respectively;
e. SEQ ID NOs:18, 28, 29, 44, 45, 46, respectively;
f. SEQ ID NOs:30, 31, 32, 47, 48, 49, respectively;
g. SEQ ID NOs:50, 51, 17, 33, 34, 35, respectively;
h. SEQ ID NOs:52, 51, 17, 33, 34, 35, respectively;
i. SEQ ID NOs:53, 54, 20, 36, 37, 38, respectively;
j. SEQ ID NOs:55, 56, 23, 39, 37, 40, respectively;
k. SEQ ID NOs:57, 58, 26, 41, 42, 43, respectively;
l. SEQ ID NOs:59, 60, 29, 44, 45, 46, respectively; or
m. SEQ ID NOs:61, 62, 32, 47, 48, 49, respectively.

79. The isolated anti-DLL3/anti-CD3 construct of 78, wherein the first antigen binding region that binds DLL3 comprises the HCDR1, the HCDR2, the HCDR3, the LCDR1, the LCDR2 and the LCDR3 of SEQ ID NOs:15, 16, 17, 33, 34, 35, respectively.

80. The isolated anti-DLL3/anti-CD3 construct of embodiment 77, wherein the first antigen binding region that binds DLL3 comprises
a. the VH of SEQ ID NO:1 and the VL of SEQ ID NO:2;
b. the VH of SEQ ID NO:3 and the VL of SEQ ID NO:4;
c. the VH of SEQ ID NO:5 and the VL of SEQ ID NO:6;
d. the VH of SEQ ID NO:7 and the VL of SEQ ID NO:8;
e. the VH of SEQ ID NO:9 and the VL of SEQ ID NO:10;
f. the VH of SEQ ID NO:11 and the VL of SEQ ID NO: 12; or
g. the VH of SEQ ID NO: 13 and the VL of SEQ ID NO: 14.

81. The isolated anti-DLL3/anti-CD3 construct of embodiment 80, wherein the first antigen binding region that binds DLL3 comprises an scFv having the amino acid sequence of SEQ ID NO:63 or 64.

82. The isolated anti-DLL3/anti-CD3 construct of embodiment 80, wherein the first antigen binding region that binds DLL3 comprises an amino acid sequence at least 80% (e.g., at least 85%, at least 90%, at least 95%, at least 99% or 100%) identical to the amino acid sequence of SEQ ID NO:64.

83. The isolated anti-DLL3/anti-CD3 construct of any one of embodiments 77-79, wherein the first antigen binding region that binds DLL3 comprises a VH which is at least 80% (e.g., at least 85%, at least 90%, at least 95%, at least 99% or 100%) identical to the VH of SEQ ID NO:3 and a VL which is at least 80% (e.g., at least 85%, at least 90%, at least 95%, at least 99% or 100%) identical to the VL of SEQ ID NO:4.

84. The isolated anti-DLL3/anti-CD3 construct of any one of embodiments 60-83, wherein the second antigen binding region that binds CD3 comprises a HCDR1 of SEQ ID NOs:95 or 98, a HCDR2 of SEQ ID NOs:96 or 99, a HCDR3 of SEQ ID NOs:97 or 100, a LCDR1 of SEQ ID NOs:101 or 106, a LCDR2 of SEQ ID NOs:102 or 107, and a LCDR3 of SEQ ID NOs:104 or 108.

85. The isolated anti-DLL3/anti-CD3 construct of embodiment 84, wherein the second antigen binding region that binds CD3 comprises a HCDR1 of SEQ ID NO:95, a HCDR2 of SEQ ID NO:96, a HCDR3 of SEQ ID NO:97, a LCDR1 of SEQ ID NO: 101, a LCDR2 of SEQ ID NO: 102, and a LCDR3 of SEQ ID NO:104.

86. The isolated anti-DLL3/anti-CD3 construct of embodiment 84, wherein the second antigen binding region that binds CD3 comprises a HCDR1 of SEQ ID NO:98, a HCDR2 of SEQ ID NO:99, a HCDR3 of SEQ ID NO: 100, a LCDR1 of SEQ ID NO: 106, a LCDR2 of SEQ ID NO: 107, and a LCDR3 of SEQ ID NO:108.

87. The isolated anti-DLL3/anti-CD3 construct of embodiment 85, wherein the second antigen binding region that binds CD3 comprises a VH which is at least 80% (e.g., at least 85%, at least 90%, at least 95%, at least 99% or 100%) identical to the VH of SEQ ID NO:77 and a VL which is at least 80% (e.g., at least 85%, at least 90%, at least 95%, at least 99% or 100%) identical to the VL of SEQ ID NO:80.

88. The isolated anti-DLL3/anti-CD3 construct of embodiment 87, wherein the second antigen binding region that binds CD3 comprises a VH of SEQ ID NO:77 and a VL of SEQ ID NO:80.

89. The isolated anti-DLL3/anti-CD3 construct of embodiment 86, wherein the second antigen binding region that binds CD3 comprises a VH which is at least 80% (e.g., at least 85%, at least 90%, at least 95%, at least 99% or 100%) identical to the VH of SEQ ID NO:84 and a VL which is at least 80% (e.g., at least 85%, at least 90%, at least 95%, at least 99% or 100%) identical to the VL of SEQ ID NO:85.

90. The isolated anti-DLL3/anti-CD3 construct of embodiment 89, wherein the second antigen binding region that binds CD3 comprises a VH of SEQ ID NO:84 and a VL of SEQ ID NO:85.

91. The isolated anti-DLL3/anti-CD3 construct of any one of embodiments 59-90, wherein the first antigen binding region that binds DLL3 is conjugated to a first immunoglobulin (Ig) constant region or a fragment of the first Ig constant region and/or the second antigen binding region that binds the lymphocyte antigen is conjugated to a second immunoglobulin (Ig) constant region or a fragment of the second Ig constant region.

92. The isolated anti-DLL3/anti-CD3 construct embodiment 91, further comprising a second linker (L2) between the first antigen binding region that binds DLL3 and the first Ig constant region or the fragment of the first Ig constant region and the second antigen binding region that binds the lymphocyte antigen and the second Ig constant region or the fragment of the second Ig constant region.

93. The isolated anti-DLL3/anti-CD3 construct of embodiment 92, wherein the L2 comprises the amino acid sequence of SEQ ID NO:27, 72, 73, 74, 75, 76, 79, 81, 82, 83, 88, 90, 91, 92, 120, 121, 122, 123, 124, 125, 126, 127, 128, 129, 130, 131, 132, 133, 134, 135, 136, 137, 138, or 139.

94. The isolated anti-DLL3/anti-CD3 construct of any one of embodiments 91-93, wherein the fragment of the Ig constant region comprises a Fc region.

95. The isolated anti-DLL3/anti-CD3 construct of embodiment 94, wherein the first Ig constant region or the fragment of the first Ig constant region and the second Ig constant region or the fragment of the second Ig constant region is an IgG1, an IgG2, and IgG3 or an IgG4 isotype.

96. The isolated anti-DLL3/anti-CD3 construct of embodiment 95, wherein the first Ig constant region or the fragment of the first Ig constant region and the second Ig constant region or the fragment of the second Ig constant region is an IgG1.

97. The isolated anti-DLL3/anti-CD3 construct of embodiment 96, wherein the first Ig constant region or the fragment of the first Ig constant region and the second Ig constant region or the fragment of the second Ig constant region comprises at least one mutation that results in reduced binding of the construct to a FcγR.

98. The isolated anti-DLL3/anti-CD3 construct of embodiment 97, wherein the at least one mutation that results in reduced binding of the construct to the FcγR is selected from the group consisting of F234A/L235A, L234A/L235A, L234A/L235A/D265 S, V234A/G237A/P238 S/H268A/V309L/A330 S/P331 S, F234A/L235A, S228P/F234A/L235A, N297A, V234A/G237A, K214T/E233P/L234V/L235A/G236-deleted/A327G/P331A/D365E/L358M, H268Q/V309L/A330 S/P331 S, S267E/L328F, L234F/L235E/D265A, L234A/L235A/G237A/P238 S/H268A/A330 S/P331 S, S228P/F234A/L235A/G237A/P238 S and S228P/F234A/L235A/G236-deleted/G237A/P238 S, wherein residue numbering is according to the EU index.

99. The isolated anti-DLL3/anti-CD3 construct of embodiment 98, wherein mutations that results in reduced binding of the construct to the FcγR are L234A_L235A_D265 S.

100. The isolated anti-DLL3/anti-CD3 construct of any one of embodiments 91-96, wherein the construct comprises at least one mutation in a CH3 domain of the Ig constant region.

101. The isolated anti-DLL3/anti-CD3 construct of embodiment 100, wherein the at least one mutation in the CH3 domain of the Ig constant region is selected from the group consisting of T350V, L351Y, F405A, Y407V, T366Y, T366W, F405W, T394W, T394 S, Y407T, Y407A, T366 S/L368A/Y407V, L351Y/F405A/Y407V, T3661/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V and T350V/T366L/K392L/T394W, wherein residue numbering is according to the EU index.

102. An isolated anti-DLL3/anti-CD3 construct comprising a first antigen binding region that binds DLL3 and a second antigen binding region that binds CD3, wherein:
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second domain that binds CD3 comprises the HCDR1, the HCDR2, the HCDR3, the LCDR1, the LCDR2 and the LCDR3 of SEQ ID NOs:95, 96, 97, 101, 102, 104, respectively; and/or
b. the first antigen binding region that binds DLL3 comprises a Fab comprising a VH of SEQ ID NO:1 and a VL of SEQ ID NO:2 and the second antigen binding region that binds CD3 comprises a scFv of SEQ ID NO:105; and/or
c. the isolated anti-DLL3/anti-CD3 construct comprises a first heavy chain (HC1) of SEQ ID NO:109, a light chain (LC1) of SEQ ID NO: 110, and a second heavy chain (HC2) of SEQ ID NO: 112.

103. An isolated anti-DLL3/anti-CD3 construct comprising a first antigen binding region that binds DLL3 and a second antigen binding region that binds CD3, wherein:
a. the first antigen binding region that binds DLL3 comprises a HCDR1, a HCDR2, a HCDR3, a LCDR1, a LCDR2 and a LCDR3 of SEQ ID NOs: 15, 16, 17, 33, 34, 35, respectively, and the second antigen binding region that binds CD3 comprises the HCDR1, the HCDR2, the HCDR3, the LCDR1, the LCDR2 and the LCDR3 of SEQ ID NOs: 95, 96, 97, 101, 102, 104, respectively; and/or
b. the first antigen binding region that binds DLL3 comprises a Fab comprising a VH of SEQ ID NO:1 and a VL of SEQ ID NO:2, and the second antigen binding region that binds CD3 comprises a scFv of SEQ ID NO:119; and/or
c. the isolated anti-DLL3/anti-CD3 construct comprises a HC1 of SEQ ID NO: 109, a LC1 of SEQ ID NO:110, and a HC2 of SEQ ID NO: 113.

104. An isolated anti-DLL3/anti-CD3 construct comprising a first antigen binding region that binds DLL3 and a second antigen binding region that binds CD3, wherein:
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second antigen binding region that binds CD3 comprises the HCDR1, the HCDR2, the HCDR3, the LCDR1, the LCDR2 and the LCDR3 of SEQ ID NOs:98, 99, 100, 106, 107, 108, respectively; and/or
b. the first antigen binding region that binds DLL3 comprises a scFv of SEQ ID NO:63, and the second antigen binding region that binds CD3 comprises a Fab comprising a VH of SEQ ID NO:84 and a VL of SEQ ID NO:85; and/or
c. the isolated anti-DLL3/anti-CD3 protein comprises a HC1 of SEQ ID NO:111, a HC2 of SEQ ID NO: 116, and a LC2 of SEQ ID NO:117.

105. An isolated anti-DLL3/anti-CD3 construct comprising a first antigen binding region that binds DLL3 and a second antigen binding region that binds a lymphocyte antigen, wherein:
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second antigen binding region that binds the lymphocyte antigen comprises the HCDR1, the HCDR2, the HCDR3, the LCDR1, the LCDR2 and the LCDR3 of SEQ ID NOs:95, 96, 97, 101, 102, 104, respectively; and/or
b. the first antigen binding region that binds DLL3 comprises a scFv of SEQ ID NO: 64 and the second antigen binding region that binds CD3 comprises a Fab comprising a VH of SEQ ID NO:77 and a VL of SEQ ID NO:80; and/or
c. the isolated anti-DLL3/anti-CD3 protein comprises a HC1 of SEQ ID NO:111, a HC2 of SEQ ID NO:114, and a LC2 of SEQ ID NO:115.

106. An isolated anti-DLL3/anti-CD3 construct comprising a first antigen binding region that binds DLL3 and a second antigen binding region that binds CD3, wherein:
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second antigen binding region that binds CD3 comprises the HCDR1, the HCDR2, the HCDR3, the LCDR1, the LCDR2 and the LCDR3 of SEQ ID NOs:98, 99, 100, 106, 107, 108, respectively; and/or
b. the first antigen binding region that binds DLL3 comprises a scFv of SEQ ID NO:64 and the second antigen binding region that binds the lymphocyte antigen comprises a Fab comprising a VH of SEQ ID NO:84 and a VL of SEQ ID NO:85; and/or
c. the isolated anti-DLL3/anti-CD3 protein comprises a HC1 of SEQ ID NO:71, a HC2 of SEQ ID NO:118, and a LC2 of SEQ ID NO:117.

107. An isolated anti-DLL3/anti-CD3 construct comprising a first antigen binding region that binds DLL3 and a second antigen binding region that binds CD3, wherein:
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second antigen binding region that binds CD3 comprises the HCDR1, the HCDR2, the HCDR3, the LCDR1, the LCDR2 and the LCDR3 of SEQ ID NOs:98, 99, 100, 106, 107, 108, respectively; and/or
b. the first antigen binding region that binds DLL3 comprises a scFv which is at least 80% (e.g., at least 85%, at least 90%, at least 95%, at least 99% or 100%) identical to the scFv of SEQ ID NO:64, and the second antigen binding region that binds CD3 comprises a Fab comprising a VH which is at least 80% (e.g., at least 85%, at least 90%, at least 95%, at least 99% or 100%) identical to the VH of SEQ ID NO:84 and a VL which is at least 80% (e.g., at least 85%, at least 90%, at least 95%, at least 99% or 100%) identical to the VL of SEQ ID NO:85; and/or
c. the isolated anti-DLL3/anti-CD3 protein comprises a HC1 which is at least 80% (e.g., at least 85%, at least 90%, at least 95%, at least 99% or 100%) identical to the HC1 of SEQ ID NO:71, a HC2 which is at least 80% (e.g., at least 85%, at least 90%, at least 95%, at least 99% or 100%) identical to the HC2 of SEQ ID NO:118, and a LC2 which is at least 80% (e.g., at least 85%, at least 90%, at least 95%, at least 99% or 100%) identical to the of SEQ ID NO: 117.

108. An isolated anti-DLL3/anti-CD3 construct comprising a first antigen binding region that binds DLL3 and a second antigen binding region that binds CD3, wherein:
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second antigen binding region that binds CD3 comprises the HCDR1, the HCDR2, the HCDR3, the LCDR1, the LCDR2 and the LCDR3 of SEQ ID NOs:98, 99, 100, 106, 107, 108, respectively;
b. the first antigen binding region that binds DLL3 comprises a scFv of SEQ ID NO:64 and the second antigen binding region that binds CD3 comprises a Fab comprising a VH of SEQ ID NO:84 and a VL of SEQ ID NO:85; and/or
c. the isolated anti-DLL3/anti-CD3 protein comprises a HC1 of SEQ ID NO:229, a HC2 of SEQ ID NO:230, and a LC2 of SEQ ID NO: 117.

109. An isolated anti-DLL3/anti-CD3 construct comprising a first antigen binding region that binds DLL3 and a second antigen binding region that binds CD3, wherein:
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, 35, respectively, and the second antigen binding region that binds CD3 comprises the HCDR1, the HCDR2, the HCDR3, the LCDR1, the LCDR2 and the LCDR3 of SEQ ID NOs: 98, 99, 100, 106, 107, 108, respectively;
b. the first antigen binding region that binds DLL3 comprises a scFv which is at least 80% (e.g., at least 85%, at least 90%, at least 95%, at least 99% or 100%) identical to the scFv of SEQ ID NO:64, and the second antigen binding region that binds CD3 comprises a Fab comprising a VH which is at least 80% (e.g., at least 85%, at least 90%, at least 95%, at least 99% or 100%) identical to the VH of SEQ ID NO:84 and a VL which is at least 80% (e.g., at least 85%, at least 90%, at least 95%, at least 99% or 100%) identical to the VL of SEQ ID NO:85; and/or
c. the isolated anti-DLL3/anti-CD3 protein comprises a HC1 which is at least 80% (e.g., at least 85%, at least 90%, at least 95%, at least 99% or 100%) identical to the HC1 of SEQ ID NO:229, a HC2 which is at least 80% (e.g., at least 85%, at least 90%, at least 95%, at least 99% or 100%) identical to the HC2 of SEQ ID NO:230, and a LC2 which is at least 80% (e.g., at least 85%, at least 90%, at least 95%, at least 99% or 100%) identical to the of SEQ ID NO: 117.

110. An isolated anti-DLL3/anti-CD3 construct comprising a first antigen binding region that binds DLL3 and a second antigen binding region that binds CD3, wherein
a. the first antigen binding region that binds DLL3 comprises a HCDR1, a HCDR2, a HCDR3, a LCDR1, a LCDR2 and a LCDR3 of SEQ ID NOs:15, 16, 17, 33, 34, and 35, respectively, and the second antigen binding region that binds CD3 comprises the HCDR1, the HCDR2, the HCDR3, the LCDR1, the LCDR2 and the LCDR3 of SEQ ID NOs:98, 99, 100, 106, 107, and 108, respectively; and/or
b. the first antigen binding region that binds DLL3 comprises a scFv of SEQ ID NO:64 and the second antigen binding region that binds CD3 comprises a VH of SEQ ID NO:77 and a VL of SEQ ID NO:80.

111. An immunoconjugate comprising the isolated protein of any one of embodiments 1-59 conjugated to a therapeutic agent or an imaging agent.

112. A pharmaceutical composition comprising the isolated protein of any one of embodiments 1-59 and a pharmaceutically acceptable carrier.

113. A polynucleotide encoding the isolated protein or the multispecific antigen-binding construct of any one of embodiments 1-59.

114. A polynucleotide
a. encoding the isolated protein or the multispecific antigen-binding construct of any one of embodiments 1-59; and/or
b. comprising a polynucleotide sequence of SEQ ID NO:86, 87, 89, 90, 93, 94, 112, 113, 114, 163, 164, 165, 166, 167, 168, 169, 170, 171, 172, 173, 174, 175, 176, 177, 202, 233, 234, 235, 236, 237, 238, 239, 255, 256, 260, 261, 262, 263, 264, 265, 266, 267, 268, 269, or 270.

115. A polynucleotide
a. encoding an isolated protein which is at least 80% (e.g., at least 85%, at least 90%, at least 95%, at least 99% or 100%) identical to the isolated protein or the multispecific antigen-binding construct of any one of embodiments 1-59
b. encoding the polypeptide of SEQ ID NO:1, 2, 3, 4, 5, 6, 7, 8, 9, 10, 11, 12, 13, 14, 63, 64, 65, 66, 67, 68, 69, 71, 77, 78, 80, 84, 85, 105, 109, 110, 111, 112, 113, 114, 115, 116, 117, 118, 119, 229, 190, 191, 192, 193, 194, 195, 196, 230, or 196; and/or
c. comprising a polynucleotide sequence which is at least 80% (e.g., at least 85%, at least 90%, at least 95%, at least 99% or 100%) identical to the polynucleotide sequence of SEQ ID NO:86, 87, 89, 93, 94, 163, 164, 165, 166, 167, 168, 169, 170, 171, 172, 173, 174, 175, 176, 177, 202, 233, 234, 235, 236, 237, 238, 239, 256, 260, 261, 262, 264, 265, 266, 267, 268, 269, or 270.

116. A vector comprising the polynucleotide of any one of embodiments 113-115.

117. A host cell comprising the vector of embodiment 116.

118. A method of producing the isolated protein or the multispecific antigen-binding construct of any one of embodiments 1-59, comprising culturing the host cell of embodiment 117 in conditions that the protein or the multispecific antigen-binding construct is expressed, and recovering the protein or the multispecific antigen-binding construct produced by the host cell.

119. An immunoconjugate comprising the isolated bispecific construct of any one of embodiments 60-110 conjugated to a therapeutic agent or an imaging agent.

120. A pharmaceutical composition comprising the isolated bispecific construct of any one of embodiments 60-110 or the immunoconjugate of embodiment 120 and a pharmaceutically acceptable carrier.

121. A polynucleotide
a. encoding an isolated bispecific construct of any one of embodiments 60-110; or
b. comprising a polynucleotide sequence of SEQ ID NO:86, 87, 89, 93, 94, 163, 164, 165, 166, 167, 168, 169, 170, 171, 172, 173, 174, 175, 176, 177, 202, 233, 234, 235, 236, 237, 238, 239, 256, 260, 261, 262, 264, 265, 266, 267, 268, 269, or 270.

122. A polynucleotide
a. encoding an isolated bispecific construct which is at least 80% (e.g., at least 85%, at least 90%, at least 95%, at least 99% or 100%) identical to the isolated bispecific construct of any one of embodiments 60-110; or
b. comprising a polynucleotide sequence which is at least 80% (e.g., at least 85%, at least 90%, at least 95%, at least 99% or 100%) identical to the polynucleotide sequence of SEQ ID NO:86, 87, 89, 93, 94, 163, 164, 165, 166, 167, 168, 169, 170, 171, 172, 173, 174, 175, 176, 177, 202, 233, 234, 235, 236, 237, 238, 239, 256, 260, 261, 262, 264, 265, 266, 267, 268, 269, or 270.

123. A vector comprising the polynucleotide of embodiment 121 or 122.

124. A host cell comprising the vector of embodiment 123.

125. A method of producing the isolated bispecific construct of any one of embodiments 60-110, comprising culturing the host cell of embodiment 124 in conditions that the bispecific construct is expressed, and recovering the bispecific construct produced by the host cell.

126. A method of treating a DLL3 expressing cancer in a subject, comprising administering a therapeutically effective amount of the isolated protein or the multispecific antigen-binding construct of any one of embodiments 1-59, the isolated bispecific protein of any one of embodiments 60-110, the immunoconjugate of embodiment 111 or 119, or the pharmaceutical composition of embodiment 112 or 120 to the subject for a time sufficient to treat the DLL3 expressing cancer.

127. A method of reducing the amount of DLL3 expressing tumor cells in a subject, comprising administering a therapeutically effective amount of the isolated protein or the multispecific antigen-binding construct of any one of embodiments 1-59, the isolated bispecific construct of any one of embodiments 60-110, the immunoconjugate of embodiment 111 or 119, or the pharmaceutical composition of embodiment 112 or 120 to the subject for a time sufficient to treat the DLL3 expressing cancer.

128. A method of preventing establishment of a DLL3 expressing cancer in a subject, comprising administering a therapeutically effective amount of the isolated protein or the multispecific antigen-binding construct of any one of embodiments 1-59, the isolated bispecific protein of any one of embodiments 60-110, the immunoconjugate of embodiment 111 or 119, or the pharmaceutical composition of embodiment 112 or 120 to the subject to prevent establishment of the DLL3 expressing cancer in the subject.

129. A method of treating a noncancerous condition in a subject at risk of developing a DLL3 expressing cancer, comprising administering a therapeutically effective amount of the isolated protein or the multispecific antigen-binding construct of any one of embodiments 1-59, the isolated bispecific protein of any one of embodiments 60-110, the immunoconjugate of embodiment 111 or 119, or the pharmaceutical composition of embodiment 111 or 119 to the subject to treat the noncancerous condition.

130. The method of any one of embodiments 126-129, wherein the DLL3 expressing cancer is selected from a group consisting of lung cancer, prostate cancer, glioma, glioblastoma, melanoma, neuroendocrine pancreatic cancer, hepatoblastoma, and hepatocellular carcinoma.

131. The method of embodiment 130, wherein the DLL3 expressing cancer is small cell lung cancer.

132. The method of embodiment 130, wherein the DLL3 expressing cancer is neuroendocrine prostate cancer.

133. The method of embodiment 130, wherein the DLL3 expressing cancer is relapsed, refractory, malignant or castration resistant prostate cancer, or any combination thereof.

134. The method of embodiment 129, wherein the noncancerous condition is an enlarged prostate, benign prostate hyperplasia (BPH) or a condition with high prostate specific antigen (PSA) levels in the absence of diagnosed prostate cancer.

135. The method of any one of embodiments 126-134 wherein the isolated protein or the isolated multispecific antigen-binding construct is administered in combination with a second therapeutic agent.

136. The method of embodiment 135, wherein the second therapeutic agent is surgery, chemotherapy, androgen deprivation therapy or radiation, or any combination thereof.

137. A method of detecting the presence of neuroendocrine prostate cancer or small cell lung cancer in a subject, comprising administering the immunoconjugate of embodiment 111 or 119 to a subject suspected to have prostate cancer or small cell lung cancer and visualizing the biological structures to which the immunoconjugate is bound, thereby detecting the presence of prostate cancer or small cell lung cancer.

138. A kit comprising the isolated protein or the multispecific antigen-binding construct of any one of embodiments 1-59, the isolated bispecific construct of any one of embodiments 60-110, the immunoconjugate of 112 or 120, or the pharmaceutical composition of embodiment 113 or 121.

139. An anti-idiotypic antibody binding to the isolated protein or multispecific antigen-binding construct of any one of embodiments 1-59.

140. The isolated bispecific construct of any one of embodiments 60-110, being an anti-DLL3/anti-CD3 construct that has a cell killing effect of at least 75%, at least 80%, at least 85% and at least 90%.

The present disclosure further provides the following particular numbered embodiments:

1. An isolated protein comprising an antigen binding region that binds Delta-like ligand 3 (DLL3), wherein said antigen binding region comprises the HCDR1, the HCDR2, the HCDR3, the LCDR1, the LCDR2 and the LCDR3 of SEQ ID NOs: 15, 16, 17, 33, 34 and 35, respectively.

2. The isolated protein of embodiment 1, wherein the antigen binding region that binds DLL3 comprises a VH which is at least 80% (e.g., at least 85%, at least 90%, at least 95%, at least 99% or 100%) identical to the VH of SEQ ID NO:3.

3. The isolated protein of embodiment 2, wherein the antigen binding region that binds DLL3 comprises a VH of SEQ ID NO:3.

4. The isolated protein of any one of embodiments 1-3, wherein the antigen binding region that binds DLL3 comprises a VL which is at least 80% (e.g., at least 85%, at least 90%, at least 95%, at least 99% or 100%) identical to the VL of SEQ ID NO:4.

5. The isolated protein of embodiment 4, wherein the antigen binding region that binds DLL3 comprises a VL of SEQ ID NO:4.

6. The isolated protein of any one of embodiments 1-5, wherein the antigen binding region that binds DLL3 is a scFv.

7. The isolated protein of any one of embodiments 1-6, wherein the antigen binding region that binds DLL3 comprises a scFv which is at least 80% (e.g., at least 85%, at least 90%, at least 95%, at least 99% or 100%) identical to the scFv of SEQ ID NO:63 or 64.

8. The isolated protein of any one of embodiments 1-7, wherein the protein is conjugated to a half-life extending moiety, wherein the half-life extending moiety is an immunoglobulin (Ig), a fragment of the Ig, an Ig constant region, or a fragment of the Ig constant region.

9. The isolated protein of embodiment 8, wherein the Ig constant region or the fragment of the Ig constant region comprises at least one mutation that results in reduced binding of the protein to a Fcγ receptor (FcγR), optionally wherein the mutations that results in reduced binding of the protein to the FcγR are L234A_L235A_D265 S.

10. The isolated protein of any one of embodiments 1-9, wherein the isolated protein comprises an amino acid sequence which is at least 80% (e.g. at least 85%, at least 90%, at least 95%, at least 99% or 100%) identical to SEQ ID NO:229.

11. The isolated protein of embodiment 10, wherein the isolated protein comprises the amino acid sequence of SEQ ID NO:229.

12. The isolated protein of any one of embodiments 1-9, wherein the isolated protein comprises an amino acid sequence which is at least 80% (e.g. at least 85%, at least 90%, at least 95%, at least 99% or 100%) identical to SEQ ID NO:71.

13. The isolated protein of embodiment 12, wherein the isolated protein comprises the amino acid sequence of SEQ ID NO:71.

14. A multispecific antigen-binding construct comprising the protein of any one of embodiment 1-13.

15. The multispecific antigen-binding construct of embodiment 14, being a bispecific construct.

16. The multispecific antigen-binding construct of embodiment 14 or 15, further comprising a second antigen binding region that binds an antigen on a lymphocyte.

17. The multispecific antigen-binding construct of embodiment 16, wherein the lymphocyte is a T cell.

18. The multispecific antigen-binding construct of embodiment 17, wherein the T cell is a CD8⁺ T cell.

19. The multispecific antigen-binding construct of embodiment 16, wherein the lymphocyte is a natural killer (NK) cell.

20. The multispecific antigen-binding construct of any one of embodiments 16-19, wherein the antigen on the lymphocyte is CD3, CD3 epsilon (CD3ε), CD8, KI2L4, NKG2E, NKG2D, NKG2F, BTNL3, CD186, BTNL8, PD-1, CD195, or NKG2C.

21. The multispecific antigen-binding construct of embodiment 20, wherein the antigen on the lymphocyte is CD3ε.

22. The multispecific antigen-binding construct of embodiment 21, wherein the second antigen binding region that binds CD3ε comprises
a. a heavy chain complementarity determining region (HCDR)1 of SEQ ID NO:95, a HCDR2 of SEQ ID NO:96, a HCDR3 of SEQ ID NO:97, a light chain complementarity determining region (LCDR)1 of SEQ ID NO:101, a LCDR2 of SEQ ID NO:102 and a LCDR3 of SEQ ID NO: 104, and/or
b. the VH of SEQ ID NO:77 and the VL of SEQ ID NO:80.

23. The multispecific antigen-binding construct of embodiment 21, wherein the second antigen binding region that binds CD3ε comprises
b. a heavy chain complementarity determining region (HCDR)1 of SEQ ID NO:98, a HCDR2 of SEQ ID NO:99, a HCDR3 of SEQ ID NO: 100, a light chain complementarity determining region (LCDR)1 of SEQ ID NO:106, a LCDR2 of SEQ ID NO:107 and a LCDR3 of SEQ ID NO: 108; and/or
c. the VH of SEQ ID NO:84 and the VL of SEQ ID NO:85.

24. The multispecific antigen-binding construct of any one of embodiments 14-23, wherein the second antigen binding region that binds CD3ε comprises a HCDR1 of SEQ ID NO:95, a HCDR2 of SEQ ID NO:96, a HCDR3 of SEQ ID NO:97, a LCDR1 of SEQ ID NO:101, a LCDR2 of SEQ ID NO:102 and a LCDR3 of SEQ ID NO:104.

25. The multispecific antigen-binding construct of any one of embodiments 14-23, wherein the second antigen binding region that binds CD3ε comprises a HCDR1 of SEQ ID NO:98, a HCDR2 of SEQ ID NO:99, a HCDR3 of SEQ ID NO:100, a LCDR1 of SEQ ID NO: 106, a LCDR2 of SEQ ID NO: 107 and a LCDR3 of SEQ ID NO: 108.

26. The multispecific antigen-binding construct of embodiment 24, wherein the second antigen binding region that binds CD3ε comprises a VH which is at least 80% (e.g., at least 85%, at least 90%, at least 95%, at least 99% or 100%) identical to the VH of SEQ ID NO:77.

27. The multispecific antigen-binding construct of embodiment 24 or 26, wherein the second antigen binding region that binds CD3ε comprises the VH of SEQ ID NO:77.

28. The multispecific antigen-binding construct of any one of embodiments 24, 26 and 27, wherein the second antigen binding region that binds CD3ε comprises a VL which is at least 80% (e.g. at least 85%, at least 90%, at least 95%, at least 99% or 100%) identical to the VL of SEQ ID NO:80.

29. The multispecific antigen-binding construct of embodiment 28, wherein the second antigen binding region that binds CD3ε comprises the VL of SEQ ID NO:80.

30. The multispecific antigen-binding construct of embodiment 25, wherein the second antigen binding region that binds CD3ε comprises a VH which is at least 80% (e.g., at least 85%, at least 90%, at least 95%, at least 99% or 100%) identical to the VH of SEQ ID NO:84.

31. The multispecific antigen-binding construct of embodiment 30, wherein the second antigen binding region that binds CD3ε comprises the VH of SEQ ID NO:84.

32. The multispecific antigen-binding construct of any one of embodiments 25, 30 and 31, wherein the second antigen binding region that binds CD3ε comprises a VL which is at least 80% (e.g., at least 85%, at least 90%, at least 95%, at least 99% or 100%) identical to the VL of SEQ ID NO:85.

33. The multispecific antigen-binding construct of embodiment 28, wherein the second antigen binding region that binds CD3ε comprises the VL of SEQ ID NO:85.

34. The multispecific antigen-binding construct of any one of embodiments 26-33, wherein the second antigen binding region that binds CD3ε is a Fab.

35. The multispecific antigen-binding construct of any one of embodiments 14-34, wherein the second antigen binding region that binds CD3ε comprises a HC which is at least 80% (e.g. at least 85%, at least 90%, at least 95%, at least 99% or 100%) identical to SEQ ID NO:230.

36. The multispecific antigen-binding construct of embodiment 35, wherein HC comprises the amino acid sequence of SEQ ID NO:230.

37. The multispecific antigen-binding construct of any one of embodiments 14-36, wherein the second antigen binding region that binds CD3ε comprises a LC which is at least 80% (e.g. at least 85%, at least 90%, at least 95%, at least 99% or 100%) identical to SEQ ID NO:117.

38. The multispecific antigen-binding construct of embodiment 37, wherein the LC comprises the amino acid sequence of SEQ ID NO: 117.

39. The multispecific antigen-binding construct of any one of embodiments 14-38, wherein the antigen binding region that binds DLL3 comprises an scFv having an amino acid sequence at least 80% (e.g. at least 85%, at least 90%, at least 95%, at least 99% or 100%) identical to SEQ ID NO:229.

40. The multispecific antigen-binding construct of embodiment 39, wherein the antigen binding region that binds DLL3 comprises an scFv having the amino acid sequence of SEQ ID NO:229.

41. The multispecific antigen-binding construct of any one of embodiments 14-34, wherein the second antigen binding region that binds CD3ε comprises a HC which is at least 80% (e.g. at least 85%, at least 90%, at least 95%, at least 99% or 100%) identical to SEQ ID NO:118.

42. The multispecific antigen-binding construct of embodiment 41, wherein the HC comprises the amino acid sequence of SEQ ID NO: 118.

43. The multispecific antigen-binding construct of any one of embodiments 14-34, 41 and 42, wherein the second antigen binding region that binds CD3ε comprises a LC which is at least 80% (e.g. at least 85%, at least 90%, at least 95%, at least 99% or 100%) identical to SEQ ID NO: 117.

44. The multispecific antigen-binding construct of embodiment 43, wherein the LC comprises the amino acid sequence of SEQ ID NO: 117.

45. The multispecific antigen-binding construct of any one of embodiments 41-44, wherein the antigen binding region that binds DLL3 comprises an scFv having an amino acid sequence that is at least 80% (e.g. at least 85%, at least 90%, at least 95%, at least 99% or 100%) identical to SEQ ID NO:71.

46. The multispecific antigen-binding construct of embodiment 45, wherein the scFv comprises the amino acid sequence of SEQ ID NO:71.

47. The multispecific antigen-binding construct of any one of embodiments 14-46, wherein the antigen binding region that binds an antigen on a lymphocyte is conjugated to a half-life extending moiety, wherein the half-life extending moiety is an immunoglobulin (Ig), a fragment of the Ig, an Ig constant region, or a fragment of the Ig constant region.

48. The multispecific antigen-binding construct of embodiment 47, wherein the Ig constant region or the fragment of the Ig constant region comprises at least one mutation that results in reduced binding of the protein to a Fcγ receptor (FcγR), optionally wherein the mutations that results in reduced binding of the protein to the FcγR are L234A_L235A_D265 S.

49. A bispecific antigen-binding construct comprising:
(1) a first antigen binding region that binds DLL3, wherein the first antigen binding region comprises a first VH having a HCDR1, a HCDR2 and a HCDR3, and a first VL having a LCDR1, a LCDR2 and a LCDR3, and the HCDR1, the HCDR2, the HCDR3, the LCDR1, the LCDR2 and the LCDR3 comprise the amino acid sequences of
   (a) SEQ ID NOs:15, 16, 17, 33, 34, 35, respectively;
   (b) SEQ ID NOs:18, 19, 20, 36, 37, 38, respectively;
   (c) SEQ ID NOs:21, 22, 23, 39, 37, 40, respectively;
   (d) SEQ ID NOs:24, 25, 26, 41, 42, 43, respectively;
   (e) SEQ ID NOs:18, 28, 29, 44, 45, 46, respectively;
   (f) SEQ ID NOs:30, 31, 32, 47, 48, 49, respectively;
   (g) SEQ ID NOs:50, 51, 17, 33, 34, 35, respectively;
   (h) SEQ ID NOs:52, 51, 17, 33, 34, 35, respectively;
   (i) SEQ ID NOs:53, 54, 20, 36, 37, 38, respectively;
   (j) SEQ ID NOs:55, 56, 23, 39, 37, 40, respectively;
   (k) SEQ ID NOs:57, 58, 26, 41, 42, 43, respectively;
   (l) SEQ ID NOs:59, 60, 29, 44, 45, 46, respectively; or
   (m)_{S}EQ ID NOs:61, 62, 32, 47, 48, 49, respectively.
(2) a second antigen binding region that binds CD3ε, wherein the second antigen binding region comprises:
   (a) a second VH having a HCDR1, a HCDR2 and a HCDR3 of the amino acid sequences of SEQ ID NOs: 95, 96 and 97, respectively, and a second VL having a LCDR1, a LCDR2 and a LCDR3 of the amino acid sequences of SEQ ID NOs: 101, 102 and 104, respectively; or
   (b) a second VH having a HCDR1, a HCDR2 and a HCDR3 of the amino acid sequences of SEQ ID NOs: 98, 99 and 100, respectively, and a second VL having a LCDR1, a LCDR2 and a LCDR3 of the amino acid sequences of SEQ ID NOs: 106, 107 and 108, respectively.

50. The bispecific antigen-binding construct of embodiment 49, wherein
a. the first VH and the first VL have amino acid sequences at least 90% (e.g., at least 90%, 91%, 92%, 93%, 94%, 95%, 96%, 97%, 98%, 99% or 100%) identical to:
   i. the VH of SEQ ID NO:1 and the VL of SEQ ID NO:2, respectively;
   ii. the VH of SEQ ID NO:3 and the VL of SEQ ID NO:4, respectively;
   iii. the VH of SEQ ID NO:5 and the VL of SEQ ID NO:6, respectively;
   iv. the VH of SEQ ID NO:7 and the VL of SEQ ID NO:8, respectively;
   v. the VH of SEQ ID NO:9 and the VL of SEQ ID NO:10, respectively;
   vi. the VH of SEQ ID NO: 11 and the VL of SEQ ID NO: 12, respectively; or
   vii. the VH of SEQ ID NO: 13 and the VL of SEQ ID NO: 14, respectively;
b. the second VH and the second VL have amino acid sequences at least 90% (e.g., at least 90%, 91%, 92%, 93%, 94%, 95%, 96%, 97%, 98%, 99% or 100%) identical to:
   i. the VH of SEQ ID NO:77 and the VL of SEQ ID NO:80; or
   ii. the VH of SEQ ID NO:84 and the VL of SEQ ID NO:85.

51. The bispecific antigen-binding construct of embodiment 49 or 50, wherein the first antigen binding region comprises the HCDR1, the HCDR2, the HCDR3, the LCDR1, the LCDR2 and the LCDR3 having the amino acid sequences of SEQ ID NOs:15, 16, 17, 33, 34, 35, respectively.

52. The bispecific antigen-binding construct of embodiment 51, wherein the first VH comprises an amino acid sequence at least 90% (e.g., at least 90%, 91%, 92%, 93%, 94%, 95%, 96%, 97%, 98%, 99% or 100%) identical to SEQ ID NO:3, and the first VL comprises an amino acid sequence at least 90% (e.g., at least 90%, 91%, 92%, 93%, 94%, 95%, 96%, 97%, 98%, 99% or 100%) identical to SEQ ID NO:4.

53. The bispecific antigen-binding construct of any one of embodiments 49-52, wherein the first antigen binding region comprises a first scFv or a first Fab containing the first VH and the first VL, and the second antigen binding region comprises a second Fab or a second scFv containing the second VH and the second VL.

54a. The bispecific antigen-binding construct of embodiment 53, wherein the first antigen binding region comprises the first scFv and the second antigen binding region comprises the second Fab.

54b. The bispecific antigen-binding construct of embodiment 53, wherein the first antigen binding region comprises the first Fab and the second antigen binding region comprises the second scFv.

55. The bispecific antigen-binding construct of any one of embodiments 49-54b being a bispecific antibody comprising a first heavy chain and a second heavy chain, wherein the first heavy chain comprises the first VH, optionally further comprises the first VL, and the second heavy chain comprises the second VH, optionally further comprises the second VL, wherein each of the first and second heavy chains further comprises an immunoglobulin (Ig) constant region which contains one or more heterodimeric mutations.

56. The bispecific antigen-binding construct of embodiment 55, comprising:
(1) a first heavy chain having an amino acid sequence that is at least 80%, such as at least 85%, 90%, 95% or 100%, identical to an amino acid sequence selected from the group consisting of SEQ ID NOs: 109, 109, 111, 111, 71 and 229;
(2) a light chain having an amino acid sequence that is at least 80%, such as at least 85%, 90%, 95% or 100%, identical to an amino acid sequence selected from the group consisting of SEQ ID NOs: 110, 110, 117, 115, 117 and 117, respectively; and
(3) a second heavy chain having an amino acid sequence that is at least 80%, such as at least 85%, 90%, 95% or 100%, identical to an amino acid sequence selected from the group consisting of SEQ ID NOs: 112, 113, 116, 114, 118 and 230, respectively.

57. The bispecific antigen-binding construct of embodiment 55, comprising a first heavy chain, a light chain and a second heavy chain having amino acid sequences that are at least 80%, such as at least 85%, 90%, 95% or 100%, identical to the amino acid sequences of SEQ ID NOs: 111, 117 and 116, respectively.

58. The bispecific antigen-binding construct of embodiment 55, comprising a first heavy chain, a light chain and a second heavy chain having amino acid sequences that are at least 80%, such as at least 85%, 90%, 95% or 100%, identical to the amino acid sequences of SEQ ID NOs: 111, 115 and 114, respectively.

59. The bispecific antigen-binding construct of embodiment 55, comprising a first heavy chain, a light chain and a second heavy chain having amino acid sequences that are at least 80%, such as at least 85%, 90%, 95% or 100%, identical to the amino acid sequences of SEQ ID NOs: 71, 117 and 118, respectively.

60. The bispecific antigen-binding construct of embodiment 55, comprising a first heavy chain, a light chain and a second heavy chain having amino acid sequences that are at least 80%, such as at least 85%, 90%, 95% or 100%, identical to the amino acid sequences of SEQ ID NOs: 229, 117 and 230, respectively.

60a. The bispecific antigen-binding construct of embodiment 55, comprising a first heavy chain, a light chain and a second heavy chain having amino acid sequences that are at least 80%, such as at least 85%, 90%, 95% or 100%, identical to the amino acid sequences of SEQ ID NOs: 109, 110 and 113, respectively.

60b. The bispecific antigen-binding construct of embodiment 55, comprising a first heavy chain, a light chain and a second heavy chain having amino acid sequences that are at least 80%, such as at least 85%, 90%, 95% or 100%, identical to the amino acid sequences of SEQ ID NOs: 109, 110 and 112, respectively.

61. An isolated nucleic acid encoding the protein of any one of embodiments 1-13, or the multispecific antigen-binding construct of any one of embodiments 8-48, or the bispecific antigen-binding construct of any one of embodiments 49-60b.

62. A vector comprising the nucleic acid of embodiment 61.

63. A host cell comprising the nucleic acid of embodiment 61 or the vector of embodiment 62.

64. A method of producing the protein of any one of embodiments 1-13, or the multispecific antigen-binding construct of any one of embodiments 8-48, or the bispecific antigen-binding construct of any one of embodiments 49-60b, comprising culturing the host cell of embodiment 26 under conditions to produce the protein, the multispecific antigen-binding construct, the fusion or conjugate or the bispecific antigen-binding construct, and recovering the same from the cell or cell culture.

65. A pharmaceutical composition comprising the protein of any one of embodiments 1-13, or the multispecific antigen-binding construct of any one of embodiments 8-48, or the bispecific antigen-binding construct of any one of embodiments 49-60b, the nucleic acid of embodiment 61, the vector of embodiment 62, or the host cell of embodiment 63, and a pharmaceutically acceptable carrier.

66. A method of treating a DLL3 expressing cancer in a subject in need thereof, comprising administering a therapeutically effective amount of the pharmaceutical composition of embodiment 65 to the subject for a time sufficient to treat the DLL3 expressing cancer, preferably.

67. The method of embodiment 66, wherein the cancer is selected from a group consisting of lung cancer, prostate cancer, glioma, glioblastoma, melanoma, neuroendocrine pancreatic cancer, hepatoblastoma, and hepatocellular carcinoma.

68. A method of reducing the amount of DLL3 expressing tumor cells in a subject, comprising administering a therapeutically effective amount of the pharmaceutical composition of embodiment 65 to the subject for a time sufficient to treat the DLL3 expressing cancer.

69. The method of embodiment 68, wherein the cancer is selected from a group consisting of lung cancer (such as small cell lung cancer), prostate cancer (such as neuroendocrine prostate cancer, or relapsed, refractory, malignant or castration resistant prostate cancer), glioma, glioblastoma, melanoma, neuroendocrine pancreatic cancer, hepatoblastoma, and hepatocellular carcinoma, or any combination thereof.

70. A method of treating a noncancerous condition in a subject at risk of developing a DLL3 expressing cancer, comprising administering a therapeutically effective amount of the pharmaceutical composition of embodiment 65 to the subject to treat the noncancerous condition.

71. The method of embodiment 70, wherein the noncancerous condition is an enlarged prostate, benign prostate hyperplasia (BPH) or a condition with high prostate specific antigen (PSA) levels in the absence of diagnosed prostate cancer.

72. A method of detecting the presence of neuroendocrine prostate cancer or small cell lung cancer in a subject, comprising administering the immunoconjugate comprising a therapeutic agent or an imaging agent conjugated to the protein of any one of embodiments 1-13, or the multispecific antigen-binding construct of any one of embodiments 8-48, or the bispecific antigen-binding construct of any one of embodiments 49-60b to a subject suspected to have prostate cancer or small cell lung cancer, and visualizing the biological structures to which the immunoconjugate is bound, thereby detecting the presence of prostate cancer or small cell lung cancer.

73. A kit comprising the protein of any one of embodiments 1-13, or the multispecific antigen-binding construct of any one of embodiments 8-48, or the bispecific antigen-binding construct of any one of embodiments 49-60b, or an immunoconjugate comprising a therapeutic agent or an imaging agent conjugated to the protein, the multispecific antigen-binding construct or the bispecific antigen-binding construct, the nucleic acid of embodiment 61, the vector of embodiment 62, or the host cell of embodiment 63.

74. The protein of any one of embodiments 1-13, or the multispecific antigen-binding construct of any one of embodiments 8-48, or the bispecific antigen-binding construct of any one of embodiments 49-60b, or an immunoconjugate comprising a therapeutic agent conjugated to the protein, the multispecific antigen-binding construct or the bispecific antigen-binding construct, the nucleic acid of embodiment 61, the vector of embodiment 62, or the host cell of embodiment 63 for use in treating a cancer, preferably the cancer is selected from a group consisting of lung cancer (such as small cell lung cancer), prostate cancer (such as neuroendocrine prostate cancer, or relapsed, refractory, malignant or castration resistant prostate cancer), glioma, glioblastoma, melanoma, neuroendocrine pancreatic cancer, hepatoblastoma, and hepatocellular carcinoma, or any combination thereof.

75. A method of treating a cancer, comprising administering to a subject in need thereof a therapeutically effective amount of the protein of any one of embodiments 1-13, or the multispecific antigen-binding construct of any one of embodiments 8-48, or the bispecific antigen-binding construct of any one of embodiments 49-60b, or an immunoconjugate comprising a therapeutic agent conjugated to the protein, the multispecific antigen-binding construct or the bispecific antigen-binding construct, the nucleic acid of embodiment 61, the vector of embodiment 62, or the host cell of embodiment 63, preferably the cancer is selected from a group consisting of lung cancer (such as small cell lung cancer), prostate cancer (such as neuroendocrine prostate cancer, or relapsed, refractory, malignant or castration resistant prostate cancer), glioma, glioblastoma, melanoma, neuroendocrine pancreatic cancer, hepatoblastoma, and hepatocellular carcinoma, or any combination thereof.

76. Use of the protein of any one of embodiments 1-13, or the multispecific antigen-binding construct of any one of embodiments 8-48, or the bispecific antigen-binding construct of any one of embodiments 49-60b, or an immunoconjugate comprising a therapeutic agent conjugated to the protein, the multispecific antigen-binding construct or the bispecific antigen-binding construct, the nucleic acid of embodiment 61, the vector of embodiment 62, or the host cell of embodiment 63 in the manufacture of a medicament for treating a cancer, preferably the cancer is selected from a group consisting of lung cancer (such as small cell lung cancer), prostate cancer (such as neuroendocrine prostate cancer, or relapsed, refractory, malignant or castration resistant prostate cancer), glioma, glioblastoma, melanoma, neuroendocrine pancreatic cancer, hepatoblastoma, and hepatocellular carcinoma, or any combination thereof.

The following examples of the invention are to further illustrate the nature of the invention. It should be understood that the following examples do not limit the invention and the scope of the invention is to be determined by the appended claims.

### EXAMPLES

### Example 1. Antigen Generation

The DLL3 construct used for immunization comprises the extracellular domain of human DLL3 (ECD) linked to a C-terminal 6×His-Tag and a linker (Construct DL3W35; SEQ ID NO: 180). Expression constructs encoding subdomains of the human DLL3 ECD were designed as a fusion protein using a C34 S variant of human serum albumin (HSA, (DL3W36-DL3W44, SEQ ID NOs:181-189). In particular, the constructs include a construct of: human DLL3 EGF6 domain+C-term ECD sequence, HSA Fusion and C-term 6 His tag (DL3W36, SEQ ID NO:181), human DLL3 EGF6 domain, HSA Fusion and C-term 6His tag (DL3W37, SEQ ID NO:182), human DLL3 EGF5 domain, HSA Fusion and C-term 6His tag (DL3W38, SEQ ID NO:183), human DLL3 EGF4 domain, HSA Fusion and C-term 6His tag (DL3W39, SEQ ID NO:184), human DLL3 EGF3 domain; HSA Fusion and C-term 6His tag (DL3W40, SEQ ID NO:185), human DLL EGF2 domain, HSA Fusion and C-term 6His tag (DL3W41, SEQ ID NO:186), human DLL3 EGF1+2 domains, HSA Fusion and C-term 6His tag (DL3W42, SEQ ID NO:187), human DLL3 EGF-1 domain, HSA Fusion and C-term 6His tag (DL3W43, SEQ ID NO:188), human DLL3 N-Term+DSL domains HSA Fusion and C-term 6His tag (DL3W44, SEQ ID NO: 189) The HSA was fused to the C-terminus of the human DLL3 ECD subdomain. A 6×His Tag was also added to the C-terminus. The constructs were based on Uniprot Accession #Q9NYJ7 and its domain annotation therein. Nine constructs were made encompassing either the N-terminal and DSL domains, the EGF1 domain, the EGF1 and EGF2 domains, the EGF2 domain, the EGF3 domain, the EGF4 domain, the EGF5 domain, the EGF6 domain and the EGF6 and C-terminal domains. The constructs encoding subdomains were used for domain mapping.

The constructs were transiently transfected into HEK293 derived cells, Expi293 (Gibco/Thermo Fisher Scientific) using Expifectamine according to manufacturer protocol. Cells were incubated 5 days at 37° C. with 8% CO₂ on an orbital shaker before harvesting. The expressed cells were removed by centrifugation and the DLL3 proteins with His-tags were purified from the media using immobilized metal affinity chromatography using Ni Sepharose 6 Fast Flow resin (GE Healthcare) followed by Superdex 200 preparative size exclusion chromatography (SEC) (GE Healthcare) in Dubelcco's Phosphate Saline buffer pH 7.2 (1× DPBS).

### Example 2. Generation of Anti-DLL3 Antibodies

### Antibody Generation Using Transgenic Mice (Ablexis®)

Anti-DLL3 antibodies were generated in Ablexis mice. Ablexis® mice generate human/mouse chimeric antibodies having human variable domains linked to human CH1 and CL domains, a chimeric human/mouse hinge region, and mouse Fc regions. Antibodies produced by the Ablexis Kappa Mouse lack sequence derived from mouse V_{H}, D_{H} and J_{H} exons and mouse V_{κ}, J_{κ} and C_{κ} exons. The endogenous mouse Igλ is active in the Kappa Mouse. The human Igκ chains comprise approximately 90-95% of the naïve repertoire and mouse Igλ chains comprise approximately 5-10% of the naïve repertoire in this strain. Antibodies produced by the Ablexis Lambda Mouse lack sequence derived from mouse VH, DH and JH exons and mouse Vλ, Jλ and Cλ exons. The endogenous mouse Ig_{κ} is active in the Lambda Mouse. The human Igλ chains comprise approximately 40% of the naïve repertoire and mouse Igλ chains comprise approximately 60% of the naïve repertoire. The preparation and use of Ablexis©, and the genomic modifications carried by such mice, is described in WO11/123708.

Ablexis mice were immunized with recombinant human DLL3 ECD protein (DL3W35, SEQ ID NO:180). Lymphocytes were extracted from secondary lymphoid organs and either fused with FO mouse myeloma cell line for hybridoma generation or subjected to single cell sorting via fluorescence-activated cell sorting (FACS). Hybridoma supernatants were screened by Meso Scale Discovery (MSD) electrochemiluminescence for binding to HEK cells over-expressing human DLL3 ECD. Identified samples were further assayed via Fluorescence-activated Cell sorting (FACS) for binding to HEK cells over-expressing human DLL3 ECD (positive signal) and to parental DLL3 negative HEK cells (negative signal). In addition, single cell sorting supernatants were screened by MSD electrochemiluminescence for binding to recombinant human DLL3 protein. Approximately >300 samples were identified to be DLL3 binders. The binding of the 300 anti-hDLL3 supernatant samples were further evaluated for binding to human DLL3 protein by single cycle kinetics method by Biacore 8K SPR.

Six DLL3 positive binders were selected and moved forward for V-region cloning.

### V Region Cloning

V-regions of heavy and light chains from hybridoma supernatants containing positive binders for human DLL3 were cloned and sequenced. mRNA was isolated from hybridoma samples. Both RNA purified by Qiagen kit (RNeasy Plus Mini Kit) and B cells lysate were used for cDNA synthesis using the Smarter cDNA synthesis kit (Clontech, Mount View, CA). To facilitate cDNA synthesis, oligodT was used to prime reverse transcription of all messenger RNAs followed by “5′ capping” with a Smarter IIA oligonucleotide. Subsequent amplification of the VH and VL fragments was performed using a 2-step PCR amplification using 5′ primers targeting the Smarter IIA cap and 3′ primers targeting consensus regions in CH1. Briefly, each 50 μl PCR reaction consisted of 20 μM of forward and reverse primer mixes, 25 μl of PrimeStar Max DNA polymerase premix (Clontech), 2 d of unpurified cDNA, and 21 μl of double-distilled H₂O. The cycling program started at 94° C. for 3 min, followed by 35 cycles (94° C. for 30 secs, 55° C. for 1 min, 68° C. for 1 min), and ended at 72° C. for 7 min. The second round PCR was performed with VL and VH second round primers containing 15 bp complementary extensions that “overlap” respective regions in their respective Lonza mother vector (VH and VL). The second round PCR was performed with the following program: 94° C. for 3 min; 35 cycles (94° C. for 30 Sec, 55° C. for 1 min, 68° C. for 1 min), and ends at 72° C. for 7 min. In-Fusion® HD Cloning Kit (Clonetech) was used for directional cloning of VL gene into Lonza huIgK or Lambda vector and VH gene into Lonza huIgG1 vector. To facilitate In-Fusion© HD Cloning, PCR products were treated with Cloning Enhancer before In-Fusion HD Cloning. Cloning and transformation were performed according to manufacturer's protocol (Clonetech). Mini-prep DNAs were subjected to Sanger sequencing to confirm that complete V-gene fragments were obtained.

Fab, mAb, scFv and scFv-Fc Formatting

The amino acid sequences of the recovered v-regions were codon optimized and cloned into an expression vector carrying an IgG1 constant region.

Antibodies were expressed either in a Fab format, a monoclonal Ab format, a scFv format in the VH-linker-VL orientation or a scFv format in the VL-linker-VH orientation. The linker sequence (GGSEGKSSGSGSESKSTGGS) of SEQ ID NO: 120 was used to conjugate the VH/VL regions.

### ExpiCHO-S™ Transfection and Purification of Anti-DLL3 Antibodies

### Protein Expression & Cell Culture

Antibodies identified from the immunization campaign were cloned and expressed as IgG1-AAS (L234A/L235A/D265 S) at 2 ml scale and purified. Antibodies were expressed in ExpiCHO-S™ cells (ThermoFisher Scientific) by transient transfection with purified plasmid DNA encoding the proteins following the manufacturer's recommendations. Briefly, ExpiCHO-S™ cells were maintained in suspension in ExpiCHO™ expression medium (ThermoFisher Scientific) in an orbital shaking incubator set at 37° C., 8% C02 and 125 RPM. The cells were passaged and diluted prior to transfection to 6.0×10⁶ cells per ml, maintaining cell viability at 99.0% or better. Transient transfections were done using the ExpiFectamine™ CHO transfection kit (ThermoFisher Scientific, Cat #A29131). For each ml of diluted cells to be transfected, 0.5 microgram of scFv-Fc fusion encoding DNA and 0.5 microgram of pAdVAntage DNA (Promega, Cat #E1711) was used and diluted into OptiPRO™ SFM complexation medium. ExpiFectamine™ CHO reagent was used at a 1:4 ratio (v/v, DNA:reagent) and diluted into OptiPRO™. The diluted DNA and transfection reagent were combined for one minute, allowing DNA/lipid complex formation, and then added to the cells. After overnight incubation, ExpiCHO™ feed and ExpiFectamine™ CHO enhancers were added to the cells as per the manufacturer's Standard protocol. Cells were incubated with orbital shaking (125 rpm) at 37° C. for seven days prior to harvesting the culture broth. The culture supernatant from the transiently transfected ExpiCHO-S™ cells was clarified by centrifugation (30 min, 3000 rcf) followed by filtration (0.2 μm PES membrane, Corning; Corning, NY).

### Protein Purification

The filtered cell culture supernatant was loaded onto a pre-equilibrated (1×DPBS, pH 7.2) MabSelect Sure Protein A column (GE Healthcare) using an AKTAXpress chromatography system. After loading, the column was washed with 10 column volumes of 1×DPBS, pH7.2. The protein was eluted with 10 column volumes of 0.1 M sodium (Na)-Acetate, pH 3.5. Protein fractions were neutralized immediately by the addition of 2.5 M Tris HC1, pH 7.5 to 20% (v/v) of the elution fraction volume. Peak fractions were pooled and filtered (0.2 μm). The quality of the purified protein was assessed by analytical size exclusion HPLC (Agilent HPLC system). The protein was further purified by preparative size exclusion chromatography using Superdex200 resin (GE Healthcare) and 1×DPBS pH7.2 as mobile phase. The peak fractions containing monomeric protein only were pooled and filtered (0.2 μm).

### Example 3. Biophysical Characterization of Anti-DLL3 Antibodies

The variable regions of the DLL3 antibodies were formatted as scFv-Fc in the VH-linker-VL orientation using the linker of SEQ ID NO: 120 and evaluated for binding to recombinant DLL3 and for thermostability. The wild-type IgG1 Fc domain with the SEQ ID NO: 120 was fused to the anti-DLL3 scFv to create the scFv-Fc molecules. These constructs were used to evaluate thermal stability of the antibodies

### Binding Affinity of Anti-DLL3 Antibodies.

The binding affinity of anti-DLL3 antibodies to the recombinant human DLL3 was determined by surface plasmon resonance (SPR) using the ProteOn instrument. SPR is a label-free technique to study the strength of an interaction between two binding partners by measuring the change in mass upon complex formation and dissociation. The antibodies were captured on a sensor chip coated with an anti-Fc antibody and titrated with 2-fold serial dilutions of DLL3 antigen (DL3W35, SEQ ID NO:180) spanning concentrations of 100 nM to 6.25 nM, or 6.25 nM to 0.39 nM.

The antibodies were also tested for binding to DLL1 (DL3W33, Recombinant Human DLL1 ECD (Ser22-Gly540), -DI-C-6xHis, TPP000049465, (R&D Systems Cat #1818-DL), SEQ ID NO:178) and DLL4 (DL3W34, human DLL4 ECD (Ser27-Pro524), C-10xHis, TPP0000494661, SEQ ID NO:179) described below at a concentration of 100 nM and 1000 nM.

The association and dissociation were monitored for 5 and 30 minutes, respectively, using a flow rate of 50 μL/min. Kinetic information (on-rate and off-rate constants) were extracted by fitting sensorgrams to the 1:1 Langmuir binding model. Binding affinity (K_{D}) are reported as the ratio of rate constants (koff/kon). K_{D} values of selected anti-DLL3 scFvs are listed in Table 4. As shown in Table 4, the antibodies bind human DLL3 with high affinity ranging from ˜70 μm to ˜2.4 nM, while no binding to homologues proteins DLL1 and DLL4 is observed.

TABLE 4
Affinities (K_{D}) of anti-DLL3 antibodies binding to human
DLL3 (DL3W35), DLL1 (DL3W33) and DLL4 (DL3W34) as obtained
by SPR. N.B. indicates no binding was observed at concentrations
of DLL1 or DLL4 as high as 1000 nM.

| | | Binding to | | |
|---|---|---|---|---|
| | | hu DLL3 | Binding to | Binding to |
| Name | Description | K_{D} (M) | huDLL1 | huDLL4 |
| DL3B569 | DL3B279 scFv-Fc | 1.47E−10 | N.B. | N.B. |
| DL3B570 | DL3B332 scFv-Fc | 6.68E−11 | N.B. | N.B. |
| DL3B571 | DL3B358 scFv-Fc | 7.34E−11 | N.B. | N.B. |
| DL3B572 | DL3B409 scFv-Fc | 2.32E−09 | N.B. | N.B. |
| DL3B574 | DL3B450 scFv-Fc | 2.37E−09 | N.B. | N.B. |
| DL3B575 | DL3B461 scFv-Fc | 3.10E−10 | N.B. | N.B. |

### Thermal Stability of Anti-DLL3 Antibodies

The thermal stability of anti-DLL3 scFv-Fc fusion antibodies was determined by Differential Scanning Fluorimetry (NanoDSF) using an automated Prometheus instrument. Proteins, such as antibodies, encompassing various domains typically exhibit different transitions corresponding to these domains. The first transition or melting temperature (Tₘ₁) is used to indicate the stability of the tested protein under physiological conditions and upon storage. The fluorescence change in proteins also reflects the aggregation onset temperature (_{Tagg}) with increasing temperatures. NanoDSF was used to measure Tm of molecules at a concentration of 0.5 mg/ml in phosphate buffered saline, pH 7.4. Measurements were made by loading sample into 24 well capillary from a 384 well sample plate. Duplicate runs were performed for each sample. The thermal scans span from 20° C. to 95° C. at a rate of 1.0° C./minute. Intrinsic tryptophan and tyrosine fluorescence were monitored at the emission wavelength of 330 nm and 350 nm, and the F350/F330 nm ratio were plotted against temperature to generate unfolding curves. Measured Tₘ and T_{agg} values are listed in Table 5.

As shown in Table 5, all anti-DLL3 molecules have a first transition (Tₘ₁) higher than 56.5° C. All tested scFv-Fc fusion antibodies, except the DL3B570, have low aggregation tendency with T_{agg} values higher than 70° C., and 5° C. or higher than their Tₘ₁ values.

TABLE 5
Thermal stability of anti-DLL3 scFv-Fc fusion antibodies
as obtained using a NanoDSF instrument.

| Name | Description | T_{agg} | Tₘ₁ |
|---|---|---|---|
| DL3B569 | DL3B279 scFv-Fc | 70.5 | 59.1 |
| DL3B570 | DL3B332 scFv-Fc | 62.1 | 61.9 |
| DL3B571 | DL3B358 scFv-Fc | 75.2 | 65.4 |
| DL3B572 | DL3B409 scFv-Fc | 75.4 | 68.8 |
| DL3B574 | DL3B450 scFv-Fc | 72.5 | 68.8 |
| DL3B575 | DL3B461 scFv-Fc | 78.6 | 56.5 |

### Example 4. Domain Mapping and Paratope Mapping of Anti-DLL3 Antibodies Domain Mapping of Anti-DLL3 Antibodies

Selected anti-DLL3 antibodies were evaluated for binding to the each recombinant DLL3 domain; the N-terminal and DSL fusion domain (DL3W44, SEQ ID NO: 189), the EGF-1+2 fusion domain (DL3W42, SEQ ID NO:187), the EGF-2 (DL3W41, SEQ ID NO:186), the EGF-3 (DL3W40, SEQ ID NO:185), the EGF-4 (DL3W39, SEQ ID NO:184), the EGF-5 (DL3W38, SEQ ID NO:183), the EGF-6 (DL3W37, SEQ ID NO:182) and EGF-6 and C-terminal fusion domain (DL3W36, SEQ ID NO: 181). Expression and purification of these constructs is described in Example 1 MesoScale Discovery high bind plates were coated overnight at 4° C. with 20 nM antigen.

The plates were washed with PBS with 0.1% Tween and then blocked with Starting block solution for 30 minutes. DLL3 antibodies were added and incubated for 60 minutes at ambient temperature and then excess antibodies were removed by washing 3 times with PBS (Gibco, #14190-136). Antigen bound antibody was detected with sulfo-tagged anti-human antibody (Meso Scale Discovery, R32AJ) for 60 minutes at ambient temperature followed by another PBS wash. Signal acquisition was done in the presence of 1× MSD read buffer T (MSD, Cat #R92TC-1) on the MSD Sector 600 imager with appropriate plate settings. Data was analyzed for the highest binding signal per domain indicating the preferential domain binding. Binding domains of each antibody tested is listed in Table 6.

TABLE 6
Binding domain of anti-DLL3 antibodies on hu DLL3.

| Name | Description | DLL3 binding domain |
|---|---|---|
| DL3B569 | DL3B279 scFv-Fc | EGF6 |
| DL3B570 | DL3B332 scFv-Fc | EGF6 |
| DL3B571 | DL3B358 scFv-Fc | EGF6 |
| DL3B572 | DL3B409 scFv-Fc | EGF6 + Cterm |
| DL3B574 | DL3B450 scFv-Fc | EGF6 + Cterm |
| DL3B575 | DL3B461 scFv-Fc | EGF6 + Cterm |

DL3B569, DL3B570 and DL3B571 were found to bind the EGF6 domain while DL3B672, DL3B574 and DL3B575 were found to bind the EGF6 and C-terminal domain.

### Paratope Mapping of Anti-DLL3 Antibodies

The paratope on selected anti-DLL3 antibodies was determined by hydrogen-deuterium exchange mass spectrometry (HDX-MS). Briefly, the antibody samples were compared in the unbound state (antibody alone) and the bound state (antibody incubated with huDLL3 (DL3W35)) at a 9:10 molar ratio for slight excess of the binding protein. These samples were stored at 0° C. The samples were labeled with D₂O for 30, 100, 1000, and 10000 seconds. The 100 second labeling was run in duplicate. For each label time and sample, 5 uL of sample was mixed with 50 uL of D20 Buffer (10 mM sodium phosphate pH 7.4) at 23° C. 50 uL of this mixture was transferred to 60 uL of prechilled Quench Buffer (4 M Urea 0.4M TCEP HC1) at 0° C. The mixture was held at 0° C. for 2 minutes. 100 uL of mixture was injected into the LEAP chilled valve box at 0° C. The Flex LC isocratic flow was used to push the sample through the inline pepsin protease 13 combo column (outside of the chilled box at room temperature) and desalt the samples through the inline trap column for 3 minutes. Then, the sample was eluted off the trap column and separated on the analytical column using the Horizon pump gradient. The samples were also run using H₂O in place of D₂O and CID, HCD, and EThcD MS/MS fragmentation to identify peptides and retention times. The paratopes were identified based on significant differences in deuterium uptake from the HDExaminer residue plots.

Incubation of anti-DLL3 antibodies, DL3B569, DL3B570, DL3B571, DL3B574, and DL3B575 with soluble DLL3 resulted in different patterns of hydrogen exchange and overall protection on the antibodies. The protected segments on the antibodies in the presence of DLL3 are shown in Table 7.

TABLE 7
Binding paratope of anti-DLL3 antibodies.

| | | Binding paratope | Binding Paratopeope |
|---|---|---|---|
| Name | Description | Residue numbering | Amino acid sequence |
| DL3B569 | DL3B279 | 49-52, 158-169, | 49-52: YAAS (SEQ ID NO: 63) |
| | scFv-Fc | 205-214 | 178-189: INPSGGSTSYAQ (SEQ ID |
| | | | NO: 63) |
| | | | 225-234: RQGPFIGDAF (SEQ ID |
| | | | NO: 63) |
| DL3B570 | DL3B332 | 88-105, 203-211 | 88-105: YCQQYGTSPITFGQGTRL |
| | scFv-Fc | | (SEQ ID NO: 65) |
| | | | 223-231: CARIGPAGF (SEQ ID |
| | | | NO: 65) |
| DL3B571 | DL3B358 | 137-142, 156-173, | 157-162: ISYYIH (SEQ ID NO: 66) |
| | scFv-Fc | 203-217 | 176-193: GIIDPSGGSKSYAQKFQG |
| | | | (SEQ ID NO: 66) |
| | | | 223-237: CARQGMIVGTTGDAF |
| | | | (SEQ ID NO: 66) |
| DL3B574 | DL3B450 | 216-225 | 236-245: YDWSYYYYGM (SEQ ID |
| | scFv-Fc | | NO: 68) |
| DL3B575 | DL3B461 | 206-216 | 226-236: YYCARDPFSDL (SEQ ID |
| | scFv-Fc | | NO: 69) |

### Example 5. Structural Characterization of Anti-DLL3 Antibodies

Sequences of the DLL3 antibody variable domains and scFv antibody fragments showing highest performance in in vitro assay are provided herein. Variable domains were expressed in a Fab format, a scFv format in the VH-linker-VL orientation or a scFv format in VL-linker-VH orientation using linker of SEQ ID N: 120 as described in Example 2.

Post-translational modifications (PTMs) of antibody have the potential to affect affinity, stability, potency and homogeneity of antibodies. In addition, antibody sequences obtained from transgenic animals may contain somatic hypermutations in the framework and CDR regions. Somatic hypermutations may result in unusual or low frequency residues in human framework regions and impact the stability and immunogenicity of biotherapeutics. The parent anti-DLL3 variable region featured in the DL3B279 mAb, contained sequence liabilities resulting from germline mutations. Specifically, the variable heavy domain contained an Asparagine at position 27, where a tyrosine residue is normally found in the IGHV1-46*03 germline. Since this residue was near the CDR, it was mutated to a glutamine residue instead (N27Q) to preserve the presence of a polar-uncharged amino acid at this position. Additionally, Met105 in the joining region was mutated to Thr (M105T) to avoid oxidation. The light chain v-region from DL3B279 also featured a germline mutation at A99 which was mutated back to Gly (A99G). Together, mutation of N27Q and M105T in the variable heavy domain and the A99G mutation in the variable light domain gave rise to an optimized variable region named DL3B279 variant. Optimized DL3B279 variant was formatted as a single-chain fragment variable (scFv) in the in VL-linker-VH orientation described above.

### Variable Domains VH, VL and CDRs

Table 8 shows the VH and VL amino acid sequences of the selected anti-DLL3 antibodies. Table 9 shows the Kabat HCDR1, HCDR2 and HCDR3 of selected anti-DLL3 antibodies. Table 10 shows the Kabat LCDR1, LCDR2 and LCDR3 of the selected anti-DLL3 antibodies. Table 11 shows the AbM HCDR1, HCDR2 and HCDR3 of selected anti-DLL3 antibodies. Table 12 shows the AbM LCDR1, LCDR2 and LCDR3 of selected anti-DLL3 antibodies. Table 13 shows the Chotia HCDR1, HCDR2 and HCDR3 of selected anti-DLL3 antibodies. Table 14 shows the Chotia LCDR1, LCDR2 and LCDR3 of selected anti-DLL3 antibodies. Table 15 shows the IMTG HCDR1, HCDR2 and HCDR3 of selected anti-DLL3 antibodies. Table 16 shows the IMTG LCDR1, LCDR2 and LCDR3 of selected anti-DLL3 antibodies. Table 17 summarizes the variable domain sequence and SEQ ID NO of selected DLL3 antibodies. Table 18 shows the protein and DNA SEQ ID NOs for the VH and VL regions.

TABLE 8
VH and VL amino acid sequences of
selected anti-DLL3 antibodies.

| mAb name | VH SEQ ID NO: | VL SEQ ID NO: |
|---|---|---|
| DL3B279 | 1 | 2 |
| DL3B279 variant | 3 | 4 |
| (DL3B279-VL-A99G-VH- | | |
| N27Q_M105T-) | | |
| DL3B332 | 5 | 6 |
| DL3B358 | 7 | 8 |
| DL3B409 | 9 | 10 |
| DL3B450 | 11 | 12 |
| DL3B461 | 13 | 14 |

TABLE 9
HCDR1, HCDR2 and HCDR3 amino acid sequences of
selected anti-DLL3 antibodies using Kabat
delineation.

| | Kabat HCDR1 | | Kabat HCDR2 | | Kabat HCDR3 | |
|---|---|---|---|---|---|---|
| | | SEQ | | SEQ | | SEQ |
| mAb | | ID | | ID | | ID |
| name | Sequence | NO: | Sequence | NO: | Sequence | NO: |
| DL3B279 | NYYIH | 15 | IINPSGGSTSYA | 16 | QGPFIGDAFD | 17 |
| | | | QKLQG | | I | |
| DL3B279 | NYYIH | 15 | IINPSGGSTSYA | 16 | QGPFIGDAFD | 17 |
| variant | | | QKLQG | | I | |
| DL3B332 | SYYWS | 18 | YIYYSGTTNYKS | 19 | IGPAGFYFDY | 20 |
| | | | SLKS | | | |
| DL3B358 | SYYIH | 21 | IIDPSGGSKSYA | 22 | QGMIVGTTGD | 23 |
| | | | QKFQG | | AFDI | |
| DL3B409 | TYYIH | 24 | IIDPSGGRTSYA | 25 | GGDGTWYYGM | 26 |
| | | | QKFLG | | DV | |
| DL3B450 | SYYWS | 18 | RIYTSGSTNYNP | 28 | DQAYSGYDWS | 29 |
| | | | SLKS | | YYYYGMDV | |
| DL3B461 | SYVIS | 30 | GIIPIFGTANYA | 31 | DPFSDL | 32 |
| | | | QKFQD | | | |

TABLE 10
LCDR1, LCDR2 and LCDR3 amino acid sequences of
selected anti-DLL3 antibodies using Kabat
delineation.

| | Kabat LCDR1 | | Kabat LCDR2 | | Kabat LCDR3 | |
|---|---|---|---|---|---|---|
| | | SEQ | | SEQ | | SEQ |
| mAb | | ID | | ID | | ID |
| name | Sequence | NO | Sequence | NO | Sequence | NO |
| DL3B279 | RASQGISNYLA | 33 | AASSLQS | 34 | QQYNSYPYT | 35 |
| DL3B279 | RASQGISNYLA | 33 | AASSLQS | 34 | QQYNSYPYT | 35 |
| variant | | | | | | |
| DL3B332 | RASQSVSRSYLA | 36 | GASSRAT | 37 | QQYGTSPIT | 38 |
| DL3B358 | RASQSASSYLA | 39 | GASSRAT | 37 | QQYNSSPYT | 40 |
| DL3B409 | RASQGISNYLA | 41 | AASTLQS | 42 | QQLNSYPLT | 43 |
| DL3B450 | RSSQSLLHSNGY | 44 | LGSNRAS | 45 | MQALQTPLT | 46 |
| | NYLD | | | | | |
| DL3B461 | RSSQSLVHSDGN | 47 | QISNPFS | 48 | MQATQFPHT | 49 |
| | TYLN | | | | | |

TABLE 11
HCDR1, HCDR2 and HCDR3 amino acid sequences of
selected anti-DLL3 antibodies using AbM
delineation.

| | AbM HCDR1 | | AbM HCDR2 | | AbM HCDR3 | |
|---|---|---|---|---|---|---|
| | | SEQ | | SEQ | | SEQ |
| mAb | | ID | | ID | | ID |
| name | Sequence | NO | Sequence | NO | Sequence | NO |
| DL3B279 | GNTFTNY | 50 | IINPSGGST | 51 | QGPFIGDAFD | 17 |
| | YIH | | S | | I | |
| DL3B279 | GQTFTNY | 52 | IINPSGGST | 51 | QGPFIGDAFD | 17 |
| variant | YIH | | S | | I | |
| DL3B332 | GDSIRSY | 53 | YIYYSGTTN | 54 | IGPAGFYFDY | 20 |
| | YWS | | | | | |
| DL3B358 | GHIFISYY | 55 | IIDPSGGSK | 56 | QGMIVGTTGD | 23 |
| | IH | | S | | AFDI | |
| DL3B409 | GYTFTTY | 57 | IIDPSGGRT | 58 | GGDGTWYYG | 26 |
| | YIH | | S | | MDV | |
| DL3B450 | GGSISSY | 59 | RIYTSGSTN | 60 | DQAYSGYDWS | 29 |
| | YWS | | | | YYYYGMDV | |
| DL3B461 | GGTLSSY | 61 | GIIPIFGTA | 62 | DPFSDL | 32 |
| | VIS | | N | | | |

TABLE 12
LCDR1, LCDR2 and LCDR3 amino acid sequences of
selected anti-DLL3 antibodies using AbM
delineation.

| | AbM LCDR1 | | AbM LCDR2 | | AbM LCDR3 | |
|---|---|---|---|---|---|---|
| | | SEQ | | SEQ | | SEQ |
| mAb | | ID | | ID | | ID |
| name | Sequence | NO | Sequence | NO | Sequence | NO |
| DL3B279 | RASQGISNYLA | 33 | AASSLQS | 34 | QQYNSYPYT | 35 |
| DL3B279 | RASQGISNYLA | 33 | AASSLQS | 34 | QQYNSYPYT | 35 |
| variant | | | | | | |
| DL3B332 | RASQSVSRSYL | 36 | GASSRAT | 37 | QQYGTSPIT | 38 |
| | A | | | | | |
| DL3B358 | RASQSASSYLA | 39 | GASSRAT | 37 | QQYNSSPYT | 40 |
| DL3B409 | RASQGISNYLA | 41 | AASTLQS | 42 | QQLNSYPLT | 43 |
| DL3B450 | RSSQSLLHSNG | 44 | LGSNRAS | 45 | MQALQTPLT | 46 |
| | YNYLD | | | | | |
| DL3B461 | RSSQSLVHSDG | 47 | QISNPFS | 48 | MQATQFPHT | 49 |
| | NTYLN | | | | | |

TABLE 13
HCDR1, HCDR2 and HCDR3 amino acid sequences of
selected anti-DLL3 antibodies using Chotia
delineation.

| | Chotia HCDR1 | | Chotia HCDR2 | | Chotia HCDR3 | |
|---|---|---|---|---|---|---|
| | | SEQ | | SEQ | | SEQ |
| mAb | | ID | | ID | | ID |
| name | Sequence | NO | Sequence | NO | Sequence | NO |
| DL3B279 | GNTFTNY | 140 | NPSGGS | 141 | QGPFIGDAFD | 142 |
| DL3B279 | GQTFTNY | 143 | NPSGGS | 141 | QGPFIGDAFD | 142 |
| variant | | | | | | |
| DL3B332 | GDSIRSY | 244 | YYSGT | 144 | IGPAGFYFD | 145 |
| DL3B358 | GHIFISY | 146 | DPSGGS | 147 | QGMIVGTTGDA | 148 |
| | | | | | FD | |
| DL3B409 | GYTFTTY | 149 | DPSGGR | 150 | GGDGTWYYGM | 151 |
| | | | | | D | |
| DL3B450 | GGSISSY | 152 | YTSGS | 153 | DQAYSGYDWS | 154 |
| | | | | | YYYYGMD | |
| DL3B461 | GGTLSSY | 155 | IPIFGT | 156 | DPFSD | 157 |

TABLE 14
LCDR1, LCDR2 and LCDR3 amino acid sequences of
selected anti-DLL3 antibodies using Chotia
delineation.

| | Chotia LCDR1 | | Chotia LCDR2 | | Chotia LCDR3 | |
|---|---|---|---|---|---|---|
| | | SEQ | | SEQ | | SEQ |
| mAb | | ID | | ID | | ID |
| name | Sequence | NO | Sequence | NO | Sequence | NO |
| DL3B279 | SQGISNY | 158 | AAS | 159 | YNSYPY | 160 |
| DL3B279 | SQGISNY | 158 | AAS | 159 | YNSYPY | 160 |
| variant | | | | | | |
| DL3B332 | SQSVSRSY | 161 | GAS | 162 | YGTSPI | 201 |
| DL3B358 | SQSASSY | 202 | GAS | 162 | YNSSPY | 245 |
| DL3B409 | SQGISNY | 158 | AAS | 159 | LNSYPL | 203 |
| DL3B450 | SQSLLHSNGYNY | 204 | LGS | 205 | ALQTPL | 207 |
| DL3B461 | SQSLVHSDGNTY | 208 | QIS | 209 | ATQFPH | 210 |

TABLE 15
HCDR1, HCDR2 and HCDR3 amino acid sequences of
selected anti-DLL3 antibodies using IMTG
delineation.

| | IMTG HCDR1 | | IMTG HCDR2 | | IMTG HCDR3 | |
|---|---|---|---|---|---|---|
| | | SEQ | | SEQ | | SEQ |
| mAb | | ID | | ID | | ID |
| name | Sequence | NO | Sequence | NO | Sequence | NO |
| DL3B279 | GNTFTNY | 211 | INPSGGST | 212 | ARQGPFIGDAFDI | 213 |
| | Y | | | | | |
| DL3B279 | GQTFTNY | 214 | INPSGGST | 212 | ARQGPFIGDAFDI | 213 |
| variant | Y | | | | | |
| DL3B332 | GDSIRSYY | 215 | IYYSGTT | 216 | ARIGPAGFYFDY | 217 |
| DL3B358 | GHIFISYY | 218 | IDPSGGSK | 219 | ARQGMIVGTTG | 220 |
| | | | | | DAFDI | |
| DL3B409 | GYTFTTY | 221 | IDPSGGRT | 222 | ARGGDGTWYYG | 223 |
| | Y | | | | MDV | |
| DL3B450 | GGSISSYY | 224 | IYTSGST | 225 | ARDQAYSGYDW | 226 |
| | | | | | SYYYYGMDV | |
| DL3B461 | GGTLSSY | 227 | IIPIFGTA | 228 | ARDPFSDL | 231 |
| | V | | | | | |

TABLE 16
LCDR1, LCDR2 and LCDR3 amino acid sequences of
selected anti-DLL3 antibodies using IMTG
delineation.

| | IMTG LCDR1 | | IMTG LCDR2 | | IMTG LCDR3 | |
|---|---|---|---|---|---|---|
| | | SEQ | | SEQ | | SEQ |
| mAb | | ID | | ID | | ID |
| name | Sequence | NO | Sequence | NO | Sequence | NO |
| DL3B279 | QGISNY | 232 | AAS | 159 | QQYNSYPYT | 35 |
| DL3B279 | QGISNY | 232 | AAS | 159 | QQYNSYPYT | 35 |
| variant | | | | | | |
| DL3B332 | QSVSRSY | 240 | GAS | 162 | QQYGTSPIT | 38 |
| DL3B358 | QSASSY | 241 | GAS | 162 | QQYNSSPYT | 40 |
| DL3B409 | QGISNY | 232 | AAS | 159 | QQLNSYPLT | 43 |
| DL3B450 | QSLLHSNGYNY | 242 | LGS | 205 | MQALQTPLT | 46 |
| DL3B461 | QSLVHSDGNTY | 243 | QIS | 209 | MQATQFPHT | 49 |

TABLE 17
Amino acid sequences and SEQ ID NO summary of the
variable domains of selected anti-DLL3 antibodies
using Kabat delineation.

| | | | SEQ |
|---|---|---|---|
| | | | ID |
| Antibody | Region | Amino acid sequence | NO: |
| DL3B279 | HCDR1 | NYYIH | 15 |
| | HCDR2 | IINPSGGSTSYAQKLQG | 16 |
| | HCDR3 | QGPFIGDAFDI | 17 |
| | LCDR1 | RASQGISNYLA | 33 |
| | LCDR2 | AASSLQS | 34 |
| | LCDR3 | QQYNSYPYT | 35 |
| | VH | QVQLVQSGAEVKKPGASVKVSCKASGN | 1 |
| | | TFTNYYIHWVRQAPGQGLEWMGIINPSG | |
| | | GSTSYAQKLQGRMTMTRDTSTSTVYMEL | |
| | | SSLRSEDTAVYFCARQGPFIGDAFDIWGQ | |
| | | GTMVTVSS | |
| | VL | DIQMTQSPSSLSASVGDRVTITCRASQGIS | 2 |
| | | NYLAWFQQKPGKAPKSLIYAASSLQSGV | |
| | | PSKFSGSGSGTDFTLTISSLQPEDFATYYC | |
| | | QQYNSYPYTFAQGTKLEIK | |
| DL3B279 | HCDR1 | NYYIH | 15 |
| | HCDR2 | IINPSGGSTSYAQKLQG | 16 |
| | HCDR3 | QGPFIGDAFDI | 17 |
| | LCDR1 | RASQGISNYLA | 33 |
| | LCDR2 | AASSLQS | 34 |
| | LCDR3 | QQYNSYPYT | 35 |
| | VH | QVQLVQSGAEVKKPGASVKVSCKASGQ | 3 |
| | | TFTNYYIHWVRQAPGQGLEWMGIINPSG | |
| | | GSTSYAQKLQGRMTMTRDTSTSTVYMEL | |
| | | SSLRSEDTAVYFCARQGPFIGDAFDIWGQ | |
| | | GTTVTVSS | |
| | VL | DIQMTQSPSSLSASVGDRVTITCRASQGIS | 4 |
| | | NYLAWFQQKPGKAPKSLIYAASSLQSGV | |
| | | PSKFSGSGSGTDFTLTISSLQPEDFATYYC | |
| | | QQYNSYPYTFGQGTKLEIK | |
| DL3B332 | HCDR1 | SYYWS | 18 |
| | HCDR2 | YIYYSGTTNYKSSLKS | 19 |
| | HCDR3 | IGPAGFYFDY | 20 |
| | LCDR1 | RASQSVSRSYLA | 36 |
| | LCDR2 | GASSRAT | 37 |
| | LCDR3 | QQYGTSPIT | 38 |
| | VH | QVQLQESGPGLVKPSETLSLSCTVSGDSIR | 5 |
| | | SYYWSWIRQPPGKGLEWIGYIYYSGTTN | |
| | | YKSSLKSRVTISLDTSKKQFSLNLDSVTA | |
| | | ADTAVYYCARIGPAGFYFDYWGQGTLV | |
| | | TVSS | |
| | VL | EIVLTQSPGTLSLSPGERATLSCRASQSVS | 6 |
| | | RSYLAWYQQKPGQAPRFLIYGASSRATGI | |
| | | PDRFSGSGSGTDFTLTISRLEPEDFAVYYC | |
| | | QQYGTSPITFGQGTRLEIK | |
| DL3B358 | HCDR1 | SYYIH | 21 |
| | HCDR2 | IIDPSGGSKSYAQKFQG | 22 |
| | HCDR3 | QGMIVGTTGDAFDI | 23 |
| | LCDR1 | RASQSASSYLA | 39 |
| | LCDR2 | GASSRAT | 37 |
| | LCDR3 | QQYNSSPYT | 40 |
| | VH | QVQLVQSGAEVKKPGASVKVSCKASGHI | 7 |
| | | FISYYIHWVRQAPGQGLEWMGIIDPSGGS | |
| | | KSYAQKFQGRVTMTRDTSTSTVYMELSS | |
| | | LRSEDTAVYYCARQGMIVGTTGDAFDIW | |
| | | GQGTMVTVSS | |
| | VL | EIVLTQSPGTLSLSPGERATLSCRASQSAS | 8 |
| | | SYLAWYQQKPGQAPRLLIYGASSRATGIP | |
| | | DRFSGSGSGTDFTLTISRLEPEDFAVYYC | |
| | | QQYNSSPYTFGQGTKLEIK | |
| DL3B409 | HCDR1 | TYYIH | 24 |
| | HCDR2 | IIDPSGGRTSYAQKFLG | 25 |
| | HCDR3 | GGDGTWYYGMDV | 26 |
| | LCDR1 | RASQGISNYLA | 41 |
| | LCDR2 | AASTLQS | 42 |
| | LCDR3 | QQLNSYPLT | 43 |
| | VH | EVQLVQSGAEVKKPGASVKVSCKASGYT | 9 |
| | | FTTYYIHWVRQAPGQGLEWMGIIDPSGG | |
| | | RTSYAQKFLGRVTMTRDTSTSTVYMELR | |
| | | SLRSEDTAVYYCARGGDGTWYYGMDV | |
| | | WGQGTTVTVSS | |
| | VL | DIVMTQSPSFLSASVGDRVTITCRASQGIS | 10 |
| | | NYLAWYQQKPGKAPKLLIYAASTLQSGV | |
| | | PSRFSGSGSGTEFTLTISSLQPEDFATYYC | |
| | | QQLNSYPLTFGGGTKVEIK | |
| DL3B450 | HCDR1 | SYYWS | 27 |
| | HCDR2 | RIYTSGSTNYNPSLKS | 28 |
| | HCDR3 | DQAYSGYDWSYYYYGMDV | 29 |
| | LCDR1 | RSSQSLLHSNGYNYLD | 44 |
| | LCDR2 | LGSNRAS | 45 |
| | LCDR3 | MQALQTPLT | 46 |
| | VH | QVQLQQSGPGLVKPSETLSLTCTVSGGSI | 11 |
| | | SSYYWSWIRQPAGKGLEWIGRIYTSGSTN | |
| | | YNPSLKSRVTMSVDTSKNQFSLKLSSVTA | |
| | | ADTAVYYCARDQAYSGYDWSYYYYGM | |
| | | DVWGQGTMVTVSS | |
| | VL | ETTLTQSPLSLPVTPGEPASISCRSSQSLLH | 12 |
| | | SNGYNYLDWYLQKPGQSPQLLIYLGSNR | |
| | | ASGVPDRFSGSGSGTDFTLKISRVEAEDV | |
| | | GVYYCMQALQTPLTFGGGTKVEIK | |
| DL3B461 | HCDR1 | SYVIS | 30 |
| | HCDR2 | GIIPIFGTANYAQKFQD | 31 |
| | HCDR3 | DPFSDL | 32 |
| | LCDR1 | RSSQSLVHSDGNTYLN | 47 |
| | LCDR2 | QISNPFS | 48 |
| | LCDR3 | MQATQFPHT | 49 |
| | VH | QVQLVQSGAEVKKPGSSVKVSCKASGGT | 13 |
| | | LSSYVISWVRQAPGQGLEWMGGIIPIFGT | |
| | | ANYAQKFQDRVTITADKSTNTAYMELTS | |
| | | LTSEDTAVYYCARDPFSDLWGRGTMVT | |
| | | VSS | |
| | VL | DIVMTQSPLSSPVTLGQPASISCRSSQSLV | 14 |
| | | HSDGNTYLNWLQQRPGQPPRLLIYQISNP | |
| | | FSGVPDRFSGSGAGTDFTLKISRVEAEDV | |
| | | GVYYCMQATQFPHTFGPGTKVEIK | |

TABLE 18
SEQ ID NOs of Protein and DNA sequences of the VH
and VL domains of selected anti-DLL3 antibodies.

| | VH Protein | VL Protein | VH cDNA | VL cDNA |
|---|---|---|---|---|
| Antibody | SEQ ID NO: | SEQ ID NO | SEQ ID NO: | SEQ ID NO: |
| DL3B279 | 1 | 2 | 163 | 164 |
| DL3B279 | 3 | 4 | 165 | 166 |
| variant | | | | |
| DL3B332 | 5 | 6 | 167 | 168 |
| DL3B358 | 7 | 8 | 169 | 170 |
| DL3B409 | 9 | 10 | 171 | 172 |
| DL3B450 | 11 | 12 | 173 | 174 |
| DL3B461 | 13 | 14 | 175 | 176 |

Fab-Fc and scFvs

The DLL3 specific VH/VL regions were engineered as Fab-Fc in the VH-CH1-hinge CH2-CH3 and VL-CL format and expressed as IgG1, IgG2 or IgG4. The DLL3 specific VH/VL were also engineered as scFvs in either the VH-Linker-VL (scFv-LH) or VL-linker-VH orientations (scFv-LH) (Table 19) using the linker of SEQ ID NO:120 (Linker 1) described in Example 2 and in Table 2. These scFv were used to generate bispecific antibodies.

TABLE 19
Amino acid sequences of the variable domain of selected
anti-DLL3 scFvs antibodies in VL-linker-VH (LH) format.

| Acronym | SEQ ID NO: |
|---|---|
| DL3B279 scFv_LH | 63 |
| DL3B279 scFv_LH variant | 64 |
| DL3B332 scFv_LH | 65 |
| DL3B358 scFv_LH | 66 |
| DL3B409 scFv_LH | 67 |
| DL3B450 scFv_LH | 68 |
| DL3B461 scFv_LH | 69 |
| DL3B279 scFv_HL | 190 |
| DL3B279 scFv variant_HL | 191 |
| DL3B332 scFv_HL | 192 |
| DL3B358 scFv_HL | 193 |
| DL3B409 scFv_HL | 194 |
| DL3B450 scFv_HL | 195 |
| DL3B461 scFv_HL | 196 |

The DNA sequences of the variable domain of selected anti-DLL3 scFv antibodies in VL-linker-VH (LH) format are: DL3B279-scFv-LH DNA (SEQ ID NO:260); DL3B279-scFv-LH variant DNA (SEQ ID NO:261); DL3B332-scFv-LH DNA (SEQ ID NO:262); DL3B358-scFv-LH DNA (SEQ ID NO:233); DL3B409-scFv-LH DNA (SEQ ID NO:234); DL3B450-scFv-LH DNA (SEQ ID NO:235); DL3B461-scFv-LH DNA (SEQ ID NO:236).

### Example 6: Generation of Anti-CD3 Antibodies

### Immunization

The generation of anti-CD3 antibody CD3B376 has been described in US20200048349, which is incorporated by reference in its entirety. The CD3B376 Fab comprises the HCDR1 of amino acid sequence NNNAAWS (SEQ ID NO:98), the HCDR2 of amino acid sequence RTYYRSKWLYDYAYSYKS (SEQ ID NO:99), and the HCDR3 of amino acid sequence GYSSSFDY (SEQ ID NO: 100) and the LCDR1 of amino acid sequence TGTSSNIGTYKFVS (SEQ ID NO: 106), the LCDR2 of amino acid sequence EVSKRPS (SEQ ID NO: 107), and the LCDR3 of amino acid sequence VSYAGSGTLL (SEQ ID NO:108) using the Kabat delineation. The VH and VL sequences of CD3B376 are: SEQ ID NO:84, VH amino acid sequence of CD3B376 Fab, SEQ ID NO:85, VL amino acid sequence of CD3B376 Fab; SEQ ID NO:93, VH nucleic acid sequence of CD3B376 Fab; and SEQ ID NO:94, VL nucleic acid sequence of CD3B376 Fab.

Alternatively, anti-CD3 antibodies were generated using Ablexis transgenic mouse platform. Ablexis mice were immunized with TRCW5 (SEQ ID NO:197), including 13 Kappa mice and 12 Lambda mice. TRCW5 is comprised of the extracellular region of CD36 fused to to the extracellular region of CD3ε with a 26 amino acid linker. A human IgG1 Fc domain with a C-terminal Avi-tag was added to the C-terminus for site-specific biotinylation

Mice were immunized twice weekly for the duration of 7 weeks. On day 42, mice were boosted for hybridoma fusion by administration of 50 μg TRCW5 and 50 μg CD40 mAb spread over 8 sites, including 6 subcutaneous and 2 intradermal injections. For a final boost, mice received 20 μL injections of Jurkat cells, a T cell line which endogenously expresses the T cell receptor complex, including CD3ε (Schneider et al (1977) Int. J. Cancer, 19 (5): 621-6), at 4.74×10⁷ cells/mL.

Lymph nodes and spleens were extracted from mice and fusions performed by cohorts. Lymph node cells were counted and combined in a 1:1 ratio with FO myeloma cells (ATCC (CRL-1646)) and incubated for 10 days at 37° C. prior to antibody screening. Supernatants from hybridoma fusion cells were then assayed by ELISA for binding to TRCW5 using TRCW5 either non-specifically immobilized on the plate (ELISA, Thermo cat. #34022) or immobilized by streptavidin conjugation to biotinylated-TRCW5 (SPARCL ELISA, Lumigen), according to manufacturers' instructions. ELISA assays were performed by coating plates with 0.5 ug/mL TRCW5 and 0.5 ug/mL HVEM-Fc (R&D cat. #365-HV) overnight at 4° C. Plates were blocked by addition of 0.4% (w/v) bovine serum albumin (BSA) in phosphate-buffered saline (PBS) overnight at 4° C. Plates were washed with 1×PBS supplemented with 0.02% (v/v) Tween 20. To each well, 50 uL of hybridoma supernatant was applied and incubated for 1 hr at room temperature. Bound antibody was detected by addition of goat anti-mouse IgG Fc conjugated to horseradish peroxidase (Jackson cat. #115-036-071) diluted 1:10,000 in blocking buffer followed by incubation for 30 min at room temperature. 3,3′,5,5′-tetramethylbenzidine (TMB) substrate buffer (Thermo cat. #34022) was added at 25 uL/well and incubated for 10 min in the dark. Reactions were stopped by addition of 25 uL/well of 4 M H₂SO₄. Luminescence was read at 450 nm using BioTek© Epoch2 Microplate Reader. Hits were selected having signal at least 3-fold higher than background.

The two assay formats resulted in 426 hits (264 hits from ELISA, 194 from SPARCL ELISA, 70 hits were identified in both assays). Of these 426 initial hits, 49 ELISA and 32 SPARCL ELISA hits were confirmed. The hyriboma fusions corresponding to the positive binders were refed and tested for their abilities to bind Jurkat cells endogenously expressing CD3, using flow cytometry. Three antibodies, including clone 003_F12, clone 036_E10 and clone 065_D03, showed significant binding to Jurkat cells, endogenously expressing CD3, based on mean fluorescence index (MFI) (Table 20). While clones 003_F12 and 036_E10 (from human kappa mice) were confirmed positive for human kappa light chain by ELISA, clone 065_D03 (from human lambda mouse) was negative for human lambda. The variable genes of these three clones were sequenced.

TABLE 20
Mean fluorescence index (MFI) for binding
of selected clones to Jurkat cells.

| Clone ID | MFI (arbitrary units) |
|---|---|
| 003_F12 | 176,147 |
| 036_E10 | 43,133 |
| 065_D03 | 136,269 |
| No Ab | 2,075.61 |
| 10 nM UCHT1 | 89,214.29 |

Next, these three clones were screened for their abilities to bind primary human and cyno T cells. Briefly, primary human and cyno pan T cells were resuspended at 1×10⁶ cells/mL in flow staining buffer and cells were plated at 50,000 cells/well. To each well, 50 uL of hybridoma supernatant were added and the mixture was incubated on ice for 30 min. After incubation, 200 μL of staining buffer was added and cells were pelleted by centrifugation at 300×G for 5 min. Anti-mouse IgG conjugated to Alexa-647 was added at 2 μg/mL in staining buffer in 50 μL total volume and incubated for 30 min on ice. 150 μL of staining buffer was added and cells were pelleted by centrifugation at 300×G for 5 min. Cells were resuspended in 30 μL of running buffer containing 1:1,000-diluated Sytox green dead cell stain and run on iQue Screener. Cells were gated on FCS vs SCS to eliminate debris. Singlets were gated on SCS-A vs SCS-H, and from singlet population, live cells were chosen using BL1 channel for low-negative with Sytox green. CD3 binding was assessed by comparing test articles to negative control by RL1 (Alexa-647) geomeans. In this assay, clone 065_D03 showed the highest cell binding signal.

The variable region of the Clone 065_D03 was then cloned into an IgG1 backbone, resulting in the antibody termed CD3B815 (sequences are shown in Table 21). CD3B815 was screened again for binding to Jurkat cells and showed positive binding to Jurkat cells.

TABLE 21
CD3B815 amino acid sequences.

| Protein | SEQ ID NO: |
|---|---|
| CD3B815 (Heavy Chain) | 198 |
| CD3B815 (Light Chain) | 199 |

Humanization and scFv Formatting of CD3 Binding Domains

The light chain (LC) of the v-region of CD3B815 was humanized in scFv format. Briefly, the LC from CD3B815 was grafted onto the human IGKV1-39*01-IGKJ2*01 germline. and position Y49K was identified for human to mouse back mutations. The LC from CD3B815 also contained an NS (Asn-Ser) motif at positions 92-93 which presents a risk for deamidation at this site. The grafting of CD3B815 CDRs into IGKV1D-39*01 and the introduction of the LC mutations Y49K and N92G resulted in the CD3W245 antibody with VH and VL sequences as shown below.
Table 22 shows the VH and the VL amino acid sequences of selected anti-CD3 antibodies.
Table 23 shows the VH and the VL DNA sequences of selected anti-CD3 antibodies.
Table 24 shows CD3 scFv amino acid sequences. Table 25 shows the Kabat HCDR1, HCDR2 and the HCDR3 amino acid sequences of selected anti-CD3 antibodies in Kabat delineation.
Table 26 shows the Kabat LCDR1, LCDR2 and the LCDR3 amino acid sequences of selected anti-CD3 antibodies in Kabat delineation. Table 27 summarizes the CDRs, VH and VL sequences of selected CD3 antibodies.

TABLE 22
VH and VL amino acid sequences of selected anti-CD3 variants.

| mAb | VH SEQ ID NO | VL SEQ ID NO |
|---|---|---|
| CD3B815 | 77 | 78 |
| CD3W245 | 77 | 80 |

TABLE 23
VH and VL nucleic acid sequences of the humanized variants.

| mAb | VH SEQ ID NO: | VL SEQ ID NO: |
|---|---|---|
| CD3B815 | 86 | 87 |
| CD3W245 | 86 | 89 |

TABLE 24
HCDR1, HCDR2 and HCDR3 amino acid sequences of
selected anti-CD3 antibodies using Kabat
delineation.

| | | SEQ | | SEQ | | SEQ |
|---|---|---|---|---|---|---|
| | | ID | | ID | | ID |
| mAb | HCDR1 | NO: | HCDR2 | NO: | HCDR3 | NO: |
| CD3B815 | RYNMN | 95 | SISTSSNYIYYADSVKG | 96 | GWGPFDY | 97 |
| CD3W245 | RYNMN | 95 | SISTSSNYIYYADSVKG | 96 | GWGPFDY | 97 |

TABLE 25
LCDR1, LCDR2 and LCDR3 amino acid sequences of
selected anti-CD3 antibodies using Kabat
delineation.

| | | SEQ | | SEQ | | SEQ |
|---|---|---|---|---|---|---|
| | | ID | | ID | | ID |
| mAb | LCDR1 | NO: | LCDR2 | NO: | LCDR3 | NO: |
| CD3B815 | RARQSIGTAIH | 101 | YASESIS | 102 | QQSNSWPYT | 103 |
| CD3W245 | RARQSIGTAIH | 101 | YASESIS | 102 | QQSGSWPYT | 104 |

TABLE 26
HCDR1, HCDR2, HCDR3, LCDR1, LCDR2, LCDR3, VH and
VL of anti-CD3 antibodies

| | | | SEQ |
|---|---|---|---|
| | | | ID |
| Antibody | Region | Amino Acid sequence | NO: |
| CD3B815 | HCDR1 | RYNMN | 95 |
| | HCDR2 | SISTSSNYIYYADSVKG | 96 |
| | HCDR3 | GWGPFDY | 97 |
| | LCDR1 | RARQSIGTAIH | 101 |
| | LCDR2 | YASESIS | 102 |
| | LCDR3 | QSNSWPYT | 103 |
| | VH | EVQLVESGGGLVKPGGSLRLSCAASGFTFS | 77 |
| | | RYNMNWVRQAPGKGLEWVSSISTSSNYIYY | |
| | | ADSVKGRFTFSRDNAKNSLDLQMSGLRAE | |
| | | DTAIYYCTRGWGPFDYWGQGTLVTVSS | |
| | VL | DILLTQSPGILSVSPGERVSFSCRARQSIGT | 78 |
| | | AIHWYQQRTNGSPRLLIKYASESISGIPSRE | |
| | | SGSGSGTDFTLTINSVESEDIADYYCQQSNS | |
| | | WPYTFGGGTKLEIK | |
| CD3BW245 | HCDR1 | RYNMN | 95 |
| | HCDR2 | SISTSSNYIYYADSVKG | 96 |
| | HCDR3 | GWGPFDY | 97 |
| | LCDR1 | RARQSIGTAIH | 101 |
| | LCDR2 | YASESIS | 102 |
| | LCDR3 | QQSGSWPYT | 104 |
| | VH | EVQLVESGGGLVKPGGSLRLSCAASGFTFS | 77 |
| | | RYNMNWVRQAPGKGLEWVSSISTSSNYIYY | |
| | | ADSVKGRFTFSRDNAKNSLDLQMSGLRAE | |
| | | DTAIYYCTRGWGPFDYWGQGTLVTVSS | |
| | VL | DIQMTQSPSSLSASVGDRVTITCRARQSIGT | 80 |
| | | AIHWYQQKPGKAPKLLIKYASESISGVPSRF | |
| | | SGSGSGTDFTLTISSLQPEDFATYYCQQSGS | |
| | | WPYTFGQGTKLEIK | |
| CD3B376 | HCDR1 | NNNAAWS | 98 |
| | HCDR2 | RTYYRSKWLYDYAYSYKS | 99 |
| | HCDR3 | GYSSSFDY | 100 |
| | LCDR1 | TGTSSNIGTYKFVS | 106 |
| | LCDR2 | EVSKRPS | 107 |
| | LCDR3 | VSYAGSGTLL | 108 |
| | VH | QVQLQQSGPRLVRPSQTLSLTCAISGDSVFN | 84 |
| | | NNAAWSWIRQSPSRGLEWLGRTYYRSKWL | |
| | | YDYAVSVKSRITVNPDTSRNQFTLQLNSVTP | |
| | | EDTALYYCARGYSSSFDYWGQGTLVTVSS | |
| | VL | QSALTQPASVSGSPGQSITISCTGTSSNIGT | 85 |
| | | YKFVSWYQQHPDKAPKVLLYEVSKRPSGVSS | |
| | | RFSGSKSGNTASLTISGLQAEDQADYHCVS | |
| | | YAGSGTLLFGGGTKLTVL | |

The VH and VL sequences of CD3W245 were also expressed in a scFv format in the VH-linker-VL orientation (scFv-HL) or in the VL-linker-VH orientation (ScFv-LH). The linker sequence (GGSEGKSSGSGSESKSTGGS) of SEQ ID NO: 120 was used to conjugate the VH/VL regions. The sequences of CD3W245 scFv LH and CD3W245 scFv HL are shown in Table 27.

TABLE 27
scFv amino acid sequences

| scFv name | SEQ ID NO: |
|---|---|
| CD3W245-scFv-LH | 105 |
| CD3W245-scFv-HL | 119 |

Binding of Humanized Anti-CD3 scFv Variants to CD3 after Heat Shock.

The variable regions of the CD3W245 was also formatted as scFv in VH-linker-VL orientation using linker GTEGKSSGSGSESKST (SEQ ID NO: 139) for expression in E. coli. A 6×his Tag was engineered at the C-terminus. This construct was used to test binding to recombinant CD3 (homodimeric CD3εγ-Fc, CD3W147, SEQ ID NO:200) and binding to T cells. The sequence of the CD3W147 and CD3W245 scFv HL expressed in E. coli is shown in SEQ ID NO:200, CD3W147 and SEQ ID NO:206, CD3W245-HL-E.c., expressed in E. coli.

Briefly, scFv-coding sequences were cloned into a pADL™-22c vector having a PelB leader sequence for secretion. E. coli cells were transformed with plasmid and grown overnight at 37° C. in 2xYT microbial growth medium supplemented with 100 μg/mL Carbenicillin.

Protein expression was induced by addition of 1 mM IPTG and cultures were grown overnight. After expression, cells were pelleted by centrifugation at 2,200×g for 5 min and supernatants were collected and tested directly for binding to biotinylated CD3W147 by ELISA.

The binding of the anti-CD3 antibody (CD3W245 scFv-HL) to CD3W147 was determined by ELISA. Biotinylated CD3W147 was immobilized on the plate in concentrations ranging from 0.039 μg/mL to 2.5 μg/mL in 2-fold dilutions followed by incubation at room temperature for 45 min. Bound scFv was detected using chicken anti-HA-horseradish peroxidase and then detected with chemiluminescence substrate. CD3W245 showed binding to CD3W147 (data not shown)

CD3W245 scFv-HL was then tested for its abilities to bind T cells, using flow cytometry. Briefly, human T cells were thawed and resuspended into flow staining buffer at 1×10\hat{ }6 cells/mL and plated at 50,000 cells/well. A positive control, CD3W36 comprised of an anti-CD3 antibody SP34 formatted as scFv-LH, and a negative control, B23, an scFv targeted against the F-glycoprotein from respiratory syncytial virus, were used for comparison. E. coli supernatant expression CD3W245 scFv-HL was added at 150 μL/well and incubated at 4° C. for 1 hr. After incubation, plates were washed with staining buffer and detected with anti-His antibody conjugated to Alexa-647 in staining buffer. After incubation, 200 μL of IntelliCyt running buffer was added to the mixture, and cells were resuspended in 30 μL running buffer containing 1:1,000 Sytox Green dead cell stain and analyzed on iQue Screener. Gating and analysis were performed as above. CD3W245 scFv-HLdisplayed mean fluorescence indices consistent with T cell binding (Table 28).

TABLE 28
T cell-based binding of humanized scFv molecules.

| Protein | MFI (n = 2) |
|---|---|
| CD3W245-HL-E.c. | 178140.0 |
| B23 | 51.8 |
| CD3W36 | 99451.6 |

### Example 7. Effect of DLL3 Epitope on the Bispecific DLL3×CD3 Mediated Cytotoxicity

To determine the effect of DLL3 epitope on bispecific DLL3×CD3 mediated killing on DLL3+ target cells, a T cell redirection was performed using human pan T cells as effectors and SHP-77 cells as targets at a 3:1 ratio for 72 hours. Various DLL3×CD3 antibodies were generated using DLL3 antibodies able to bind to individual DLL3 subdomains to study the effect of domain binding on cytotoxicity. The DLL3 antibodies binding to various DLL3 subdomains were combined with three different CD3 arms, CD3B376 and CD3W245 described in Example 5 and CD3B219 described in US20200048349 to generate the bispecific antibodies used in this study.

DLL3×CD3 mediated killing experiments were run using an equal volume (100 ul) of 2× test sample, in ½ log dilutions from 20 nM (final starting at 10 nM) that was added to 50,000 CSFE-labelled SHP-77 cells and mixed with 150,000 pan T cells in a final volume of 200 ul RPMI, 10% FBS for 72 hr at 37° C. After 72 hours, plates were washed 1× with PBS, incubated for 20 minutes with Near IR L/D stain and BV421-labeled anti-CD25 antibody in stain buffer. The cells were washed twice with stain buffer, resuspended in 25 ul Accutase for 10 minutes, and then 25 ul of QSol buffer was added. The plates were read on an IQue plus and cells were gated on CSFE positive populations (Tumor cells) and CSFE-negative cells (T cells) and both populations were subsequently gated on live/dead staining. Live T cells were further gated on CD25 staining. Outputs calculated were % Tumor killing, % CD25 T cell activation, and T cell viability. A ruby red stained control (mock 100% dead) and T cell only/SHP-77 only were used to gate nuclei containing cells from debris and then the individual cell populations. Data was analyzed in GeneData Screenr using 4 parameter curve fits.

Tables 31-33 show the maximal percent lysis of SHP-77 cells observed at the end of 72 hours for each DLL3 binder paired with the various CD3 arms. Inventors have unexpectedly discovered that an interesting trend appears where maximum killing in each domain increases as the binding domain within the DLL3 moves towards the C-terminus in the primary sequence or proximal to the tumor membrane. The results indicated that the % tumor killing is dependent on the binding epitope on DLL3. and that cell lysis decreases as the antibodies binds to a DLL3 subdomain further away from the membrane (Tables 29-31). The % tumor killing was improved as the DLL3 binding epitopes became more membrane proximal. This trend is relatively consistent and independent of the CD3 arm.

In particular, maximum killing efficiency improves when the DLL3-CD3 bispecific antibody binds from EGF2 to EGF6 subdomain of DLL3 and reaches the highest percentage, when the tested antibody binds at the EGF-6 domain or closer to the C-terminus.

TABLE 29
% lysis of SHP-77 on day 3 after coculture with human
pan T-cells and bispecific anti-DLL3 × CD3W245
antibodies at 3:1 ET ratio (CD3:target cells).

| | Bispecific | | DLL3 | % Max. |
|---|---|---|---|---|
| Name | description | DLL3 Arm | Epitope | Killing |
| CD3B1706 | CD3W245-Fab-RF; | DL3B279-scFv | EGF6 | 89.7 |
| | DL3B279-scFv | | | |
| CD3B1506 | CD3W245-Fab-RF; | DL3B463-scFv | EGF3/ | 94.5 |
| | DL3B463-scFv | | EGF4 | |
| CD3B1346 | CD3W245-Fab-RF; | DL3B419-scFv | EGF2/ | 85.2 |
| | DL3B419-scFv | | EGF3 | |
| CD3B1586 | CD3W245-Fab-RF; | DL3B470-scFv | DSL | 55.5 |
| | DL3B470-scFv | | | |

TABLE 30
% lysis of SHP-77 on day 3 after coculture with human
pan T-cells and bispecific anti-DLL3 × CD3B376
antibodies at 3:1 ET ratio (CD3:target cells).

| | Bispecific | | DLL3 | % Max. |
|---|---|---|---|---|
| Name | description | DLL3 Arm | Epitope | Killing |
| CD3B1738 | CD3B376-Fab-RF; | DL3B279-scFv | EGF6 | 74.3 |
| | DL3B279-scFv | | | |
| CD3B1538 | CD3B376-Fab-RF; | DL3B463-scFv | EGF3/ | 25.9 |
| | DL3B463-scFv | | EGF4 | |
| CD3B1378 | CD3B376-Fab-RF; | DL3B419-scFv | EGF2/ | 49.1 |
| | DL3B419-scFv | | EGF3 | |
| CD3B1618 | CD3B376-Fab-RF; | DL3B470-scFv | DSL | 3.4 |
| | DL3B470-scFv | | | |

TABLE 31
% lysis of SHP-77 on day 3 after coculture with human
pan T-cells and bispecific anti-DLL3 × CD3B219
antibodies at 3:1 ET ratio (CD3:target cells).

| | Bispecific | | DLL3 | % Max. |
|---|---|---|---|---|
| Name | description | DLL3 Arm | Epitope | Killing |
| CD3B1737 | CD3B219-Fab-RF; | DL3B279-scFv | EGF6 | 86.4 |
| | DL3B279-scFv | | | |
| CD3B1377 | CD3B219-Fab-RF; | DL3B419-scFv | EGF2/ | 73.1 |
| | DL3B419-scFv | | EGF3 | |
| CD3B1617 | CD3B219-Fab-RF; | DL3B470-scFv | DSL | 21.9 |
| | DL3B470-scFv | | | |

### Example 8. Generation of Bispecific DLL3×CD3

DL3B279 and DL3B279 variant (DL3B279-VL-A99G-VH-N27Q_M105T-LH-scFv) were selected for generation of DLL3×CD3 bispecific. The VH/VL regions of the anti-DLL3 antibodies and the VH/VL regions of the anti-CD3 antibodies CD3B376 and CD3W245 were engineered into bispecific format and expressed as IgG1.

Engineering of CD3 and DLL3 scFvs for Bispecific DLL3×CD3 Generation

CD3 VH/VL regions were engineered as scFvs in either VH-Linker-VL or VL-linker-VH orientations using the linker of SEQ ID NO: 120 (Table 2). The VH-Linker-VL or VL-linker-VH scFv molecules binding CD3 were further engineered as IgG1 into a scFv-hinge-CH2-CH3 format comprising Fc silencing mutation (L234A/L235A/D265 S) and dimerization mutations to allow for heterodimerization of the DLL3 and CD3 heavy chains.

DLL3 VH/VL regions were engineered as scFvs in a VL-linker-VH orientation using the same linker as for CD3 scFv generation described above of SEQ ID NO:120 (Table 2). The VL-linker-VH scFv molecules binding DLL3 were further engineered as IgG1 into a scFv-hinge-CH2-CH3 format comprising the Fc silencing mutation (L234A/L235A/D265 S). Mutations designed to promote selective heterodimerization of the Fc domain were also engineered in the Fc domain.

### Engineering of CD3 and DLL3 Fabs for DLL3/CD3 Bispecific Generation

The CD3 and DLL3 specific VH and VL regions were also engineered in VH-CH1-hinge-CH2-CH3 and VL-CL formats respectively, and expressed as IgG1. The Fc silencing mutation L234A/L235A/D265 S were introduced in the Fc region. Mutations designed to promote selective heterodimerization of the Fc domain were also engineered in the Fc domain.

### Expression of Bispecific DLL3×CD3 Antibodies

The bispecific antibodies were expressed in ExpiCHO-S™ cells by transient transfection with purified plasmid DNA following the manufacturer's recommendations. Briefly, ExpiCHO-S™ cells were maintained in suspension in ExpiCHO™ expression medium (ThermoFisher Scientific, Cat #A29100) in an orbital shaking incubator set at 37° C., 8% CO₂ and 125 RPM. The cells were passaged and diluted prior to transfection to 6.0×10⁶ cells per ml, maintaining cell viability at 99.0% or better. Transient transfections were done using the ExpiFectamine™ CHO transfection kit (e.g. ThermoFisher Scientific, Cat #A29131). For each ml of diluted cells to be transfected, 0.5 microgram of each bispecific antibody encoding DNA in ratios of HC1:LC1:HC2=1:2:2 and 0.5 microgram of pAdVAntage DNA (Promega, Cat #E1711) was used and diluted into OptiPRO™ SFM complexation medium. For each liter of cells, 2.56 mL of ExpiFectamine™ CHO reagent was diluted into 8 mL of OptiPRO™. The diluted DNA and transfection reagent were combined for one minute, allowing DNA/lipid complex formation, and then added to the cells. After overnight incubation, ExpiCHO™ feed and ExpiFectamine™ CHO enhancers were added to the cells as per the manufacturer's Standard protocol. Cells were incubated with orbital shaking (125 rpm) at 37° C. for seven days prior to harvesting the culture broth. The culture supernatant from the transiently transfected ExpiCHO-S™ cells was clarified by centrifugation (30 min, 3000rcf) followed by filtration (0.2 μm PES membrane, Corning; Corning, NY).

### Purification of Bispecific DLL3×CD3

The filtered cell culture supernatant was loaded onto a pre-equilibrated (1×DPBS, pH 7.2) HiTrap MabSelect SuRe Protein A column (GE Healthcare) using an AKTA Avant 150 chromatography system. After loading, the column was washed with 5 column volumes of 1×DPBS, pH7.2. The protein was eluted with 8 column volumes of 0.1 M sodium (Na)-Acetate, pH 3.5. Protein fractions were completely neutralized by the addition of 2.5 M Tris HC1, pH 7.2 to 15% (v/v) of the final volume and syringe filtered (0.2 μm). The neutralized protein solution was loaded onto 2×5 mL prepacked CaptureSelect™ IgG-CH1 Affinity Matrix (Thermo Fisher Scientific). The column was washed with 10 column volumes of 1×DPBS, pH7.2. The protein was eluted with 10 column volumes of 0.1 M sodium (Na)-Acetate, pH 3.5. Protein fractions were completely neutralized by the addition of 2.5 M Tris HC1, pH 7.2 to 15% (v/v) of the final volume. The major peak fractions were pooled, dialyzed into 1×DPBS, pH 7.2 with a total of 3 dialysis changes and filtered (0.2 μm).

### Structural Characterization of a DLL3×CD3 Bispecific Antibodies

Table 32 describes the HC and LC amino acid SEQ ID NOs of selected DLL3/CD3 bispecific antibodies. Table 33 shows the HC and LC amino acid sequences of the selected DLL3/CD3 bispecific antibodies. Table 34 described the Kabat CDR SEQ NOs of selected DLL3/CD3 bispecific antibodies. Table 35 describes the HC and LC nucleotide sequence ID NOs of selected DLL3/CD3 bispecific antibodies.

TABLE 32
HC and LC amino acid SEQ ID NOs of DLL3/CD3 bispecific antibodies

| | DLL3 arm | | | | | |
|---|---|---|---|---|---|---|
| | | HC1 or | | CD3 arm | | |
| | | scFv -Fc | LC1 | | HC2 or scFv - | |
| Bispecific | | SEQ ID | SEQ | | Fc SEQ ID | LC2 SEQ |
| Name | Name | NO: | ID NO: | Name | NO: | ID NO: |
| DL3B582 | DL3B279-Fab-Fc | 109 | 110 | CD3W245-LH- | 112 | |
| | | | | scFv-Fc | | |
| DL3B583 | DL3B279-Fab-Fc | 109 | 110 | CD3W245-HL- | 113 | |
| | | | | scFv-Fc | | |
| DL3B585 | DL3B279-LH- | 111 | | CD3B376-Fab- | 116 | 117 |
| | scFv-Fc | | | Fc | | |
| DL3B587 | DL3B279-LH- | 111 | | CD3W245- | 114 | 115 |
| | scFv-Fc | | | Fab-Fc | | |
| D3C3B80 | DL3B279-VL- | 71 | | CD3B376- | 118 | 117 |
| | A99G-VH- | | | K477-Fab-Fc | | |
| | N27Q_M105T- | | | | | |
| | LH-scFv-Fc | | | | | |
| | (ZW) | | | | | |
| D3C3BB3 | DL3B279-VL- | 229 | | CD3B376-Fab- | 230 | 117 |
| | A99G-VH- | | | Fc | | |
| | N27Q_M105T- | | | | | |
| | LH-scFv-Fc | | | | | |
| | (KIH) | | | | | |

TABLE 33
Amino acid sequences of selected bispecific antibodies

| Protein | SEQ ID NO: |
|---|---|
| DL3B279-Fab-Fc HC1 | 109 |
| DL3B279-Fab-Fc LC1 | 110 |
| DL3B279-LH-scFv | 111 |
| DL3B279-VL-A99G-VH- | 71 |
| N27Q_M105T-LH-scFv-Fc (ZW) | |
| CD3W245-LH-scFv-Fc | 112 |
| CD3W245-HL-scFv-Fc | 113 |
| CD3W245-Fab-Fc HC2 | 114 |
| CD3W245-Fab-Fc LC2 | 115 |
| CD3B376-Fab-Fc HC2 | 116 |
| CD3B376-Fab-Fc LC2 | 117 |
| CD3B376-Fab-Fc K477 HC2 | 118 |
| CD3B376-Fab-K477 LC2 | 117 |
| DL3B279-VL-A99G-VH- | 229 |
| N27Q_M105T-LH-scFv-Fc (KIH) | |
| CD3B376-Fab-Fc HC2 | 230 |

TABLE 34
Kabat CDR SEQ ID NOs of bispecific DLL3/CD3 antibodies

| | Parental | | | | | | |
|---|---|---|---|---|---|---|---|
| Bispecific | (DLL3 | | | | | | |
| antibody | arm/CD3 arm) | HCDR1 | HCDR2 | HCDR3 | LCDR1 | LCDR2 | LCDR3 |
| DL3B582 | DL3B279-Fab | 15 | 16 | 17 | 33 | 34 | 35 |
| | CD3W245 | 95 | 96 | 97 | 101 | 102 | 104 |
| | LH-scFv | | | | | | |
| DL3B583 | DL3B279 Fab | 15 | 16 | 17 | 33 | 34 | 35 |
| | CD3W245- | 95 | 96 | 97 | 101 | 102 | 104 |
| | HL-scFv | | | | | | |
| DL3B585 | DL3B279-LH- | 15 | 16 | 17 | 33 | 34 | 35 |
| | scFv | | | | | | |
| | CD3B376-Fab | 98 | 99 | 100 | 106 | 107 | 108 |
| DL3B587 | DL3B279-scFv | 15 | 16 | 17 | 33 | 34 | 35 |
| | CD3W245-Fab | 95 | 96 | 97 | 101 | 102 | 104 |
| D3C3B80 | DL3B279-VL- | 15 | 16 | 17 | 33 | 34 | 35 |
| | A99G-VH- | | | | | | |
| | N27Q_M105T- | | | | | | |
| | LH-scFv | | | | | | |
| | (ZWB) | | | | | | |
| | CD3B376- | 98 | 99 | 100 | 106 | 107 | 108 |
| | K477-Fab | | | | | | |
| D3C3BB3 | DL3B279-VL- | 15 | 16 | 17 | 33 | 34 | 35 |
| | A99G-VH- | | | | | | |
| | N27Q_M105T- | | | | | | |
| | LH-scFv | | | | | | |
| | (KIH) | | | | | | |
| | CD3B376-Fab | 98 | 99 | 100 | 106 | 107 | 108 |
| | (KIH) | | | | | | |

TABLE 35
HC and LC DNA SEQ ID NOs of DLL3/CD3 bispecific antibodies

| | DLL3 arm | | | CD3 arm | | |
|---|---|---|---|---|---|---|
| | | HC1 | LC1 | | HC2 or | |
| | | or scFv -Fc | SEQ | | scFv - | LC2 |
| Bispecific | | SEQ | ID | | Fc SEQ | SEQ ID |
| Name | Name | ID NO: | NO: | Name | ID NO: | NO: |
| DL3B582 | DL3B279-Fab-Fc | 267 | 268 | CD3W245-LH- | 269 | |
| | | | | scFv-Fc | | |
| DL3B583 | DL3B279-Fab-Fc | 267 | 268 | CD3W245-HL- | 270 | |
| | | | | scFv-Fc | | |
| DL3B585 | DL3B279-LH- | 265 | | CD3B376-Fab- | 235 | 236 |
| | scFv-Fc | | | Fc | | |
| DL3B587 | DL3B279-LH- | 265 | | CD3W245-Fab- | 177 | 202 |
| | scFv-Fc | | | Fc | | |
| D3C3B80 | DL3B279-VL- | 266 | | CD3B376- | 256 | 264 |
| | A99G-VH- | | | K477-Fab-Fc | | |
| | N27Q_M105T-LH- | | | | | |
| | scFv (ZWB) | | | | | |
| D3C3BB3 | DL3B279-scFv-Fc | 239 | | CD3B376-Fab- | 237 | 238 |
| | (KIH) | | | Fc (KIH) | | |

In particular, the HC and LC DNA sequences for the DLL3/CD3 bispecific antibodies include, e.g., SEQ ID NO:267 (DL3B279-Fab-Fc HC1 cDNA in DL31B582 and DL31B583); SEQ ID NO:268 (DL3B279-Fab-Fc LC1 cDNA in DL31B582 and DL31B583); SEQ ID NO:265 (DL31B279 LH scFv-Fc cDNA in DL31B585 and DL31B587); SEQ ID NO:266 (DL31B279 LH scFv variant-Fc cDNA); SEQ ID NO:239 (DL31B279 scFv-Fc variant KIH cDNA); SEQ ID NO:269 (CD3W245 LH scFv-Fc cDNA); SEQ ID NO:270 (CD3W245 HL scFv-Fc cDNA); SEQ ID NO: 177 (CD3W245 Fab-Fc HC2 cDNA); SEQ ID NO:202 (CD3W245 Fab-Fc LC2 cDNA); SEQ ID NO:256 (CD31B376 Fab-Fc HC2 cDNA); SEQ ID NO:264 (CD31B376 Fab-Fc LC2 cDNA); SEQ ID NO:237 (CD31B376 Fab-Fc HC2 KIH cDNA); and SEQ ID NO:238 (CD31B376 Fab-Fc LC KIH cDNA).

### Example 9. Characterization of Bispecific DLL3×CD3 Antibodies

### Binding Affinity of Bispecific Anti-DLL3×CD3 Antibodies to DLL3

The binding affinity of anti-DLL3×CD3 antibodies to the recombinant human DLL3 was determined by surface plasmon resonance (SPR) using a Biacore T200 instrument. The antibodies were captured on a goat anti-Fc antibody-modified C1 chip and titrated with 3-fold serial dilutions of DLL3 antigen spanning concentrations of 90 nM to 1.1 nM. The association was monitored for 2 minutes and the and dissociation for 5 or 60 minutes, using a flow rate of 100 μL/min. Raw binding data was referenced by subtracting the analyte binding signals from blanks and analyzed using a 1:1 Langmuir binding model using the Biacore Insight evaluation software to obtain the kinetics which were used to calculate the binding affinity. Binding affinities of anti-DLL3×CD3 antibodies to the recombinant human DLL3 are summarized in Table 36.

TABLE 36
Affinities (K_{D}) for the interaction of anti-DLL3 ×
CD3 bispecific antibodies with human DLL3.

| Name | Description | k_{D} (pM) |
|---|---|---|
| DL3B582 | CD3W245-LH-scFv; DL3B279-Fab | 16 |
| DL3B583 | CD3W245-HL-scFv; DL3B279-Fab | 16 |
| DL3B585 | CD3B376-Fab; DL3B279-LH-scFv | 24 |
| DL3B587 | CD3W245-Fab; DL3B279-LH-scFv | 31 |

In order to ensure the N to Q mutation in the HCDR1 region (or near the HCDR1 region depending on the delineation used) of the DL3B279 variant, as described in Example 5, did not result in change in binding to DLL3, the binding affinity of the DL3B279 variant to the recombinant human DLL3 was determined by surface plasmon resonance (SPR) using a Biacore T200 instrument as described above and compared to the parental DL3B279. The results (Table 37) showed that the binding affinity of the DLL3×CD3 bispecific (D3C3B80) containing the DL3B279 variant (DL3B279-VL-A99G-VH-N27Q_M105T-LH-scFV) is comparable to that of the original DLL3×CD3 bispecific (DL3B585) containing the original DL3B279-LH-scFV molecule (DL3B585: 24 pM).

TABLE 37
Affinities (K_{D}) for the interaction of bispecific
anti-DLL3 × CD3 antibody with human DLL3.

| Name | Description | k_{D} (pM) |
|---|---|---|
| DL3B585 | CD3B376-Fab; DL3B279-LH-scFv | 24 |
| D3C3B80 | CD3B376-Fab; | 33 |
| | DL3B279-VL-A99G-VH-N27Q_M105T-LH-scFV | |

### Thermal Stability of Bispecific Anti-DLL3×CD3 Antibodies

The thermal stability (conformational stability) bispecific anti-DLL3×CD3 antibodies was determined by NanoDSF method using an automated Prometheus instrument. Measurements were made by loading sample into 24 well capillary from a 384 well sample plate. Duplicate runs were performed. The thermal scans span from 20° C. to 95° C. at a rate of 1.0° C./minute. The data was proceed to obtain integrated data and first derivation analysis for 330 nm, 350 nm, Ratio 330/350, and scatter data from which thermal transitions, onset of unfolding, Tₘ and T_{agg} were obtained.

The results show that the bispecific anti-DLL3×CD3 antibodies have a first transition (Tₘ₁) higher than 59° C. The results also show that most proteins, except DL3B585 have low aggregation potential with T_{agg} above 70° C. and 5 degrees or more higher than Tₘ₁ (Table 38).

TABLE 38
Thermal stability data for bispecific anti-DLL3 ×
CD3 antibodies as obtained using a NanoDSF instrument.

| Name | Description | T_{agg} | Tₘ₁ |
|---|---|---|---|
| DL3B582 | CD3W245-LH-scFv; DL3B279-Fab | 74.7° C. | 63.3° C. |
| DL3B583 | CD3W245-HL-scFv; DL3B279-Fab | 75.4° C. | 63.1° C. |
| DL3B585 | CD3B376-Fab; DL3B279-LH-scFv | 62.7° C. | 60.8° C. |
| DL3B587 | CD3W245-Fab; DL3B279-LH-scFv | 74.6° C. | 62.4° C. |

The thermal stability of the bispecific anti-DLL3 CD3 antibody containing the DL3B279 variant (D3C3B80) was also determined. The results (Table 39) showed that the thermostability of the bispecific DLL3×CD3 antibody with DL3B279-VL-A99G-VH-N27Q_M105T-LH-scFV variant (D3C3B80) is comparable to that in the original bispecific molecule with the original DL3B279-LH-scFv sequence (DL3B585:T_{agg}=62.7, Tₘ₁=60.8 shown in Table 39).

TABLE 39
Thermal stability data for anti-DLL3 antibodies
as obtained using a nanoDSF instrument.

| Name | Description | T_{agg} | Tₘ₁ |
|---|---|---|---|
| DL3B585 | CD3B376-Fab; DL3B279-LH-scFv | 62.7° C. | 60.8° C. |
| D3C3B80 | CD3B376-Fab; DL3B279-VL- | 62.4° C. | 60.9° C. |
| | A99G-VH-N27Q_M105T-LH-scFV | | |

### Binding of Bispecific Anti-DLL3×CD3 Antibodies on DLL3+ Tumor Cells

The cell binding profiles of the anti-DLL3×CD3 antibodies to DLL3+ human tumor cell lines (HCC1833 and SHP-77) was also determined. The adherent SCLC HCC1833 cells were washed with DPBS and 0.25% trypsin was added to allow cells to detach. The media was added to neutralize trypsin and the cells were transferred to a 15 mL conical tube. The suspension SCLC SHP77 cells were transferred to a 15 mL conical tube and were centrifuged 1200 rpm for 3 minutes. The media was aspirated and the cells were washed once more with DPBS. The cells were counted using the Vi-cell XR cell viability analyzer and were plated at 100K/well in 100 uL DPBS. The plate was centrifuged 1200 rpm for 3 minutes and washed 2× with DPBS. The cells were stained with Violet Live/Dead stain (Thermo-Fisher) and incubated at RT in the dark for 25 min. The cells were centrifuged and washed 2× with FACS staining buffer (BD Pharmingen).

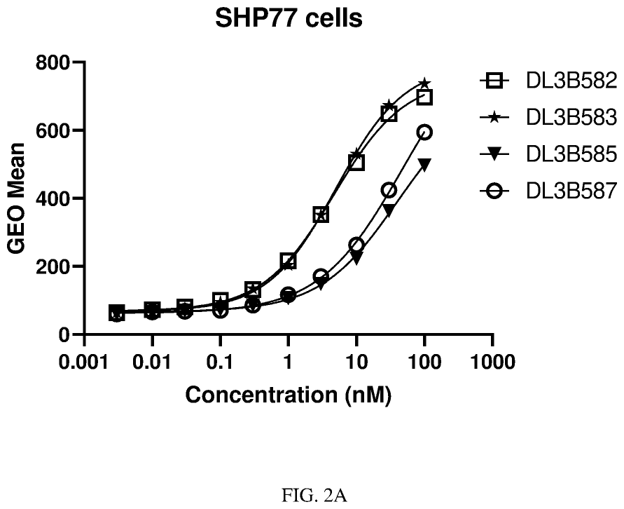

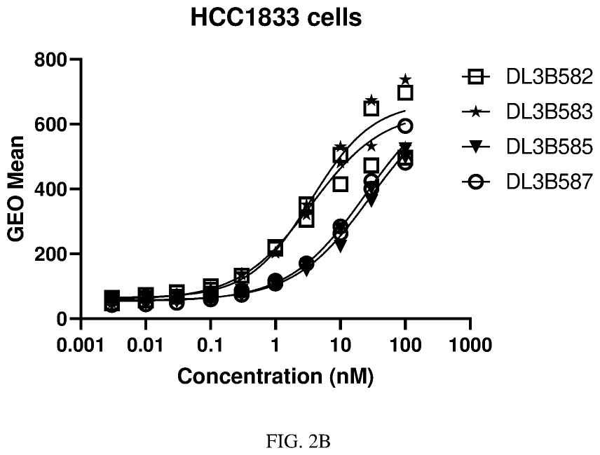

The test antibodies were diluted to a final starting concentration of 100 nM in FACS staining buffer and 3-fold serial dilutions were prepared from the starting concentration for a total of 10 dilution points. The serially diluted test antibodies (100 μL/well) were added to the cells and incubated for 30 min at 37°. The cells were washed 2× with FACS staining buffer and AlexaFluor 647-conjugated Donkey anti-human secondary antibody (Jackson Immunoresearch) was added and allowed to incubate with the cells for 30 min at 4°. Then the cells were washed 2× with FACS staining buffer and re-suspended in 100 uL FACS Buffer. The cells were run on BD Celesta using FACS Diva software and analyzed using FlowJo software. As shown in FIG. 2A and FIG. 2B, the binding profiles between the DLL3-Fab arms (DL3B582 and DL3B583) and DLL3-scFv arms (DL3B585 and DL3B587) are moderately different.

### Binding of Bispecific Anti-DLL3×CD3 Antibodies on Pan T-Cells

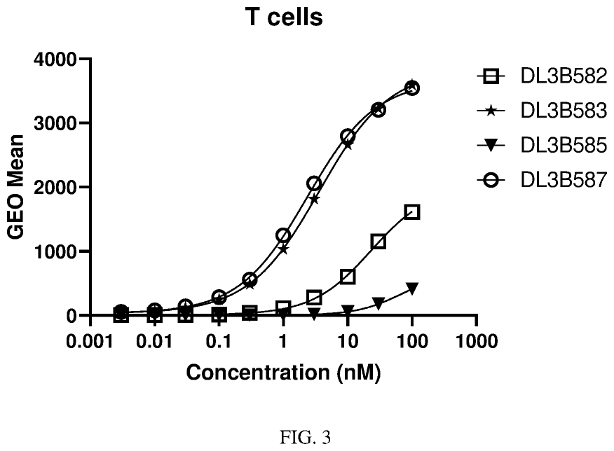

The cell binding profiles of the anti-DLL3×CD3 antibodies to normal human T cells were also evaluated. Human Pan T Cells (Biological Specialty Corporation, Colmar, PA) were thawed and transferred to a 15 mL conical with DPBS (Dulbecco's Phosphate Saline Buffer). The cells were centrifuged 1300 rpm for 5 minutes. DPBS was aspirated and the cells were re-suspended in DPBS. The cells were counted using the Vi-cell XR cell viability analyzer and were plated at 100K/well in 100 μL DPBS. The plate was centrifuged 1200 rpm for 3 minutes and washed 2× with DPBS. The cells were stained with Violet Live/Dead stain (Thermo-Fisher) and incubated at RT in the dark for 25 min. The cells were centrifuged and washed 2× with FACS staining buffer (BD Pharmingen). Test antibodies were diluted to a final starting concentration of 100 nM in FACS staining buffer and 3-fold serial dilutions were prepared from the starting concentration for a total of 10 dilution points. The serially diluted test antibodies (100 uL/well) were added to the cells and incubated for 30 min at 37°. Cells were washed 2× with FACS staining buffer and AlexaFluor 647-conjugated Donkey anti-human secondary antibody (Jackson Immunoresearch) was added and allowed to incubate with the cells for 30 min at 4°. Cells were washed 2× with FACS staining buffer and re-suspended in 100 uL FACS Buffer. Cells were run on BD Celesta using FACS Diva software and analyzed using FlowJo software. As shown in FIG. 3, the cell binding profiles are different across the various CD3 arms.

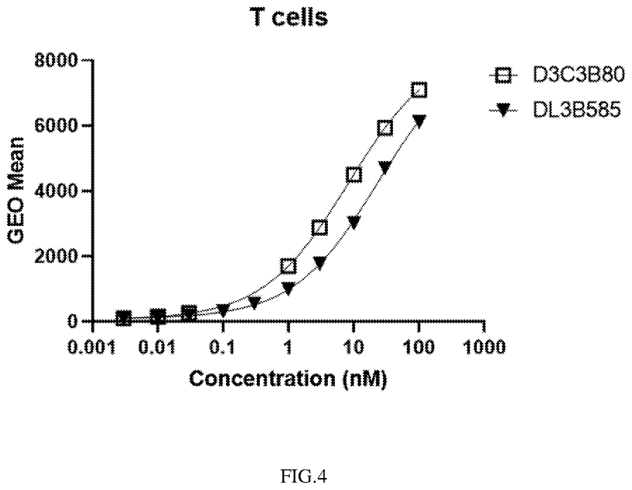

The cell binding profile of the anti-DLL3×CD3 antibody containing DL3B279 variant (DL3B279-VL-A99G-VH-N27Q_M105T-LH-scFV) to normal human T cells was also evaluated and compared to the original DLL3×CD3 bispecific (DL3B585) containing the original DL3B279-LH-scFV molecule. As shown in FIG. 4, the bispecific DLL3×CD3 antibody with DL3B279-VL-A99G-VH-N27Q_M105T-LH-scFV variant (D3C3B80) has comparable binding on T-cells as the original bispecific molecule with the original DL3B279-LH-scFv sequence (DL3B585).

### Bispecific DLL3×CD3 Mediated Cytotoxicity Against DLL3+ Target Cell Lines in Pan T-cells

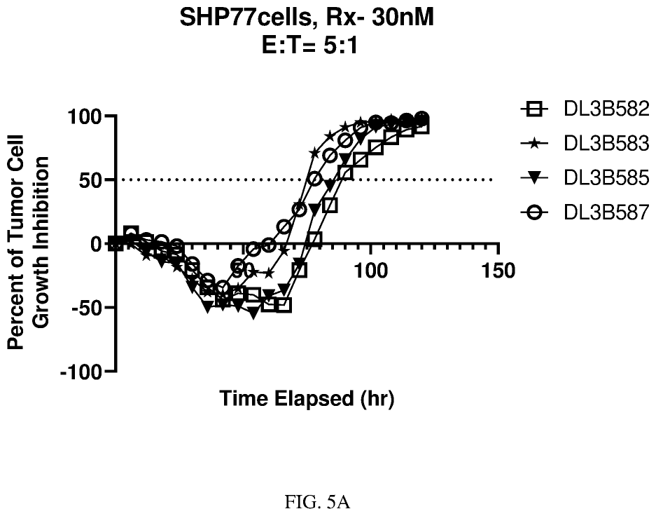

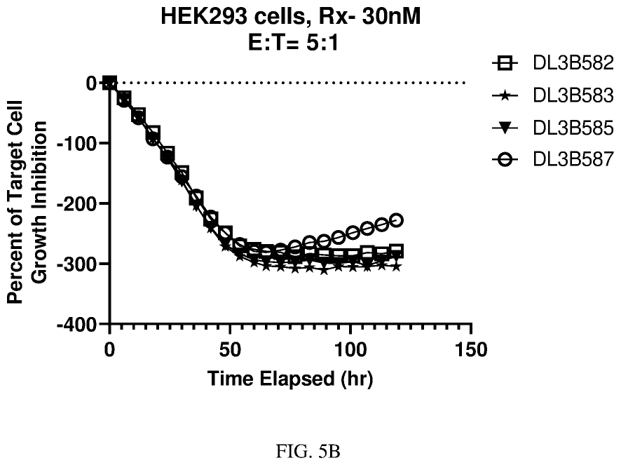

The T-cell mediated killing potential of the bispecific anti-DLL3×CD3 antibodies in DLL3⁺ and DLL3⁻ cell lines was also evaluated. DLL3⁺ SHP77 and DLL3⁻ HEK293 stably expressing red nuclear dye were generated to be used in the IncuCyte-based cytotoxicity assay. Frozen vials of healthy donor T-cells (Biological Specialty Corporation, Colmar, PA) were thawed in a 37° C. water bath, transferred to a 15 mL conical tube, and washed once with 5 mL phenol-red-free RPMI/10% HI FBS medium. The cells were counted using the Viacell XR cell viability analyzer and the T-cells were combined with target cells for a final effector T-cell to target cell (E:T) ratio of 5:1. The cell mixture (100 uL/well) was combined in a 50 mL conical tube and added to a clear 96-well flat-bottom plate. The test antibodies were then diluted to a final starting concentration of 60 nM in phenol-red-free RPMI/10% HI FBS medium and 3-fold serial dilutions were prepared from the starting concentration for a total of 11 dilution points. The serially diluted test antibodies (100 uL/well) were added to the combined cells. The plates were placed in either an IncuCyte© Zoom or an IncuCyte S3© (Essen) at 37° C. with 5% CO₂ for 120 hours. The target cell lines stably express red nuclear dye which was used to track the kinetics of target cell lysis. Percent cell growth inhibition (%)=(Initial viable target cell number−Current viable target cell number)/Initial viable cell number*100%. As shown in FIG. 5A and FIG. 5B, the T cell cytotoxicity assay results demonstrate that all bispecific anti-DLL3×CD3 antibodies are capable of achieving >95% tumor lysis by 5 days.

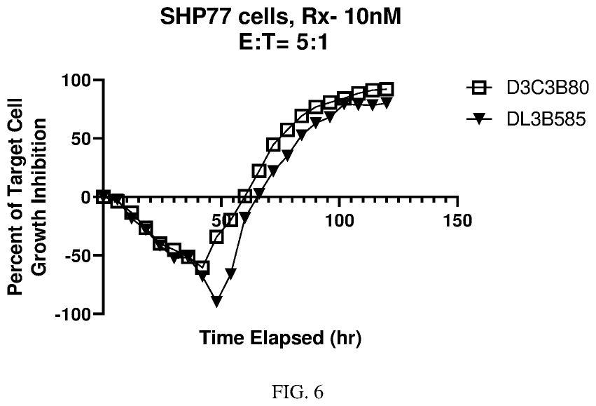

The T-cell mediated killing potential of the anti-DLL3×CD3 antibody containing DL3B279 variant (DL3B279-VL-A99G-VH-N27Q_M105T-LH-scFV) was also evaluated and compared to the original DLL3×CD3 bispecific (DL3B585) containing the original DL3B279-LH-scFV molecule. As shown in FIG. 6, the bispecific DLL3×CD3 antibody with DL3B279-VL-A99G-VH-N27Q_M105T-LH-scFV variant (D3C3B80) has comparable cell growth inhibition as the original bispecific molecule with the original DL3B279-LH-scFv sequence (DL3B585).

### Cytokine Induction Mediated by Bispecific DLL3×CD3 Antibodies in Pan T-Cells

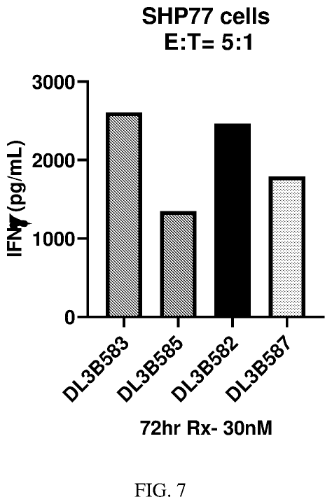

The cytokine release profiles of the bispecific anti-DLL3×CD3 antibodies was evaluated in a DLL3+ human tumor cell line. The supernatants were analyzed using the Human Proinflammatory Panel I tissue culture kit (Meso Scale Discovery) and were thawed on wet ice, spun at 1,500 rpm for 5 minutes at 4° C., then placed on ice. The MULT-SPOT assay plates were pre-washed per the manufacturer's protocol. A standard curve was prepared by serial dilution of the provided calibrators in MSD Diluent 1. The standards and test antibody samples (25 uL/well) were added to the pre-washed plates. Assay plates were read on the SECTOR Imager 6000. As shown in FIG. 7, the results of the cytokine profiling experiment demonstrate that IFN-gamma production correlates with the CD3 affinity of the bispecific anti-DLL3 xCD3 antibodies.

### Bispecific DLL3×CD3 Mediated Cytotoxicity Against DLL3+ Target Cell Lines in PBMCs

In order to test the efficacy of the bispecifics against DLL3+ target cells with varying levels of antigen expression, DLL3 high expression (SHP-77 and HCC1833) and DLL3 low expression cell line (G361) were tested in the cytotoxicity assay. SHP-77 and HCC1833 are lung epithelial and lung adenocarcinoma cell lines, respectively. G361 cells are derived from malignant skin melanoma. DLL3+ SHP-77 cell line stably expressing the nuclear restricted NucLight Red (NLR) protein was used in the cytotoxicity assay. On the day of the assay, SHP-77-NLR cells were collected into a 50 ml falcon tube and spun down at 1300 rpm for 5 min. The cell pellet was then resuspended in modified RPMI 1640 media+10% FBS (complete media) and cell count was estimated using trypan blue live dead marker using a hemocytometer. SHP-77-NLR cells were then plated onto a collagen coated 96 well plate at 10,000 cells/well/90 μl of complete media. The cells were evenly distributed by gentle agitation and allowed to settle for 1 hour in a 5% CO₂ incubator. In the case of HCC1833 and G361 target cell lines, 3000 cells/well/90 μl complete media were plated in a 96 well flat bottom tissue culture plates one day prior to the PBMC addition.

The vials of PBMCs frozen from healthy donors (Clinigene) were rapidly thawed in a 37° C. water bath, transferred to a 15 mL conical tube, and washed once with 10 mL complete medium. The cells were stained with anti-human CD3 antibody and analyzed by flow cytometer to determine the CD3% within PBMCs. PBMCs from each donor were counted using trypan blue live dead marker using a hemocytometer and the number of PBMCs required to get required effector to target (ET) ratios (CD3:target cell) were added to the plated target cells in 90 μl complete media. The test antibodies were then prepared as 10× stocks in complete media and 3-fold serial dilutions were prepared. The serially diluted test antibodies were added to the PBMC-tumor coculture at 20 μl/well so that the final concentration of antibody became 1×. Wells with no antibody (NBS) were used as control for the basal cytotoxicity. The plates were placed in an IncuCyte S3© (Essen BioScience) at 37° C. with 5% CO₂ for 5 days. Increase in red signal corresponds to target cell proliferation and a decrease in signal corresponds to target cell death. Results are summarized in Table 40. % lysis was calculated as ={100−(red signal intensity at a specific time point with Antibody/red signal intensity at that time point in NBS wells)*100}.

TABLE 40
% lysis of SHP-77, HCC1833 and G361 cells on day 5 after
coculture with whole PBMCs and bispecific anti-DLL3 ×
CD3 antibodies at the indicated concentrations using a
1:1 ET ratio (CD3:target cells). NA indicates not tested.

| | | Cytotoxicity (% Lysis at | | |
|---|---|---|---|---|
| | | Day 6, 1:1 ET ratio) | | |
| Molecules | | SHP-77 | G361 | HCC1833 |
| Name | Description | 30 nM | 30 nM | 30 nM |
| DL3B582 | CD3W245-LH-scFv; | 87.3 | 98.9 | 93.7 |
| | DL3B279-Fab | | | |
| DL3B583 | CD3W245-HL-scFv; | 99.8 | 98.9 | 88.4 |
| | DL3B279-Fab | | | |
| DL3B585 | CD3B376-Fab; | 58.1 | NA | NA |
| | DL3B279-LH-scFv | | | |
| DL3B587 | CD3W245-Fab; | 83.3 | NA | NA |
| | DL3B279-LH-ScFv | | | |

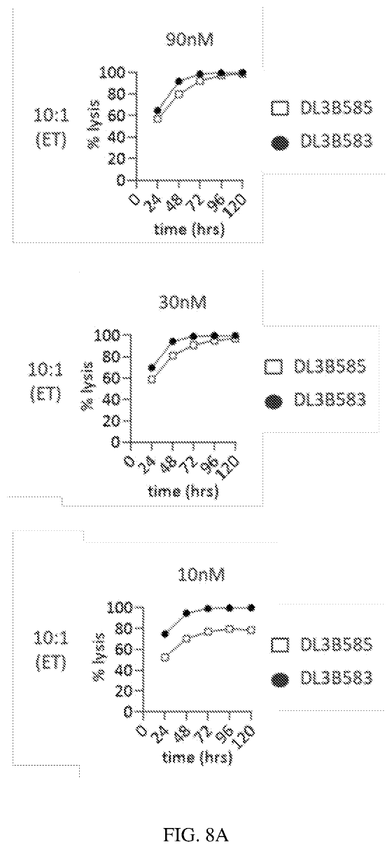

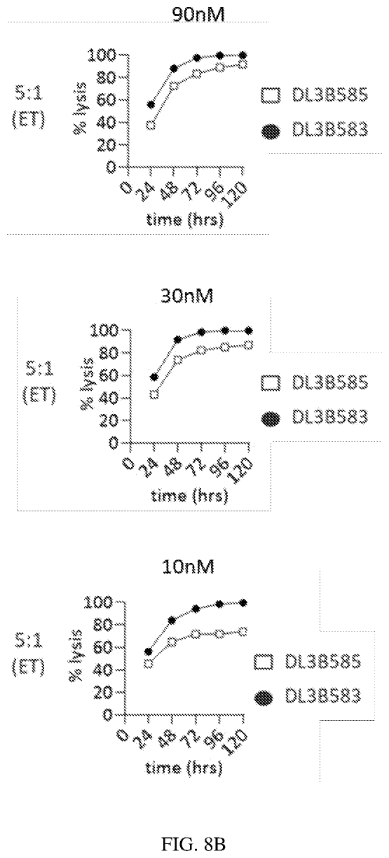

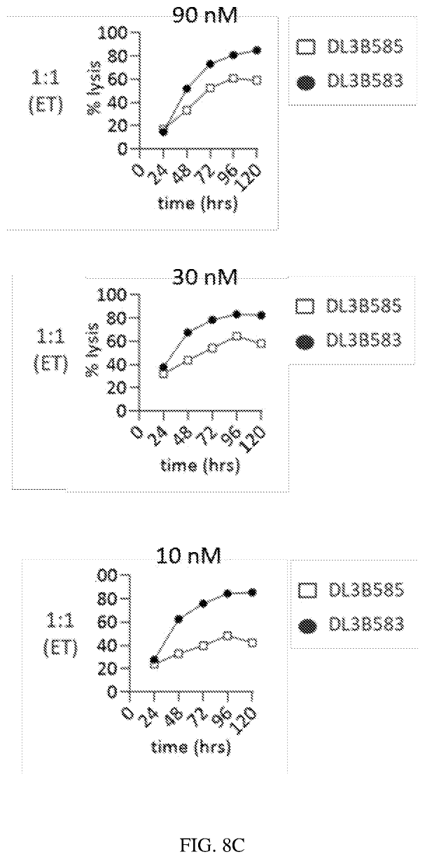

Potent tumor cell lysis was observed with bispecifics DLL3×CD3 antibodies across cell lines of different origin and antigen densities. To compare the efficacy of the high affinity CD3 bispecific (DL3B583) with the low affinity CD3 bispecific (DL3B585), the cytotoxicity against DLL3 high expression SHP-77 cells was tested at various ET ratios. The whole PBMCs from 3 donors were cultured with DLL3+ SHP-77-NLR cells at the indicated ET ratios (CD3:SHP-77) in the presence of the bispecific DLL3×CD3 antibodies. Wells with PBMCs and target cells but no antibody were used as control for basal cytotoxicity. Plates were scanned for up to 120 hours in an IncuCyte S3© (Essen BioScience) in a 37° C. with 5% CO2 incubator. % lysis was calculated as ={100−(red signal intensity at a specific time point with Antibody/red signal intensity at that time point in NBS wells)*100}. Each point on the graph represents an average of 3 donors. As shown in FIG. 8A, FIG. 8B and FIG. 8C, bispecific DLL3×CD3 antibodies with both the high affinity CD3 (DL3B583) and low affinity CD3 (DL3B585) arms showed robust cytotoxicity against SHP-77 cells. Target cell lysis at 90 nM and 30 nM antibody concentration was similar between the high and low affinity CD3 antibody for 10:1 ET ratio.

### Proliferation of CD3+ T Cells in Response to Bispecific DLL3×CD3 Antibodies in Whole PBMC Cytotoxicity Assay

In order to test if the binding of bispecific DLL3×CD3 antibodies to CD8⁺ T cells can induce proliferation and expansion of CD8⁺ T cells, the time course analysis of CD8⁺ T cell proliferation was performed. DLL3+ SHP-77 cells were used for the assay. On the day of the assay, SHP-77 cells were collected into a 50 ml falcon tube and spun down at 1300 rpm for 5 min. The cell pellet was then resuspended in 1 ml modified RPMI 1640 media+10% FBS (complete media) and cell count was estimated using trypan blue live dead marker using a hemocytometer. SHP77 cells were then plated in a U-bottom 96 well plate at 10,000 cells/well/90 μl of complete media.

The vials of PBMCs frozen from healthy donors (Clinigene) were rapidly thawed in a 37° C. water bath, transferred to a 15 mL conical tube, and washed once with 10 mL complete medium. The cells were stained with anti-human CD3 antibody and analyzed by flow cytometer to determine the CD3% within PBMCs. PBMCs were stained Cell Trace Violet dye (C34571, Thermo Fisher Scientific). PBMCs from each donor were counted using trypan blue live dead marker using a hemocytometer and the number of PBMCs required to get effector to target (ET) ratio of 10:1 (CD3:target cell) were added to the plated target cells in 90 μl complete media.

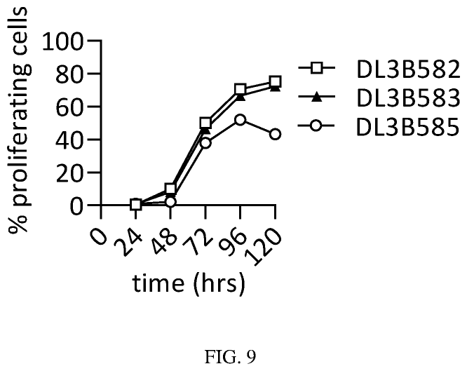

The test antibodies were then prepared as 10× stocks in complete media and 3-fold serial dilutions were prepared from the starting concentration for a total of 3 dilution points. The serially diluted test antibodies were added to the PBMC-tumor coculture at 20 μl/well so that the final concentration of antibody became 1×. Wells with no antibody (NBS) were used as control for the basal cytotoxicity. The plates were incubated in a 5% CO₂ incubator for the indicated time periods. At the end of the incubation period, the cells suspension was transferred to a v-bottom plate and was spun down at 1500 rpm for 5 min. The pellet was resuspended in 100 μl of DPBS. 10 μl of the cell suspension was taken for determining the total cell count at each antibody concentration using Trypan blue with a hemocytometer. The rest of the cell suspension was subjected to LIVE/DEAD™ Fixable Near-IR Dead Cell Stain Kit (L10119) and incubated for 20 min on ice. The viability stain was inactivated using FACS buffer and was spun down at 1500 rpm for 5 min. Cells were stained with BD Fc block (564220, BD Pharmingen) for 10 min followed by staining with CD3 and CD8 antibodies and acquired on a flow cytometer. Gating on CD8 T cells was performed to estimate the expansion of the cytotoxic CD8 T cell population within the CD3 T cells. As shown in FIG. 9, binding of the bispecific DLL3×CD3 antibodies to T cells potently mediates the expansion of cytotoxic CD8 T cells.

### Activation Profile of CD8 T Cells by Bispecific DLL3×CD3 Antibodies in Whole PBMC Assay

In order to assess the activation status of the cytotoxic CD8 T cell population in response to the binding of the DLL3×CD3 bispecifics, kinetic analysis of CD25, CD69 and CD71 markers was performed. DLL3+ SHP-77 cells were used for the assay. SHP-77 cells were collected into a 50 ml falcon tube and spun down at 1300 rpm for 5 min. The cell pellet was then resuspended in 1 ml modified RPMI 1640 media+10% FBS (complete media) and cell count was estimated using trypan blue live dead marker using a hemocytometer. SHP-77 cells were then plated in a U-bottom 96 well plate at 10,000 cells/well/90 μl of complete media.

Vials of PBMCs frozen from healthy donors (Clinigene) were rapidly thawed in a 37° C. water bath, transferred to a 15 mL conical tube, and washed once with 10 mL complete medium. The cells were stained with anti-human CD3 antibody and analyzed by flow cytometer to determine the CD3% within PBMCs. PBMCs from each donor were counted using trypan blue live dead marker using a hemocytometer and the number of PBMCs required to get effector to target (ET) ratio of 10:1 (CD3:target cell) were added to the plated target cells in 90 μl complete media.

The test antibodies were prepared as 10× stocks in complete media and 3-fold serial dilutions were prepared from the starting concentration for a total of 3 dilution points. The serially diluted test antibodies were added to the PBMC-tumor coculture at 20 μl/well so that the final concentration of antibody became 1×. Wells with no antibody (NBS) were used as control for the basal cytotoxicity. The plates were incubated in a 5% CO2 incubator for the indicated time periods. At the end of the incubation period the cells suspension was transferred to a v-bottom plate and was spun down at 1500 rpm for 5 min. The pellet was resuspended in 100 μl of DPBS. 10 μl of the cell suspension was taken for determining the total cell count at each antibody concentration using Trypan blue with a hemocytometer.

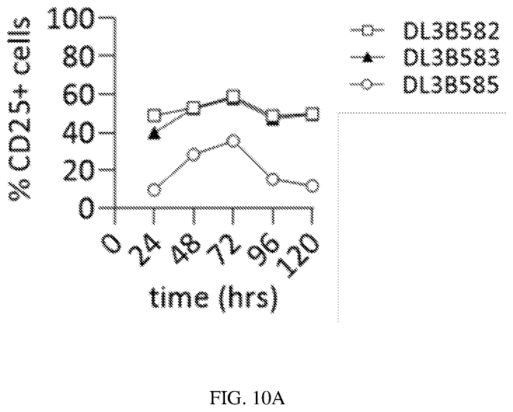

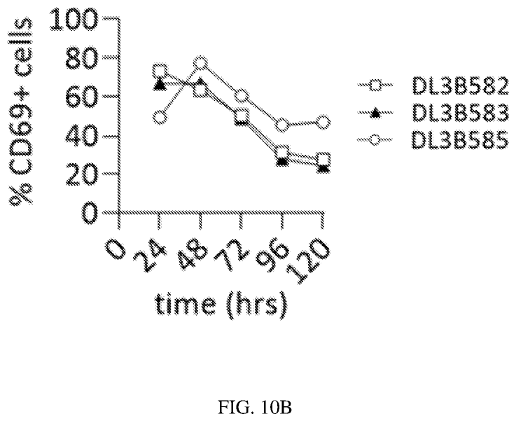

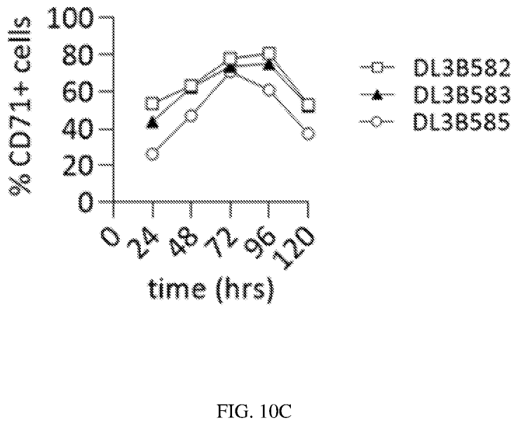

The rest of the cell suspension was subjected to LIVE/DEAD™ Fixable Near-IR Dead Cell Stain Kit (L10119) and incubated for 20 min on ice. The viability stain was inactivated using FACS buffer and was spun down at 1500 rpm for 5 min. The cells were stained with BD Fc block (564220, BD Pharmingen) for 10 min followed by staining with CD3, CD8, CD25, CD69 and CD71 antibodies and acquired on a flow cytometer. As shown in FIG. 10A, FIG. 10B and FIG. 10C, potent activation of cytotoxic CD8 T cells was seen with the bispecific DLL3×CD3 antibodies as indicated by the upregulation of CD25, CD69 and CD71 expression on the surface of CD8 T cells.

DLL3×CD3 Bispecific Antibody Induced Activity on T Cells in Co-Culture with DLL3+ Cells

The cytotoxic effect of DLL3×CD3 bispecific antibodies were tested on SHP-77, a small cell lung cancer cell line expressing DLL3 in replicate experiments on three PAN-T donors at a 5:1 Effector to Target ratio (E:T). On day 0 of the experiment, assay plates were seeded with 20,000 SHP-77 (50 uL out of 0.4e6 cells/mL) cells per well, 50 uL of growth media and, 100,000 PAN CD3+ T cells (50 uL of 2e6 cell/mL). T cells are stained with Encoder Dye B/Green (Sartorius) beforehand for measurement of proliferation, and 50 uL of the appropriate diluted DLL3×CD3 bispecific antibody is added to the appropriate wells in duplicate. DLL3×null was used as a negative control. Final antibody concentrations were 100 nM, 33.3 nM, 11.1 nM, 3.70 nM, 1.23 nM, 0.41 nM, 0.14 nM, 0.046 nM, 0.015 nM, and 0 nM. Plates were then incubated for 48, 72, and 120 hours. After each subsequent timepoint, supernatants (for cytokine enumeration) and T cells (for proliferation and activation) were harvested. The supernatants containing the cytokines were analyzed for IFNy CD3, CD8, CD25, and the T cells were analyzed for proliferation using the T Cell Activation Cell and Cytokine Profiling Kit (Sartorius Catalog #90561).

IFNy cytokine was measured at 48 and 120 hours using a prepared standard from the T Cell Activation Cell and Cytokine Profiling Kit from Sartorius. The results from the three separate PAN CD3+T donor experiments (duplicate wells) were averaged for a n=6. At 48 and 120 hours, IFNy was observed to be released at higher concentrations for DLL3×CD3 compared to DLL3× null in a dose dependent manner (FIGS. 11A and 11B).

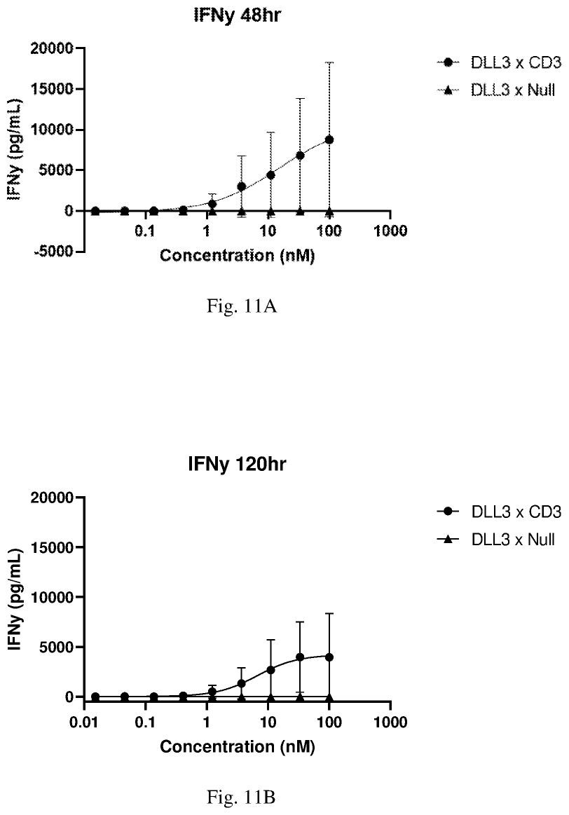

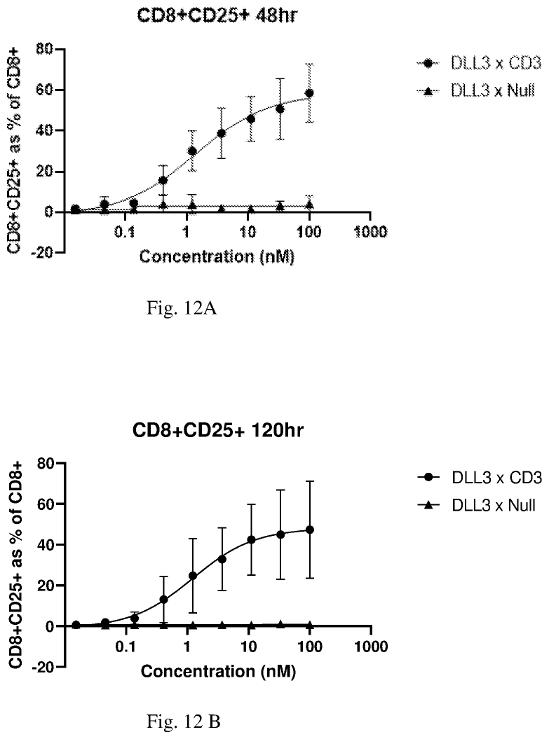

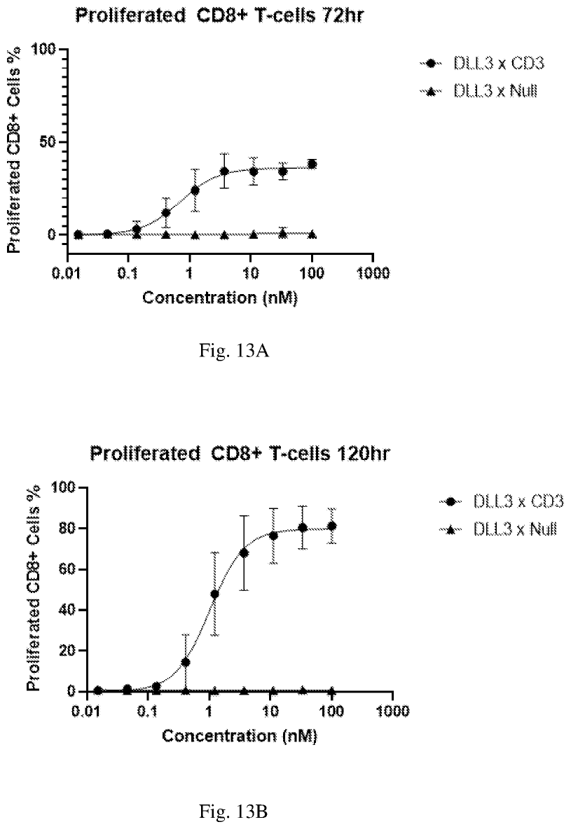

T-cell activation was measured by gating CD8⁺ and the CD25⁺ marker within the CD3+ population. The results of the three PAN-T donor experimental results (in duplicate) were averaged for a n=6. Compared to the DLL3× null, DLL3×CD3 induced greater activation of the CD8⁺ CD25⁺ T-cells at both 48 and 120 hours in a dose dependent manner (FIGS. 12A and 12B). Proliferation of CD8⁺ T-cells was measured at 72 and 120 hours. From 72 to 120 hours, DLL3×CD3 proliferation of CD8⁺ T-cells increased in a dose dependent manner compared to the DLL3× null (FIG. 13A and FIG. 13B).

### Cytokine Induction Mediated by Bispecific DLL3×CD3 Antibodies in Whole PBMC Assay

T cell redirecting bispecific antibodies can cause toxicity because of the induction of cytokine release syndrome. These cytokines can be produced by T cell themselves or myeloid cells and results in a feedback loop of more cytokine production. In order to understand the release of cytokines such as IL-6, TNF-a, IL-10, GMCSF and other T cell cytokines by the addition of DLL3×CD3 bispecifics, culture supernatants from cytotoxicity assays were tested for the levels of these cytokines. DLL3+ SHP-77 cells were used for the assay. SHP-77 cells were collected into a 50 ml falcon tube and spun down at 1300 rpm for 5 min. The cell pellet was then resuspended in 1 ml modified RPMI 1640 media+10% FBS (complete media) and the cell count was estimated using trypan blue live dead marker using a hemocytometer. SHP-77 cells were then plated in a U-bottom 96 well plate at 10,000 cells/well/90 μl of complete media.

The vials of PBMCs frozen from healthy donors (Clinigene) were rapidly thawed in a 37° C. water bath, transferred to a 15 mL conical tube, and washed once with 10 mL complete medium. The cells were stained with anti-human CD3 antibody and analyzed by flow cytometer to determine the CD3% within PBMCs. PBMCs from each donor were counted using trypan blue live dead marker using a hemocytometer and the number of PBMCs required to get effector to target (ET) ratio of 10:1 (CD3:target cell) were added to the plated target cells in 90 μl complete media.

The test antibodies were prepared as 10× stocks in complete media and added to the PBMC-tumor coculture at 20 μl/well so that the final concentration of antibody became 1×. Wells with no antibody (NBS) were used as control for the basal cytotoxicity. The plates were incubated in a 5% CO2 incubator for the indicated time periods. At the end of the incubation period the cells suspension was transferred to a v-bottom plate and was spun down at 1500 rpm for 5 min. The supernatant was collected and stored at −20° C. to perform Luminex using the MILLIPLEX MAP Human CD8⁺ T Cell Magnetic Bead Panel (HCD8MAG-15K, Millipore). Plate was analyzed using MAGPIX with eXPONENT software. Results are summarized in Table 41.

TABLE 41
Cytokine release mediated by bispecific DLL3 × CD3 antibodies
in whole PBMC cytotoxicity assay: Whole PBMCs from 3 donors were
cultured with DLL3⁺ SHP-77 cells at a 10:1 ET ratio
(CD3:SHP-77) in the presence of the CD3 × DLL3 antibodies
at 30 nM concentration for DL3B582 and DL3B583 and 90 nM
for DL3B585. Supernatant was collected at indicated time
points and analyzed for cytokine release using Luminex.
Each point on the graph is an average of 3 donors.

| Cytokines | Bispecific DLL3 × CD3 antibodies | | |
|---|---|---|---|
| (ng/ml) | DL3B582 | DL3B583 | DL3B585 |
| TNFα | 2.7 | 2.0 | 1.1 |
| GMCSF | 0.8 | 1.0 | 0.5 |
| IL-10 | 13.5 | 20.7 | 1.9 |
| IL-13 | 0.5 | 0.4 | 0.5 |
| Gzm B | 9.7 | 9.6 | 1.0 |
| IL-2 | 1.0 | 0.8 | 0.0 |
| IL-4 | 0.2 | 0.2 | 0.0 |
| IL-5 | 0.1 | 0.1 | 0.1 |
| IL-6 | 4.2 | 4.8 | 0.9 |

Low levels of cytokine release was observed with the bispecific DLL3×CD3 antibody with lower affinity CD3 (DL3B585) as compared to the ones with higher affinity CD3 arms (DL3B582 and DL3B583), in particular, IL-10, IL-6, IL-2 and IL-4, while the cytotoxic potency of these bispecific DLL3×CD3 was comparable.

The present examples demonstrate that the isolated multispecific antigen-binding constructs disclosed herein are particularly effective at mediating T cell mediated cytotoxicity, promoting T cell activation and proliferation, increasing T cell cytokine release and/or displaying increased anti-tumor efficacy. These activities are a reflection of the combination of antigen binding regions targeting DLL3 on the target cell and CD3 on the T cell. The skilled person would understand that such activity would be expected from assembling the binding domains into a bispecific antibody, irrespective of the mechanism by which the bispecific antibody is assembled.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1-2. (canceled)

3. An isolated protein comprising an antigen binding region that binds delta-like protein 3 (DLL3), wherein the antigen binding region comprises a heavy chain variable region complementarity determining region 1 (HCDR1), HCDR2, and HCDR3, and a light chain variable region complementarity determining region 1 (LCDR1), LCDR2, and, LCDR3, comprising the amino acid sequences of:
a. SEQ ID NOs:_15, 16, 17, 33, 34, and 35, respectively;
b. SEQ ID NOs:_18, 19, 20, 36, 37, and 38, respectively;
c. SEQ ID NOs:_21, 22, 23, 39, 37, and 40, respectively;
d. SEQ ID NOs:_24, 25, 26, 41, 42, and 43, respectively;
e. SEQ ID NOs:_18, 28, 29, 44, 45, and 46, respectively;
f. SEQ ID NOs:_30, 31, 32, 47, 48, and 49, respectively;
g. SEQ ID NOs:_50, 51, 17, 33, 34, and 35, respectively;
h. SEQ ID NOs:_52, 51, 17, 33, 34, and 35, respectively;
i. SEQ ID NOs:_53, 54, 20, 36, 37, and 38, respectively;
j. SEQ ID NOs:_55, 56, 23, 39, 37, and 40, respectively;
k. SEQ ID NOs:_57, 58, 26, 41, 42, and 43, respectively;
l. SEQ ID NOs:_59, 60, 29, 44, 45, and 46, respectively; or
m. SEQ ID NOs: 61, 62, 32, 47, 48, and 49, respectively.

4. The isolated protein of claim 3, wherein the antigen binding region comprises a heavy chain variable region (VH) comprising the amino acid sequence of SEQ ID NO: 1, 3, 5, 7, 9, 11, or 13, and a light chain variable region (VL) comprising the amino acid sequence of SEQ ID NO: 2, 4, 6, 8, 10, 12, or 14.

5. The isolated protein of claim 4, wherein the antigen binding region comprises:
a. a VH comprising the amino acid sequence of SEQ ID NO:_1 and a VL comprising the amino acid sequence of SEQ ID NO:_2;
b. a VH comprising the amino acid sequence of SEQ ID NO:_3 and a VL comprising the amino acid sequence of SEQ ID NO:_4;
c. a VH comprising the amino acid sequence of SEQ ID NO:_5 and a VL comprising the amino acid sequence of SEQ ID NO:_6;
d. a VH comprising the amino acid sequence of SEQ ID NO:_7 and a VL comprising the amino acid sequence of SEQ ID NO:_8;
e. a VH comprising the amino acid sequence of SEQ ID NO:_9 and a VL comprising the amino acid sequence of SEQ ID NO:_10;
f. a VH comprising the amino acid sequence of SEQ ID NO:_11 and a VL comprising the amino acid sequence of SEQ ID NO:_12; or
g. a VH comprising the amino acid sequence of SEQ ID NO:_13 and a VL comprising the amino acid sequence of SEQ ID NO:_14.

6. The isolated protein of claim 5, wherein the antigen binding region comprises an scFv comprising the amino acid sequence of SEQ ID NO:_63 or 64.

7. An immunoconjugate comprising the isolated protein of claim 3 conjugated to a therapeutic agent or an imaging agent.

8. A multispecific antigen-binding construct comprising the protein of claim 3.

9. The multispecific antigen-binding construct of claim 8, further comprising a second antigen binding region that binds an antigen on a lymphocyte.

10. The multispecific antigen-binding construct of claim 9, wherein the antigen on the lymphocyte is CD3, CD3 epsilon (CD3ε), CD8, K12L4, NKG2E, NKG2D, NKG2F, BTNL3, CD186, BTNL8, PD-1, CD195, or NKG2C.

11. The multispecific antigen-binding construct of claim 10, wherein the second antigen binding region binds CD3$ and comprises an HCDR1, an HCDR2, an HCDR3, an LCDR1, an LCDR2, and an LCDR3, comprising the amino acid sequences of:
a. SEQ ID NOs: 98, 99, 100, 106, 107, and 108, respectively; or
b. SEQ ID NOs: 95, 96, 97, 101, 102, and 104, respectively.

12. The multispecific antigen-binding construct of claim 11, wherein the second antigen binding region comprises:
a. a VH comprising the amino acid sequence of SEQ ID NO:_84, and a VL comprising the amino acid sequence of SEQ ID NO:_85; or
b. a VH comprising the amino acid sequence of SEQ ID NO:_77, and a VL comprising the amino acid sequence of SEQ ID NO:_80.

13. The multispecific antigen-binding construct of claim 8, wherein the antigen binding domain that binds DLL3 comprises an HCDR1, an HCDR2, an HCDR3, an LCDR1, an LCDR2, and an LCDR3 comprising the amino acid sequences of SEQ ID NOs:_15, 16, 17, 33, 34, and 35, respectively.

14. The isolated protein of claim 1, further comprising an immunoglobulin (Ig), a fragment of the Ig, an Ig constant region, a fragment of the Ig constant region, an Fc region, transferrin, albumin, an albumin binding domain, or polyethylene glycol.

15. The isolated protein of claim 14, further comprising:
a fragment of the Ig constant region; or
an Ig constant region comprising at least one mutation selected from T350V, L351Y, F405A, Y407V, T366Y, T366W, F405W, T394W, T394 S, Y407T, Y407A, T366 S/L368A/Y407V, L351Y/F405A/Y407V, T3661/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V, T350V/T366L/K392L/T394W, and L234A/L235A/D265 S, wherein residue numbering is according to the EU index.

16. A bispecific antigen-binding construct comprising:
(1) A first antigen binding region that binds DLL3, wherein the first antigen binding region comprises an HCDR1, an HCDR2, aid an HCDR3, an LCDR1, an LCDR2, and an LCDR3 comprising the amino acid sequences of:
(a) SEQ ID NOs:_15, 16, 17, 33, 34, and 35, respectively;
(b) SEQ ID NOs:_18, 19, 20, 36, 37, and 38, respectively;
(c) SEQ ID NOs:_21, 22, 23, 39, 37, and 40, respectively;
(d) SEQ ID NOs:_24, 25, 26, 41, 42, and 43, respectively;
(e) SEQ ID NOs:_18, 28, 29, 44, 45, and 46, respectively;
(f) SEQ ID NOs:_30, 31, 32, 47, 48, and 49, respectively;
(g) SEQ ID NOs:_50, 51, 17, 33, 34, and 35, respectively;
(h) SEQ ID NOs:_52, 51, 17, 33, 34, and 35, respectively;
(i) SEQ ID NOs: 53, 54, 20, 36, 37, and 38, respectively;
(j) SEQ ID NOs:_55, 56, 23, 39, 37, and 40, respectively;
(k) SEQ ID NOs: 57, 58, 26, 41, 42, and 43, respectively;
(l) SEQ ID NOs:_59, 60, 29, 44, 45, and 46, respectively; or
(m) SEQ ID NOs:_61, 62, 32, 47, 48, and 49, respectively; and
(2) a second antigen binding region that binds CD38, wherein the second antigen binding region comprises:
(a) an HCDR1, an HCDR2, and an HCDR3 comprising the amino acid sequences of SEQ ID NOs: 95, 96, and 97, respectively, and an LCDR1, an LCDR2, and an LCDR3 comprising the amino acid sequences of SEQ ID NOs: 101, 102, and 104, respectively; or
(b) an HCDR1, an HCDR2, and an HCDR3 comprising the amino acid sequences of SEQ ID NOs: 98, 99, and 100, respectively, and an LCDR1, an LCDR2 and, an LCDR3 comprising the amino acid sequences of SEQ ID NOs: 106, 107 and 108, respectively.

17. The bispecific antigen-binding construct of claim 16, wherein
a. the first antigen binding region comprises a VH and a VL comprising the amino acid sequences of:
i. SEQ ID NO:_1 and SEQ ID NO:_2, respectively;
ii. SEQ ID NO:_3 and SEQ ID NO:_4, respectively;
iii. SEQ ID NO:_5 and SEQ ID NO:_6, respectively;
iv. SEQ ID NO:_7 and SEQ ID NO:_8, respectively;
v. SEQ ID NO:_9 and SEQ ID NO:_10, respectively;
vi. SEQ ID NO:_11 and SEQ ID NO:_12, respectively; or
vii. SEQ ID NO:_13 and SEQ ID NO:_14, respectively; and
b. the second antigen binding region comprises a VH and a VL comprising the amino acid sequences of:
i. SEQ ID NO:_77 and SEQ ID NO:_80, respectively; or
ii. SEQ ID NO:_84 and SEQ ID NO:_85, respectively.

18. The bispecific antigen-binding construct of claim 16, wherein the first antigen binding region comprises an HCDR1, an HCDR2, an HCDR3, an LCDR1, an LCDR2, and an LCDR3 comprising the amino acid sequences of SEQ ID NOs:_15, 16, 17, 33, 34, and 35, respectively.

19. The bispecific antigen-binding construct of claim 17, wherein the first antigen binding region comprises a VH comprising the amino acid sequence of SEQ ID NO:_3; and a VL comprising the amino acid sequence of SEQ ID NO:_4.

20. The bispecific antigen-binding construct of claim 16, wherein
the first antigen binding region comprises a first scFv or a first Fab comprising a VH and a VL; and
the second antigen binding region comprises a second Fab or a second scFv comprising a VH and a VL.

21. The bispecific antigen-binding construct of claim 20, wherein the first antigen binding region comprises the first scFv and the second antigen binding region comprises the second Fab.

22. The bispecific antigen-binding construct of claim 16, wherein the first antigen binding region and the second antigen binding region further comprise an immunoglobulin (Ig) constant region comprising one or more heterodimeric mutations.

23. The bispecific antigen-binding construct of claim 22, comprising:
(1) a first heavy chain comprising an amino acid sequence selected from SEQ ID NOs: 111, 111, 71, and 229;
(2) a light chain comprising an amino acid sequence selected from SEQ ID NOs: 117, 115, 117, and 117; and
(3) a second heavy chain comprising an amino acid sequence selected from SEQ ID NOs: 116, 114, 118, and 230.

24. An isolated nucleic acid encoding the isolated protein of claim 3.

25. A vector comprising the nucleic acid of claim 24.

26. A host cell comprising the nucleic acid of claim 24.

27. A method of producing the isolated protein of claim 3, the method comprising culturing a host cell that comprises a nucleic acid encoding the isolated protein under conditions to produce the isolated protein.

28. A pharmaceutical composition comprising:
the isolated protein of claim 3, an immunoconjugate comprising the isolated protein conjugated to a therapeutic agent or an imaging agent, a nucleic acid encoding the isolated protein, a vector comprising the nucleic acid, a host cell comprising the nucleic acid, or a host cell comprising the vector; and
a pharmaceutically acceptable carrier.

29. A method of treating a DLL3 expressing cancer in a subject in need thereof, the method comprising administering a therapeutically effective amount of the pharmaceutical composition of claim 28 to the subject for a time sufficient to treat the DLL3 expressing cancer.

30. A method of reducing the amount of DLL3 expressing tumor cells in a subject, the method comprising administering a therapeutically effective amount of the pharmaceutical composition of claim 28 to the subject for a time sufficient to reduce the amount of DLL3 expressing tumor cells.

31. A method of treating a noncancerous condition in a subject at risk of developing a DLL3 expressing cancer, the method comprising administering a therapeutically effective amount of the pharmaceutical composition of claim 28 to the subject to treat the noncancerous condition.

32. A method of detecting the presence of neuroendocrine prostate cancer or small cell lung cancer in a subject, the method comprising administering the immunoconjugate of claim 7 to a subject suspected to have prostate cancer or small cell lung cancer and visualizing the biological structures to which the immunoconjugate is bound, thereby detecting the presence of prostate cancer or small cell lung cancer.

33. The multispecific antigen-binding construct of claim 13, wherein the antigen binding domain that binds DLL3 comprises a VH comprising the amino acid sequence of SEQ ID NO: 3 and a VL comprising the amino acid sequence of SEQ ID NO: 4.

34. The method of claim 29, wherein the DLL3 expressing cancer is selected from lung cancer, prostate cancer, glioma, glioblastoma, melanoma, neuroendocrine pancreatic cancer, hepatoblastoma, and hepatocellular carcinoma, or any combination thereof.

35. The method of claim 30, wherein the DLL3 expressing tumor cells are from lung cancer, prostate cancer, glioma, glioblastoma, melanoma, neuroendocrine pancreatic cancer, hepatoblastoma, and hepatocellular carcinoma, or any combination thereof.

36. The method of claim 31, wherein the noncancerous condition is an enlarged prostate, benign prostate hyperplasia (BPH), or a condition with high prostate specific antigen (PSA) levels in the absence of diagnosed prostate cancer.
